(19) United States
(12) Patent Application Publication
(10) Pub. No.: US 2024/0216288 A1
(43) Pub. Date: Jul. 4, 2024

(54) LIPID NANOPARTICLES CONTAINING POLYNUCLEOTIDES ENCODING PROPIONYL-COA CARBOXYLASE ALPHA AND BETA SUBUNITS AND USES THEREOF

(71) Applicant: ModernaTX, Inc., Cambridge, MA (US)

(72) Inventors: Kerry Benenato, Sudbury, MA (US); Mark Cornebise, Arlington, MA (US); Edward Hennessy, Westwood, MA (US)

(21) Appl. No.: 18/282,640

(22) PCT Filed: Mar. 24, 2022
(86) PCT No.: PCT/US2022/021713

§ 371 Date: Sep. 18, 2023

Related U.S. Application Data
(60) Provisional application No. 63/165,695, filed on Mar. 24, 2021.

(51) Int. Cl. A61K 9/51; A61K 48/00; C12N 9/00; C12N 15/88

(52) U.S. Cl. CPC A61K 9/5123; A61K 48/0041; C12N 9/93; C12N 15/88; C12Y 604/01003

(57) ABSTRACT

This disclosure relates to ionizable amino lipid-based lipid nanoparticles for delivery of mRNA encoding propionyl-CoA carboxylase alpha (PCCA) and propionyl-CoA carboxylase beta (PCCB). Lipid nanoparticle/mRNA therapies of the invention increase and/or restore deficient levels of PCCA and/or PCCB expression and/or activity in subjects and are useful for the treatment of propionic academia.

### CROSS-REFERENCE TO RELATED APPLICATIONS

This application claims the priority benefit of U.S. Provisional Application No. 63/165,695, filed Mar. 24, 2021, the content of which is incorporated by reference in its entirety herein.

### SEQUENCE LISTING

The instant application contains a Sequence Listing which has been submitted electronically in ASCII format and is hereby incorporated by reference in its entirety. Said ASCII copy, created on Mar. 21, 2022, is named 45817-0107W01_SL.txt and is 65,418 bytes in size.

### BACKGROUND

Propionic acidemia (PA) is a rare, autosomal recessive metabolic disorder with significant morbidity and mortality that is caused by a deficiency in propionyl-CoA carboxylase (PCC) that prevents the enzyme from catalyzing the carboxylation of propionyl-CoA to methylmalonyl-CoA. Wongkittichote et al., Mol. Genet. Metab., Epub ahead of print. (2017). Disruption of PCC function causes propionyl-CoA and metabolites of propionate metabolism (breakdown of certain amino acids and fats) to accumulate in the blood, urine and other fluids and tissues/cells, which can lead to metabolic acidosis and hyperammonemia. Propionylcarnitine (C3), the levocarnitine ester of propionyl-CoA, 2-methylcitirc acid (2-MC), 3-hydroxypropionic acid (30HPA), propionylglycine, glycine, lactate and ammonia are also elevated in individuals with PA, and can serve as biomarkers for the disorder. Classical PA, caused by a complete loss of PCC function, usually presents in neonates in the first few hours or days after birth, with symptoms resulting from metabolic decompensation, including poor feeding, vomiting, hyper- or hypotonia, temperature instability, irritability, and lethargy. In rarer cases, late onset PA can occur after infancy, triggered by physical stress, such as infection. Mistreatment of acute metabolic discompensation, or lack of treatment, can lead to coma or death. The risk of mortality in this disorder is significant, as each acute metabolic decompensation is life-threatening and can lead to irreversible sequelae. Long-term complications of PA include neurodevelopmental sequelae, including significant cognitive deficits and developmental delays in motor and language skills, cardiomyopathy, arrhythmia, and pancreatitis. PA has an estimated incidence of 1:105,000 to 1:130,000 in the United States, but is higher in parts of the Middle East. Shchelochkov et al., GeneReviews (2016).

PCC (E.C. 6.4.1.3) is a heterodecamer composed of six propionyl-CoA carboxylase alpha subunits, encoded by PCCA (OMIM 232000), and 6 propionyl-CoA beta subunits, encoded by PCCB (OMIM 232050). The PCC enzyme is expressed in several tissues, and localizes to mitochondria where it engages with its necessary co-factor, biotin. There are three PCCA isoforms. The first isoform (NM_000282.3) encodes a protein (NP_000273.2) that is 728 amino acids in length, while isoform 2 (NM_001127692.2) encodes a protein (NP_001121164.1) that is 702 amino acids long, and isoform 3 (NM_001178004.1) encodes a protein (NP_001171475.1) that is 681 amino acids long. PCCA null variants, such as R288X and S537X, result in severe phenotypes, while splice type variants can result in milder disease. PCCB isoform 1 (NM_000532.4 encodes a protein (NP_000523.2) that is 539 amino acids in length, while isoform 2 (NM_001178014.1) encodes a protein (NP_001171485.1) that is 559 amino acids long. PCCB requires PCCA for stability, and can be absent in individuals lacking functional PCCA. Some PCCB gene variants disturb the interaction between PCCA and PCCB.

Elevated levels of intermediaries such as C3, 2-MC, 3-OHPA, and/or ammonia can be used as diagnostic markers for PA. Prenatal testing of 2-MC levels in amniotic fluids and newborn screening for elevated levels of C3 in dried blood spot can be used to diagnose PA prior to clinical presentation of the disease postpartum.

There are no approved drugs that directly address the underlying enzymatic defect of PA. Maintenance of patients with PA includes dietary intervention/supplementation, antibiotics to reduce propionate product by gut flora, and supportive care during metabolic decompensation events. Limited reports describing long-term benefits in patients with PA after liver transplantation indicate that metabolic decompensation events are absent, progressive neurological deterioration may stop or possibly improve, and cardiomyopathy might improve (Silva et al 2017, Quintero et al 2018, Critelli et al 2018, Charbit-Henrion et al 2015, Vara et al 2011). However, liver transplantation is not curative. Thus, there remains a high unmet medical need in this disorder, and alternative treatment options to increase PCC enzyme activity are needed.

### SUMMARY

The present disclosure provides ionizable amino lipid-based lipid nanoparticles for delivery of messenger RNA (mRNA) encoding propionyl-CoA carboxylase alpha (PCCA) and propionyl-CoA carboxylase beta (PCCB) in vivo. The lipid nanoparticle/mRNA therapeutics of the invention are particularly well-suited for the treatment of propionic acidemia, as the technology provides for the intracellular delivery of mRNAs encoding PCCA and PCCB followed by de novo synthesis of functional PCCA and PCCB proteins within target cells.

In one aspect, the disclosure features a lipid nanoparticle comprising a first mRNA comprising a first open reading frame (ORF) encoding a PCCA polypeptide and a second mRNA comprising a second ORF encoding a PCCB polypeptide, wherein the lipid nanoparticle comprises a compound of Formula (II):

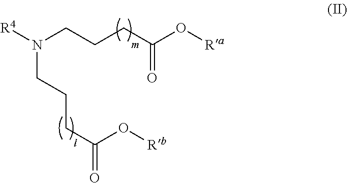

or its N-oxide, or a salt or isomer thereof,
wherein R′^{a} is R′^{branched} or R′^{cyclic}; wherein
R′^{branched} is:

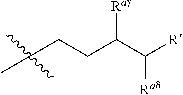

and R′^{cyclic} is:

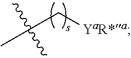

and
R′^{b} is:

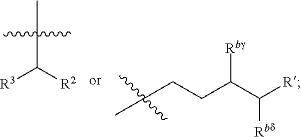

wherein

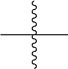

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH wherein n is selected from the group consisting of 1, 2, 3, 4, and 5, and

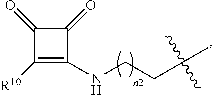

wherein

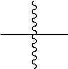

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the lipid nanoparticle comprises a compound of Formula (II-a):

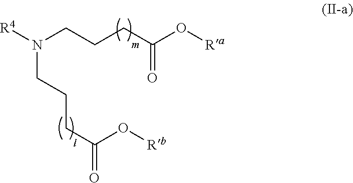

or its N-oxide, or a salt or isomer thereof,
wherein R′^{a} is R′^{branched} or R′^{cyclic}; wherein
R′^{branched} is:

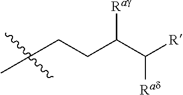

and R′^{b} is:

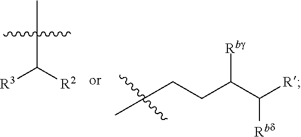

wherein

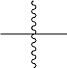

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH wherein n is selected from the group consisting of 1, 2, 3, 4, and 5, and

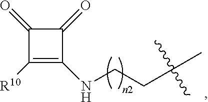

wherein

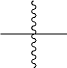

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the lipid nanoparticle comprises a compound of Formula

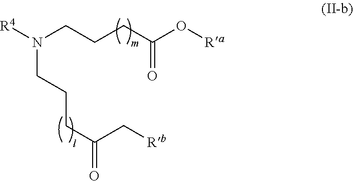

or its N-oxide, or a salt or isomer thereof,
wherein R′^{a} is R′^{branched} or R′^{cyclic}; wherein
R′^{branched} is:

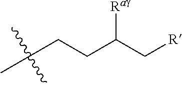

and R′^{b} is:

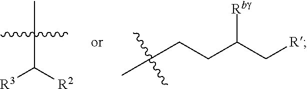

wherein

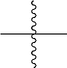

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH wherein n is selected from the group consisting of 1, 2, 3, 4, and 5, and

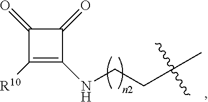

wherein

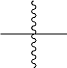

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the lipid nanoparticle comprises a compound of Formula (II-c):

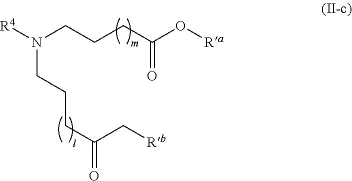

or its N-oxide, or a salt or isomer thereof,
wherein R′^{a} is R′^{branched} or R′^{cyclic}; wherein
R′^{branched} is:

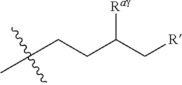

and R′^{b} is:

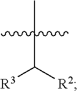

wherein

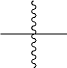

denotes a point of attachment; wherein R^{aγ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH wherein n is selected from the group consisting of 1, 2, 3, 4, and 5, and

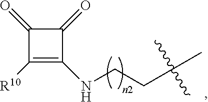

wherein

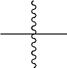

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the lipid nanoparticle comprises a compound of Formula (II-e):

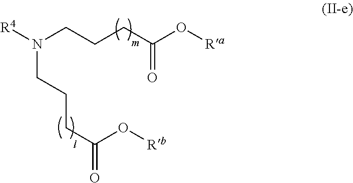

or its N-oxide, or a salt or isomer thereof,
wherein R′^{a} is R′^{branched} or R′^{cyclic}; wherein
R′^{branched} is:

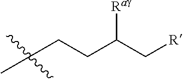

and R′^{b} is:

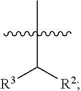

wherein

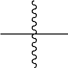

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the lipid nanoparticle comprises a compound of Formula

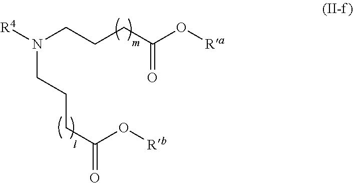

or its N-oxide, or a salt or isomer thereof,
wherein R′^{a} is R′^{branched} or R′^{cyclic}; wherein
R′^{branched} is:

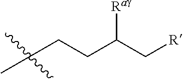

and R′^{b} is:

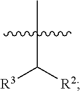

wherein

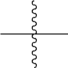

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments, the compound is

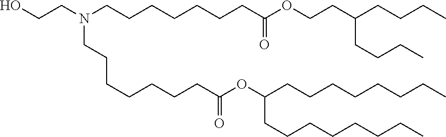 [SMILES: CCCCCCCCC(CCCCCCCC)OC(=O)CCCCCCCN(CCO)CCCCCCCC(=O)OCCC(CCCC)CCCC]

or its N-oxide, or a salt or isomer thereof.

In some embodiments, the compound is

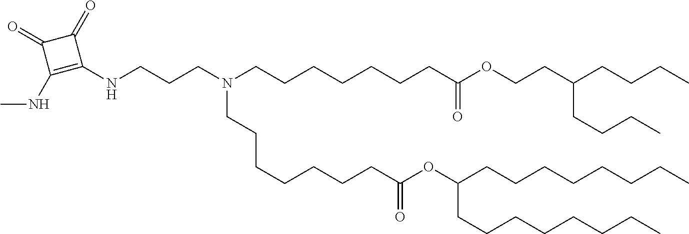 [SMILES: CCCCCCCCC(CCCCCCCC)OC(=O)CCCCCCCN(CCCCCCCC(=O)OCCC(CCCC)CCCC)CCCNc1c(NC)c(=O)c1=O]

or its N-oxide, or a salt or isomer thereof.

In some embodiments, the compound is

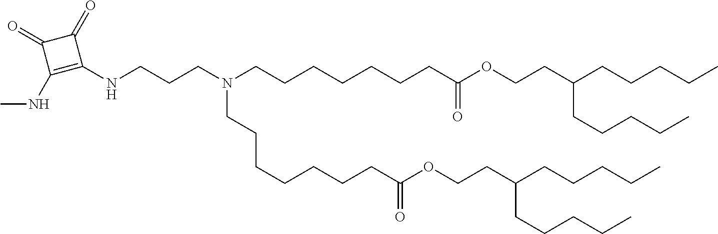 [SMILES: CCCCCC(CCCCC)CCOC(=O)CCCCCCCN(CCCCCCCC(=O)OCCC(CCCCC)CCCCC)CCCNc1c(NC)c(=O)c1=O]

or its N-oxide, or a salt or isomer thereof.

In some embodiments, the compound is

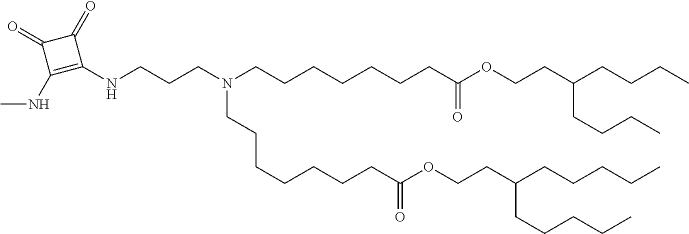 [SMILES: CCCCCC(CCCCC)CCOC(=O)CCCCCCCN(CCCCCCCC(=O)OCCC(CCCC)CCCC)CCCNc1c(NC)c(=O)c1=O]

or its N-oxide, or a salt or isomer thereof.

In some embodiments, the PCCA polypeptide comprises the amino acid sequence of SEQ ID NO:1 and the PCCB polypeptide comprises the amino acid sequence of SEQ ID NO:2.

In some embodiments, the first mRNA and the second mRNA are present in the lipid nanoparticle at a ratio of 1:1.

In some embodiments, the lipid nanoparticle further comprises a phospholipid, a structural lipid, and a PEG-lipid.

In some embodiments, the PEG-lipid is Compound I.

In some embodiments, the lipid nanoparticle comprises:
(i) 40-50 mol % of the compound of Formula (II), 30-45 mol % of the structural lipid, 5-15 mol % of the phospholipid, and 1-5 mol % of the PEG-lipid; or
(ii) 45-50 mol % of the compound of Formula (II), 35-45 mol % of the structural lipid, 8-12 mol % of the phospholipid, and 1.5 to 3.5 mol % of the PEG-lipid

In some embodiments, the first ORF is at least 80%, at least 85%, at least 90%, at least 91%, at least 92%, at least 93%, at least 94%, at least 95%, at least 96%, at least 97%, at least 98%, or at least 99% identical to the nucleotide sequence of SEQ ID NO:5, and the second ORF is at least 80%, at least 85%, at least 90%, at least 91%, at least 92%, at least 93%, at least 94%, at least 95%, at least 96%, at least 97%, at least 98%, or at least 99% identical to the nucleotide sequence of SEQ ID NO:6.

In some embodiments, the first ORF is at least 95% identical to the nucleotide sequence of SEQ ID NO:5 and the second ORF is at least 95% identical to the nucleotide sequence of SEQ ID NO:6.

In some embodiments, the first ORF is 100% identical to the nucleotide sequence of SEQ ID NO:5, and the second ORF is 100% identical to the nucleotide sequence of SEQ ID NO:6.

In some embodiments, the first mRNA comprises a 5′ UTR comprising a nucleic acid sequence at least 90%, at least 95%, at least 96%, at least 97%, at least 98%, at least 99%, or 100% identical to SEQ ID NO:55, and the second mRNA comprises a 5′ UTR comprising a nucleic acid sequence at least 90%, at least 95%, at least 96%, at least 97%, at least 98%, at least 99%, or 100% identical to SEQ ID NO:55.

In some embodiments, first the mRNA comprises a 3′ UTR comprising a nucleic acid sequence at least 90%, at least 95%, at least 96%, at least 97%, at least 98%, at least 99%, or 100% identical to SEQ ID NO:114, and second the mRNA comprises a 3′ UTR comprising a nucleic acid sequence at least 90%, at least 95%, at least 96%, at least 97%, at least 98%, at least 99%, or 100% identical to SEQ ID NO:114.

In some embodiments, the first mRNA comprises the nucleic acid sequence of SEQ ID NO:7 and the second mRNA comprises the nucleic acid sequence of SEQ ID NO:8.

In some embodiments, the first mRNA and the second mRNA each comprise a 5′ terminal cap.

In some embodiments, the 5′ terminal cap comprises a Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In some embodiments, the first mRNA and the second mRNA each comprise a poly-A region.

In some embodiments, the poly-A region is at least about 10, at least about 20, at least about 30, at least about 40, at least about 50, at least about 60, at least about 70, at least about 80, at least about 90 nucleotides in length, or at least about 100 nucleotides in length.

In some embodiments, the poly-A region is at least about 100 nucleotides in length.

In some embodiments, all of the uracils of the first mRNA and the second mRNA are N1-methylpseudouracils.

In some embodiments, the first ORF is 100% identical to SEQ ID NO:5, wherein the second ORF is 100% identical to SEQ ID NO:6, wherein the first mRNA and the second mRNA each comprise a poly-A region at least about 100 nucleotides in length, and wherein all of the uracils of the first mRNA and the second mRNA are N1-methylpseudouracils.

In some embodiments, the first mRNA and the second mRNA each comprise a 5′ terminal cap comprising a guanine cap nucleotide containing an N7 methylation and the 5′-terminal nucleotide of the mRNA contains a 2′-O-methyl, wherein the first mRNA comprises the nucleic acid sequence of SEQ ID NO:7 and the second mRNA comprises the nucleic acid sequence of SEQ ID NO:8, wherein the first mRNA and the second mRNA each comprise a poly-A region at least about 100 nucleotides in length, and wherein all of the uracils of the first mRNA and the second mRNA are N1-methylpseudouracils.

In another aspect, the disclosure features a method of expressing a PCCA polypeptide and a PCCB polypeptide in a human subject in need thereof, comprising administering to the subject an effective amount of a lipid nanoparticle described herein.

In another aspect, the disclosure features a method of treating, preventing, or delaying the onset and/or progression of propionic academia in a human subject in need thereof, the method comprising administering to the human subject an effective amount of a lipid nanoparticle described herein.

In another aspect, the disclosure features a method of reducing propionic acid blood level in a human subject in need thereof, comprising administering to the subject an effective amount of a lipid nanoparticle described herein.

In another aspect, the disclosure features a method of reducing C3, 2-MC, 3OHTPA, and/or ammonia plasma, serum, whole blood, and/or liver level in a human subject in need thereof, comprising administering to the subject an effective amount of a lipid nanoparticle described herein.

In another aspect, the disclosure features a method of increasing PCC activity in a human subject in need thereof, comprising administering to the subject an effective amount of a lipid nanoparticle described herein.

In some embodiments of any of the methods described herein, the human subject has a loss of function mutation in the PCCA gene.

In some embodiments of any of the methods described herein, the human subject has a loss of function mutation in the PCCB gene.

In some embodiments of any of the methods described herein, the human subject has a loss of function mutation in the PCCA gene and a loss of function mutation in the PCCB gene.

In some embodiments of any of the methods described herein, the method comprises multiple administrations of the lipid nanoparticle to the human subject. In some embodiments, the multiple administrations to the human subject are about once a week, about once every two weeks, or about once a month.

In some embodiments of any of the methods described herein, the lipid nanoparticle is administered intravenously.

### BRIEF DESCRIPTION OF THE DRAWINGS

FIG. 1A is a graph showing the amount of UCE protein detected (as a percentage of wild type) 2 days post-dosing with PBS or the indicated lipid nanoparticles encapsulating UCE-encoding mRNAs (0.5 mg/kg).

FIG. 1B is a graph showing UCE activity 2 days post-dosing with PBS or the indicated lipid nanoparticles encapsulating UCE-encoding mRNAs (0.5 mg/kg).

FIG. 1C is a graph showing the UCE immunohistochemistry H-score 2 days post-administration of PBS or the indicated lipid nanoparticles encapsulating UCE-encoding mRNAs (0.5 mg/kg).

FIG. 2 is a graph showing fasting blood glucose levels in GE deficient mice injected with 0.25 mg/kg of GE mRNA in various lipid nanoparticles. Control wild-type mice were injected with PBS. Control GE deficient mice were injected with mRNA encoding eGFP (0.25 mg/kg). The dotted horizontal line denotes the therapeutic threshold blood glucose level (60 mg/dl) to maintain normal physiology.

FIGS. 3A-3B are bar graphs showing hepatic human GE protein levels of female (FIG. 3A) and male (FIG. 3B) wild-type mice 24 hours after injection with 0.125 mg/kg, 0.25 mg/kg, or 0.5 mg/kg of GE mRNA in lipid nanoparticles. Results were compared to the hepatic human GE protein level in wild-type mice injected with PBS.

FIGS. 4A-4B are bar graphs showing hepatic GE activity of female (FIG. 4A) and male (FIG. 4B) wild-type mice 24 hours after injection with 0.125 mg/kg, 0.25 mg/kg, or 0.5 mg/kg of GE mRNA in lipid nanoparticles. Results were compared to the hepatic GE activity in wild-type mice injected with PBS.

### DETAILED DESCRIPTION

The present disclosure provides lipid nanoparticle/mRNA therapeutics for the treatment of propionic acidemia. Lipid nanoparticles are an ideal platform for the safe and effective delivery of mRNAs to target cells. Lipid nanoparticles have the unique ability to deliver nucleic acids by a mechanism involving cellular uptake, intracellular transport and endosomal release or endosomal escape.

### Propionyl-CoA Carboxylase (PCC)

Propionyl-CoA carboxylase (PCC; EC 6.4.1.3) catalyzes the carboxylation of propionyl-CoA with bicarbonate, producing methylmalonyl-CoA. Methylmalonyl-CoA is then converted to succinyl-CoA, which is an intermediate in the tricarboxylic acid cycle (TCA). In the cell, PCC exists as a heterododecamer composed of six propionyl-CoA carboxylase alpha subunits, encoded by PCCA (OMIM 232000), and 6 propionyl-CoA beta subunits, encoded by PCCB (OMIM 232050).

Propionic acidemia is an autosomal recessive metabolic disorder associated with PCC function. PA results when bi-allelic variants eliminate or reduce the function of the PCCA or PCCB subunits of PCC. Propionyl-CoA accumulates intracellularly in human subjects with PA, which has many metabolic effects, including, e.g., the inhibition of mitochondrial respiratory function and reduced synthesis of citrate, GTP and ATP. A variety of variant PCCA and PCCB proteins have different levels of activity, with the severity of PA being correlated with the severity of the enzymes' mutations. For example, PCCA null mutations, such as R288X and S537X, result in severe phenotypes, while certain splice site variants can result in milder disease. Generally, variations in the PCCA N and C terminal regions can cause PA because regions are necessary for PCC's holocarboxylase synthase interaction. PCCB mutations, e.g., A497V, R512C, L519P, and W531X, often affect the interaction between PCCA and PCCB, thereby disturbing PCC stability and function.

In certain aspects, the disclosure provides a polynucleotide (e.g., a RNA, e.g., a mRNA) comprising a nucleotide sequence (e.g., an open reading frame (ORF)) encoding a PCCA or PCCB polypeptide. In some embodiments, the PCCA or PCCB polypeptide is a wild type full length human PCCA or PCCB protein. In some embodiments, the PCCA or PCCB polypeptide is a variant, a peptide or a polypeptide containing a substitution, and insertion and/or an addition, a deletion and/or a covalent modification with respect to a wild-type PCCA or PCCB sequence. In some embodiments, the PCCA or PCCB polypeptide is a truncated human PCCA or PCCB protein.

In some embodiments, the polynucleotide (e.g., a RNA, e.g., an mRNA) comprising a nucleotide sequence (e.g., an ORF) encodes a substitutional variant of a human PCCA or PCCB sequence, which can comprise one, two, three, four, five, six, seven, eight, nine, ten or more than ten substitutions. In some embodiments, the substitutional variant can comprise one or more conservative amino acids substitutions. In other embodiments, the variant is an insertional variant. In other embodiments, the variant is a deletional variant.

In some embodiments, a polynucleotide disclosed herein comprises a sequence encoding the PCCA protein of SEQ ID NO:1. In some embodiments, a polynucleotide disclosed herein comprises a sequence encoding the PCCB protein of SEQ ID NO:2.

### Polynucleotides and Open Reading Frames (ORFs)

The instant invention features mRNAs for use in treating or preventing propionic acidemia. The mRNAs featured for use in the invention are administered to subjects and encode human PCCA or PCCB protein in vivo. Accordingly, the invention relates to polynucleotides, e.g., mRNA, comprising an open reading frame of linked nucleosides encoding human PCCA or PCCB (e.g., SEQ ID NO:1 or SEQ ID NO:2), functional fragments thereof, and fusion proteins comprising PCCA or PCCB. Specifically, the invention provides sequence-optimized polynucleotides comprising nucleotides encoding the polypeptide sequence of human PCCA or PCCB (e.g., SEQ ID NO:1 or SEQ ID NO:2), or sequence having high sequence identity with those sequence optimized polynucleotides.

In certain aspects, the invention provides polynucleotides (e.g., a RNA such as an mRNA) that comprise a nucleotide sequence (e.g., an ORF) encoding one or more PCCA or PCCB polypeptides. In some embodiments, the encoded PCCA or PCCB polypeptide of the invention can be selected from:
(i) a full length PCCA or PCCB polypeptide (e.g., having the same or essentially the same length as SEQ ID NO:1 or SEQ ID NO:2);
(ii) (a functional fragment of PCCA or PCCB described herein (e.g., a truncated (e.g., deletion of carboxy, amino terminal, or internal regions) sequence shorter than PCCA or PCCB; but still retaining PCCA or PCCB enzymatic activity);
(iii) (a variant thereof (e.g., full length or truncated PCCA or PCCB proteins in which one or more amino acids have been replaced, e.g., variants that retain all or most of the PCCA or PCCB activity of the polypeptide with respect to a reference protein (e.g., any natural or artificial variants known in the art or described herein (e.g., SEQ ID NO:2))); or
(iv) a fusion protein comprising (i) a full length PCCA or PCCB protein (e.g., SEQ ID NO:1 or SEQ ID NO:2), and (ii) a heterologous protein.

In certain embodiments, the encoded PCCA or PCCB polypeptide is a mammalian PCCA or PCCB polypeptide, such as a human PCCA or PCCB polypeptide, a functional fragment or a variant thereof.

In some embodiments, the polynucleotide (e.g., a RNA, e.g., an mRNA) of the invention increases PCCA or PCCB protein expression levels and/or detectable PCCA or PCCB enzymatic activity levels in cells when introduced in those cells, e.g., by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, compared to PCCA or PCCB protein expression levels and/or detectable PCCA or PCCB enzymatic activity levels in the cells prior to the administration of the polynucleotide of the invention. PCCA or PCCB protein expression levels and/or PCCA or PCCB enzymatic activity can be measured according to methods know in the art. In some embodiments, the polynucleotide is introduced to the cells in vitro. In some embodiments, the polynucleotide is introduced to the cells in vivo.

In some embodiments, the polynucleotides (e.g., a RNA, e.g., an mRNA) of the invention comprise a nucleotide sequence (e.g., an ORF) that encodes SEQ ID NO:1.

In some embodiments, the polynucleotides (e.g., a RNA, e.g., an mRNA) of the invention comprise a nucleotide sequence (e.g., an ORF) that encodes SEQ ID NO:2.

The polynucleotide (e.g., a RNA, e.g., an mRNA) of the invention comprises a codon optimized nucleic acid sequence, wherein the open reading frame (ORF) of the codon optimized nucleic acid sequence is derived from a wild-type PCCA or PCCB sequence (e.g., wild-type human PCCA or PCCB). For example, for polynucleotides of invention comprising a sequence optimized ORF encoding PCCA or PCCB, the corresponding wild type sequence is the native human PCCA or PCCB. Similarly, for a sequence optimized mRNA encoding a functional fragment of human PCCA or PCCB, the corresponding wild type sequence is the corresponding fragment from human PCCA or PCCB.

In some embodiments, the polynucleotides (e.g., a RNA, e.g., an mRNA) of the invention comprise a nucleotide sequence encoding PCCA or PCCB having the full-length sequence of human PCCA or PCCB.

In some embodiments, the polynucleotides (e.g., a RNA, e.g., an mRNA) of the invention comprise a nucleotide sequence (e.g., an ORF) encoding a mutant PCCA or PCCB polypeptide. In some embodiments, the polynucleotides of the invention comprise an ORF encoding an PCCA or PCCB polypeptide that comprises at least one point mutation in the PCCA or PCCB amino acid sequence and retains PCC enzymatic activity. In some embodiments, the mutant PCCA or PCCB polypeptide has an PCC activity which is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the PCC activity of the corresponding wild-type PCCA or PCCB. In some embodiments, the polynucleotide (e.g., a RNA, e.g., an mRNA) of the invention comprising an ORF encoding a mutant PCCA or PCCB polypeptide is sequence optimized.

In some embodiments, the polynucleotide (e.g., a RNA, e.g., an mRNA) of the invention comprises a nucleotide sequence (e.g., an ORF) that encodes an PCCA or PCCB polypeptide with mutations that do not alter PCC enzymatic activity. Such mutant PCCA or PCCB polypeptides can be referred to as function-neutral. In some embodiments, the polynucleotide comprises an ORF that encodes a mutant PCCA or PCCB polypeptide comprising one or more function-neutral point mutations.

In some embodiments, the mutant PCCA or PCCB polypeptide has higher PCC enzymatic activity than the corresponding wild-type PCCA or PCCB. In some embodiments, the mutant PCCA or PCCB polypeptide has an PCC activity that is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than the activity of the corresponding wild-type PCCA or PCCB (i.e., the same PCCA or PCCB protein but without the mutation(s)).

In some embodiments, the polynucleotides (e.g., a RNA, e.g., an mRNA) of the invention comprise a nucleotide sequence (e.g., an ORF) encoding a functional PCCA or PCCB fragment, e.g., where one or more fragments correspond to a polypeptide subsequence of a wild type PCCA or PCCB polypeptide and retain PCC enzymatic activity. In some embodiments, the PCCA or PCCB fragment has an PCC activity which is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the PCC activity of the corresponding full length PCCA or PCCB. In some embodiments, the polynucleotides (e.g., a RNA, e.g., an mRNA) of the invention comprising an ORF encoding a functional PCCA or PCCB fragment is sequence optimized.

In some embodiments, the polynucleotide (e.g., a RNA, e.g., an mRNA) of the invention comprises a nucleotide sequence (e.g., an ORF) encoding an PCCA or PCCB fragment that has higher PCC enzymatic activity than the corresponding full length PCCA or PCCB. Thus, in some embodiments the PCCA or PCCB fragment has an PCC activity which is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than the PCC activity of the corresponding full length PCCA or PCCB.

In some embodiments, the polynucleotide (e.g., a RNA, e.g., an mRNA) of the invention comprises a nucleotide sequence (e.g., an ORF) encoding an PCCA or PCCB fragment that is at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25% shorter than wild-type PCCA or PCCB.

In some embodiments, the polynucleotide (e.g., a RNA, e.g., an mRNA) of the invention comprises a nucleotide sequence (e.g., an ORF) encoding an PCCA or PCCB polypeptide (e.g., the wild-type sequence, functional fragment, or variant thereof), wherein the nucleotide sequence is at least 90%, at least 95%, at least 97%, at least 98%, at least 99%, or 100% identical to the sequence of SEQ ID NO:5 or SEQ ID NO:6.

In some embodiments, the polynucleotide (e.g., a RNA, e.g., an mRNA) of the invention comprises from about 1,00 to about 100,000 nucleotides (e.g., from 1,000 to 2,500, from 1,000 to 2,600, from 1,000 to 2,700, from 1,000 to 2,800, from 1,000 to 2,900, from 1,000 to 3,000, from 1,000 to 5,000, from 1,000 to 10,000, from 1,000 to 25,000, from 1,000 to 50,000, from 1,000 to 70,000, from 1,000 to 100,000, from 1062 to 2,700, from 1062 to 2,800, from 1062 to 2,900, from 1062 to 5,000, from 1062 to 7,000, from 1062 to 10,000, from 1062 to 25,000, from 1062 to 50,000, from 1062 to 70,000, or from 1062 to 100,000).

In some embodiments, the polynucleotide of the invention (e.g., a RNA, e.g., an mRNA) comprises a nucleotide sequence (e.g., an ORF) encoding an PCCA or PCCB polypeptide (e.g., the wild-type sequence, functional fragment, or variant thereof), wherein the length of the nucleotide sequence (e.g., an ORF) is at least 500 nucleotides in length (e.g., at least or greater than about 500, 600, 700, 80, 900, 1,000, 1,050, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,635, 4,700, 4,800, 4,900, 5,000, 5,100, 5,200, 5,300, 5,400, 5,500, 5,600, 5,700, 5,800, 5,900, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the polynucleotide of the invention (e.g., a RNA, e.g., an mRNA) comprises a nucleotide sequence (e.g., an ORF, e.g., SEQ ID NO:5 or SEQ ID NO:6) encoding an PCCA or PCCB polypeptide (e.g., the wild-type sequence, functional fragment, or variant thereof) further comprises a 5′-UTR (e.g., SEQ ID NO:55) and a 3′-UTR (e.g., SEQ ID NO:114). In some embodiments, the polynucleotide (e.g., a RNA, e.g., an mRNA) of the invention comprises the sequence of SEQ ID NO:5 or SEQ ID NO:6. In a further embodiment, the polynucleotide (e.g., a RNA, e.g., an mRNA) comprises a 5′ terminal cap (e.g., m⁷Gp-ppGm-A, Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof) and a poly-A-tail region (e.g., about 100 nucleotides in length (SEQ ID NO: 195)). In some embodiments, the mRNA comprises a polyA tail. In some instances, the poly A tail is 50-150 (SEQ ID NO:197), 75-150 (SEQ ID NO:198), 85-150 (SEQ ID NO:199), 90-120 (SEQ ID NO:193), 90-130 (SEQ ID NO:194), or 90-150 (SEQ ID NO:192) nucleotides in length. In some instances, the poly A tail is 100 nucleotides in length (SEQ ID NO:195). In some instances, the poly A tail is protected (e.g., with an inverted deoxy-thymidine). In some instances, the poly A tail comprises A100-UCUAG-A20-inverted deoxy-thymidine (SEQ ID NO: 211). In some instances, the poly A tail is A100-UCUAG-A20-inverted deoxy-thymidine (SEQ ID NO: 211).

In some embodiments, the polynucleotide of the invention (e.g., a RNA, e.g., an mRNA) comprises a nucleotide sequence (e.g., an ORF) encoding an PCCA or PCCB polypeptide (e.g., the wild-type sequence, functional fragment, or variant thereof) further comprises at least one nucleic acid sequence that is noncoding, e.g., a microRNA binding site. In some embodiments, the polynucleotide (e.g., a RNA, e.g., an mRNA) of the invention further comprises a 5′-UTR (e.g., SEQ ID NO:55) and a 3′ UTR (e.g., SEQ ID NO:114). In some embodiments, the polynucleotide (e.g., a RNA, e.g., an mRNA) of the invention comprises the sequence of SEQ ID NO:5 or SEQ ID NO:6. In a further embodiment, the polynucleotide (e.g., a RNA, e.g., an mRNA) comprises a 5′ terminal cap (e.g., m⁷Gp-ppGm-A, Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof) and a poly-A-tail region (e.g., about 100 nucleotides in length (SEQ ID NO: 195), e.g., A100-UCUAG-A20-inverted deoxy-thymidine (SEQ ID NO: 211)). In some embodiments, the mRNA comprises a polyA tail. In some instances, the poly A tail is 50-150 (SEQ ID NO:197), 75-150 (SEQ ID NO:198), 85-150 (SEQ ID NO:199), 90-120 (SEQ ID NO:193), 90-130 (SEQ ID NO:194), or 90-150 (SEQ ID NO:192) nucleotides in length. In some instances, the poly A tail is 100 nucleotides in length (SEQ ID NO:195). In some instances, the poly A tail is protected (e.g., with an inverted deoxy-thymidine). In some instances, the poly A tail comprises A100-UCUAG-A20-inverted deoxy-thymidine (SEQ ID NO:211). In some instances, the poly A tail is A100-UCUAG-A20-inverted deoxy-thymidine (SEQ ID NO:211).

In some embodiments, the polynucleotide of the invention (e.g., a RNA, e.g., an mRNA) comprises a nucleotide sequence (e.g., an ORF) encoding an PCCA or PCCB polypeptide (e.g., the wild-type sequence, functional fragment, or variant thereof) further comprises at least one nucleic acid sequence that is noncoding, e.g., a microRNA binding site. In some embodiments, the polynucleotide (e.g., a RNA, e.g., an mRNA) of the invention further comprises a 5′-UTR (e.g., selected from the sequences of SEQ ID Nos: 50-79) and a 3′UTR (e.g., selected from the sequences of SEQ ID Nos: 100-136). In some embodiments, the polynucleotide (e.g., a RNA, e.g., an mRNA) of the invention comprises the sequence of SEQ ID NO:5 or SEQ ID NO:6. In a further embodiment, the polynucleotide (e.g., a RNA, e.g., an mRNA) comprises a 5′ terminal cap (e.g., m⁷Gp-ppGm-A, Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof) and a poly-A-tail region (e.g., about 100 nucleotides in length, e.g., SEQ ID NO:195). In some embodiments, the mRNA comprises a 3′ UTR comprising a nucleic acid sequence of SEQ ID NO:114. In some embodiments, the mRNA comprises a polyA tail. In some instances, the poly A tail is 50-150 (SEQ ID NO:197), 75-150 (SEQ ID NO:198), 85-150 (SEQ ID NO:199), 90-120 (SEQ ID NO:193), 90-130 (SEQ ID NO:194), or 90-150 (SEQ ID NO:192) nucleotides in length. In some instances, the poly A tail is 100 nucleotides in length (SEQ ID NO:195). In some instances, the poly A tail is protected (e.g., with an inverted deoxy-thymidine). In some instances, the poly A tail comprises A100-UCUAG-A20-inverted deoxy-thymidine (SEQ ID NO:211). In some instances, the poly A tail is A100-UCUAG-A20-inverted deoxy-thymidine (SEQ ID NO:211).

In some embodiments, the polynucleotide of the invention (e.g., a RNA, e.g., an mRNA) comprises a nucleotide sequence (e.g., an ORF, e.g., the sequence of SEQ ID NO:5 or SEQ ID NO:6) encoding an PCCA or PCCB polypeptide (e.g., the wild-type sequence, functional fragment, or variant thereof) further comprises a 5′-UTR (e.g., selected from the sequences of SEQ ID Nos:50-79) and a 3′-UTR (e.g., selected from the sequences of SEQ ID Nos: 100-136). In some embodiments, the polynucleotide (e.g., a RNA, e.g., an mRNA) of the invention comprises the sequence of SEQ ID NO:5 or SEQ ID NO:6. In a further embodiment, the polynucleotide (e.g., a RNA, e.g., an mRNA) comprises a 5′ terminal cap (e.g., m⁷Gp-ppGm-A, Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof) and a poly-A-tail region (e.g., about 100 nucleotides in length (SEQ ID NO: 195)). In some embodiments, the mRNA comprises a polyA tail. In some instances, the poly A tail is 50-150 (SEQ ID NO:197), 75-150 (SEQ ID NO:198), 85-150 (SEQ ID NO:199), 90-120 (SEQ ID NO:193), 90-130 (SEQ ID NO:194), or 90-150 (SEQ ID NO:192) nucleotides in length. In some instances, the poly A tail is 100 nucleotides in length (SEQ ID NO:195). In some instances, the poly A tail is protected (e.g., with an inverted deoxy-thymidine). In some instances, the poly A tail comprises A100-UCUAG-A20-inverted deoxy-thymidine (SEQ ID NO:211). In some instances, the poly A tail is A100-UCUAG-A20-inverted deoxy-thymidine (SEQ ID NO:211).

In some embodiments, the polynucleotide of the invention (e.g., a RNA, e.g., an mRNA) comprises a 5′ UTR comprising the nucleotide sequence of SEQ ID NO:55 and a nucleotide sequence (e.g., an ORF) encoding the PCCA polypeptide of SEQ ID NO:1. In some embodiments, the polynucleotide (e.g., a RNA, e.g., an mRNA) comprises a 5′ UTR comprising the nucleotide sequence of SEQ ID NO:55, a nucleotide sequence (e.g., an ORF) encoding the PCCA polypeptide of SEQ ID NO:1, and a 3′ UTR comprising the nucleotide sequence of SEQ ID NO:114.

In some embodiments, the polynucleotide of the invention (e.g., a RNA, e.g., an mRNA) comprises a 5′ UTR comprising the nucleotide sequence of SEQ ID NO:55 and a nucleotide sequence (e.g., an ORF) encoding the PCCB polypeptide of SEQ ID NO:2. In some embodiments, the polynucleotide (e.g., a RNA, e.g., an mRNA) comprises a 5′ UTR comprising the nucleotide sequence of SEQ ID NO:55, a nucleotide sequence (e.g., an ORF) encoding the PCCB polypeptide of SEQ ID NO:2, and a 3′ UTR comprising the nucleotide sequence of SEQ ID NO:114.

In some embodiments, the polynucleotide of the invention (e.g., a RNA, e.g., an mRNA) comprises a nucleotide sequence (e.g., an ORF) encoding an PCCA or PCCB polypeptide is single stranded or double stranded.

In some embodiments, the polynucleotide of the invention comprising a nucleotide sequence (e.g., an ORF) encoding an PCCA or PCCB polypeptide (e.g., the wild-type sequence, functional fragment, or variant thereof) is DNA or RNA. In some embodiments, the polynucleotide of the invention is RNA. In some embodiments, the polynucleotide of the invention is, or functions as, a mRNA. In some embodiments, the mRNA comprises a nucleotide sequence (e.g., an ORF) that encodes at least one PCCA or PCCB polypeptide, and is capable of being translated to produce the encoded PCCA or PCCB polypeptide in vitro, in vivo, in situ or ex vivo.

In some embodiments, the polynucleotide of the invention (e.g., a RNA, e.g., an mRNA) comprises a sequence-optimized nucleotide sequence (e.g., an ORF) encoding an PCCA or PCCB polypeptide (e.g., the wild-type sequence, functional fragment, or variant thereof, see e.g., SEQ ID NO:5 or SEQ ID NO:6), wherein the polynucleotide comprises at least one chemically modified nucleobase, e.g., N1-methylpseudouracil or 5-methoxyuracil. In certain embodiments, all uracils in the polynucleotide are N1-methylpseudouracils. In other embodiments, all uracils in the polynucleotide are 5-methoxyuracils. In some embodiments, the polynucleotide further comprises a miRNA binding site, e.g., a miRNA binding site that binds to miR-142 and/or a miRNA binding site that binds to miR-126.

In some embodiments, the polynucleotide (e.g., a RNA, e.g., a mRNA) disclosed herein is Formulated with a delivery agent comprising, e.g., a compound having the Formula (II), e.g., Compound B. In some embodiments, the delivery agent comprises an ionizable amino lipid (e.g., Compound B), a helper lipid (e.g., DSPC), a sterol (e.g., Cholesterol), and a PEG lipid (e.g., Compound I or PEG-DMG), e.g., with a mole ratio in the range of about (i) 40-50 mol % ionizable amino lipid (e.g., Compound B), optionally 45-50 mol % ionizable amino lipid, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %; (ii) 30-45 mol % sterol (e.g., cholesterol), optionally 35-42 mol % sterol, for example, 30-31 mol %, 31-32 mol %, 32-33 mol %, 33-34 mol %, 35-35 mol %, 35-36 mol %, 36-37 mol %, 37-38 mol %, 38-39 mol %, or 39-40 mol %, or 40-42 mol % sterol; (iii) 5-15 mol % helper lipid (e.g., DSPC), optionally 10-15 mol % helper lipid, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % helper lipid; and (iv) 1-5% PEG lipid (e.g., Compound I or PEG-DMG), optionally 1-5 mol % PEG lipid, for example 1.5 to 2.5 mol %, 1-2 mol %, 2-3 mol %, 3-4 mol %, or 4-5 mol % PEG lipid. In some embodiments, the delivery agent comprises Compound B, Cholesterol, DSPC, and Compound I with a mole ratio of 47:39:11:3.

In some embodiments, the polynucleotide of the disclosure is an mRNA that comprises a 5′-terminal cap (e.g., Cap1, e.g., m⁷Gp-ppGm-A), a 5′UTR (e.g., any one of SEQ ID NOs:50-79), an ORF sequence of SEQ ID NO:5 or SEQ ID NO:6, a 3′UTR (e.g., any one of SEQ ID NOs:100-136), and a poly A tail (e.g., about 100 nt in length, e.g., SEQ ID NO:195), wherein all uracils in the polynucleotide are N1-methylpseudouracils or 5-methoxyuracil.

In some embodiments, the polynucleotide of the disclosure is an mRNA that comprises a 5′-terminal cap (e.g., Cap1, e.g., m⁷Gp-ppGm-A), a 5′UTR (e.g., SEQ ID NO:55), an ORF sequence of SEQ ID NO:5, a 3′UTR (e.g., SEQ ID NO:114), and a poly A tail (e.g., about 100 nt in length (SEQ ID NO: 195)), wherein all uracils in the polynucleotide are N1-methylpseudouracils or 5-methoxyuracil. In some embodiments, the delivery agent comprises Compound B as the ionizable amino lipid and PEG-DMG or Compound I as the PEG lipid.

In some embodiments, the polynucleotide of the disclosure is an mRNA that comprises a 5′-terminal cap (e.g., Cap1, e.g., m⁷Gp-ppGm-A), a 5′UTR (e.g., SEQ ID NO:55), an ORF sequence of SEQ ID NO:6, a 3′UTR (e.g., SEQ ID NO:114), and a poly A tail (e.g., about 100 nt in length (SEQ ID NO: 195)), wherein all uracils in the polynucleotide are N1-methylpseudouracils or 5-methoxyuracil. In some embodiments, the delivery agent comprises Compound B as the ionizable amino lipid and PEG-DMG or Compound I as the PEG lipid.

Exemplary ORFs encoding a PCCA or PCCB polypeptide are described in WO 2019/104195, the content of which is incorporated herein by reference.

### Signal Sequences

The polynucleotides (e.g., a RNA, e.g., an mRNA) of the invention can also comprise nucleotide sequences that encode additional features that facilitate trafficking of the encoded polypeptides to therapeutically relevant sites. One such feature that aids in protein trafficking is the signal sequence, or targeting sequence. The peptides encoded by these signal sequences are known by a variety of names, including targeting peptides, transit peptides, and signal peptides. In some embodiments, the polynucleotide (e.g., a RNA, e.g., an mRNA) comprises a nucleotide sequence (e.g., an ORF) that encodes a signal peptide operably linked to a nucleotide sequence that encodes an PCCA or PCCB polypeptide described herein.

In some embodiments, the “signal sequence” or “signal peptide” is a polynucleotide or polypeptide, respectively, which is from about 30-210, e.g., about 45-80 or 15-60 nucleotides (e.g., about 20, 30, 40, 50, 60, or 70 amino acids) in length that, optionally, is incorporated at the 5′ (or N-terminus) of the coding region or the polypeptide, respectively. Addition of these sequences results in trafficking the encoded polypeptide to a desired site, such as the endoplasmic reticulum or the mitochondria through one or more targeting pathways. Some signal peptides are cleaved from the protein, for example by a signal peptidase after the proteins are transported to the desired site.

In some embodiments, the polynucleotide of the invention comprises a nucleotide sequence encoding an PCCA or PCCB polypeptide, wherein the nucleotide sequence further comprises a 5′ nucleic acid sequence encoding a heterologous signal peptide.

### Fusion Proteins

In some embodiments, the polynucleotide of the invention (e.g., a RNA, e.g., an mRNA) can comprise more than one nucleic acid sequence (e.g., an ORF) encoding a polypeptide of interest. In some embodiments, polynucleotides of the invention comprise a single ORF encoding an PCCA or PCCB polypeptide, a functional fragment, or a variant thereof. However, in some embodiments, the polynucleotide of the invention can comprise more than one ORF, for example, a first ORF encoding an PCCA or PCCB polypeptide (a first polypeptide of interest), a functional fragment, or a variant thereof, and a second ORF expressing a second polypeptide of interest. In some embodiments, two or more polypeptides of interest can be genetically fused, i.e., two or more polypeptides can be encoded by the same ORF. In some embodiments, the polynucleotide can comprise a nucleic acid sequence encoding a linker (e.g., a G₄S (SEQ ID NO: 200) peptide linker or another linker known in the art) between two or more polypeptides of interest.

In some embodiments, a polynucleotide of the invention (e.g., a RNA, e.g., an mRNA) can comprise two, three, four, or more ORFs, each expressing a polypeptide of interest.

In some embodiments, the polynucleotide of the invention (e.g., a RNA, e.g., an mRNA) can comprise a first nucleic acid sequence (e.g., a first ORF) encoding an PCCA or PCCB polypeptide and a second nucleic acid sequence (e.g., a second ORF) encoding a second polypeptide of interest.

### Linkers and Cleavable Peptides

In certain embodiments, the mRNAs of the disclosure encode more than one PCCA or PCCB domain or a heterologous domain, referred to herein as multimer constructs. In certain embodiments of the multimer constructs, the mRNA further encodes a linker located between each domain. The linker can be, for example, a cleavable linker or protease-sensitive linker. In certain embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In certain embodiments, the linker is an F2A linker. In certain embodiments, the linker is a GGGS (SEQ ID NO: 201) linker. In certain embodiments, the linker is a (GGGS)n (SEQ ID NO: 202) linker, wherein n=2, 3,4, or 5. In certain embodiments, the multimer construct contains three domains with intervening linkers, having the structure: domain-linker-domain-linker-domain e.g., PCCA or PCCB domain-linker-PCCA or PCCB domain-linker-PCCA or PCCB domain.

In one embodiment, the cleavable linker is an F2A linker (e.g., having the amino acid sequence GSGVKQTLNFDLLKLAGDVESNPGP (SEQ ID NO:189)). In other embodiments, the cleavable linker is a T2A linker (e.g., having the amino acid sequence GSGEGRGSLLTCGDVEENPGP (SEQ ID NO:190)), a P2A linker (e.g., having the amino acid sequence GSGATNFSLLKQAGDVEENPGP (SEQ ID NO:191)) or an E2A linker (e.g., having the amino acid sequence GSGQCTNYALLKLAGDVESNPGP (SEQ ID NO: 215)). The skilled artisan will appreciate that other art-recognized linkers may be suitable for use in the constructs of the invention (e.g., encoded by the polynucleotides of the invention). The skilled artisan will likewise appreciate that other multicistronic constructs may be suitable for use in the invention. In exemplary embodiments, the construct design yields approximately equimolar amounts of intrabody and/or domain thereof encoded by the constructs of the invention.

In one embodiment, the self-cleaving peptide may be, but is not limited to, a 2A peptide. A variety of 2A peptides are known and available in the art and may be used, including e.g., the foot and mouth disease virus (FMDV) 2A peptide, the equine rhinitis A virus 2A peptide, the Thosea asigna virus 2A peptide, and the porcine teschovirus-1 2A peptide. 2A peptides are used by several viruses to generate two proteins from one transcript by ribosome-skipping, such that a normal peptide bond is impaired at the 2A peptide sequence, resulting in two discontinuous proteins being produced from one translation event. As a non-limiting example, the 2A peptide may have the protein sequence of SEQ ID NO: 191, fragments or variants thereof. In one embodiment, the 2A peptide cleaves between the last glycine and last proline. As another non-limiting example, the polynucleotides of the present invention may include a polynucleotide sequence encoding the 2A peptide having the protein sequence of fragments or variants of SEQ ID NO: 191. One example of a polynucleotide sequence encoding the 2A peptide is: GGAAGCGGAGCUACUAACUUCAGCCUGCUGAAGCAGGCUGGAGACGUGG AGGAGAACCCUGGACCU (SEQ ID NO: 216). In one illustrative embodiment, a 2A peptide is encoded by the following sequence: 5′-UCCGGACUCAGAUCCGGGGAUCUCAAAAUUGUCGCUCCUGUCAAACAAAC UCUUAACUUUGAUUUACUCAAACUGGCTGGGGAUGUAGAAAGCAAUCCAG GTCCACUC-3′(SEQ ID NO: 217). The polynucleotide sequence of the 2A peptide may be modified or codon optimized by the methods described herein and/or are known in the art.

In one embodiment, this sequence may be used to separate the coding regions of two or more polypeptides of interest. As a non-limiting example, the sequence encoding the F2A peptide may be between a first coding region A and a second coding region B (A-F2Apep-B). The presence of the F2A peptide results in the cleavage of the one long protein between the glycine and the proline at the end of the F2A peptide sequence (NPGP (SEQ ID NO:205) is cleaved to result in NPG and P) thus creating separate protein A (with 21 amino acids of the F2A peptide attached, ending with NPG) and separate protein B (with 1 amino acid, P, of the F2A peptide attached). Likewise, for other 2A peptides (P2A, T2A and E2A), the presence of the peptide in a long protein results in cleavage between the glycine and proline at the end of the 2A peptide sequence (NPGP (SEQ ID NO:205) is cleaved to result in NPG and P). Protein A and protein B may be the same or different peptides or polypeptides of interest (e.g., an PCCA or PCCB polypeptide such as full length human PCCA or PCCB).

### Sequence Optimization of Nucleotide Sequence Encoding an PCCA or PCCB Polypeptide

The polynucleotide (e.g., a RNA, e.g., an mRNA) of the invention is sequence optimized. In some embodiments, the polynucleotide (e.g., a RNA, e.g., an mRNA) of the invention comprises a nucleotide sequence (e.g., an ORF) encoding an PCCA or PCCB polypeptide, optionally, a nucleotide sequence (e.g, an ORF) encoding another polypeptide of interest, a 5′-UTR, a 3′-UTR, the 5′ UTR or 3′ UTR optionally comprising at least one microRNA binding site, optionally a nucleotide sequence encoding a linker, a polyA tail, or any combination thereof), in which the ORF(s) are sequence optimized.

A sequence-optimized nucleotide sequence, e.g., a codon-optimized mRNA sequence encoding an PCCA or PCCB polypeptide, is a sequence comprising at least one synonymous nucleobase substitution with respect to a reference sequence (e.g., a wild type nucleotide sequence encoding an PCCA or PCCB polypeptide).

A sequence-optimized nucleotide sequence can be partially or completely different in sequence from the reference sequence. For example, a reference sequence encoding polyserine uniformly encoded by UCU codons can be sequence-optimized by having 100% of its nucleobases substituted (for each codon, U in position 1 replaced by A, C in position 2 replaced by G, and U in position 3 replaced by C) to yield a sequence encoding polyserine which would be uniformly encoded by AGC codons. The percentage of sequence identity obtained from a global pairwise alignment between the reference polyserine nucleic acid sequence and the sequence-optimized polyserine nucleic acid sequence would be 0%. However, the protein products from both sequences would be 100% identical.

Some sequence optimization (also sometimes referred to codon optimization) methods are known in the art (and discussed in more detail below) and can be useful to achieve one or more desired results. These results can include, e.g., matching codon frequencies in certain tissue targets and/or host organisms to ensure proper folding; biasing G/C content to increase mRNA stability or reduce secondary structures; minimizing tandem repeat codons or base runs that can impair gene construction or expression; customizing transcriptional and translational control regions; inserting or removing protein trafficking sequences; removing/adding post translation modification sites in an encoded protein (e.g., glycosylation sites); adding, removing or shuffling protein domains; inserting or deleting restriction sites; modifying ribosome binding sites and mRNA degradation sites; adjusting translational rates to allow the various domains of the protein to fold properly; and/or reducing or eliminating problem secondary structures within the polynucleotide. Sequence optimization tools, algorithms and services are known in the art, non-limiting examples include services from GeneArt (Life Technologies), DNA2.0 (Menlo Park CA) and/or proprietary methods.

Codon options for each amino acid are given in TABLE 1.

TABLE 1
Codon Options

| | Single Letter | |
|---|---|---|
| Amino Acid | Code | Codon Options |
| Isoleucine | I | AUU, AUC, AUA |
| Leucine | L | CUU, CUC, CUA, CUG, UUA, UUG |
| Valine | V | GUU, GUC, GUA, GUG |
| Phenylalanine | F | UUU, UUC |
| Methionine | M | AUG |
| Cysteine | C | UGU, UGC |
| Alanine | A | GCU, GCC, GCA, GCG |
| Glycine | G | GGU, GGC, GGA, GGG |
| Proline | P | CCU, CCC, CCA, CCG |
| Threonine | T | ACU, ACC, ACA, ACG |
| Serine | S | UCU, UCC, UCA, UCG, AGU, AGC |
| Tyrosine | Y | UAU, UAC |
| Tryptophan | W | UGG |
| Glutamine | Q | CAA, CAG |
| Asparagine | N | AAU, AAC |
| Histidine | H | CAU, CAC |
| Glutamic acid | E | GAA, GAG |
| Aspartic acid | D | GAU, GAC |
| Lysine | K | AAA, AAG |
| Arginine | R | CGU, CGC, CGA, CGG, AGA, AGG |
| Selenocysteine | Sec | UGA in mRNA in presence of |
| | | Selenocysteine insertion element |
| | | (SECIS) |
| Stop codons | Stop | UAA, UAG, UGA |

In some embodiments, a polynucleotide (e.g., a RNA, e.g., an mRNA) of the invention comprises a sequence-optimized nucleotide sequence (e.g., an ORF) encoding an PCCA or PCCB polypeptide, a functional fragment, or a variant thereof, wherein the PCCA or PCCB polypeptide, functional fragment, or a variant thereof encoded by the sequence-optimized nucleotide sequence has improved properties (e.g., compared to an PCCA or PCCB polypeptide, functional fragment, or a variant thereof encoded by a reference nucleotide sequence that is not sequence optimized), e.g., improved properties related to expression efficacy after administration in vivo. Such properties include, but are not limited to, improving nucleic acid stability (e.g., mRNA stability), increasing translation efficacy in the target tissue, reducing the number of truncated proteins expressed, improving the folding or prevent misfolding of the expressed proteins, reducing toxicity of the expressed products, reducing cell death caused by the expressed products, increasing and/or decreasing protein aggregation.

In some embodiments, the sequence-optimized nucleotide sequence (e.g., an ORF) is codon optimized for expression in human subjects, having structural and/or chemical features that avoid one or more of the problems in the art, for example, features which are useful for optimizing Formulation and delivery of nucleic acid-based therapeutics while retaining structural and functional integrity; overcoming a threshold of expression; improving expression rates; half-life and/or protein concentrations; optimizing protein localization; and avoiding deleterious bio-responses such as the immune response and/or degradation pathways.

In some embodiments, the polynucleotides of the invention comprise a nucleotide sequence (e.g., a nucleotide sequence (e.g., an ORF) encoding an PCCA or PCCB polypeptide, a nucleotide sequence (e.g., an ORF) encoding another polypeptide of interest, a 5′-UTR, a 3′-UTR, a microRNA binding site, a nucleic acid sequence encoding a linker, or any combination thereof) that is sequence-optimized according to a method comprising:
(i) substituting at least one codon in a reference nucleotide sequence (e.g., an ORF encoding an PCCA or PCCB polypeptide) with an alternative codon to increase or decrease uridine content to generate a uridine-modified sequence;
(ii) substituting at least one codon in a reference nucleotide sequence (e.g., an ORF encoding an PCCA or PCCB polypeptide) with an alternative codon having a higher codon frequency in the synonymous codon set;
(iii) substituting at least one codon in a reference nucleotide sequence (e.g., an ORF encoding an PCCA or PCCB polypeptide) with an alternative codon to increase G/C content; or
(iv) a combination thereof.

In some embodiments, the sequence-optimized nucleotide sequence (e.g., an ORF encoding an PCCA or PCCB polypeptide) has at least one improved property with respect to the reference nucleotide sequence.

In some embodiments, the sequence optimization method is multiparametric and comprises one, two, three, four, or more methods disclosed herein and/or other optimization methods known in the art.

Features, which can be considered beneficial in some embodiments of the invention, can be encoded by or within regions of the polynucleotide and such regions can be upstream (5′) to, downstream (3′) to, or within the region that encodes the PCCA or PCCB polypeptide. These regions can be incorporated into the polynucleotide before and/or after sequence-optimization of the protein encoding region or open reading frame (ORF). Examples of such features include, but are not limited to, untranslated regions (UTRs), microRNA sequences, Kozak sequences, oligo(dT) sequences, poly-A tail, and detectable tags and can include multiple cloning sites that can have XbaI recognition.

In some embodiments, the polynucleotide of the invention comprises a 5′ UTR, a 3′ UTR and/or a microRNA binding site. In some embodiments, the polynucleotide comprises two or more 5′ UTRs and/or 3′ UTRs, which can be the same or different sequences. In some embodiments, the polynucleotide comprises two or more microRNA binding sites, which can be the same or different sequences. Any portion of the 5′ UTR, 3′ UTR, and/or microRNA binding site, including none, can be sequence-optimized and can independently contain one or more different structural or chemical modifications, before and/or after sequence optimization.

In some embodiments, after optimization, the polynucleotide is reconstituted and transformed into a vector such as, but not limited to, plasmids, viruses, cosmids, and artificial chromosomes. For example, the optimized polynucleotide can be reconstituted and transformed into chemically competent E. coli, yeast, neurospora, maize, drosophila, etc. where high copy plasmid-like or chromosome structures occur by methods described herein.

### Sequence-Optimized Nucleotide Sequences Encoding PCCA or PCCB Polypeptides

In some embodiments, the polynucleotide of the invention comprises a sequence-optimized nucleotide sequence encoding an PCCA or PCCB polypeptide disclosed herein. In some embodiments, the polynucleotide of the invention comprises an open reading frame (ORF) encoding an PCCA or PCCB polypeptide, wherein the ORF has been sequence optimized.

An exemplary sequence-optimized nucleotide sequence encoding human full length PCCA or PCCB is set forth as SEQ ID NO:5 or SEQ ID NO:6. In some embodiments, the sequence optimized PCCA or PCCB sequence, fragment, and variant thereof are used to practice the methods disclosed herein.

In some embodiments, a polynucleotide of the present disclosure, for example a polynucleotide comprising an mRNA nucleotide sequence encoding an PCCA polypeptide, comprises from 5′ to 3′ end:
(i) a 5′ cap such as provided herein, for example, m⁷Gp-ppGm-A;
(ii) a 5′ UTR, such as the sequences provided herein, for example, SEQ ID NO:55;
(iii) an open reading frame encoding an PCCA polypeptide, e.g., a sequence optimized nucleic acid sequence encoding PCCA set forth as SEQ ID NO:5;
(iv) at least one stop codon (if not present at 5′ terminus of 3′UTR);
(v) a 3′ UTR, such as the sequences provided herein, for example, SEQ ID NO:114; and
(vi) a poly-A tail provided above (e.g., SEQ ID NO:195).

In some embodiments, a polynucleotide of the present disclosure, for example a polynucleotide comprising an mRNA nucleotide sequence encoding an PCCB polypeptide, comprises from 5′ to 3′ end:
(i) a 5′ cap such as provided herein, for example, m7Gp-ppGm-A;
(ii) a 5′ UTR, such as the sequences provided herein, for example, SEQ ID NO:55;
(iii) an open reading frame encoding an PCCB polypeptide, e.g., a sequence optimized nucleic acid sequence encoding PCCB set forth as SEQ ID NO:6;
(iv) at least one stop codon (if not present at 5′ terminus of 3′UTR);
(v) a 3′ UTR, such as the sequences provided herein, for example, SEQ ID NO:114; and
(vi) a poly-A tail provided above (e.g., SEQ ID NO:195).

In some embodiments, a polynucleotide of the present disclosure, for example a polynucleotide comprising an mRNA nucleotide sequence encoding an PCCA polypeptide, comprises from 5′ to 3′ end:
(i) a 5′ cap such as provided herein, for example, m⁷Gp-ppGm-A;
(ii) a 5′ UTR, such as the sequences provided herein, for example, one of SEQ ID NOs:50-79;
(iii) an open reading frame encoding an PCCA polypeptide, e.g., a sequence optimized nucleic acid sequence encoding PCCA set forth as SEQ ID NO:5;
(iv) at least one stop codon (if not present at 5′ terminus of 3′UTR);
(v) a 3′ UTR, such as the sequences provided herein, for example, one of SEQ ID NOs:100-136; and
(vi) a poly-A tail provided above (e.g., SEQ ID NO:195).

In some embodiments, a polynucleotide of the present disclosure, for example a polynucleotide comprising an mRNA nucleotide sequence encoding an PCCB polypeptide, comprises from 5′ to 3′ end:
(i) a 5′ cap such as provided herein, for example, m⁷Gp-ppGm-A;
(ii) a 5′ UTR, such as the sequences provided herein, for example, one of SEQ ID NOs:50-79;
(iii) an open reading frame encoding an PCCB polypeptide, e.g., a sequence optimized nucleic acid sequence encoding PCCB set forth as SEQ ID NO:6;
(iv) at least one stop codon (if not present at 5′ terminus of 3′UTR);
(v) a 3′ UTR, such as the sequences provided herein, for example, one of SEQ ID NOs:100-136; and
(vi) a poly-A tail provided above (e.g., SEQ ID NO:195).

In certain embodiments, all uracils in the polynucleotide are N1-methylpseudouracil (G5). In certain embodiments, all uracils in the polynucleotide are 5-methoxyuracil (G6).

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the percentage of uracil or thymine nucleobases in a sequence-optimized nucleotide sequence (e.g., encoding an PCCA or PCCB polypeptide, a functional fragment, or a variant thereof) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the invention is greater than the uracil or thymine content in the reference wild-type sequence and still maintain beneficial effects, e.g., increased expression and/or reduced Toll-Like Receptor (TLR) response when compared to the reference wild-type sequence.

Methods for optimizing codon usage are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

### Characterization of Sequence Optimized Nucleic Acids

In some embodiments of the invention, the polynucleotide (e.g., a RNA, e.g., an mRNA) comprising a sequence optimized nucleic acid disclosed herein encoding an PCCA or PCCB polypeptide can be tested to determine whether at least one nucleic acid sequence property (e.g., stability when exposed to nucleases) or expression property has been improved with respect to the non-sequence optimized nucleic acid.

As used herein, “expression property” refers to a property of a nucleic acid sequence either in vivo (e.g., translation efficacy of a synthetic mRNA after administration to a subject in need thereof) or in vitro (e.g., translation efficacy of a synthetic mRNA tested in an in vitro model system). Expression properties include but are not limited to the amount of protein produced by an mRNA encoding an PCCA or PCCB polypeptide after administration, and the amount of soluble or otherwise functional protein produced. In some embodiments, sequence optimized nucleic acids disclosed herein can be evaluated according to the viability of the cells expressing a protein encoded by a sequence optimized nucleic acid sequence (e.g., a RNA, e.g., an mRNA) encoding an PCCA or PCCB polypeptide disclosed herein.

In a given embodiment, a plurality of sequence optimized nucleic acids disclosed herein (e.g., a RNA, e.g., an mRNA) containing codon substitutions with respect to the non-optimized reference nucleic acid sequence can be characterized functionally to measure a property of interest, for example an expression property in an in vitro model system, or in vivo in a target tissue or cell.

a. Optimization of Nucleic Acid Sequence Intrinsic Properties

In some embodiments of the invention, the desired property of the polynucleotide is an intrinsic property of the nucleic acid sequence. For example, the nucleotide sequence (e.g., a RNA, e.g., an mRNA) can be sequence optimized for in vivo or in vitro stability. In some embodiments, the nucleotide sequence can be sequence optimized for expression in a given target tissue or cell. In some embodiments, the nucleic acid sequence is sequence optimized to increase its plasma half-life by preventing its degradation by endo and exonucleases.

In other embodiments, the nucleic acid sequence is sequence optimized to increase its resistance to hydrolysis in solution, for example, to lengthen the time that the sequence optimized nucleic acid or a pharmaceutical composition comprising the sequence optimized nucleic acid can be stored under aqueous conditions with minimal degradation.

In other embodiments, the sequence optimized nucleic acid can be optimized to increase its resistance to hydrolysis in dry storage conditions, for example, to lengthen the time that the sequence optimized nucleic acid can be stored after lyophilization with minimal degradation.

b. Nucleic Acids Sequence Optimized for Protein Expression

In some embodiments of the invention, the desired property of the polynucleotide is the level of expression of an PCCA or PCCB polypeptide encoded by a sequence optimized sequence disclosed herein. Protein expression levels can be measured using one or more expression systems. In some embodiments, expression can be measured in cell culture systems, e.g., CHO cells or HEK293 cells. In some embodiments, expression can be measured using in vitro expression systems prepared from extracts of living cells, e.g., rabbit reticulocyte lysates, or in vitro expression systems prepared by assembly of purified individual components. In other embodiments, the protein expression is measured in an in vivo system, e.g., mouse, rabbit, monkey, etc.

In some embodiments, protein expression in solution form can be desirable. Accordingly, in some embodiments, a reference sequence can be sequence optimized to yield a sequence optimized nucleic acid sequence having optimized levels of expressed proteins in soluble form. Levels of protein expression and other properties such as solubility, levels of aggregation, and the presence of truncation products (i.e., fragments due to proteolysis, hydrolysis, or defective translation) can be measured according to methods known in the art, for example, using electrophoresis (e.g., native or SDS-PAGE) or chromatographic methods (e.g., HPLC, size exclusion chromatography, etc.).

c. Optimization of Target Tissue or Target Cell Viability

In some embodiments, the expression of heterologous therapeutic proteins encoded by a nucleic acid sequence can have deleterious effects in the target tissue or cell, reducing protein yield, or reducing the quality of the expressed product (e.g., due to the presence of protein fragments or precipitation of the expressed protein in inclusion bodies), or causing toxicity.

Accordingly, in some embodiments of the invention, the sequence optimization of a nucleic acid sequence disclosed herein, e.g., a nucleic acid sequence encoding an PCCA or PCCB polypeptide, can be used to increase the viability of target cells expressing the protein encoded by the sequence optimized nucleic acid.

Heterologous protein expression can also be deleterious to cells transfected with a nucleic acid sequence for autologous or heterologous transplantation. Accordingly, in some embodiments of the present disclosure the sequence optimization of a nucleic acid sequence disclosed herein can be used to increase the viability of target cells expressing the protein encoded by the sequence optimized nucleic acid sequence. Changes in cell or tissue viability, toxicity, and other physiological reaction can be measured according to methods known in the art.

d. Reduction of Immune and/or Inflammatory Response

In some cases, the administration of a sequence optimized nucleic acid encoding PCCA or PCCB polypeptide or a functional fragment thereof can trigger an immune response, which could be caused by (i) the therapeutic agent (e.g., an mRNA encoding an PCCA or PCCB polypeptide), or (ii) the expression product of such therapeutic agent (e.g., the PCCA or PCCB polypeptide encoded by the mRNA), or (iv) a combination thereof. Accordingly, in some embodiments of the present disclosure the sequence optimization of nucleic acid sequence (e.g., an mRNA) disclosed herein can be used to decrease an immune or inflammatory response triggered by the administration of a nucleic acid encoding an PCCA or PCCB polypeptide or by the expression product of PCCA or PCCB encoded by such nucleic acid.

In some cases, an inflammatory response can be measured by detecting increased levels of one or more inflammatory cytokines using methods known in the art, e.g., ELISA. The term “inflammatory cytokine” refers to cytokines that are elevated in an inflammatory response. Examples of inflammatory cytokines include interleukin-6 (IL-6), CXCL1 (chemokine (C—X—C motif) ligand 1; also known as GROα, interferon-γ (IFNγ), tumor necrosis factor α (TNFα), interferon γ-induced protein 10 (IP-10), or granulocyte-colony stimulating factor (G-CSF). The term inflammatory cytokines includes also other cytokines associated with inflammatory responses known in the art, e.g., interleukin-1 (IL-1), interleukin-8 (IL-8), interleukin-12 (IL-12), interleukin-13 (Il-13), interferon α (IFN-α), etc.

### Modified Nucleotide Sequences Encoding PCCA or PCCB Polypeptides

In some embodiments, the polynucleotide (e.g., a RNA, e.g., an mRNA) of the invention comprises a chemically modified nucleobase, for example, a chemically modified uracil, e.g., pseudouracil, N1-methylpseudouracil, 5-methoxyuracil, or the like. In some embodiments, the mRNA is a uracil-modified sequence comprising an ORF encoding an PCCA or PCCB polypeptide, wherein the mRNA comprises a chemically modified nucleobase, for example, a chemically modified uracil, e.g., pseudouracil, N1-methylpseudouracil, or 5-methoxyuracil.

In certain aspects of the invention, when the modified uracil base is connected to a ribose sugar, as it is in polynucleotides, the resulting modified nucleoside or nucleotide is referred to as modified uridine. In some embodiments, uracil in the polynucleotide is at least about 25%, at least about 30%, at least about 40%, at least about 50%, at least about 60%, at least about 70%, at least about 80%, at least 90%, at least 95%, at least 99%, or about 100% modified uracil. In one embodiment, uracil in the polynucleotide is at least 95% modified uracil. In another embodiment, uracil in the polynucleotide is 100% modified uracil.

In embodiments where uracil in the polynucleotide is at least 95% modified uracil overall uracil content can be adjusted such that an mRNA provides suitable protein expression levels while inducing little to no immune response. In some embodiments, the uracil content of the ORF is between about 100% and about 150%, between about 100% and about 110%, between about 105% and about 115%, between about 110% and about 120%, between about 115% and about 125%, between about 120% and about 130%, between about 125% and about 135%, between about 130% and about 140%, between about 135% and about 145%, between about 140% and about 150% of the theoretical minimum uracil content in the corresponding wild-type ORF (% Um). In other embodiments, the uracil content of the ORF is between about 121% and about 136% or between 123% and 134% of the % U_{TM}. In some embodiments, the uracil content of the ORF encoding an PCCA or PCCB polypeptide is about 115%, about 120%, about 125%, about 130%, about 135%, about 140%, about 145%, or about 150% of the % UM. In this context, the term “uracil” can refer to modified uracil and/or naturally occurring uracil.

In some embodiments, the uracil content in the ORF of the mRNA encoding an PCCA or PCCB polypeptide of the invention is less than about 30%, about 25%, about 20%, about 15%, or about 10% of the total nucleobase content in the ORF. In some embodiments, the uracil content in the ORF is between about 10% and about 20% of the total nucleobase content in the ORF. In other embodiments, the uracil content in the ORF is between about 10% and about 25% of the total nucleobase content in the ORF. In one embodiment, the uracil content in the ORF of the mRNA encoding an PCCA or PCCB polypeptide is less than about 20% of the total nucleobase content in the open reading frame. In this context, the term “uracil” can refer to modified uracil and/or naturally occurring uracil.

In further embodiments, the ORF of the mRNA encoding an PCCA or PCCB polypeptide having modified uracil and adjusted uracil content has increased Cytosine (C), Guanine (G), or Guanine/Cytosine (G/C) content (absolute or relative). In some embodiments, the overall increase in C, G, or G/C content (absolute or relative) of the ORF is at least about 2%, at least about 3%, at least about 4%, at least about 5%, at least about 6%, at least about 7%, at least about 10%, at least about 15%, at least about 20%, at least about 40%, at least about 50%, at least about 60%, at least about 70%, at least about 80%, at least about 90%, at least about 95%, or at least about 100% relative to the G/C content (absolute or relative) of the wild-type ORF. In some embodiments, the G, the C, or the G/C content in the ORF is less than about 100%, less than about 90%, less than about 85%, or less than about 80% of the theoretical maximum G, C, or G/C content of the corresponding wild type nucleotide sequence encoding the PCCA or PCCB polypeptide (% G_{TMX}; % C_{TMX}, or % G/C_{TMX}). In some embodiments, the increases in G and/or C content (absolute or relative) described herein can be conducted by replacing synonymous codons with low G, C, or G/C content with synonymous codons having higher G, C, or G/C content. In other embodiments, the increase in G and/or C content (absolute or relative) is conducted by replacing a codon ending with U with a synonymous codon ending with G or C.

In further embodiments, the ORF of the mRNA encoding an PCCA or PCCB polypeptide of the invention comprises modified uracil and has an adjusted uracil content containing less uracil pairs (UU) and/or uracil triplets (UUU) and/or uracil quadruplets (UUUU) than the corresponding wild-type nucleotide sequence encoding the PCCA or PCCB polypeptide. In some embodiments, the ORF of the mRNA encoding an PCCA or PCCB polypeptide of the invention contains no uracil pairs and/or uracil triplets and/or uracil quadruplets. In some embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding the PCCA or PCCB polypeptide. In a particular embodiment, the ORF of the mRNA encoding the PCCA or PCCB polypeptide of the invention contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the ORF of the mRNA encoding the PCCA or PCCB polypeptide contains no non-phenylalanine uracil pairs and/or triplets.

In further embodiments, the ORF of the mRNA encoding an PCCA or PCCB polypeptide of the invention comprises modified uracil and has an adjusted uracil content containing less uracil-rich clusters than the corresponding wild-type nucleotide sequence encoding the PCCA or PCCB polypeptide. In some embodiments, the ORF of the mRNA encoding the PCCA or PCCB polypeptide of the invention contains uracil-rich clusters that are shorter in length than corresponding uracil-rich clusters in the corresponding wild-type nucleotide sequence encoding the PCCA or PCCB polypeptide.

In further embodiments, alternative lower frequency codons are employed. At least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the PCCA or PCCB polypeptide-encoding ORF of the modified uracil-comprising mRNA are substituted with alternative codons, each alternative codon having a codon frequency lower than the codon frequency of the substituted codon in the synonymous codon set. The ORF also has adjusted uracil content, as described above. In some embodiments, at least one codon in the ORF of the mRNA encoding the PCCA or PCCB polypeptide is substituted with an alternative codon having a codon frequency lower than the codon frequency of the substituted codon in the synonymous codon set.

In some embodiments, the adjusted uracil content, PCCA or PCCB polypeptide-encoding ORF of the modified uracil-comprising mRNA exhibits expression levels of PCCA or PCCB when administered to a mammalian cell that are higher than expression levels of PCCA or PCCB from the corresponding wild-type mRNA. In some embodiments, the mammalian cell is a mouse cell, a rat cell, or a rabbit cell. In other embodiments, the mammalian cell is a monkey cell or a human cell. In some embodiments, the human cell is a HeLa cell, a BJ fibroblast cell, or a peripheral blood mononuclear cell (PBMC). In some embodiments, PCCA or PCCB is expressed at a level higher than expression levels of PCCA or PCCB from the corresponding wild-type mRNA when the mRNA is administered to a mammalian cell in vivo. In some embodiments, the mRNA is administered to mice, rabbits, rats, monkeys, or humans. In one embodiment, mice are null mice. In some embodiments, the mRNA is administered to mice in an amount of about 0.01 mg/kg, about 0.05 mg/kg, about 0.1 mg/kg, or 0.2 mg/kg or about 0.5 mg/kg. In some embodiments, the mRNA is administered intravenously or intramuscularly. In other embodiments, the PCCA or PCCB polypeptide is expressed when the mRNA is administered to a mammalian cell in vitro. In some embodiments, the expression is increased by at least about 2-fold, at least about 5-fold, at least about 10-fold, at least about 50-fold, at least about 500-fold, at least about 1500-fold, or at least about 3000-fold. In other embodiments, the expression is increased by at least about 10%, about 20%, about 30%, about 40%, about 50%, 60%, about 70%, about 80%, about 90%, or about 100%.

In some embodiments, adjusted uracil content, PCCA or PCCB polypeptide-encoding ORF of the modified uracil-comprising mRNA exhibits increased stability. In some embodiments, the mRNA exhibits increased stability in a cell relative to the stability of a corresponding wild-type mRNA under the same conditions. In some embodiments, the mRNA exhibits increased stability including resistance to nucleases, thermal stability, and/or increased stabilization of secondary structure. In some embodiments, increased stability exhibited by the mRNA is measured by determining the half-life of the mRNA (e.g., in a plasma, serum, cell, or tissue sample) and/or determining the area under the curve (AUC) of the protein expression by the mRNA over time (e.g., in vitro or in vivo). An mRNA is identified as having increased stability if the half-life and/or the AUC is greater than the half-life and/or the AUC of a corresponding wild-type mRNA under the same conditions.

In some embodiments, the mRNA of the present invention induces a detectably lower immune response (e.g., innate or acquired) relative to the immune response induced by a corresponding wild-type mRNA under the same conditions. In other embodiments, the mRNA of the present disclosure induces a detectably lower immune response (e.g., innate or acquired) relative to the immune response induced by an mRNA that encodes for an PCCA or PCCB polypeptide but does not comprise modified uracil under the same conditions, or relative to the immune response induced by an mRNA that encodes for an PCCA or PCCB polypeptide and that comprises modified uracil but that does not have adjusted uracil content under the same conditions. The innate immune response can be manifested by increased expression of pro-inflammatory cytokines, activation of intracellular PRRs (RIG-I, MDA5, etc), cell death, and/or termination or reduction in protein translation. In some embodiments, a reduction in the innate immune response can be measured by expression or activity level of Type 1 interferons (e.g., IFN-α, IFN-β, IFN-κ, IFN-δ, IFN-ε, IFN-τ, IFN-ω, and IFN-ζ) or the expression of interferon-regulated genes such as the toll-like receptors (e.g., TLR7 and TLR8), and/or by decreased cell death following one or more administrations of the mRNA of the invention into a cell.

In some embodiments, the expression of Type-1 interferons by a mammalian cell in response to the mRNA of the present disclosure is reduced by at least 10%, 20%, 30%, 40%, 50%, 60%, 70%, 80%, 90%, 95%, 99%, 99.9%, or greater than 99.9% relative to a corresponding wild-type mRNA, to an mRNA that encodes an PCCA or PCCB polypeptide but does not comprise modified uracil, or to an mRNA that encodes an PCCA or PCCB polypeptide and that comprises modified uracil but that does not have adjusted uracil content. In some embodiments, the interferon is IFN-β. In some embodiments, cell death frequency caused by administration of mRNA of the present disclosure to a mammalian cell is 10%, 25%, 50%, 75%, 85%, 90%, 95%, or over 95% less than the cell death frequency observed with a corresponding wild-type mRNA, an mRNA that encodes for an PCCA or PCCB polypeptide but does not comprise modified uracil, or an mRNA that encodes for an PCCA or PCCB polypeptide and that comprises modified uracil but that does not have adjusted uracil content. In some embodiments, the mammalian cell is a BJ fibroblast cell. In other embodiments, the mammalian cell is a splenocyte. In some embodiments, the mammalian cell is that of a mouse or a rat. In other embodiments, the mammalian cell is that of a human. In one embodiment, the mRNA of the present disclosure does not substantially induce an innate immune response of a mammalian cell into which the mRNA is introduced.

### Methods for Modifying Polynucleotides

The disclosure includes modified polynucleotides comprising a polynucleotide described herein (e.g., a polynucleotide, e.g. mRNA, comprising a nucleotide sequence encoding an PCCA or PCCB polypeptide). The modified polynucleotides can be chemically modified and/or structurally modified. When the polynucleotides of the present invention are chemically and/or structurally modified the polynucleotides can be referred to as “modified polynucleotides.”

The present disclosure provides for modified nucleosides and nucleotides of a polynucleotide (e.g., RNA polynucleotides, such as mRNA polynucleotides) encoding an PCCA or PCCB polypeptide. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides. Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some embodiments, the modified polynucleotides contain one, two, or more (optionally different) nucleoside or nucleotide modifications. In some embodiments, a modified polynucleotide, introduced to a cell can exhibit one or more desirable properties, e.g., improved protein expression, reduced immunogenicity, or reduced degradation in the cell, as compared to an unmodified polynucleotide.

In some embodiments, a polynucleotide of the present invention (e.g., a polynucleotide comprising a nucleotide sequence encoding an PCCA or PCCB polypeptide) is structurally modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” can be chemically modified to “AT-5meC-G”. The same polynucleotide can be structurally modified from “ATCG” to “ATCCCG”. Here, the dinucleotide “CC” has been inserted, resulting in a structural modification to the polynucleotide.

Therapeutic compositions of the present disclosure comprise, in some embodiments, at least one nucleic acid (e.g., RNA) having an open reading frame encoding PCCA or PCCB (e.g., SEQ ID NO:1 or SEQ ID NO:2), wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

In some embodiments, at least one RNA (e.g., mRNA) of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises N1-methyl-pseudouridine (m1ψ) substitutions at one or more or all uridine positions of the nucleic acid.

In some embodiments, a RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

Untranslated regions (UTRs) are nucleic acid sections of a polynucleotide before a start codon (5′ UTR) and after a stop codon (3′ UTR) that are not translated. In some embodiments, a polynucleotide (e.g., a ribonucleic acid (RNA), e.g., a messenger RNA (mRNA)) of the invention comprising an open reading frame (ORF) encoding an PCCA or PCCB polypeptide further comprises UTR (e.g., a 5′ UTR or functional fragment thereof, a 3′ UTR or functional fragment thereof, or a combination thereof).

A UTR (e.g., 5′ UTR or 3′ UTR) can be homologous or heterologous to the coding region in a polynucleotide. In some embodiments, the UTR is homologous to the ORF encoding the PCCA or PCCB polypeptide. In some embodiments, the UTR is heterologous to the ORF encoding the PCCA or PCCB polypeptide.

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO: 214), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Additional exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 a polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human a or R actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the R subunit of mitochondrial H⁺-ATP synthase); a growth hormone e (e.g., bovine (bGH) or human (hGH)); an elongation factor (e.g., elongation factor 1 al (EEF1A1)); a manganese superoxide dismutase (MnSOD); a myocyte enhancer factor 2A (MEF2A); a 3-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′UTR containing a strong Kozak translational initiation signal; a cytochrome b-245 a polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a R subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation.

a. 5′ UTR sequences

5′ UTR sequences are important for ribosome recruitment to the mRNA and have been reported to play a role in translation (Hinnebusch A, et al., (2016) Science, 352:6292: 1413-6).

Disclosed herein, inter alia, is a polynucleotide, e.g., mRNA, comprising an open reading frame encoding an PCCA or PCCB polypeptide (e.g., SEQ ID NO:1 or SEQ ID NO:2), which polynucleotide has a 5′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as provided in Table 2 or a variant or fragment thereof); (b) a coding region comprising a stop element (e.g., as described herein); and (c) a 3′-UTR (e.g., as described herein), and LNP compositions comprising the same. In an embodiment, the polynucleotide comprises a 5′-UTR comprising a sequence provided in Table 2 or a variant or fragment thereof (e.g., a functional variant or fragment thereof).

In an embodiment, the polynucleotide having a 5′ UTR sequence provided in Table 2 or a variant or fragment thereof, has an increase in the half-life of the polynucleotide, e.g., about 1.5-20-fold increase in half-life of the polynucleotide. In an embodiment, the increase in half-life is about 1.5, 2, 3, 4, 5, 6, 7, 8, 9, 10, 11, 12, 13, 14, 15, 16, 17, 18, 19 or 20-fold, or more. In an embodiment, the increase in half life is about 1.5-fold or more. In an embodiment, the increase in half life is about 2-fold or more. In an embodiment, the increase in half life is about 3-fold or more. In an embodiment, the increase in half life is about 4-fold or more. In an embodiment, the increase in half life is about 5-fold or more.

In an embodiment, the polynucleotide having a 5′ UTR sequence provided in Table 2 or a variant or fragment thereof, results in an increased level and/or activity, e.g., output, of the polypeptide encoded by the polynucleotide. In an embodiment, the 5′UTR results in about 1.5-20-fold increase in level and/or activity, e.g., output, of the polypeptide encoded by the polynucleotide. In an embodiment, the increase in level and/or activity is about 1.5, 2, 3, 4, 5, 6, 7, 8, 9, 10, 11, 12, 13, 14, 15, 16, 17, 18, 19 or 20-fold, or more. In an embodiment, the increase in level and/or activity is about 1.5-fold or more. In an embodiment, the increase in level and/or activity is about 2-fold or more. In an embodiment, the increase in level and/or activity is about 3-fold or more. In an embodiment, the increase in level and/or activity is about 4-fold or more. In an embodiment, the increase in level and/or activity is about 5-fold or more.

In an embodiment, the increase is compared to an otherwise similar polynucleotide which does not have a 5′ UTR, has a different 5′ UTR, or does not have a 5′ UTR described in Table 2 or a variant or fragment thereof.

In an embodiment, the increase in half-life of the polynucleotide is measured according to an assay that measures the half-life of a polynucleotide.

In an embodiment, the increase in level and/or activity, e.g., output, of the polypeptide encoded by the polynucleotide is measured according to an assay that measures the level and/or activity of a polypeptide.

In an embodiment, the 5′ UTR comprises a sequence provided in Table 2 or a sequence with at least 80%, 85%, 90%, 95%, 96%, 97%, 98%, 99% or 100% identity to a 5′ UTR sequence provided in Table 2, or a variant or a fragment thereof. In an embodiment, the 5′ UTR comprises a sequence with at least 80%, 85%, 90%, 95%, 96%, 97%, 98%, 99% or 100% identity to SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or SEQ ID NO: 78.

In an embodiment, the 5′ UTR comprises a sequence with at least 80%, 85%, 90%, 95%, 96%, 97%, 98%, 99% or 100% identity to SEQ ID NO: 50. In an embodiment, the 5′ UTR comprises a sequence with at least 80%, 85%, 90%, 95%, 96%, 97%, 98%, 99% or 100% identity to SEQ ID NO: 51. In an embodiment, the 5′ UTR comprises a sequence with at least 80%, 85%, 90%, 95%, 96%, 97%, 98%, 99% or 100% identity to SEQ ID NO: 52. In an embodiment, the 5′ UTR comprises a sequence with at least 80%, 85%, 90%, 95%, 96%, 97%, 98%, 99% or 100% identity to SEQ ID NO: 53. In an embodiment, the 5′ UTR comprises a sequence with at least 80%, 85%, 90%, 95%, 96%, 97%, 98%, 99% or 100% identity to SEQ ID NO: 54. In an embodiment, the 5′ UTR comprises a sequence with at least 80%, 85%, 90%, 95%, 96%, 97%, 98%, 99% or 100% identity to SEQ ID NO: 55. In an embodiment, the 5′ UTR comprises a sequence with at least 80%, 85%, 90%, 95%, 96%, 97%, 98%, 99% or 100% identity to SEQ ID NO: 56. In an embodiment, the 5′ UTR comprises a sequence with at least 80%, 85%, 90%, 95%, 96%, 97%, 98%, 99% or 100% identity to SEQ ID NO: 57. In an embodiment, the 5′ UTR comprises a sequence with at least 80%, 85%, 90%, 95%, 96%, 97%, 98%, 99% or 100% identity to SEQ ID NO: 58. In an embodiment, the 5′ UTR comprises a sequence with at least 80%, 85%, 90%, 95%, 96%, 97%, 98%, 99% or 100% identity to SEQ ID NO: 78.

In an embodiment, the 5′ UTR comprises the sequence of SEQ ID NO:78. In an embodiment, the 5′ UTR consists of the sequence of SEQ ID NO:78.

In an embodiment, the 5′ UTR comprises the sequence of SEQ ID NO:55. In an embodiment, the 5′ UTR consists of the sequence of SEQ ID NO:55.

In an embodiment, the 5′ UTR comprises the sequence of SEQ ID NO:56. In an embodiment, the 5′ UTR consists of the sequence of SEQ ID NO:56.

In an embodiment, a 5′ UTR sequence provided in Table 2 has a first nucleotide which is an A. In an embodiment, a 5′ UTR sequence provided in Table 2 has a first nucleotide which is a G.

TABLE 2
5′ UTR sequences

| SEQ ID | Sequence | |
|---|---|---|
| NO: | name | Sequence |
| 50 | A1 | GGAAAUCGCAAAAUUUGCUCUUCGCGUUAGAUUUCUUUUAG |
| | | UUUUCUCGCAACUAGCAAGCUUUUUGUUCUCGCC |
| 51 | A5 | GGAAAUCCCCACAACCGCCUCAUAUCCAGGCUCAAGAAUAG |
| | | AGCUCAGUGUUUUGUUGUUUAAUCAUUCCGACGUGUUUUGC |
| | | GAUAUUCGCGCAAAGCAGCCAGUCGCGCGCUUGCUUUUAAG |
| | | UAGAGUUGUUUUUCCACCCGUUUGCCAGGCAUCUUUAAUUU |
| | | AACAUAUUUUUAUUUUUCAGGCUAACCUACGCCGCCACC |
| 52 | A6 | GGAAAUAAGAGAGAAAAGAAGAGUAAGAAGAAAUAUAAGAU |
| | | CUCCCUGAGCUUCAGGGAGCCCCGGCGCCGCCACC |
| 53 | A7 | GGAAACCCCCCACCCCCGUAAGAGAGAAAAGAAGAGUAAGA |
| | | AGAAAUAUAAGAUCUCCCUGAGCUUCAGGGAGCCCCGGCGC |
| | | CGCCACC |
| 54 | A8 | GGAGAACUUCCGCUUCCGUUGGCGCAAGCGCUUUCAUUUUU |
| | | UCUGCUACCGUGACUAAG |
| 55 | A9 | GGAAAUAAGAGAGAAAAGAAGAGUAAGAAGAAAUAUAAGAG |
| | | CCACC |
| 56 | A11 | GGAAAUAAGAGAGAAAAGAAGAGUAAGAAGAAAUAUAAGAC |
| | (Reference) | CCCGGCGCCGCCACC |
| 57 | A2 | GGAAAUCGCAAAAUUUGCUCUUCGCGUUAGAUUUCUUUUAG |
| | | UUUUCUCGCAACUAGCAAGCUUUUUGUUCUCGCCGCCGCC |
| 58 | A3 | GGAAAUCGCAAAAUUUUCUUUUCGCGUUAGAUUUCUUUUAG |
| | | UUUUCUUUCAACUAGCAAGCUUUUUGUUCUCGCCGCCGCC |
| 59 | A4 | G G A A A U C G C A A A A (N₂)ₓ (N₃)ₓ C U |
| | | (N₄)_{X} (N₅)_{X} C G C G U U A G A U U U C U U U |
| | | U A G U U U U C U N₆ N₇ C A A C U A G C A |
| | | A G C U U U U U G U U C U C G C C (N8 C |
| | | C) x |
| | | (N₂)ₓ is a uracil and x is an integer from 0 to 5, e.g., wherein |
| | | x = 3 or 4; |
| | | (N₃)ₓ is a guanine and x is an integer from 0 to 1; |
| | | (N₄)ₓ is a cytosine and x is an integer from 0 to 1; |
| | | (N₅)ₓ is a uracil and x is an integer from 0 to 5, e.g., wherein |
| | | x = 2 or 3; |
| | | N₆ is a uracil or cytosine; |
| | | N₇ is a uracil or guanine; |
| | | N₈ is adenine or guanine and x is an integer from 0 to 1. |
| 60 | A27 | GGAAAAUUUUAGCCUGGAACGUUAGAUAACUGUCCUGUUGU |
| | | CUUUAUAUACUUGGUCCCCAAGUAGUUUGUCUUCCAAA |
| 61 | A12 | GGAAACUUUAUUUAGUGUUACUUUAUUUUCUGUUUAUUUGU |
| | | GUUUCUUCAGUGGGUUUGUUCUAAUUUCCUUGGCCGCC |
| 62 | A13 | GGAAAAUCUGUAUUAGGUUGGCGUGUUCUUUGGUCGGUUGU |
| | | UAGUAUUGUUGUUGAUUCGUUUGUGGUCGGUUGCCGCC |
| 63 | A14 | GGAAAAUUAUUAACAUCUUGGUAUUCUCGAUAACCAUUCGU |
| | | UGGAUUUUAUUGUAUUCGUAGUUUGGGUUCCUGCCGCC |
| 64 | A15 | GGAAAUUAUUAUUAUUUCUAGCUACAAUUUAUCAUUGUAUU |
| | | AUUUUAGCUAUUCAUCAUUAUUUACUUGGUGAUCAACA |
| 65 | A16 | GGAAAUAGGUUGUUAACCAAGUUCAAGCCUAAUAAGCUUGG |
| | | AUUCUGGUGACUUGCUUCACCGUUGGCGGGCACCGAUC |
| 66 | A17 | GGAAAUCGUAGAGAGUCGUACUUAGUACAUAUCGACUAUCG |
| | | GUGGACACCAUCAAGAUUAUAAACCAGGCCAGA |
| 67 | A18 | GGAAACCCGCCCAAGCGACCCCAACAUAUCAGCAGUUGCCC |
| | | AAUCCCAACUCCCAACACAAUCCCCAAGCAACGCCGCC |
| 68 | A19 | GGAAAGCGAUUGAAGGCGUCUUUUCAACUACUCGAUUAAGG |
| | | UUGGGUAUCGUCGUGGGACUUGGAAAUUUGUUGUUUCC |
| 69 | A20 | GGAAACUAAUCGAAAUAAAAGAGCCCCGUACUCUUUUAUUU |
| | | CUAUUAGGUUAGGAGCCUUAGCAUUUGUAUCUUAGGUA |
| 70 | A21 | GGAAAUGUGAUUUCCAGCAACUUCUUUUGAAUAUAUUGAAU |
| | | UCCUAAUUCAAAGCGAACAAAUCUACAAGCCAUAUACC |
| 71 | A22 | GGAAAUCGUAGAGAGUCGUACUUACGUGGUCGCCAUUGCAU |
| | | AGCGCGCGAAAGCAACAGGAACAAGAACGCGCC |
| 72 | A23 | GGAAAUCGUAGAGAGUCGUACUUAGAAUAAACAGAGUCGGG |
| | | UCGACUUGUCUCUGAUACUACGACGUCACAAUC |
| 73 | A24 | GGAAAAUUUGCCUUCGGAGUUGCGUAUCCUGAACUGCCCAG |
| | | CCUCCUGAUAUACAACUGUUCCGCUUAUUCGGGCCGCC |
| 74 | A25 | GGAAAUCUGAGCAGGAAUCCUUUGUGCAUUGAAGACUUUAG |
| | | AUUCCUCUCUGCGGUAGACGUGCACUUAUAAGUAUUUG |
| 75 | A26 | GGAAAGCGAUUGAAGGCGUCUUUUCAACUACUCGAUUAAGG |
| | | UUGGGUAUCGUCGUGGGACUUGGAAAUUUGUUGCCACC |
| 76 | A28 | GGAAAUUUUUUUUUGAUAUUAUAAGAGUUUUUUUUUGAUAU |
| | | UAAGAAAAUUUUUUUUUGAUAUUAGAAGAGUAAGAAGAAAU |
| | | AUAAGACCCCGGCGCCGCCACC |
| 77 | A29 | GGAAAUAAGAGAGAAAAGAAGAGUAAGAAGAAAUAUAAGAG |
| | | CCAAAAAAAAAAAACC |
| 78 | A30 | GGAAAUCUCCCUGAGCUUCAGGGAGUAAGAGAGAAAAGAAG |
| | | AGUAAGAAGAAAUAUAAGACCCCGGCGCCGCCACC |
| 79 | A31 | GCCRCC, wherein R = A or G |

In an embodiment, the 5′ UTR comprises a variant of SEQ TD NO: 50. In an embodiment, the variant of SEQ ID NO: 50 comprises a nucleic acid sequence of Formula A:

(SEQ ID NO: 59)
G G A A A U C G C A A A A (N₂)x (N₃)x C U (N₄)x
(N₅)x C G C G U U A G A U U U C U U U U A G U U U
U C U N₆ N₇ C A A C U A G C A A G C U U U U U G U
U C U C G C C (N₈ C C)x,

wherein:
(N₂)ₓ is a uracil and x is an integer from 0 to 5, e.g., wherein x=3 or 4;
(N₃)ₓ is a guanine and x is an integer from 0 to 1;
(N₄)ₓ is a cytosine and x is an integer from 0 to 1;
(N₅)ₓ is a uracil and x is an integer from 0 to 5, e.g., wherein x=2 or 3;
N₆ is a uracil or cytosine;
N₇ is a uracil or guanine;
N₈ is adenine or guanine and x is an integer from 0 to 1.

In an embodiment (N₂)ₓ is a uracil and x is 0. In an embodiment (N₂)ₓ is a uracil and x is 1. In an embodiment (N₂)ₓ is a uracil and x is 2. In an embodiment (N₂)ₓ is a uracil and x is 3. In an embodiment, (N₂)ₓ is a uracil and x is 4. In an embodiment (N₂)ₓ is a uracil and x is 5.

In an embodiment, (N₃)ₓ is a guanine and x is 0. In an embodiment, (N₃)ₓ is a guanine and x is 1.

In an embodiment, (N₄)ₓ is a cytosine and x is 0. In an embodiment, (N₄)ₓ is a cytosine and x is 1.

In an embodiment (N₅)ₓ is a uracil and x is 0. In an embodiment (N₅)ₓ is a uracil and x is 1. In an embodiment (N₅)ₓ is a uracil and x is 2. In an embodiment (N₅)ₓ is a uracil and x is 3. In an embodiment, (N₅)ₓ is a uracil and x is 4. In an embodiment (N₅)ₓ is a uracil and x is 5.

In an embodiment, N₆ is a uracil. In an embodiment, N₆ is a cytosine.

In an embodiment, N₇ is a uracil. In an embodiment, N₇ is a guanine.

In an embodiment, N₈ is an adenine and x is 0. In an embodiment, N₈ is an adenine and x is 1.

In an embodiment, N₈ is a guanine and x is 0. In an embodiment, N₈ is a guanine and x is 1.

In an embodiment, the 5′ UTR comprises a variant of SEQ ID NO: 50. In an embodiment, the variant of SEQ ID NO: 50 comprises a sequence with at least 50%, 60%, 70%, 80%, 85%, 90%, 95%, 96%, 97%, 98%, or 99% identity to SEQ ID NO: 50. In an embodiment, the variant of SEQ ID NO: 50 comprises a sequence with at least 50% identity to SEQ ID NO: 50. In an embodiment, the variant of SEQ ID NO: 50 comprises a sequence with at least 60% identity to SEQ ID NO: 50. In an embodiment, the variant of SEQ ID NO: 50 comprises a sequence with at least 70% identity to SEQ ID NO: 50. In an embodiment, the variant of SEQ ID NO: 50 comprises a sequence with at least 80% identity to SEQ ID NO: 50. In an embodiment, the variant of SEQ ID NO: 50 comprises a sequence with at least 90% identity to SEQ ID NO: 50. In an embodiment, the variant of SEQ ID NO: 50 comprises a sequence with at least 95% identity to SEQ ID NO: 50. In an embodiment, the variant of SEQ ID NO: 50 comprises a sequence with at least 96% identity to SEQ ID NO: 50. In an embodiment, the variant of SEQ ID NO: 50 comprises a sequence with at least 97% identity to SEQ ID NO: 50. In an embodiment, the variant of SEQ ID NO: 50 comprises a sequence with at least 98% identity to SEQ ID NO: 50. In an embodiment, the variant of SEQ ID NO: 50 comprises a sequence with at least 99% identity to SEQ ID NO: 50.

In an embodiment, the variant of SEQ ID NO: 50 comprises a uridine content of at least 5%, 10%, 20%, 30%, 40%, 50%, 60%, 70%, or 80%. In an embodiment, the variant of SEQ ID NO: 50 comprises a uridine content of at least 5%. In an embodiment, the variant of SEQ ID NO: 50 comprises a uridine content of at least 10%. In an embodiment, the variant of SEQ ID NO: 50 comprises a uridine content of at least 20%. In an embodiment, the variant of SEQ ID NO: 50 comprises a uridine content of at least 30%. In an embodiment, the variant of SEQ ID NO: 50 comprises a uridine content of at least 40%. In an embodiment, the variant of SEQ ID NO: 50 comprises a uridine content of at least 50%. In an embodiment, the variant of SEQ ID NO: 50 comprises a uridine content of at least 60%. In an embodiment, the variant of SEQ ID NO: 50 comprises a uridine content of at least 70%. In an embodiment, the variant of SEQ ID NO: 50 comprises a uridine content of at least 80%.

In an embodiment, the variant of SEQ ID NO: 50 comprises at least 2, 3, 4, 5, 6 or 7 consecutive uridines (e.g., a polyuridine tract). In an embodiment, the polyuridine tract in the variant of SEQ ID NO: 50 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO: 50 comprises 4 consecutive uridines. In an embodiment, the polyuridine tract in the variant of SEQ ID NO: 50 comprises 5 consecutive uridines.

In an embodiment, the variant of SEQ ID NO: 50 comprises 1, 2, 3, 4, 5, 6, 7, 8, 9, 10, 11, 12, 13, 14, or 15 polyuridine tracts. In an embodiment, the variant of SEQ ID NO: 50 comprises 3 polyuridine tracts. In an embodiment, the variant of SEQ ID NO: 50 comprises 4 polyuridine tracts. In an embodiment, the variant of SEQ ID NO: 50 comprises 5 polyuridine tracts.

In an embodiment, one or more of the polyuridine tracts are adjacent to a different polyuridine tract. In an embodiment, each of, e.g., all, the polyuridine tracts are adjacent to each other, e.g., all of the polyuridine tracts are contiguous.

In an embodiment, one or more of the polyuridine tracts are separated by 1, 2, 3, 4, 5, 6, 7, 8, 9, 10, 11, 2, 13, 14, 15, 16, 17, 18, 19, 20, 30, 40, 50 or 60 nucleotides. In an embodiment, each of, e.g., all of, the polyuridine tracts are separated by 1, 2, 3, 4, 5, 6, 7, 8, 9, 10, 11, 2, 13, 14, 15, 16, 17, 18, 19, 20, 30, 40, 50 or 60 nucleotides.

In an embodiment, a first polyuridine tract and a second polyuridine tract are adjacent to each other.

In an embodiment, a subsequent, e.g., third, fourth, fifth, sixth or seventh, eighth, ninth, or tenth, polyuridine tract is separated by 1, 2, 3, 4, 5, 6, 7, 8, 9, 10, 11, 2, 13, 14, 15, 16, 17, 18, 19, 20, 30, 40, 50 or 60 nucleotides from the first polyuridine tract, the second polyuridine tract, or any one of the subsequent polyuridine tracts.

In an embodiment, a first polyuridine tract is separated by 1, 2, 3, 4, 5, 6, 7, 8, 9, 10, 11, 2, 13, 14, 15, 16, 17, 18, 19, 20, 30, 40, 50 or 60 nucleotides from a subsequent polyuridine tract, e.g., a second, third, fourth, fifth, sixth or seventh, eighth, ninth, or tenth polyuridine tract. In an embodiment, one or more of the subsequent polyuridine tracts are adjacent to a different polyuridine tract.

In an embodiment, the 5′ UTR comprises a Kozak sequence, e.g., a GCCRCC nucleotide sequence (SEQ ID NO: 79) wherein R is an adenine or guanine. In an embodiment, the Kozak sequence is disposed at the 3′ end of the 5′UTR sequence.

In an aspect, the polynucleotide (e.g., mRNA) comprising an open reading frame encoding an PCCA or PCCB polypeptide (e.g., SEQ ID NO:1 or SEQ ID NO:2) and comprising a 5′ UTR sequence disclosed herein is formulated as an LNP. In an embodiment, the LNP composition comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating propionic acidemia in a subject.

In an aspect, an LNP composition comprising a polynucleotide disclosed herein encoding an PCCA or PCCB polypeptide, e.g., as described herein, can be administered with an additional agent, e.g., as described herein.

### b. 3′ UTR Sequences

3′UTR sequences have been shown to influence translation, half-life, and subcellular localization of mRNAs (Mayr C., Cold Spring Harb Persp Biol 2019 Oct. 1;11(10):a034728).

Disclosed herein, inter alia, is a polynucleotide, e.g., mRNA, comprising an open reading frame encoding an PCCA or PCCB polypeptide (e.g., SEQ ID NO:1 or SEQ ID NO:2), which polynucleotide has a 3′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as described herein); (b) a coding region comprising a stop element (e.g., as described herein); and (c) a 3′-UTR (e.g., as provided in Table 3 or a variant or fragment thereof), and LNP compositions comprising the same. In an embodiment, the polynucleotide comprises a 3′-UTR comprising a sequence provided in Table 3 or a variant or fragment thereof.

In an embodiment, the polynucleotide having a 3′ UTR sequence provided in Table 3 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more. In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in Table 3 or a variant or fragment thereof, results in a polynucleotide with a mean half-life score of greater than 10.

In an embodiment, the polynucleotide having a 3′ UTR sequence provided in Table 3 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of Table 3 or a variant or fragment thereof.

In an embodiment, the polynucleotide comprises a 3′ UTR sequence provided in Table 3 or a sequence with at least 80%, 85%, 90%, 95%, 96%, 97%, 98%, 99% or 100% identity to a 3′ UTR sequence provided in Table 3, or a fragment thereof. In an embodiment, the 3′ UTR comprises a sequence with at least 80%, 85%, 90%, 95%, 96%, 97%, 98%, 99% or 100% identity to SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO:115, or SEQ ID NO:136.

In an embodiment, the 3′ UTR comprises the sequence of SEQ ID NO: 100, or a sequence with at least 80%, 85%, 90%, 95%, 96%, 97%, 98%, 99% or 100% identity to SEQ ID NO: 100. In an embodiment, the 3′ UTR comprises the sequence of SEQ ID NO: 101, or a sequence with at least 80%, 85%, 90%, 95%, 96%, 97%, 98%, 99% or 100% identity to SEQ ID NO: 101. In an embodiment, the 3′ UTR comprises the sequence of SEQ ID NO: 102, or a sequence with at least 80%, 85%, 90%, 95%, 96%, 97%, 98%, 99% or 100% identity to SEQ ID NO: 102. In an embodiment, the 3′ UTR comprises the sequence of SEQ ID NO: 103, or a sequence with at least 80%, 85%, 90%, 95%, 96%, 97%, 98%, 99% or 100% identity to SEQ ID NO: 103. In an embodiment, the 3′ UTR comprises the sequence of SEQ ID NO: 104, or a sequence with at least 80%, 85%, 90%, 95%, 96%, 97%, 98%, 99% or 100% identity to SEQ ID NO: 104. In an embodiment, the 3′ UTR comprises the sequence of SEQ ID NO: 105, or a sequence with at least 80%, 85%, 90%, 95%, 96%, 97%, 98%, 99% or 100% identity to SEQ ID NO: 105. In an embodiment, the 3′ UTR comprises the sequence of SEQ ID NO: 106, or a sequence with at least 80%, 85%, 90%, 95%, 96%, 97%, 98%, 99% or 100% identity to SEQ ID NO: 106. In an embodiment, the 3′ UTR comprises the sequence of SEQ ID NO: 107, or a sequence with at least 80%, 85%, 90%, 95%, 96%, 97%, 98%, 99% or 100% identity to SEQ ID NO: 107. In an embodiment, the 3′ UTR comprises the sequence of SEQ ID NO: 108, or a sequence with at least 80%, 85%, 90%, 95%, 96%, 97%, 98%, 99% or 100% identity to SEQ ID NO: 108. In an embodiment, the 3′ UTR comprises the sequence of SEQ ID NO: 109, or a sequence with at least 80%, 85%, 90%, 95%, 96%, 97%, 98%, 99% or 100% identity to SEQ ID NO: 109. In an embodiment, the 3′ UTR comprises the sequence of SEQ ID NO: 110, or a sequence with at least 80%, 85%, 90%, 95%, 96%, 97%, 98%, 99% or 100% identity to SEQ ID NO: 110. In an embodiment, the 3′ UTR comprises the sequence of SEQ ID NO: 111, or a sequence with at least 80%, 85%, 90%, 95%, 96%, 97%, 98%, 99% or 100% identity to SEQ ID NO: 111. In an embodiment, the 3′ UTR comprises the sequence of SEQ ID NO: 112, or a sequence with at least 80%, 85%, 90%, 95%, 96%, 97%, 98%, 99% or 100% identity to SEQ ID NO: 112. In an embodiment, the 3′ UTR comprises the sequence of SEQ ID NO: 113, or a sequence with at least 80%, 85%, 90%, 95%, 96%, 97%, 98%, 99% or 100% identity to SEQ ID NO: 113. In an embodiment, the 3′ UTR comprises the sequence of SEQ TD NO: 114, or a sequence with at least 80%, 85%, 90%, 95%, 96%, 97%, 98%, 99% or 100% identity to SEQ ID NO: 114. In an embodiment, the 3 UTR comprises the sequence of SEQ ID NO: 115, or a sequence with at least 80%, 85%, 90%, 95%, 96%, 97%, 98%, 99% or 100% identity to SEQ ID NO: 115. In an embodiment, the 3′ UTR comprises the sequence of SEQ TD NO: 136, or a sequence with at least 80%, 85%, 90%, 95%, 96%, 97%, 98%, 99% or 100% identity to SEQ ID NO: 136.

TABLE 3
3′ UTR sequences

| SEQ | Sequence | |
|---|---|---|
| ID NO | information | Sequence |
| 100 | B1 | UAAUAGUAAGCUGGAGCCUCCUGAGAGACCUGUGUGAACUA |
| | | UUGAGAAGAUCGGAACAGCUCCUUACUCUGAGGAAGUUGGU |
| | | ACCCCCGUGGUCUUUGAAUAAAGUCUGAGUGGGCGGC |
| 101 | B3 | UAAUAGUAAGCUGGAGCCUCACUCUCCUCUCCAUCCCGUAU |
| | | CCAGGCUGUGAAUUUUUCAAGGAAUAUAAAGAUCGGGAUGU |
| | | ACCCCCGUGGUCUUUGAAUAAAGUCUGAGUGGGCGGC |
| 102 | B4 | UGAUAGUAAGCUGGAGCCUCUAGUGACGGCAACAGGGCUUG |
| | | GUUUUUCCUUGUUGUGAAAUCGACAUCUCUGAAGACAGGGU |
| | | ACCCCCGUGGUCUUUGAAUAAAGUCUGAGUGGGCGGC |
| 103 | B5 | UGAUAGUAAGCUGGAGCCUCCUUCCAUCUAGUCACAAAGAC |
| | | UCCUUCGUCCCCAGUUGCCGUCUAGGAUUGGGCCUCCCAGU |
| | | ACCCCCGUGGUCUUUGAAUAAAGUCUGAGUGGGCGGC |
| 104 | B6 | UGAUAGUAAGCUGGAGCCUCCCAUAACAUGACAUAUCUGGA |
| | | UUUUGUGCUUAGAACCUUAAAUUGGAAGCAUUCUUAAUUGU |
| | | ACCCCCGUGGUCUUUGAAUAAAGUCUGAGUGGGCGGC |
| 105 | B7 | UAAUAGUAAGCUGGAGCCUCCGGAAAACUAAAAUAGAGAUA |
| | | UUUCAAGAUUUUAUAAUUUUCAAAGACCUUUGAAAUAUUGU |
| | | ACCCCCGUGGUCUUUGAAUAAAGUCUGAGUGGGCGGC |
| 106 | B8 | UAAUAGUAAGCUGGAGCCUCUACACAUUGCUUCUAGUUGGC |
| | | AGAAAUAAUUGAUUAAAAGACCAGAAACUGUGAUAACUGGU |
| | | ACCCCCGUGGUCUUUAAAUAAAGUCUAAGUGGGCGGC |
| 107 | B9 | UGAUAAUAGGCUGGAGCCUCGGUGGCCAUGCUUCUUGCCCC |
| | | UUGGGCCUCCCCCCAGCCCCUCCUCCCCUUCCUGCACCCGU |
| | | ACCCCCGUGGUCUUUGAAUAAAGUCUGAGUGGGGGGC |
| 108 | B10 | UGAUAAUAGGCUGGAGCCUCGGUGGCCUAGCUUCUUGCCCC |
| | | UUGGGCCUCCCCCCAGCCCCUCCUCCCCUUCCUGCACCCGU |
| | | ACCCCCGUGGUCUUUGAAUAAAGUCUGAGUGGGGGGC |
| 109 | B11 | UGAUAAUAGGCUGGAGCCUCGGUGGCCAUGCUUCUUGCCCC |
| | | UUGGGCCUCCCCCCAGCCCCUCCUCCCCUUCCUGCACCCGU |
| | | ACCCCCCAAACACCAUUGUCACACUCCAGUGGUCUUUGAAU |
| | | AAAGUCUGAGUGGGCGGC |
| 110 | B12 | UGAUAAUAGGCUGGAGCCUCGGUGGCCUAGCUUCUUGCCCC |
| | | UUGGGCCUCCCCCCAGCCCCUCCUCCCCUUCCUGCACCCGU |
| | | ACCCCCCAAACACCAUUGUCACACUCCAGUGGUCUUUGAAU |
| | | AAAGUCUGAGUGGGGGGC |
| 111 | B13 | UGAUAAUAGGCUGGAGCCUCGGUGGCCUAGCUUCUUGCCCC |
| | | UUGGGCCUCCCCCCAGCCCCUCCUCCCCUUCCUGCACCCGU |
| | | ACCCCCUCCAUAAAGUAGGAAACACUACAGUGGUCUUUGAA |
| | | UAAAGUCUGAGUGGGGGGC |
| 112 | B14 | UGAUAAUAGGCUGGAGCCUCGGUGGCCUAGCUUCUUGCCCC |
| | | UUGGGCCUCCCCCCAGCCCCUCCUCCCCUUCCUGCACCCGU |
| | | ACCCCCCGCAUUAUUACUCACGGUACGAGUGGUCUUUGAAU |
| | | AAAGUCUGAGUGGGCGGC |
| 113 | B15 | UGAUAAUAGUCCAUAAAGUAGGAAACACUACAGCUGGAGCC |
| | | UCGGUGGCCAUGCUUCUUGCCCCUUGGGCCUCCCCCCAGCC |
| | | CCUCCUCCCCUUCCUGCACCCGUACCCCCCGCAUUAUUACU |
| | | CACGGUACGAGUGGUCUUUGAAUAAAGUCUGAGUGGGGGGC |
| 114 | B16 | UGAUAAUAGUCCAUAAAGUAGGAAACACUACAGCUGGAGCC |
| | | UCGGUGGCCUAGCUUCUUGCCCCUUGGGCCUCCAUAAAGUA |
| | | GGAAACACUACAUCCCCCCAGCCCCUCCUCCCCUUCCUGCA |
| | | CCCGUACCCCCUCCAUAAAGUAGGAAACACUACAGUGGUCU |
| | | UUGAAUAAAGUCUGAGUGGGGGGC |
| 115 | B2 | CUGAGAGACCUGUGUGAACUAUUGAGAAGAUCGGAACAGCU |
| | | CCUUACUCUGAGGAAGUUG |
| 116 | B17 | UGAUAAUAGGCUGGAGCCUCUCACACACCUCUGCCCCUUGG |
| | | GCCUCCCACUCCCAUGGCUCUGGGCGGUCCAGAAGGAGCGU |
| | | ACCCCCGUGGUCUUUGAAUAAAGUCUGAGUGGGGGGC |
| 117 | B18 | UGAUAAUAGGCUGGAGCCUCCACCGCGUUAUCCGUUCCUCG |
| | | UAGGCUGGUCCUGGGGAACGGGUCGGCGGGUACCCCCGUGG |
| | | UCUUUGAAUAAAGUCUGAGUGGGCGGC |
| 118 | B19 | UGAUAAUAGGCUGGAGCCUCUGCCCGGCAACGGCCAGGUCU |
| | | GUGCCAAGUGUUUGCUGACGCAACCCCCACUGGCUGGGGCU |
| | | UGGUCAUGGGCCAUCAGCGCGUGCGUGGAACCUUUUCGGCU |
| | | CCUCUGCCGAUCCAUACUGCGGAACUCCUAGCCGCUUGUUU |
| | | UGCUCGCAGCAGGUCUGGAGCAAACAUUAUCGGGACUGAUA |
| | | ACUCUGUUGUCCUGUACCCCCGUGGUCUUUGAAUAAAGUCU |
| | | GAGUGGGCGGC |
| 119 | B20 | UGAUAAUAGGCUGGAGCCUCGGUGGCCUAGCUUCUUGCCCC |
| | | UUGGGCCUCCCCCCAGCCCCUCCUCCCCUUCCUGCACCCGU |
| | | ACCCUUUUUUUUUUUUUUUUUUUCUUCUUUUCUUUUUUUUC |
| | | UUUUUUUUUUUUCUUUCUUUUUUUCUUUUUUUUUCUUUUCU |
| | | UUUUUCUUUUUUUUUUUUUUUUCCGUGGUCUUUGAAUAAAG |
| | | UCUGAGUGGGGGC |
| 120 | B21 | UGAUAAUAGGCUGGAGCCUCGGUGGCCUAGCUUCUUGCCCC |
| | | UUGGGCCUCCCCCCAGCCCCUCCUCCCCUUCCUGCACCCGU |
| | | ACCCUUUUUUUUUUUUUUUUUUUUUUUUUUUUUUUUUUUUU |
| | | UUUUUUUUUUUUUUUUUUUUUUUUUUUUUUUUUUUUUUUUU |
| | | UUUUUUUUUUUUUUUUUUUUUUCCGUGGUCUUUGAAUAAAG |
| | | UCUGAGUGGGCGGC |
| 133 | B22 | UAAGUCUCCAUAAAGUAGGAAACACUACAGCUGGAGCCUCG |
| | | GUGGCCUAGCUUCUUGCCCCUUGGGCCUCCAUAAAGUAGGA |
| | | AACACUACAUCCCCCCAGCCCCUCCUCCCCUUCCUGCACCC |
| | | GUACCCCCUCCAUAAAGUAGGAAACACUACAGUGGUCUUUG |
| | | AAUAAAGUCUGAGUGGGCGGC |
| 134 | B23 | UAAAGCGCUGGAGCCUCGGUGGCCUAGCUUCUUGCCCCUUG |
| | | GGCCUCCCCCCAGCCCCUCCUCCCCUUCCUGCACCCGUACC |
| | | CCCUCCAUAAAGUAGGAAACACUACAGUGGUCUUUGAAUAA |
| | | AGUCUGAGUGGGCGGC |
| 135 | B24 | UAAAGCUCCCCGGGGUCCAUAAAGUAGGAAACACUACAGCU |
| | | GGAGCCUCCUGAGAGACCUGUGUGAACUAUUGAGAAGAUCG |
| | | GAACAGCUCCUUACUCUGAGGAAGUUGUCCAUAAAGUAGGA |
| | | AACACUACAGUACCCCCUCCAUAAAGUAGGAAACACUACAG |
| | | UGGUCUUUGAAUAAAGUCUGAGUGGGCGGC |
| 136 | B25 | UAAUAGUAAACCUCACUCACGGCCACAUUGAGUGCCAGGCU |
| | | CCGGGCUGGUUUAUAGUAGUGUAGAGCAUUGCAGCACUUAG |
| | | ACUGGGGUGCUGUAGUCUUUAUUGUAGUCUUUCCACAUACC |
| | | UGAUAAUUCUUAGAUAAUUUCUUAUUUUAAUUCCAUAAAGU |
| | | AGGAAACACUACAUAAAUCUCCAUAAAGUAGGAAACACUAC |
| | | AUAUUCUUCCAUAAAGUAGGAAACACUACAUAGGCU |

In an embodiment, the 3′ UTR comprises a micro RNA (miRNA) binding site, e.g., as described herein, which binds to a miR present in a human cell. In an embodiment, the 3′ UTR comprises a miRNA binding site of SEQ ID NO: 212, SEQ ID NO: 174, SEQ ID NO: 152 or a combination thereof. In an embodiment, the 3′ UTR comprises a plurality of miRNA binding sites, e.g., 2, 3, 4, 5, 6, 7 or 8 miRNA binding sites. In an embodiment, the plurality of miRNA binding sites comprises the same or different miRNA binding sites.

(SEQ ID NO: 212)
miR122 bs = CAAACACCAUUGUCACACUCCA
(SEQ ID NO: 174)
miR-142-3p bs = UCCAUAAAGUAGGAAACACUACA
(SEQ ID NO: 152)
miR-126 bs = CGCAUUAUUACUCACGGUACGA

In an aspect, disclosed herein is a polynucleotide encoding a polypeptide, wherein the polynucleotide comprises: (a) a 5′-UTR, e.g., as described herein; (b) a coding region comprising a stop element (e.g., as described herein); and (c) a 3′-UTR (e.g., as described herein).

In an aspect, an LNP composition comprising a polynucleotide comprising an open reading frame encoding an PCCA or PCCB polypeptide (e.g., SEQ ID NO:1 or SEQ ID NO:2) and comprising a 3′ UTR disclosed herein comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating propionic acidemia in a subject.

In an aspect, an LNP composition comprising a polynucleotide disclosed herein encoding an PCCA or PCCB polypeptide, e.g., as described herein, can be administered with an additional agent, e.g., as described herein.

MicroRNA (miRNA) Binding Sites

Polynucleotides of the invention can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, polynucleotides including such regulatory elements are referred to as including “sensor sequences”.

In some embodiments, a polynucleotide (e.g., a ribonucleic acid (RNA), e.g., a messenger RNA (mRNA)) of the invention comprises an open reading frame (ORF) encoding a polypeptide of interest and further comprises one or more miRNA binding site(s). Inclusion or incorporation of miRNA binding site(s) provides for regulation of polynucleotides of the invention, and in turn, of the polypeptides encoded therefrom, based on tissue-specific and/or cell-type specific expression of naturally-occurring miRNAs.

The present invention also provides pharmaceutical compositions and Formulations that comprise any of the polynucleotides described above. In some embodiments, the composition or Formulation further comprises a delivery agent.

In some embodiments, the composition or Formulation can contain a polynucleotide comprising a sequence optimized nucleic acid sequence disclosed herein which encodes a polypeptide. In some embodiments, the composition or Formulation can contain a polynucleotide (e.g., a RNA, e.g., an mRNA) comprising a polynucleotide (e.g., an ORF) having significant sequence identity to a sequence optimized nucleic acid sequence disclosed herein which encodes a polypeptide. In some embodiments, the polynucleotide further comprises a miRNA binding site, e.g., a miRNA binding site that binds

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA.

microRNAs derive enzymatically from regions of RNA transcripts that fold back on themselves to form short hairpin structures often termed a pre-miRNA (precursor-miRNA). A pre-miRNA typically has a two-nucleotide overhang at its 3′ end, and has 3′ hydroxyl and 5′ phosphate groups. This precursor-mRNA is processed in the nucleus and subsequently transported to the cytoplasm where it is further processed by DICER (a RNase III enzyme), to form a mature microRNA of approximately 22 nucleotides. The mature microRNA is then incorporated into a ribonuclear particle to form the RNA-induced silencing complex, RISC, which mediates gene silencing. Art-recognized nomenclature for mature miRNAs typically designates the arm of the pre-miRNA from which the mature miRNA derives; “5p” means the microRNA is from the 5 prime arm of the pre-miRNA hairpin and “3p” means the microRNA is from the 3 prime end of the pre-miRNA hairpin. A miR referred to by number herein can refer to either of the two mature microRNAs originating from opposite arms of the same pre-miRNA (e.g., either the 3p or 5p microRNA). All miRs referred to herein are intended to include both the 3p and 5p arms/sequences, unless particularly specified by the 3p or 5p designation.

As used herein, the term “microRNA (miRNA or miR) binding site” refers to a sequence within a polynucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polynucleotide of the invention comprising an ORF encoding a polypeptide of interest and further comprises one or more miRNA binding site(s). In exemplary embodiments, a 5′ UTR and/or 3′ UTR of the polynucleotide (e.g., a ribonucleic acid (RNA), e.g., a messenger RNA (mRNA)) comprises the one or more miRNA binding site(s).

A miRNA binding site having sufficient complementarity to a miRNA refers to a degree of complementarity sufficient to facilitate miRNA-mediated regulation of a polynucleotide, e.g., miRNA-mediated translational repression or degradation of the polynucleotide. In exemplary aspects of the invention, a miRNA binding site having sufficient complementarity to the miRNA refers to a degree of complementarity sufficient to facilitate miRNA-mediated degradation of the polynucleotide, e.g., miRNA-guided RNA-induced silencing complex (RISC)-mediated cleavage of mRNA. The miRNA binding site can have complementarity to, for example, a 19-25 nucleotide long miRNA sequence, to a 19-23 nucleotide long miRNA sequence, or to a 22 nucleotide long miRNA sequence. A miRNA binding site can be complementary to only a portion of a miRNA, e.g., to a portion less than 1, 2, 3, or 4 nucleotides of the full length of a naturally-occurring miRNA sequence, or to a portion less than 1, 2, 3, or 4 nucleotides shorter than a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with an miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In other embodiments, the sequence is not completely complementary. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polynucleotide of the invention, the polynucleotide can be targeted for degradation or reduced translation, provided the miRNA in question is available. This can reduce off-target effects upon delivery of the polynucleotide. For example, if a polynucleotide of the invention is not intended to be delivered to a tissue or cell but ends up is said tissue or cell, then a miRNA abundant in the tissue or cell can inhibit the expression of the gene of interest if one or multiple binding sites of the miRNA are engineered into the 5′ UTR and/or 3′ UTR of the polynucleotide. Thus, in some embodiments, incorporation of one or more miRNA binding sites into an mRNA of the disclosure may reduce the hazard of off-target effects upon nucleic acid molecule delivery and/or enable tissue-specific regulation of expression of a polypeptide encoded by the mRNA. In yet other embodiments, incorporation of one or more miRNA binding sites into an mRNA of the disclosure can modulate immune responses upon nucleic acid delivery in vivo. In further embodiments, incorporation of one or more miRNA binding sites into an mRNA of the disclosure can modulate accelerated blood clearance (ABC) of lipid-comprising compounds and compositions described herein.

Conversely, miRNA binding sites can be removed from polynucleotide sequences in which they naturally occur to increase protein expression in specific tissues. For example, a binding site for a specific miRNA can be removed from a polynucleotide to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune-response to infection, inflammation, as well as unwanted immune response after gene therapy and tissue/organ transplantation. Immune cells specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing one or more (e.g., one, two, or three) miR-142 binding sites into the 5′ UTR and/or 3′UTR of a polynucleotide of the invention can selectively repress gene expression in antigen presenting cells through miR-142 mediated degradation, limiting antigen presentation in antigen presenting cells (e.g., dendritic cells) and thereby preventing antigen-mediated immune response after the delivery of the polynucleotide. The polynucleotide is then stably expressed in target tissues or cells without triggering cytotoxic elimination.

In some embodiments, it may be beneficial to target the same cell type with multiple miRs and to incorporate binding sites to each of the 3p and 5p arm if both are abundant (e.g., both miR-142-3p and miR142-5p are abundant in hematopoietic stem cells). Thus, in certain embodiments, polynucleotides of the invention contain two or more (e.g., two, three, four or more) miR bindings sites from: (i) the group consisting of miR-142, miR-144, miR-150, miR-155 and miR-223 (which are expressed in many hematopoietic cells); or (ii) the group consisting of miR-142, miR150, miR-16 and miR-223 (which are expressed in B cells); or the group consisting of miR-223, miR-451, miR-26a, miR-16 (which are expressed in progenitor hematopoietic cells).

In some embodiments, it may also be beneficial to combine various miRs such that multiple cell types of interest are targeted at the same time (e.g., miR-142 and miR-126 to target many cells of the hematopoietic lineage and endothelial cells). Thus, for example, in certain embodiments, polynucleotides of the invention comprise two or more (e.g., two, three, four or more) miRNA bindings sites, wherein: (i) at least one of the miRs targets cells of the hematopoietic lineage (e.g., miR-142, miR-144, miR-150, miR-155 or miR-223) and at least one of the miRs targets plasmacytoid dendritic cells, platelets or endothelial cells (e.g., miR-126); or (ii) at least one of the miRs targets B cells (e.g., miR-142, miR150, miR-16 or miR-223) and at least one of the miRs targets plasmacytoid dendritic cells, platelets or endothelial cells (e.g., miR-126); or (iii) at least one of the miRs targets progenitor hematopoietic cells (e.g., miR-223, miR-451, miR-26a or miR-16) and at least one of the miRs targets plasmacytoid dendritic cells, platelets or endothelial cells (e.g., miR-126); or (iv) at least one of the miRs targets cells of the hematopoietic lineage (e.g., miR-142, miR-144, miR-150, miR-155 or miR-223), at least one of the miRs targets B cells (e.g., miR-142, miR150, miR-16 or miR-223) and at least one of the miRs targets plasmacytoid dendritic cells, platelets or endothelial cells (e.g., miR-126); or any other possible combination of the foregoing four classes of miR binding sites (i.e., those targeting the hematopoietic lineage, those targeting B cells, those targeting progenitor hematopoietic cells and/or those targeting plasmacytoid dendritic cells/platelets/endothelial cells).

In one embodiment, to modulate immune responses, polynucleotides of the present invention can comprise one or more miRNA binding sequences that bind to one or more miRs that are expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells). It has now been discovered that incorporation into an mRNA of one or more miRs that are expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells) reduces or inhibits immune cell activation (e.g., B cell activation, as measured by frequency of activated B cells) and/or cytokine production (e.g., production of IL-6, IFN-γ and/or TNFα). Furthermore, it has now been discovered that incorporation into an mRNA of one or more miRs that are expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells) can reduce or inhibit an anti-drug antibody (ADA) response against a protein of interest encoded by the mRNA.

In another embodiment, to modulate accelerated blood clearance of a polynucleotide delivered in a lipid-comprising compound or composition, polynucleotides of the invention can comprise one or more miR binding sequences that bind to one or more miRNAs expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells). It has now been discovered that incorporation into an mRNA of one or more miR binding sites reduces or inhibits accelerated blood clearance (ABC) of the lipid-comprising compound or composition for use in delivering the mRNA. Furthermore, it has now been discovered that incorporation of one or more miR binding sites into an mRNA reduces serum levels of anti-PEG anti-IgM (e.g, reduces or inhibits the acute production of IgMs that recognize polyethylene glycol (PEG) by B cells) and/or reduces or inhibits proliferation and/or activation of plasmacytoid dendritic cells following administration of a lipid-comprising compound or composition comprising the mRNA.

In some embodiments, miR sequences may correspond to any known microRNA expressed in immune cells, including but not limited to those taught in US Publication US2005/0261218 and US Publication US2005/0059005, the contents of which are incorporated herein by reference in their entirety. Non-limiting examples of miRs expressed in immune cells include those expressed in spleen cells, myeloid cells, dendritic cells, plasmacytoid dendritic cells, B cells, T cells and/or macrophages. For example, miR-142-3p, miR-142-5p, miR-16, miR-21, miR-223, miR-24 and miR-27 are expressed in myeloid cells, miR-155 is expressed in dendritic cells, B cells and T cells, miR-146 is upregulated in macrophages upon TLR stimulation and miR-126 is expressed in plasmacytoid dendritic cells. In certain embodiments, the miR(s) is expressed abundantly or preferentially in immune cells. For example, miR-142 (miR-142-3p and/or miR-142-5p), miR-126 (miR-126-3p and/or miR-126-5p), miR-146 (miR-146-3p and/or miR-146-5p) and miR-155 (miR-155-3p and/or miR155-5p) are expressed abundantly in immune cells. These microRNA sequences are known in the art and, thus, one of ordinary skill in the art can readily design binding sequences or target sequences to which these microRNAs will bind based upon Watson-Crick complementarity.

In one embodiment, the polynucleotide of the invention comprises three copies of the same miRNA binding site. In certain embodiments, use of three copies of the same miR binding site can exhibit beneficial properties as compared to use of a single miRNA binding site.

In another embodiment, the polynucleotide of the invention comprises two or more (e.g., two, three, four) copies of at least two different miR binding sites expressed in immune cells.

In another embodiment, the polynucleotide of the invention comprises at least two miR binding sites for microRNAs expressed in immune cells, wherein one of the miR binding sites is for miR-142-3p. In various embodiments, the polynucleotide of the invention comprises binding sites for miR-142-3p and miR-155 (miR-155-3p or miR-155-5p), miR-142-3p and miR-146 (miR-146-3 or miR-146-5p), or miR-142-3p and miR-126 (miR-126-3p or miR-126-5p).

In another embodiment, the polynucleotide of the invention comprises at least two miR binding sites for microRNAs expressed in immune cells, wherein one of the miR binding sites is for miR-126-3p. In various embodiments, the polynucleotide of the invention comprises binding sites for miR-126-3p and miR-155 (miR-155-3p or miR-155-5p), miR-126-3p and miR-146 (miR-146-3p or miR-146-5p), or miR-126-3p and miR-142 (miR-142-3p or miR-142-5p).

In another embodiment, the polynucleotide of the invention comprises at least two miR binding sites for microRNAs expressed in immune cells, wherein one of the miR binding sites is for miR-142-5p. In various embodiments, the polynucleotide of the invention comprises binding sites for miR-142-5p and miR-155 (miR-155-3p or miR-155-5p), miR-142-5p and miR-146 (miR-146-3 or miR-146-5p), or miR-142-5p and miR-126 (miR-126-3p or miR-126-5p).

In yet another embodiment, the polynucleotide of the invention comprises at least two miR binding sites for microRNAs expressed in immune cells, wherein one of the miR binding sites is for miR-155-5p. In various embodiments, the polynucleotide of the invention comprises binding sites for miR-155-5p and miR-142 (miR-142-3p or miR-142-5p), miR-155-5p and miR-146 (miR-146-3 or miR-146-5p), or miR-155-5p and miR-126 (miR-126-3p or miR-126-5p).

In some embodiments, a polynucleotide of the invention comprises a miRNA binding site, wherein the miRNA binding site comprises one or more nucleotide sequences selected from Table 4, including one or more copies of any one or more of the miRNA binding site sequences. In some embodiments, a polynucleotide of the invention further comprises at least one, two, three, four, five, six, seven, eight, nine, ten, or more of the same or different miRNA binding sites selected from Table 4, including any combination thereof.

In some embodiments, the miRNA binding site binds to miR-142 or is complementary to miR-142. In some embodiments, the miR-142 comprises SEQ ID NO:172. In some embodiments, the miRNA binding site binds to miR-142-3p or miR-142-5p. In some embodiments, the miR-142-3p binding site comprises SEQ ID NO:174. In some embodiments, the miR-142-5p binding site comprises SEQ ID NO:210. In some embodiments, the miRNA binding site comprises a nucleotide sequence at least 80%, at least 85%, at least 90%, at least 95%, or 100% identical to SEQ ID NO:174 or SEQ ID NO:210.

In some embodiments, the miRNA binding site binds to miR-126 or is complementary to miR-126. In some embodiments, the miR-126 comprises SEQ ID NO: 150. In some embodiments, the miRNA binding site binds to miR-126-3p or miR-126-5p. In some embodiments, the miR-126-3p binding site comprises SEQ ID NO: 152. In some embodiments, the miR-126-5p binding site comprises SEQ ID NO: 154. In some embodiments, the miRNA binding site comprises a nucleotide sequence at least 80%, at least 85%, at least 90%, at least 95%, or 100% identical to SEQ ID NO: 152 or SEQ ID NO: 154.

In one embodiment, the 3′ UTR comprises two miRNA binding sites, wherein a first miRNA binding site binds to miR-142 and a second miRNA binding site binds to miR-126.

TABLE 4
miR-142, miR-126, and miR-142 and miR-126 binding sites

| SEQ ID NO. | Description | Sequence |
|---|---|---|
| 172 | miR-142 | GACAGUGCAGUCACCCAUAAAGUAGAAAGCACUACU |
| | | AACAGCACUGGAGGGUGUAGUGUUUCCUACUUUAUG |
| | | GAUGAGUGUACUGUG |
| 173 | miR-142-3p | UGUAGUGUUUCCUACUUUAUGGA |
| 174 | miR-142-3p binding site | UCCAUAAAGUAGGAAACACUACA |
| 175 | miR-142-5p | CAUAAAGUAGAAAGCACUACU |
| 210 | miR-142-5p binding site | AGUAGUGCUUUCUACUUUAUG |
| 150 | miR-126 | CGCUGGCGACGGGACAUUAUUACUUUUGGUACGCGC |
| | | UGUGACACUUCAAACUCGUACCGUGAGUAAUAAUGC |
| | | GCCGUCCACGGCA |
| 151 | miR-126-3p | UCGUACCGUGAGUAAUAAUGCG |
| 152 | miR-126-3p binding site | CGCAUUAUUACUCACGGUACGA |
| 153 | miR-126-5p | CAUUAUUACUUUUGGUACGCG |
| 154 | miR-126-5p binding site | CGCGUACCAAAAGUAAUAAUG |

In some embodiments, a miRNA binding site is inserted in the polynucleotide of the invention in any position of the polynucleotide (e.g., the 5′ UTR and/or 3′ UTR). In some embodiments, the 5′ UTR comprises a miRNA binding site. In some embodiments, the 3′ UTR comprises a miRNA binding site. In some embodiments, the 5′ UTR and the 3′ UTR comprise a miRNA binding site. The insertion site in the polynucleotide can be anywhere in the polynucleotide as long as the insertion of the miRNA binding site in the polynucleotide does not interfere with the translation of a functional polypeptide in the absence of the corresponding miRNA; and in the presence of the miRNA, the insertion of the miRNA binding site in the polynucleotide and the binding of the miRNA binding site to the corresponding miRNA are capable of degrading the polynucleotide or preventing the translation of the polynucleotide.

In some embodiments, a miRNA binding site is inserted in at least about 30 nucleotides downstream from the stop codon of an ORF in a polynucleotide of the invention comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the invention. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a polynucleotide of the invention.

In some embodiments, a miRNA binding site is inserted within the 3′ UTR immediately following the stop codon of the coding region within the polynucleotide of the invention, e.g., mRNA. In some embodiments, if there are multiple copies of a stop codon in the construct, a miRNA binding site is inserted immediately following the final stop codon. In some embodiments, a miRNA binding site is inserted further downstream of the stop codon, in which case there are 3′ UTR bases between the stop codon and the miR binding site(s).

In some embodiments, one or more miRNA binding sites can be positioned within the 5′ UTR at one or more possible insertion sites.

In one embodiment, a codon optimized open reading frame encoding a polypeptide of interest comprises a stop codon and the at least one microRNA binding site is located within the 3′ UTR 1-100 nucleotides after the stop codon. In one embodiment, the codon optimized open reading frame encoding the polypeptide of interest comprises a stop codon and the at least one microRNA binding site for a miR expressed in immune cells is located within the 3′ UTR 30-50 nucleotides after the stop codon. In another embodiment, the codon optimized open reading frame encoding the polypeptide of interest comprises a stop codon and the at least one microRNA binding site for a miR expressed in immune cells is located within the 3′ UTR at least 50 nucleotides after the stop codon. In other embodiments, the codon optimized open reading frame encoding the polypeptide of interest comprises a stop codon and the at least one microRNA binding site for a miR expressed in immune cells is located within the 3′ UTR immediately after the stop codon, or within the 3′ UTR 15-20 nucleotides after the stop codon or within the 3′ UTR 70-80 nucleotides after the stop codon. In other embodiments, the 3′ UTR comprises more than one miRNA binding site (e.g., 2-4 miRNA binding sites), wherein there can be a spacer region (e.g., of 10-100, 20-70 or 30-50 nucleotides in length) between each miRNA binding site. In another embodiment, the 3′ UTR comprises a spacer region between the end of the miRNA binding site(s) and the poly A tail nucleotides. For example, a spacer region of 10-100, 20-70 or 30-50 nucleotides in length can be situated between the end of the miRNA binding site(s) and the beginning of the poly A tail.

In one embodiment, a codon optimized open reading frame encoding a polypeptide of interest comprises a start codon and the at least one microRNA binding site is located within the 5′ UTR 1-100 nucleotides before (upstream of) the start codon. In one embodiment, the codon optimized open reading frame encoding the polypeptide of interest comprises a start codon and the at least one microRNA binding site for a miR expressed in immune cells is located within the 5′ UTR 10-50 nucleotides before (upstream of) the start codon. In another embodiment, the codon optimized open reading frame encoding the polypeptide of interest comprises a start codon and the at least one microRNA binding site for a miR expressed in immune cells is located within the 5′ UTR at least 25 nucleotides before (upstream of) the start codon. In other embodiments, the codon optimized open reading frame encoding the polypeptide of interest comprises a start codon and the at least one microRNA binding site for a miR expressed in immune cells is located within the 5′ UTR immediately before the start codon, or within the 5′ UTR 15-20 nucleotides before the start codon or within the 5′ UTR 70-80 nucleotides before the start codon. In other embodiments, the 5′ UTR comprises more than one miRNA binding site (e.g., 2-4 miRNA binding sites), wherein there can be a spacer region (e.g., of 10-100, 20-70 or 30-50 nucleotides in length) between each miRNA binding site.

In one embodiment, the 3′ UTR comprises more than one stop codon, wherein at least one miRNA binding site is positioned downstream of the stop codons. For example, a 3′ UTR can comprise 1, 2 or 3 stop codons. Non-limiting examples of triple stop codons that can be used include: UGAUAAUAG (SEQ ID NO:182), UGAUAGUAA (SEQ ID NO:183), UAAUGAUAG (SEQ ID NO:184), UGAUAAUAA (SEQ ID NO:185), UGAUAGUAG (SEQ ID NO:186), UAAUGAUGA (SEQ ID NO:187), UAAUAGUAG (SEQ ID NO:188), UGAUGAUGA (SEQ ID NO:179), UAAUAAUAA (SEQ ID NO:180), and UAGUAGUAG (SEQ ID NO:181). Within a 3′ UTR, for example, 1, 2, 3 or 4 miRNA binding sites, e.g., miR-142-3p binding sites, can be positioned immediately adjacent to the stop codon(s) or at any number of nucleotides downstream of the final stop codon. When the 3′ UTR comprises multiple miRNA binding sites, these binding sites can be positioned directly next to each other in the construct (i.e., one after the other) or, alternatively, spacer nucleotides can be positioned between each binding site.

In one embodiment, the 3′ UTR comprises three stop codons with a single miR-142-3p binding site located downstream of the 3rd stop codon.

In one embodiment, the polynucleotide of the invention comprises a 5′ UTR, a codon optimized open reading frame encoding a polypeptide of interest, a 3′ UTR comprising the at least one miRNA binding site for a miR expressed in immune cells, and a 3′ tailing region of linked nucleosides. In various embodiments, the 3′ UTR comprises 1-4, at least two, one, two, three or four miRNA binding sites for miRs expressed in immune cells, preferably abundantly or preferentially expressed in immune cells.

In one embodiment, the at least one miRNA expressed in immune cells is a miR-142-3p microRNA binding site. In one embodiment, the miR-142-3p microRNA binding site comprises the sequence shown in SEQ ID NO: 174.

In one embodiment, the at least one miRNA expressed in immune cells is a miR-126 microRNA binding site. In one embodiment, the miR-126 binding site is a miR-126-3p binding site. In one embodiment, the miR-126-3p microRNA binding site comprises the sequence shown in SEQ ID NO: 152.

Non-limiting exemplary sequences for miRs to which a microRNA binding site(s) of the disclosure can bind include the following: miR-142-3p (SEQ ID NO: 173), miR-142-5p (SEQ ID NO: 175), miR-146-3p (SEQ ID NO: 155), miR-146-5p (SEQ ID NO: 156), miR-155-3p (SEQ ID NO: 157), miR-155-5p (SEQ ID NO: 158), miR-126-3p (SEQ ID NO: 151), miR-126-5p (SEQ ID NO: 153), miR-16-3p (SEQ ID NO: 159), miR-16-5p (SEQ ID NO: 160), miR-21-3p (SEQ ID NO: 161), miR-21-5p (SEQ ID NO: 162), miR-223-3p (SEQ ID NO: 163), miR-223-5p (SEQ ID NO: 164), miR-24-3p (SEQ ID NO: 165), miR-24-5p (SEQ ID NO: 166), miR-27-3p (SEQ ID NO: 167) and miR-27-5p (SEQ ID NO: 168). Other suitable miR sequences expressed in immune cells (e.g., abundantly or preferentially expressed in immune cells) are known and available in the art, for example at the University of Manchester's microRNA database, miRBase. Sites that bind any of the aforementioned miRs can be designed based on Watson-Crick complementarity to the miR, typically 100% complementarity to the miR, and inserted into an mRNA construct of the disclosure as described herein.

In another embodiment, a polynucleotide of the present invention (e.g., and mRNA, e.g., the 3′ UTR thereof) can comprise at least one miRNA bindingsite to thereby reduce or inhibit accelerated blood clearance, for example by reducing or inhibiting production of IgMs, e.g., against PEG, by B cells and/or reducing or inhibiting proliferation and/or activation of pDCs, and can comprise at least one miRNA bindingsite for modulating tissue expression of an encoded protein of interest.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′ UTR of the same sequence type.

In one embodiment, other regulatory elements and/or structural elements of the 5′ UTR can influence miRNA mediated gene regulation. One example of a regulatory element and/or structural element is a structured IRES (Internal Ribosome Entry Site) in the 5′ UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The polynucleotides of the invention can further include this structured 5′ UTR in order to enhance microRNA mediated gene regulation.

At least one miRNA binding site can be engineered into the 3′ UTR of a polynucleotide of the invention. In this context, at least two, at least three, at least four, at least five, at least six, at least seven, at least eight, at least nine, at least ten, or more miRNA binding sites can be engineered into a 3′ UTR of a polynucleotide of the invention. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a polynucleotide of the invention. In one embodiment, miRNA binding sites incorporated into a polynucleotide of the invention can be the same or can be different miRNA sites. A combination of different miRNA binding sites incorporated into a polynucleotide of the invention can include combinations in which more than one copy of any of the different miRNA sites are incorporated. In another embodiment, miRNA binding sites incorporated into a polynucleotide of the invention can target the same or different tissues in the body. As a non-limiting example, through the introduction of tissue-, cell-type-, or disease-specific miRNA binding sites in the 3′-UTR of a polynucleotide of the invention, the degree of expression in specific cell types (e.g., myeloid cells, endothelial cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′ UTR in a polynucleotide of the invention. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′ UTR. As yet another non-limiting example, a miRNA binding site can be engineered near the 5′ terminus of the 3′ UTR and near the 3′ terminus of the 3′ UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In some embodiments, the expression of a polynucleotide of the invention can be controlled by incorporating at least one sensor sequence in the polynucleotide and Formulating the polynucleotide for administration. As a non-limiting example, a polynucleotide of the invention can be targeted to a tissue or cell by incorporating a miRNA binding site and Formulating the polynucleotide in a lipid nanoparticle comprising an ionizable amino lipid, including any of the lipids described herein.

A polynucleotide of the invention can be engineered for more targeted expression in specific tissues, cell types, or biological conditions based on the expression patterns of miRNAs in the different tissues, cell types, or biological conditions. Through introduction of tissue-specific miRNA binding sites, a polynucleotide of the invention can be designed for optimal protein expression in a tissue or cell, or in the context of a biological condition.

In some embodiments, a polynucleotide of the invention can be designed to incorporate miRNA binding sites that either have 100% identity to known miRNA seed sequences or have less than 100% identity to miRNA seed sequences. In some embodiments, a polynucleotide of the invention can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the polynucleotide. In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment the miRNA sequence in the 5′ UTR can be used to stabilize a polynucleotide of the invention described herein.

In another embodiment, a miRNA sequence in the 5′ UTR of a polynucleotide of the invention can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A polynucleotide of the invention can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site.

In some embodiments, a polynucleotide of the invention can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a polynucleotide of the invention can be specific to the hematopoietic system. As another non-limiting example, a miRNA incorporated into a polynucleotide of the invention to dampen antigen presentation is miR-142-3p.

In some embodiments, a polynucleotide of the invention can include at least one miRNA in order to dampen expression of the encoded polypeptide in a tissue or cell of interest. As a non-limiting example a polynucleotide of the invention can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a polynucleotide of the invention can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a polynucleotide of the invention more unstable in antigen presenting cells. Non-limiting examples of these miRNAs include miR-142-5p, miR-142-3p, miR-146a-5p, and miR-146-3p.

In one embodiment, a polynucleotide of the invention comprises at least one miRNA sequence in a region of the polynucleotide that can interact with a RNA binding protein.

In some embodiments, the polynucleotide of the invention (e.g., a RNA, e.g., an mRNA) comprising (i) a sequence-optimized nucleotide sequence (e.g., an ORF) encoding an PCCA or PCCB polypeptide (e.g., the wild-type sequence, functional fragment, or variant thereof) and (ii) a miRNA binding site (e.g., a miRNA binding site that binds to miR-142) and/or a miRNA binding site that binds to miR-126.

Regions having a 5′ Cap

The disclosure also includes a polynucleotide that comprises both a 5′ Cap and a polynucleotide of the present invention (e.g., a polynucleotide comprising a nucleotide sequence encoding an PCCA or PCCB polypeptide to be expressed).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding an PCCA or PCCB polypeptide) incorporate a cap moiety.

In some embodiments, polynucleotides of the present invention comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m⁷G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

Another exemplary cap is m⁷G-ppp-Gm-A (i.e., N7,guanosine-5′-triphosphate-2′-O-dimethyl-guanosine-adenosine).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′-O}G(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

Polynucleotides of the invention can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N1pN2p (cap 0), 7mG(5′)ppp(5′)N1mpNp (cap 1), and 7mG(5′)-ppp(5′)N1mpN2mp (cap 2).

As a non-limiting example, capping chimeric polynucleotides post-manufacture can be more efficient as nearly 100% of the chimeric polynucleotides can be capped. This is in contrast to ˜80% when a cap analog is linked to a chimeric polynucleotide in the course of an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Also provided herein are exemplary caps including those that can be used in co-transcriptional capping methods for ribonucleic acid (RNA) synthesis, using RNA polymerase, e.g., wild type RNA polymerase or variants thereof, e.g., such as those variants described herein. In one embodiment, caps can be added when RNA is produced in a “one-pot” reaction, without the need for a separate capping reaction. Thus, the methods, in some embodiments, comprise reacting a polynucleotide template with an RNA polymerase variant, nucleoside triphosphates, and a cap analog under in vitro transcription reaction conditions to produce RNA transcript.

As used here the term “cap” includes the inverted G nucleotide and can comprise one or more additional nucleotides 3′ of the inverted G nucleotide, e.g., 1, 2, 3, or more nucleotides 3′ of the inverted G nucleotide and 5′ to the 5′ UTR, e.g., a 5′ UTR described herein.

Exemplary caps comprise a sequence of GG, GA, or GGA, wherein the underlined, italicized G is an in inverted G nucleotide followed by a 5′-5′-triphosphate group.

In one embodiment, a cap comprises a compound of formula (I)

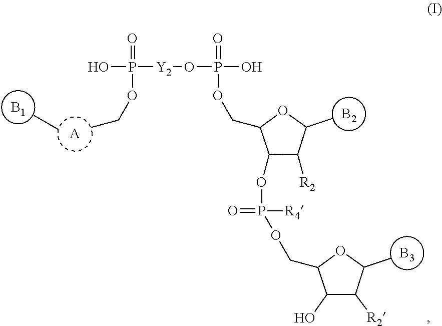

or a stereoisomer, tautomer or salt thereof, wherein

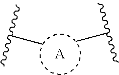

is

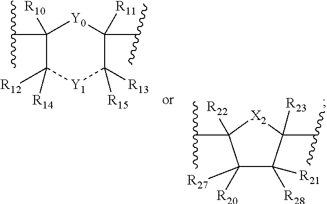

ring B₁ is a modified or unmodified Guanine;
ring B₂ and ring B₃ each independently is a nucleobase or a modified nucleobase;
X² is O, S(O)ₚ, NR₂₄ or CR₂₅R₂₆ in which p is 0, 1, or 2;
Y⁰ is O or CR₆R₇;
Y1 is O, S(O)n, CR₆R₇, or NR₈, in which n is 0, 1, or 2;
each [symbol] is a single bond or absent, wherein when each [symbol] is a single bond, Yi is O, S(O)ₙ, CR₆R₇, or NR₈; and when each [symbol] is absent, Y₁ is void;
Y₂ is (OP(O)R₄)ₘ in which m is 0, 1, or 2, or —O—(CR₄₀R₄₁)α-Q₀-(CR₄₂R₄₃)v-, in which Q₀ is a bond, O, S(O)ᵣ, NR₄₄, or CR₄₅R₄₆, r is 0, 1, or 2, and each of u and v independently is 1, 2, 3 or 4;
each R₂ and R₂′ independently is halo, LNA, or OR₃;
each R₃ independently is H, C₁-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₄ alkyl;
each R₄ and R₄′ independently is H, halo, C₁-C₆ alkyl, OH, SH, SeH, or BH₃⁻;
each of R₆, R₇, and R₈, independently, is -Q₁-T₁, in which Q₁ is a bond or 3 alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₄ alkoxyl, C(O)O—C₁-C₆ alkyl, C₃-C₈ cycloalkyl, C₁-C₁₀ aryl, NR₃₁R₃₂, (NR₃₁R₃₂R₃₃), 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)+, C₃-C₈ cycloalkyl, C₆-C₁₀ aryl, 4 to 12-membered heterocycloalkyl, and 5- or 6-membered heteroaryl;
each of R₁₀, R₁₁, R₁₂, R₁₃, R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R₃₂R₃₃)+, 4 to 12-membered heterocycloalkyl, or 5- or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)+, C₃-C₆ cycloalkyl, C₆-C₁₀ aryl, 4 to 12-membered heterocycloalkyl, and 5- or 6-membered heteroaryl; or alternatively R₁₂ together with R₁₄ is oxo, or R₁₃ together with R₁₅ is oxo,
each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, R_{S3}, or OR_{S3}, in which R_{S3} is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and R_{S3} is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
   each of R₂₄, R₂₅, and R₂₆ independently is H or C₁-C₆ alkyl;
each of R₂₇ and R₂₈ independently is H or OR₂₉; or R₂₇ and R₂₈ together form O—R₃₀—O; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
R₃₀ is C₁-C₆ alkylene optionally substituted with one or more of halo, OH and C₁-C₆ alkoxyl;
each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl;
each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;
R₄₄ is H, C₁-C₆ alkyl, or an amine protecting group;
each of R₄₅ and R₄₆ independently is H, OP(O)R₄₇R₄₈, or C₁-C₆ alkyl optionally substituted with one or more OP(O)R₄₇R₄₈, and
each of R₄₇ and R₄₈, independently is H, halo, C₁-C₆ alkyl, OH, SH, SeH, or BH₃⁻.

It should be understood that a cap analog, as provided herein, may include any of the cap analogs described in international publication WO 2017/066797, published on 20 Apr. 2017, incorporated by reference herein in its entirety.

In some embodiments, the B2 middle position can be a non-ribose molecule, such as arabinose.

In some embodiments R₂ is ethyl-based.

Thus, in some embodiments, a cap comprises the following structure:

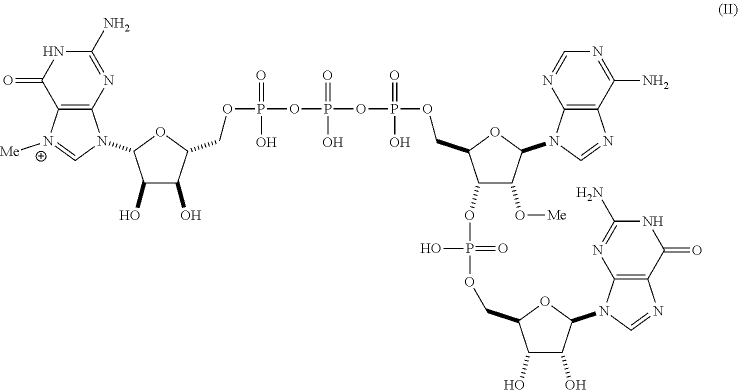

In other embodiments, a cap comprises the following structure:

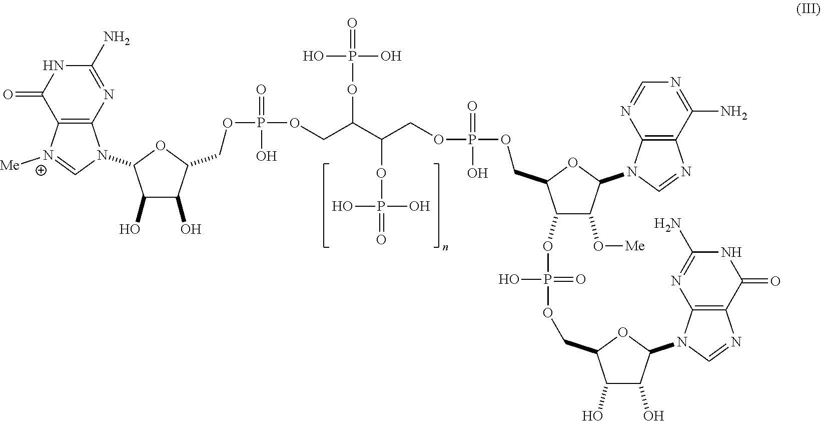

In yet other embodiments, a cap comprises the following structure:

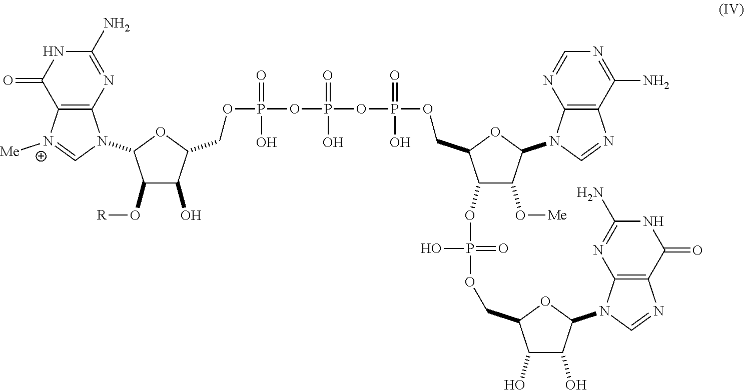

In still other embodiments, a cap comprises the following structure:

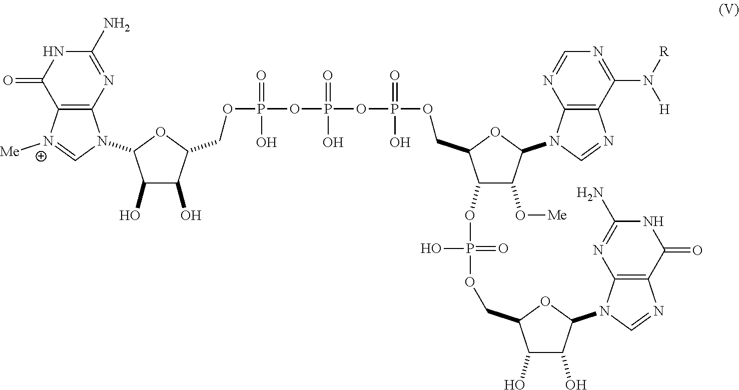

In some embodiments, R is an alkyl (e.g., C₁-C₆ alkyl). In some embodiments, R is a methyl group (e.g., C₁ alkyl). In some embodiments, R is an ethyl group (e.g., C₂ alkyl).

In some embodiments, a cap comprises a sequence selected from the following sequences: GAA, GAC, GAG, GAU, GCA, GCC, GCG, GCU, GGA, GGC, GGG, GGU, GUA, GUC, GUG, and GUU. In some embodiments, a cap comprises GAA. In some embodiments, a cap comprises GAC. In some embodiments, a cap comprises GAG.

In some embodiments, a cap comprises GAU. In some embodiments, a cap comprises GCA. In some embodiments, a cap comprises GCC. In some embodiments, a cap comprises GCG. In some embodiments, a cap comprises GCU. In some embodiments, a cap comprises GGA. In some embodiments, a cap comprises GGC. In some embodiments, a cap comprises GGG. In some embodiments, a cap comprises GGU. In some embodiments, a cap comprises GUA. In some embodiments, a cap comprises GUC. In some embodiments, a cap comprises GUG. In some embodiments, a cap comprises GUU.

In some embodiments, a cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

In some embodiments, a cap comprises m⁷GpppApA. In some embodiments, a cap comprises m⁷GpppApC. In some embodiments, a cap comprises m⁷GpppApG. In some embodiments, a cap comprises m⁷GpppApU. In some embodiments, a cap comprises m⁷GpppCpA. In some embodiments, a cap comprises m⁷GpppCpC. In some embodiments, a cap comprises m⁷GpppCpG. In some embodiments, a cap comprises m⁷GpppCpU. In some embodiments, a cap comprises m⁷GpppGpA. In some embodiments, a cap comprises m⁷GpppGpC. In some embodiments, a cap comprises m⁷GpppGpG. In some embodiments, a cap comprises m⁷GpppGpU. In some embodiments, a cap comprises m⁷GpppUpA. In some embodiments, a cap comprises m⁷GpppUpC. In some embodiments, a cap comprises m⁷GpppUpG. In some embodiments, a cap comprises m⁷GpppUpU.

A cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

In some embodiments, a cap comprises m⁷G_{3′OMe}pppApA. In some embodiments, a cap comprises m⁷G_{3′OMe}pppApC. In some embodiments, a cap comprises m⁷G_{3′OMe}pppApG. In some embodiments, a cap comprises m⁷G_{3′OMe}pppApU. In some embodiments, a cap comprises m⁷G_{3′OMe}pppCpA. In some embodiments, a cap comprises m⁷G_{3′OMe}pppCpC. In some embodiments, a cap comprises m⁷G_{3′OMe}pppCpG. In some embodiments, a cap comprises m⁷G_{3′OMe}pppCpU. In some embodiments, a cap comprises m⁷G_{3′OMe}pppGpA. In some embodiments, a cap comprises m7G_{3′OMe}pppGpC. In some embodiments, a cap comprises m⁷G_{3′OMe}pppGpG. In some embodiments, a cap comprises m⁷G_{3′OMe}pppGpU. In some embodiments, a cap comprises m7G_{3′OMe}pppUpA. In some embodiments, a cap comprises m⁷G_{3′OMe}pppUpC. In some embodiments, a cap comprises m⁷G_{3′OMe}pppUpG. In some embodiments, a cap comprises m⁷G_{3′OMe}pppUpU.

A cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

In some embodiments, a cap comprises m⁷G_{3′OMe}pppA_{2′OMe}pA. In some embodiments, a cap comprises m⁷G_{3′OMe}pppA_{2′OMe}pC. In some embodiments, a cap comprises m⁷G_{3′OMe}pppA_{2′OMe}pG. In some embodiments, a cap comprises m⁷G_{3′OMe}pppA_{2′OMe}pU. In some embodiments, a cap comprises m⁷G_{3′OMe}pppC_{2′OMe}pA. In some embodiments, a cap comprises m⁷G_{3′OMe}pppC_{2′OMe}pC. In some embodiments, a cap comprises m⁷G_{3′OMe}pppC_{2′OMe}pG. In some embodiments, a cap comprises m⁷G_{3′OMe}pppC_{2′OMe}pU. In some embodiments, a cap comprises m⁷G_{3′OMe}pppG_{2′OMe}pA. In some embodiments, a cap comprises m⁷G_{3′OMe}pppG_{2′OMe}pC. In some embodiments, a cap comprises m⁷G_{3′OMe}pppG_{2′OMe}pG. In some embodiments, a cap comprises m⁷G_{3′OMe}pppG_{2′OMe}pU. In some embodiments, a cap comprises m⁷G_{3′OMe}pppU_{2′OMe}pA. In some embodiments, a cap comprises m⁷G_{3′OMe}pppU_{2′OMe}pC. In some embodiments, a cap comprises m⁷G_{3′OMe}pppU_{2′OMe}pG. In some embodiments, a cap comprises m⁷G_{3′OMe}pppU_{2′OMe}pU.

A cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

In some embodiments, a cap comprises m⁷GpppA_{2′OMe}pA. In some embodiments, a cap comprises m⁷GpppA_{2′OMe}pC. In some embodiments, a cap comprises m⁷GpppA_{2′OMe}pG. In some embodiments, a cap comprises m⁷GpppA_{2′OMe}pU. In some embodiments, a cap comprises m⁷GpppC_{2′OMe}pA. In some embodiments, a cap comprises m⁷GpppC_{2′OMe}pC. In some embodiments, a cap comprises m7GpppC_{2′OMe}pG. In some embodiments, a trinucleotide cap comprises m⁷GpppC_{2′OMe}pU. In some embodiments, a cap comprises m⁷GpppG_{2′OMe}pA. In some embodiments, a cap comprises m⁷GpppG_{2′OMe}pC. In some embodiments, a cap comprises m7GpppG_{2′OMe}pG. In some embodiments, a cap comprises m⁷GpppG_{2′OMe}pU. In some embodiments, a cap comprises m⁷GpppU_{2′OMe}pA. In some embodiments, a cap comprises m⁷GpppU_{2′OMe}pC. In some embodiments, a cap comprises m⁷GpppU_{2′OMe}pG. In some embodiments, a cap comprises m⁷GpppU_{2′OMe}pU.

In some embodiments, a cap comprises m⁷Gpppm⁶A_{2′OMe}pG. In some embodiments, a cap comprises m⁷Gpppe⁶A_{2′OMe}pG.

In some embodiments, a cap comprises GAG. In some embodiments, a cap comprises GCG. In some embodiments, a cap comprises GUG. In some embodiments, a cap comprises GGG.

In some embodiments, a cap comprises any one of the following structures:

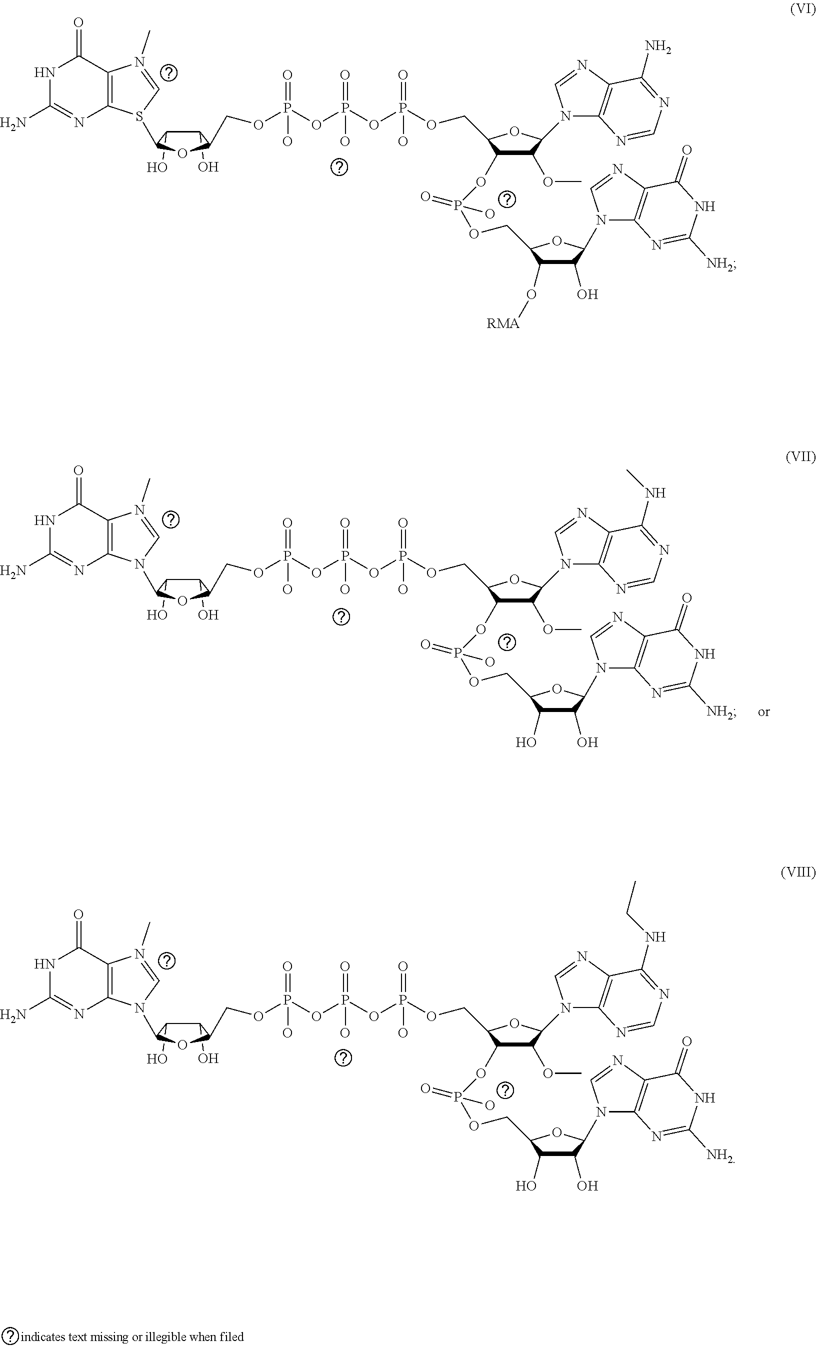

In some embodiments, the cap comprises ^{m7}GpppN₁N₂N₃, where N₁, N₂, and N₃ are optional (i.e., can be absent or one or more can be present) and are independently a natural, a modified, or an unnatural nucleoside base. In some embodiments, ^{m7}G is further methylated, e.g., at the 3′ position. In some embodiments, the ^{m7}G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position.

In some embodiments, the cap comprises the following structure:

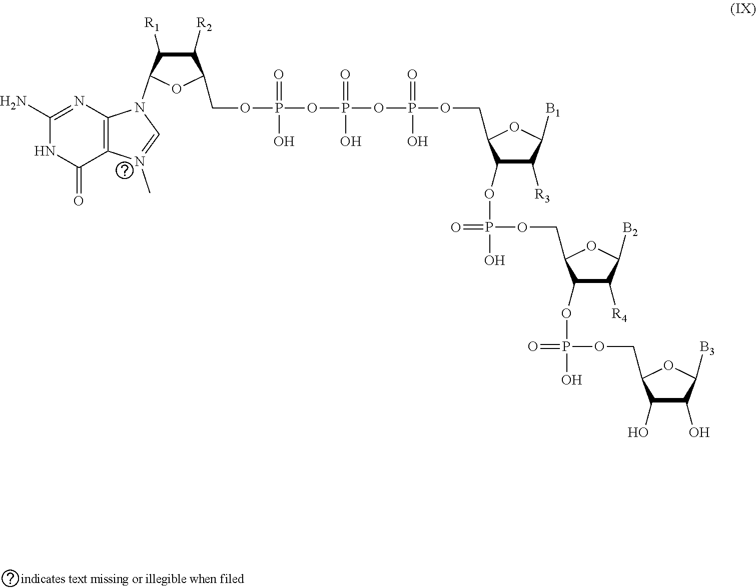

wherein B₁, B₂, and B₃ are independently a natural, a modified, or an unnatural nucleoside based; and R₁, R₂, R₃, and R₄ are independently OH or O-methyl. In some embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH. In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B1, B3, and B3 are natural nucleoside bases. In some embodiments, at least one of B1, B2, and B3 is a modified or unnatural base. In some embodiments, at least one of B1, B2, and B3 is N₆-methyl adenine. In some embodiments, B₁ is adenine, cytosine, thymine, or uracil. In some embodiments, B₁ is adenine, B2 is uracil, and B3 is adenine. In some embodiments, R₁ and R₂ are OH, R₃ and R₄ are O-methyl, B₁ is adenine, B2 is uracil, and B3 is adenine.

In some embodiments the cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A cap, in some embodiments, comprises a sequence selected from the following sequences: m⁷G_{3′OMe}pppApApN, m⁷G_{3′OMe}pppApCpN, m⁷G_{3′OMe}pppApGpN, m⁷G_{3′OMe}pppApUpN, m⁷G_{3′OMe}pppCpApN, m⁷G_{3′OMe}pppCpCpN, m⁷G_{3′OMe}pppCpGpN, m⁷G_{3′OMe}pppCpUpN, m⁷G_{3′OMe}pppGpApN, m⁷G_{3′OMe}pppGpCpN, m⁷G_{3′OMe}pppGpGpN, m⁷G_{3′OMe}pppGpUpN, m⁷G_{3′OMe}pppUpApN, m⁷G_{3′OMe}pppUpCpN, m⁷G_{3′OMe}pppUpGpN, and m⁷G_{3′OMe}pppUpUpN, where N is a natural, a modified, or an unnatural nucleoside base.

A cap, in other embodiments, comprises a sequence selected from the following sequences: m⁷G_{3′OMe}pppA_{2′OMe}pApN, m⁷G_{3′OMe}pppA_{2′OMe}pCpN, m⁷G_{3′OMe}pppA_{2′OMe}pGpN, m⁷G_{3′OMe}pppA_{2′OMe}pUpN, m⁷G_{3′OMe}pppC_{2′OMe}pApN, m⁷G_{3′OMe}pppC_{2′OMe}pCpN, m⁷G_{3′OMe}pppC_{2′OMe}pGpN, m⁷G_{3′OMe}pppC_{2′OMe}pUpN, m⁷G_{3′OMe}pppG_{2′OMe}pApN, m⁷G_{3′OMe}pppG_{2′OMe}pCpN, m⁷G_{3′OMe}pppG_{2′OMe}pGpN, m⁷G_{3′OMe}pppG_{2′OMe}pUpN, m⁷G_{3′OMe}pppU_{2′OMe}pApN, m⁷G_{3′OMe}pppU_{2′OMe}pCpN, m⁷G_{3′OMe}pppU_{2′OMe}pGpN, and m⁷G_{3′OMe}pppU_{2′OMe}pUpN, where N is a natural, a modified, or an unnatural nucleoside base.

A cap, in still other embodiments, comprises a sequence selected from the following sequences: m⁷GpppA_{2′OMe}pApN, m⁷GpppA_{2′OMe}pCpN, m⁷GpppA_{2′OMe}pGpN, m⁷GpppA_{2′OMe}pUpN, m⁷GpppC_{2′OMe}pApN, m⁷GpppC_{2′OMe}pCpN, m⁷GpppC_{2′OMe}pGpN, m⁷GpppC_{2′OMe}pUpN, m⁷GpppG_{2′OMe}pApN, m⁷GpppG_{2′OMe}pCpN, m⁷GpppG_{2′OMe}pGpN, m⁷GpppG_{2′OMe}pUpN, m⁷GpppU_{2′OMe}pApN, m⁷GpppU_{2′OMe}pCpN, m⁷GpppU_{2′OMe}pGpN, and m⁷GpppU_{2′OMe}pUpN, where N is a natural, a modified, or an unnatural nucleoside base.

A cap, in other embodiments, comprises a sequence selected from the following sequences: m⁷G_{3′OMe}pppA_{2′OMe}pA_{2′OMe}pN, m⁷G_{3′OMe}pppA_{2′OMe}pC_{2′OMe}pN, m⁷G_{3′OMe}pppA_{2′OMe}pG_{2′OMe}pN, m⁷G_{3′OMe}pppA_{2′OMe}pU_{2′OMe}pN, m⁷G_{3′OMe}pppC_{2′OMe}pA_{2′OMe}pN, m⁷G_{3′OMe}pppC_{2′OMe}pC_{2′OMe}pN, m⁷G_{3′OMe}pppC_{2′OMe}pG_{2′OMe}pN, m⁷G_{3′OMe}pppC_{2′OMe}pU_{2′OMe}pN, m⁷G_{3′OMe}pppG_{2′OMe}pA_{2′OMe}pN, m⁷G_{3′OMe}pppG_{2′OMe}pC_{2′OMe}pN, m⁷G_{3′OMe}pppG_{2′OMe}pG_{2′OMe}pN, m⁷G_{3′OMe}pppG_{2′OMe}pU_{2′OMe}pN, m⁷G_{3′OMe}pppU_{2′OMe}pA_{2′OMe}pN, m⁷G_{3′OMe}pppU_{2′OMe}pC_{2′OMe}pN, m⁷G_{3′OMe}pppU_{2′OMe}pG_{2′OMe}pN, and m⁷G_{3′OMe}pppU_{2′OMe}pU_{2′OMe}pN, where N is a natural, a modified, or an unnatural nucleoside base.

A cap, in still other embodiments, comprises a sequence selected from the following sequences: m⁷GpppA_{2′OMe}pA_{2′OMe}pN, m⁷GpppA_{2′OMe}pC_{2′OMe}pN, m⁷GpppA_{2′OMe}pG_{2′OMe}pN, m⁷GpppA_{2′OMe}pU_{2′OMe}pN, m⁷GpppC_{2′OMe}pA_{2′OMe}pN, m⁷GpppC_{2′OMe}pC_{2′OMe}pN, m⁷GpppC_{2′OMe}pG_{2′OMe}pN, m⁷GpppC_{2′OMe}pU_{2′OMe}pN, m⁷GpppG_{2′OMe}pA_{2′OMe}pN, m⁷GpppG_{2′OMe}pC_{2′OMe}pN, m⁷GpppG_{2′OMe}pG_{2′OMe}pN, m⁷GpppG_{2′OMe}pU_{2′OMe}pN, m⁷GpppU_{2′OMe}pA_{2′OMe}pN, m⁷GpppU_{2′OMe}pC_{2′OMe}pN, m⁷GpppU_{2′OMe}pG_{2′OMe}pN, and m⁷GpppU_{2′OMe}pU_{2′OMe}pN, where N is a natural, a modified, or an unnatural nucleoside base.

In some embodiments, a cap comprises GGAG. In some embodiments, a cap comprises the following structure:

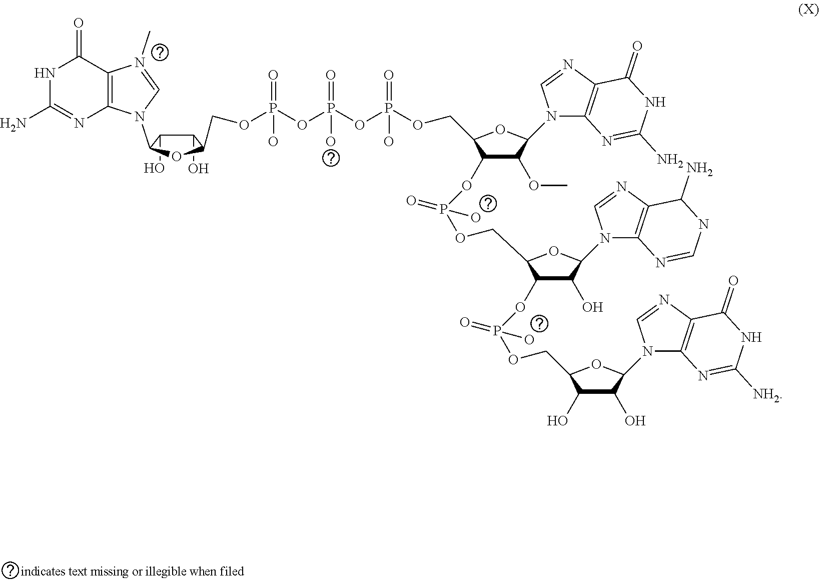

### Poly-A Tails

In some embodiments, the polynucleotides of the present disclosure (e.g., a polynucleotide comprising a nucleotide sequence encoding an PCCA or PCCB polypeptide) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule in order to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO. 195).

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO:196).

In some embodiments, the polyA tail comprises an alternative nucleoside, e.g., inverted thymidine. PolyA tails comprising an alternative nucleoside, e.g., inverted thymidine, may be generated as described herein. For instance, mRNA constructs may be modified by ligation to stabilize the poly(A) tail. Ligation may be performed using 0.5-1.5 mg/mL mRNA (5′ Cap1, 3′ A100), 50 mM Tris-HCl pH 7.5, 10 mM MgCl₂, 1 mM TCEP, 1000 units/mL T4 RNA Ligase 1, 1 mM ATP, 20% w/v polyethylene glycol 8000, and 5:1 molar ratio of modifying oligo to mRNA. Modifying oligo has a sequence of 5′-phosphate-AAAAAAAAAAAAAAAAAAAA-(inverted deoxythymidine (idT) (SEQ ID NO:209)) (see below). Ligation reactions are mixed and incubated at room temperature (˜22° C.) for, e.g., 4 hours. Stable tail mRNA are purified by, e.g., dT purification, reverse phase purification, hydroxyapatite purification, ultrafiltration into water, and sterile filtration. The resulting stable tail-containing mRNAs contain the following structure at the 3′end, starting with the polyA region: A₁₀₀-UCUAGAAAAAAAAAAAAAAAAAAAA-inverted deoxythymidine (SEQ ID NO:211).

Modifying oligo to stabilize tail (5′-phosphate-AAAAAAAAAAAAAAAAAAAA-(inverted deoxythymidine)(SEQ ID NO:209)):

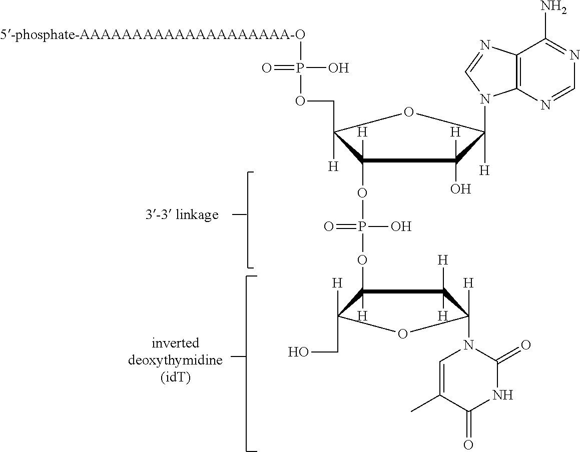

In some instances, the polyA tail comprises A100-UCUAG-A20-inverted deoxy-thymidine (SEQ ID NO:211). In some instances, the polyA tail consists of A100-UCUAG-A20-inverted deoxy-thymidine (SEQ ID NO:211).

### Start Codon Region

The invention also includes a polynucleotide that comprises both a start codon region and the polynucleotide described herein (e.g., a polynucleotide comprising a nucleotide sequence encoding an PCCA or PCCB polypeptide). In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As yet another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide in order to increase the likelihood that translation will initiate on an alternative start codon. In some embodiments, a masking agent can be used to mask a first start codon or alternative start codon in order to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence in order to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

### Stop Codon Region

The invention also includes a polynucleotide that comprises both a stop codon region and the polynucleotide described herein (e.g., a polynucleotide comprising a nucleotide sequence encoding an PCCA or PCCB polypeptide). In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

Combination of mRNA Elements

Any of the polynucleotides disclosed herein can comprise one, two, three, or all of the following elements: (a) a 5′-UTR, e.g., as described herein; (b) a coding region comprising a stop element (e.g., as described herein); (c) a 3′-UTR (e.g., as described herein) and; optionally (d) a 3′ stabilizing region, e.g., as described herein. Also disclosed herein are LNP compositions comprising the same.

In an embodiment, a polynucleotide of the disclosure comprises (a) a 5′ UTR described in Table 2 or a variant or fragment thereof and (b) a coding region comprising a stop element provided herein. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, a polynucleotide of the disclosure comprises (a) a 5′ UTR described in Table 2 or a variant or fragment thereof and (c) a 3′ UTR described in Table 3 or a variant or fragment thereof. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, a polynucleotide of the disclosure comprises (c) a 3′ UTR described in Table 3 or a variant or fragment thereof and (b) a coding region comprising a stop element provided herein. In an embodiment, the polynucleotide comprises a sequence provided in Table 5. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, a polynucleotide of the disclosure comprises (a) a 5′ UTR described in Table 2 or a variant or fragment thereof; (b) a coding region comprising a stop element provided herein; and (c) a 3′ UTR described in Table 3 or a variant or fragment thereof. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

TABLE 5
Exemplary 3′ UTR and stop element sequences

| SEQ ID | Sequence | |
|---|---|---|
| NO | information | Sequence |
| 121 | 3′ UTR with stop | UAGGGUUAAGCUGGAGCCUCGGUGGCCUAGCUUCUU |
| | C11 (underlined) | GCCCCUUGGGCCUCCCCCCAGCCCCUCCUCCCCUUC |
| | | CUGCACCCGUACCCCCGUGGUCUUUGAAUAAAGUCU |
| | | GAGUGGGCGGC |
| 122 | 3′ UTR with stop | UAAAGCUCCGCUGGAGCCUCGGUGGCCUAGCUUCUU |
| | C10 (underlined) | GCCCCUUGGGCCUCCCCCCAGCCCCUCCUCCCCUUC |
| | | CUGCACCCGUACCCCCGUGGUCUUUGAAUAAAGUCU |
| | | GAGUGGGCGGC |
| 123 | 3′ UTR with stop | UAAGCCCCUGCUGGAGCCUCGGUGGCCUAGCUUCUU |
| | C9 (underlined) | GCCCCUUGGGCCUCCCCCCAGCCCCUCCUCCCCUUC |
| | | CUGCACCCGUACCCCCGUGGUCUUUGAAUAAAGUCU |
| | | GAGUGGGCGGC |
| 124 | 3′ UTR with stop | UAAGCACCCGCUGGAGCCUCGGUGGCCUAGCUUCUU |
| | C8 (underlined) | GCCCCUUGGGCCUCCCCCCAGCCCCUCCUCCCCUUC |
| | | CUGCACCCGUACCCCCGUGGUCUUUGAAUAAAGUCU |
| | | GAGUGGGCGGC |
| 125 | 3′ UTR with stop | UAAGCCCCUCCGGGGGCCUCGGUGGCCUAGCUUCUU |
| | C7 (underlined) | GCCCCUUGGGCCUCCCCCCAGCCCCUCCUCCCCUUC |
| | | CUGCACCCGUACCCCCGUGGUCUUUGAAUAAAGUCU |
| | | GAGUGGGCGGC |
| 126 | 3′ UTR with stop | UAAGGCUAAGCUGGAGCCUCGGUGGCCUAGCUUCUU |
| | C6 (underlined) | GCCCCUUGGGCCUCCCCCCAGCCCCUCCUCCCCUUC |
| | | CUGCACCCGUACCCCCGUGGUCUUUGAAUAAAGUCU |
| | | GAGUGGGCGGC |
| 127 | 3′ UTR with stop | UAAGUCUCCGCUGGAGCCUCGGUGGCCUAGCUUCUU |
| | C5 (underlined) | GCCCCUUGGGCCUCCCCCCAGCCCCUCCUCCCCUUC |
| | | CUGCACCCGUACCCCCGUGGUCUUUGAAUAAAGUCU |
| | | GAGUGGGCGGC |
| 128 | 3′ UTR with stop | UAAAGCUAAGCUGGAGCCUCGGUGGCCUAGCUUCUU |
| | C4 (underlined) | GCCCCUUGGGCCUCCCCCCAGCCCCUCCUCCCCUUC |
| | | CUGCACCCGUACCCCCGUGGUCUUUGAAUAAAGUCU |
| | | GAGUGGGCGGC |
| 129 | 3′ UTR with stop | UAAGUCUAAGCUGGAGCCUCGGUGGCCUAGCUUCUU |
| | C3 (underlined) | GCCCCUUGGGCCUCCCCCCAGCCCCUCCUCCCCUUC |
| | | CUGCACCCGUACCCCCGUGGUCUUUGAAUAAAGUCU |
| | | GAGUGGGCGGC |
| 130 | 3′ UTR with C10 | UAAAGCUCCAUAAAGUAGGAAACACUACAGCUGGAG |
| | stop (underlined) | CCUCGGUGGCCUAGCUUCUUGCCCCUUGGGCCUCCA |
| | | UAAAGUAGGAAACACUACAUCCCCCCAGCCCCUCCU |
| | | CCCCUUCCUGCACCCGUACCCCCUCCAUAAAGUAGG |
| | | AAACACUACAGUGGUCUUUGAAUAAAGUCUGAGUGG |
| | | GCGGC |
| 131 | 3′ UTR with C7 | UAAGCCCCUCCGGGGUCCAUAAAGUAGGAAACACUA |
| | stop (underlined) | CAGCCUCGGUGGCCUAGCUUCUUGCCCCUUGGGCCU |
| | | CCAUAAAGUAGGAAACACUACAUCCCCCCAGCCCCU |
| | | CCUCCCCUUCCUGCACCCGUACCCCCUCCAUAAAGU |
| | | AGGAAACACUACAGUGGUCUUUGAAUAAAGUCUGAG |
| | | UGGGCGGC |
| 132 | 3′ UTR with C8 | UAAAGCUCCCCGGGGUCCAUAAAGUAGGAAACACUA |
| | stop (underlined) | CAGCCUCGGUGGCCUAGCUUCUUGCCCCUUGGGCCU |
| | | CCAUAAAGUAGGAAACACUACAUCCCCCCAGCCCCU |
| | | CCUCCCCUUCCUGCACCCGUACCCCCUCCAUAAAGU |
| | | AGGAAACACUACAGUGGUCUUUGAAUAAAGUCUGAG |
| | | UGGGCGGC |

Polynucleotide Comprising an mRNA Encoding an PCCA or PCCB Polypeptide

In certain embodiments, a polynucleotide of the present disclosure, for example a polynucleotide comprising an mRNA nucleotide sequence encoding an PCCA or PCCB polypeptide, comprises from 5′ to 3′ end:
(i) a 5′ cap such as provided above;
(ii) a 5′ UTR, such as the sequences provided above;
(iii) an ORF encoding a human PCCA or PCCB polypeptide (e.g., SEQ ID NO:1 or SEQ ID NO:2), wherein the ORF has at least 90%, at least 95%, at least 97%, at least 98%, at least 99%, or 100% sequence identity to the sequence of SEQ ID NO:5 or SEQ ID NO:6;
(iv) at least one stop codon;
(v) a 3′ UTR, such as the sequences provided above; and
(vi) a poly-A tail provided above.

In some embodiments, the polynucleotide further comprises a miRNA binding site, e.g., a miRNA binding site that binds to miRNA-142. In some embodiments, the 5′ UTR comprises the miRNA binding site. In some embodiments, the 3′ UTR comprises the miRNA binding site.

In some embodiments, a polynucleotide of the present disclosure comprises a nucleotide sequence encoding a polypeptide sequence at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the protein sequence of a human PCCA having the amino acid sequence of SEQ ID NO:1.

In some embodiments, a polynucleotide of the present disclosure comprises a nucleotide sequence encoding a polypeptide sequence at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the protein sequence of a human PCCB having the amino acid sequence of SEQ ID NO:2.

In some embodiments, a polynucleotide of the present disclosure, for example a polynucleotide comprising an mRNA nucleotide sequence encoding a polypeptide, comprises (1) a 5′ cap such as provided above, for example, m⁷Gp-ppGm-A, (2) a 5′ UTR, (3) a nucleotide sequence ORF of SEQ ID NO:5, (3) a stop codon, (4) a 3′UTR, and (5) a poly-A tail provided above, for example, a poly-A tail of SEQ ID NO:195 or A100-UCUAG-A20-inverted deoxy-thymidine (SEQ ID NO:211).

In some embodiments, a polynucleotide of the present disclosure, for example a polynucleotide comprising an mRNA nucleotide sequence encoding a polypeptide, comprises (1) a 5′ cap such as provided above, for example, m⁷Gp-ppGm-A, (2) a 5′ UTR, (3) a nucleotide sequence ORF of SEQ ID NO:6, (3) a stop codon, (4) a 3′UTR, and (5) a poly-A tail provided above, for example, a poly-A tail of SEQ ID NO:195 or A100-UCUAG-A20-inverted deoxy-thymidine (SEQ ID NO:211).

Exemplary PCCA or PCCB nucleotide constructs are described below:

SEQ ID NO:7 consists from 5′ to 3′ end: 5′ UTR of SEQ ID NO:55, ORF Sequence of SEQ ID NO:5, and 3′ UTR of SEQ ID NO:114.

SEQ ID NO:8 consists from 5′ to 3′ end: 5′ UTR of SEQ ID NO:55, ORF Sequence of SEQ ID NO:6, and 3′ UTR of SEQ ID NO:114.

In certain embodiments, in a construct with SEQ ID NO:7, all uracils therein are replaced by N1-methylpseudouracil.

In certain embodiments, in constructs with SEQ ID NO:7, all uracils therein are replaced by 5-methoxyuracil.

In certain embodiments, in a construct with SEQ ID NO:8, all uracils therein are replaced by N1 methylpseudouracil.

In certain embodiments, in constructs with SEQ ID NO:8, all uracils therein are replaced by 5-methoxyuracil.

In some embodiments, a polynucleotide of the present disclosure, for example a polynucleotide comprising an mRNA nucleotide sequence encoding an PCCA or PCCB polypeptide, comprises (1) a 5′ cap such as provided above, for example, m⁷Gp-ppGm-A, (2) a nucleotide sequence of SEQ ID NO:7, and (3) a poly-A tail provided above, for example, a poly A tail of ˜100 residues, e.g., SEQ ID NO:195 or A100-UCUAG-A20-inverted deoxy-thymidine (SEQ ID NO:211). In certain embodiments, in constructs with SEQ ID NO:7, all uracils therein are replaced by N1 methylpseudouracil. In certain embodiments, in constructs with SEQ ID NO:7, all uracils therein are replaced by 5-methoxyuracil.

In some embodiments, a polynucleotide of the present disclosure, for example a polynucleotide comprising an mRNA nucleotide sequence encoding an PCCA or PCCB polypeptide, comprises (1) a 5′ cap such as provided above, for example, m⁷Gp-ppGm-A, (2) a nucleotide sequence of SEQ ID NO:8, and (3) a poly-A tail provided above, for example, a poly A tail of ˜100 residues, e.g., SEQ ID NO:195 or A100-UCUAG-A20-inverted deoxy-thymidine (SEQ ID NO:211). In certain embodiments, in constructs with SEQ ID NO:8, all uracils therein are replaced by N1 methylpseudouracil. In certain embodiments, in constructs with SEQ ID NO:8, all uracils therein are replaced by 5-methoxyuracil.

### Methods of Making Polynucleotides

The present disclosure also provides methods for making a polynucleotide of the invention (e.g., a polynucleotide comprising a nucleotide sequence encoding an PCCA or PCCB polypeptide) or a complement thereof.

In some aspects, a polynucleotide (e.g., a RNA, e.g., an mRNA) disclosed herein, and encoding an PCCA or PCCB polypeptide, can be constructed using in vitro transcription (IVT). In other aspects, a polynucleotide (e.g., a RNA, e.g., an mRNA) disclosed herein, and encoding an PCCA or PCCB polypeptide, can be constructed by chemical synthesis using an oligonucleotide synthesizer.

In other aspects, a polynucleotide (e.g., a RNA, e.g., an mRNA) disclosed herein, and encoding an PCCA or PCCB polypeptide is made by using a host cell. In certain aspects, a polynucleotide (e.g., a RNA, e.g., an mRNA) disclosed herein, and encoding an PCCA or PCCB polypeptide is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., a RNA, e.g., an mRNA) encoding an PCCA or PCCB polypeptide. The resultant polynucleotides, e.g., mRNAs, can then be examined for their ability to produce protein and/or produce a therapeutic outcome.

In Vitro Transcription/Enzymatic Synthesis

The present disclosure also provides methods for making a polynucleotide disclosed herein or a complement thereof. In some aspects, a polynucleotide (e.g., an mRNA) disclosed herein can be constructed using in vitro transcription.

In other aspects, a polynucleotide (e.g., an mRNA) disclosed herein can be constructed by chemical synthesis using an oligonucleotide synthesizer. In other aspects, a polynucleotide (e.g., an mRNA) disclosed herein is made by using a host cell. In certain aspects, a polynucleotide (e.g., an mRNA) disclosed herein is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding an PCCA or PCCB polypeptide. The resultant mRNAs can then be examined for their ability to produce PCCA or PCCB and/or produce a therapeutic outcome.

While RNA can be made synthetically using methods well known in the art, in one embodiment an RNA transcript (e.g., mRNA transcript) is synthesized by contacting a DNA template with a RNA polymerase (e.g., a T7 RNA polymerase or a T7 RNA polymerase variant) under conditions that result in the production of RNA transcript.

In some aspects, the present disclosure provides methods of performing an IVT (in vitro transcription) reaction, comprising contacting a DNA template with the RNA polymerase (e.g., a T7 RNA polymerase, such as a T7 RNA polymerase variant) in the presence of nucleoside triphosphates and buffer under conditions that result in the production of RNA transcripts.

Other aspects of the present disclosure provide capping methods, e.g., co-transcriptional capping methods or other methods known in the art. In one embodiment, a capping method comprises reacting a polynucleotide template with a T7 RNA polymerase variant, nucleoside triphosphates, and a cap analog under in vitro transcription reaction conditions to produce RNA transcript.

IVT conditions typically require a purified linear DNA template containing a promoter, nucleoside triphosphates, a buffer system that includes dithiothreitol (DTT) and magnesium ions, and a RNA polymerase. The exact conditions used in the transcription reaction depend on the amount of RNA needed for a specific application. Typical IVT reactions are performed by incubating a DNA template with a RNA polymerase and nucleoside triphosphates, including GTP, ATP, CTP, and UTP (or nucleotide analogs) in a transcription buffer. A RNA transcript having a 5′ terminal guanosine triphosphate is produced from this reaction.

A deoxyribonucleic acid (DNA) is simply a nucleic acid template for RNA polymerase. A DNA template may include a polynucleotide encoding an PCCA or PCCB polypeptide. A DNA template, in some embodiments, includes a RNA polymerase promoter (e.g., a T7 RNA polymerase promoter) located 5′ from and operably linked to polynucleotide encoding an PCCA or PCCB polypeptide. A DNA template may also include a nucleotide sequence encoding a polyadenylation (polyA) tail located at the 3′ end of the gene of interest.

Polypeptides of interest include, but are not limited to, biologics, antibodies, antigens (vaccines), and therapeutic proteins. The term “protein” encompasses peptides.

A RNA transcript, in some embodiments, is the product of an IVT reaction and, as will be understood by one of ordinary skill in the art, the DNA template for making an RNA molecule is known based on base complementarity. A RNA transcript, in some embodiments, is a messenger RNA (mRNA) that includes a nucleotide sequence encoding a polypeptide of interest linked to a polyA tail. In some embodiments, the mRNA is modified mRNA (mmRNA), which includes at least one modified nucleotide.

A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, a RNA transcript (e.g., mRNA transcript) of the present disclosure may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, a RNA transcript (e.g., mRNA transcript) includes a combination of at least two (e.g., 2, 3, 4 or more) of the foregoing modified nucleobases.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The concentration of nucleoside triphosphates and cap analog present in an IVT reaction may vary. In some embodiments, NTPs and cap analog are present in the reaction at equimolar concentrations. In some embodiments, the molar ratio of cap analog (e.g., trinucleotide cap) to nucleoside triphosphates in the reaction is greater than 1:1. For example, the molar ratio of cap analog to nucleoside triphosphates in the reaction may be 2:1, 3:1, 4:1, 5:1, 6:1, 7:1, 8:1, 9:1, 10:1, 15:1, 20:1, 25:1, 50:1, or 100:1. In some embodiments, the molar ratio of cap analog (e.g., trinucleotide cap) to nucleoside triphosphates in the reaction is less than 1:1. For example, the molar ratio of cap analog (e.g., trinucleotide cap) to nucleoside triphosphates in the reaction may be 1:2, 1:3, 1:4, 1:5, 1:6, 1:7, 1:8, 1:9, 1:10, 1:15, 1:20, 1:25, 1:50, or 1:100.

The composition of NTPs in an IVT reaction may also vary. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. The same IVT reaction may include 3.75 millimolar cap analog (e.g., trinucleotide cap). In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5.

In some embodiments, a RNA transcript (e.g., mRNA transcript) includes a modified nucleobase selected from pseudouridine (ψ), 1-methylpseudouridine (m¹ψ), 5-methoxyuridine (mo⁵U), 5-methylcytidine (m⁵C), α-thio-guanosine and α-thio-adenosine. In some embodiments, a RNA transcript (e.g., mRNA transcript) includes a combination of at least two (e.g., 2, 3, 4 or more) of the foregoing modified nucleobases.

In some embodiments, a RNA transcript (e.g., mRNA transcript) includes pseudouridine (ψ). In some embodiments, a RNA transcript (e.g., mRNA transcript) includes 1-methylpseudouridine (m¹ω). In some embodiments, a RNA transcript (e.g., mRNA transcript) includes 5-methoxyuridine (mo⁵U). In some embodiments, a RNA transcript (e.g., mRNA transcript) includes 5-methylcytidine (m⁵C). In some embodiments, a RNA transcript (e.g., mRNA transcript) includes α-thio-guanosine. In some embodiments, a RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment of the present invention.

In some embodiments, the buffer system contains tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:1, 1:2, 1:3, 1:4 or 1:5.

In some embodiments, the molar ratio of NTP plus cap analog (e.g., trinucleotide cap, such as GAG) to magnesium ions (Mg²⁺; e.g., MgCl₂) present in an IVT reaction is 1:1 to 1:5. For example, the molar ratio of NTP+trinucleotide cap (e.g., GAG) to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

The addition of nucleoside triphosphates (NTPs) to the 3′ end of a growing RNA strand is catalyzed by a polymerase, such as T7 RNA polymerase, for example, any one or more of the T7 RNA polymerase variants (e.g., G47A) of the present disclosure. In some embodiments, the RNA polymerase (e.g., T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

In some embodiments, the polynucleotide of the present disclosure is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded PCCA or PCCB polypeptide. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding an PCCA or PCCB polypeptide can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of an PCCA or PCCB polypeptide, or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture and methods of making a polynucleotide are disclosed in International PCT application WO 2017/201325, filed on 18 May 2017, the entire contents of which are hereby incorporated by reference.

Chemical Synthesis

Standard methods can be applied to synthesize an isolated polynucleotide sequence encoding an isolated polypeptide of interest, such as a polynucleotide of the invention (e.g., a polynucleotide comprising a nucleotide sequence encoding an PCCA or PCCB polypeptide). For example, a single DNA or RNA oligomer containing a codon-optimized nucleotide sequence coding for the particular isolated polypeptide can be synthesized. In other aspects, several small oligonucleotides coding for portions of the desired polypeptide can be synthesized and then ligated. In some aspects, the individual oligonucleotides typically contain 5′ or 3′ overhangs for complementary assembly.

A polynucleotide disclosed herein (e.g., a RNA, e.g., an mRNA) can be chemically synthesized using chemical synthesis methods and potential nucleobase substitutions known in the art. See, for example, International Publication Nos. WO2014093924, WO2013052523; WO2013039857, WO2012135805, WO2013151671; U.S. Publ. No. US20130115272; or U.S. Pat. Nos. U.S. Pat. No. 8,999,380 or U.S. Pat. No. 8,710,200, all of which are herein incorporated by reference in their entireties.

Quantification of Expressed Polynucleotides Encoding PCCA or PCCB

In some embodiments, the polynucleotides of the present invention (e.g., a polynucleotide comprising a nucleotide sequence encoding an PCCA or PCCB polypeptide), their expression products, as well as degradation products and metabolites can be quantified according to methods known in the art.

In some embodiments, the polynucleotides of the present invention can be quantified in exosomes or when derived from one or more bodily fluid. As used herein “bodily fluids” include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes can be retrieved from an organ selected from the group consisting of lung, heart, pancreas, stomach, intestine, bladder, kidney, ovary, testis, skin, colon, breast, prostate, brain, esophagus, liver, and placenta.

In the exosome quantification method, a sample of not more than 2 mL is obtained from the subject and the exosomes isolated by size exclusion chromatography, density gradient centrifugation, differential centrifugation, nanomembrane ultrafiltration, immunoabsorbent capture, affinity purification, microfluidic separation, or combinations thereof. In the analysis, the level or concentration of a polynucleotide can be an expression level, presence, absence, truncation or alteration of the administered construct. It is advantageous to correlate the level with one or more clinical phenotypes or with an assay for a human disease biomarker.

The assay can be performed using construct specific probes, cytometry, qRT-PCR, real-time PCR, PCR, flow cytometry, electrophoresis, mass spectrometry, or combinations thereof while the exosomes can be isolated using immunohistochemical methods such as enzyme linked immunosorbent assay (ELISA) methods. Exosomes can also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of polynucleotides remaining or delivered. This is possible because the polynucleotides of the present invention differ from the endogenous forms due to the structural or chemical modifications.

In some embodiments, the polynucleotide can be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified polynucleotide can be analyzed in order to determine if the polynucleotide can be of proper size, check that no degradation of the polynucleotide has occurred. Degradation of the polynucleotide can be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Pharmaceutical Compositions and Formulations

The present invention provides pharmaceutical compositions and formulations that comprise any of the polynucleotides described above. In some embodiments, the composition or formulation further comprises a delivery agent.

In some embodiments, the composition or formulation can contain a polynucleotide comprising a sequence optimized nucleic acid sequence disclosed herein which encodes an PCCA and/or PCCB polypeptide. In some embodiments, the composition or formulation can contain a polynucleotide (e.g., a RNA, e.g., an mRNA) comprising a polynucleotide (e.g., an ORF) having significant sequence identity to a sequence optimized nucleic acid sequence disclosed herein which encodes an PCCA and/or PCCB polypeptide. In some embodiments, the polynucleotide further comprises a miRNA binding site, e.g., a miRNA binding site that binds miR-126, miR-142, miR-144, miR-146, miR-150, miR-155, miR-16, miR-21, miR-223, miR-24, miR-27 and miR-26a.

Pharmaceutical compositions or formulation can optionally comprise one or more additional active substances, e.g., therapeutically and/or prophylactically active substances. Pharmaceutical compositions or formulation of the present invention can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005 (incorporated herein by reference in its entirety). In some embodiments, compositions are administered to humans, human patients or subjects. For the purposes of the present disclosure, the phrase “active ingredient” generally refers to polynucleotides to be delivered as described herein.

Formulations and pharmaceutical compositions described herein can be prepared by any method known or hereafter developed in the art of pharmacology. In general, such preparatory methods include the step of associating the active ingredient with an excipient and/or one or more other accessory ingredients, and then, if necessary and/or desirable, dividing, shaping and/or packaging the product into a desired single- or multi-dose unit.

A pharmaceutical composition or formulation in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered.

In some embodiments, the compositions and formulations described herein can contain at least one polynucleotide of the invention. As a non-limiting example, the composition or formulation can contain 1, 2, 3, 4 or 5 polynucleotides of the invention. In some embodiments, the compositions or formulations described herein can comprise more than one type of polynucleotide. In some embodiments, the composition or formulation can comprise a polynucleotide in linear and circular form. In another embodiment, the composition or formulation can comprise a circular polynucleotide and an in vitro transcribed (IVT) polynucleotide. In yet another embodiment, the composition or formulation can comprise an IVT polynucleotide, a chimeric polynucleotide and a circular polynucleotide.

Although the descriptions of pharmaceutical compositions and formulations provided herein are principally directed to pharmaceutical compositions and formulations that are suitable for administration to humans, it will be understood by the skilled artisan that such compositions are generally suitable for administration to any other animal, e.g., to non-human animals, e.g. non-human mammals.

The present invention provides pharmaceutical formulations that comprise a polynucleotide described herein (e.g., a polynucleotide comprising a nucleotide sequence encoding an PCCA and/or PCCB polypeptide). The polynucleotides described herein can be Formulated using one or more excipients to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In some embodiments, the pharmaceutical formulation further comprises a delivery agent comprising, e.g., a compound having the Formula (II), e.g., Compound B. In some embodiments, the delivery agent comprises an ionizable amino lipid (e.g., Compound B), a helper lipid (e.g., DSPC), a sterol (e.g., Cholesterol), and a PEG lipid (e.g., Compound I or PEG-DMG), e.g., with a mole ratio in the range of about (i) 40-50 mol % ionizable amino lipid (e.g., Compound B), optionally 45-50 mol % ionizable amino lipid, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %; (ii) 30-45 mol % sterol (e.g., cholesterol), optionally 35-42 mol % sterol, for example, 30-31 mol %, 31-32 mol %, 32-33 mol %, 33-34 mol %, 35-35 mol %, 35-36 mol %, 36-37 mol %, 37-38 mol %, 38-39 mol %, or 39-40 mol %, or 40-42 mol % sterol; (iii) 5-15 mol % helper lipid (e.g., DSPC), optionally 10-15 mol % helper lipid, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % helper lipid; and (iv) 1-5% PEG lipid (e.g., Compound I or PEG-DMG), optionally 1-5 mol % PEG lipid, for example 1.5 to 2.5 mol %, 1-2 mol %, 2-3 mol %, 3-4 mol %, or 4-5 mol % PEG lipid. In some embodiments, the delivery agent comprises Compound B, Cholesterol, DSPC, and Compound I with a mole ratio of 47:39:11:3.

A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for Formulating pharmaceutical compositions and techniques for preparing the composition are known in the art (see Remington: The Science and Practice of Pharmacy, 21st Edition, A. R. Gennaro (Lippincott, Williams & Wilkins, Baltimore, M D, 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ®30]), PLUORINC®F 68, POLOXAMER®188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Oxidation is a potential degradation pathway for mRNA, especially for liquid mRNA formulations. In order to prevent oxidation, antioxidants can be added to the formulations. Exemplary antioxidants include, but are not limited to, alpha tocopherol, ascorbic acid, ascorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

In some embodiments, the pH of polynucleotide solutions is maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition or formulation described here can contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

The pharmaceutical composition or formulation described here can contain a bulking agent in lyophilized polynucleotide formulations to yield a “pharmaceutically elegant” cake, stabilize the lyophilized polynucleotides during long term (e.g., 36 month) storage. Exemplary bulking agents of the present invention can include, but are not limited to sucrose, trehalose, mannitol, glycine, lactose, raffinose, and combinations thereof.

In some embodiments, the pharmaceutical composition or formulation further comprises a delivery agent. The delivery agent of the present disclosure can include, without limitation, liposomes, lipid nanoparticles, lipidoids, polymers, lipoplexes, microvesicles, exosomes, peptides, proteins, cells transfected with polynucleotides, hyaluronidase, nanoparticle mimics, nanotubes, conjugates, and combinations thereof.

### Delivery Agents

### Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In certain embodiments, the present application provides pharmaceutical compositions comprising:
(a) a polynucleotide comprising a nucleotide sequence encoding an PCCA and/or PCCB polypeptide; and
(b) a delivery agent.

### Lipid Nanoparticle Formulations

In some embodiments, nucleic acids of the invention (e.g., PCCA and/or PCCB mRNA) are Formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Nucleic acids of the present disclosure (e.g., PCCA and/or PCCB mRNA) are typically Formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 40-50 mol %, optionally 45-50 mol %, for example, 45-46 mol %, 46-47 mol %, 47-48 mol %, 48-49 mol %, or 49-50 mol %, for example about 45 mol %, 45.5 mol %, 46 mol %, 46.5 mol %, 47 mol %, 47.5 mol %, 48 mol %, 48.5 mol %, 49 mol %, or 49.5 mol % ionizable cationic lipid.

In some embodiments, the lipid nanoparticle comprises a molar ratio of 5-25% non-cationic lipid. For example, the lipid nanoparticle may comprise a molar ratio of 5-15 mol %, optionally 10-12 mol %, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % non-cationic lipid.

In some embodiments, the lipid nanoparticle comprises a molar ratio of 25-55% sterol. For example, the lipid nanoparticle may comprise a molar ratio of 30-45 mol %, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol % sterol.

In some embodiments, the lipid nanoparticle comprises a molar ratio of 0.5-15% PEG-modified lipid. For example, the lipid nanoparticle may comprise a molar ratio of 1-5%, optionally 1-3 mol %, for example 1.5 to 2.5 mol %, 1-2 mol %, 2-3 mol %, 3-4 mol %, or 4-5 mol % PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the lipid nanoparticle comprises a molar ratio of 40-50% ionizable cationic lipid, 5-15% non-cationic lipid, 30-45% sterol, and 1-5% PEG-modified lipid.

In some embodiments, the lipid nanoparticle comprises a molar ratio of 45-50% ionizable cationic lipid, 10-12% non-cationic lipid, 35-40% sterol, and 1-3% PEG-modified lipid.

In some embodiments, the lipid nanoparticle comprises a molar ratio of 45-50% ionizable cationic lipid, 10-12% non-cationic lipid, 35-40% sterol, and 1.5-2.5% PEG-modified lipid.

### Ionizable Amino Lipids

In some aspects, the disclosure relates to a compound of Formula (I):

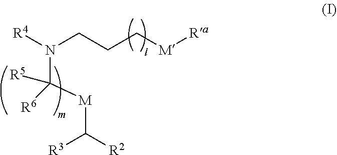

or its N-oxide, or a salt or isomer thereof,
wherein R′^{a} is R′^{branched}; wherein
   R′^{branched} is:

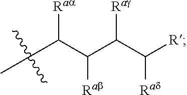

wherein

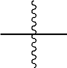

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and
C₂₋₁₄ alkenyl;
   R⁴ is selected from the group consisting of —(CH₂)ₙOH, wherein n is selected from the group consisting of 1, 2, 3, 4, and 5, and

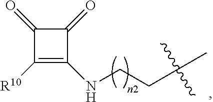

wherein

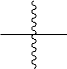

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and
—OC(O)—;
   R′ is a C₁₋₁₂ alkyl or C₂₋₁₂ alkenyl;
   l is selected from the group consisting of 1, 2, 3, 4, and 5; and
   m is selected from the group consisting of 5, 6, 7, 8, 9, 10, 11, 12, and 13.

In some embodiments of the compounds of Formula (I), R′^{a} is R′^{branched};
R′^{branched} is

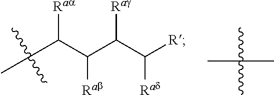

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched};
R′^{branched} is

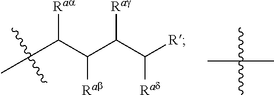

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (I), R′^{a} is R′^{branched};
R′^{branched} is

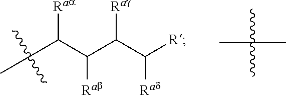

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

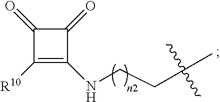

R¹⁰NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched};
R′^{branched} is

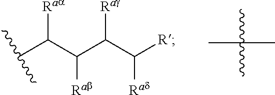

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (I) is selected from:

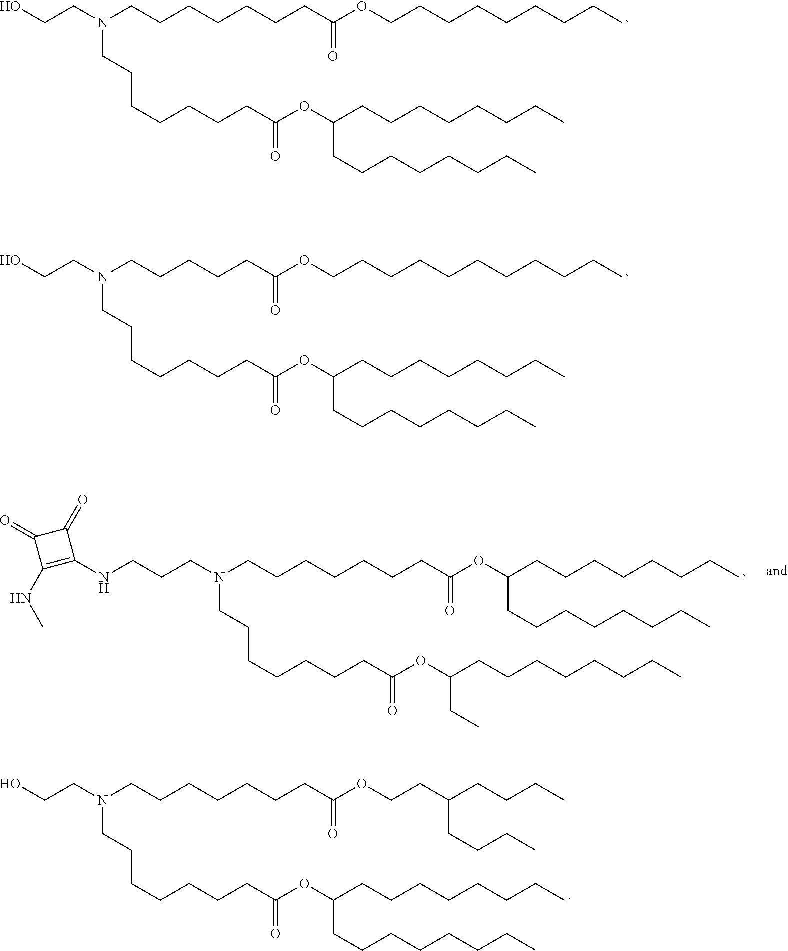 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the compound of Formula (I) is:

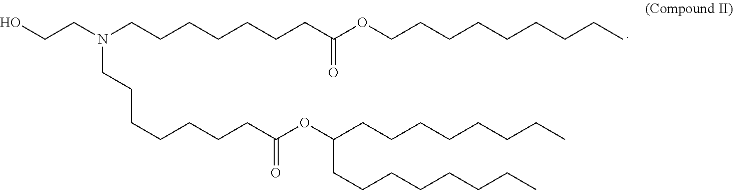 [SMILES: CCCCCCCCCOC(=O)CCCCCCCN(CCO)CCCCCCCC(=O)OC(CCCCCCCC)CCCCCCCC]

In some embodiments, the compound of Formula (I) is:

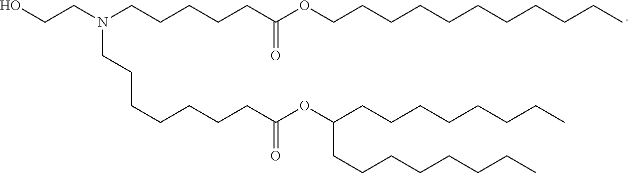 [SMILES: CCCCCCCCCCCOC(=O)CCCCCN(CCO)CCCCCCCC(=O)OC(CCCCCCCC)CCCCCCCC]

In some embodiments, the compound of Formula (I) is:

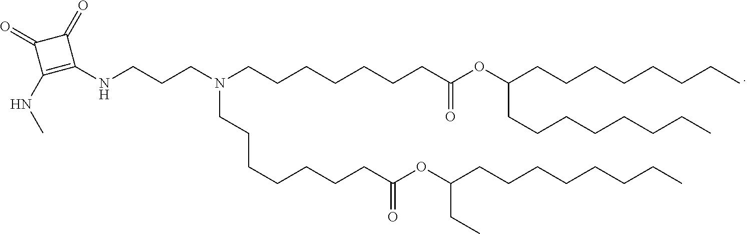 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the compound of Formula (I) is:

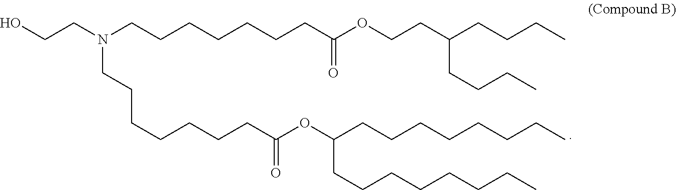 [SMILES: CCCCCCCCC(CCCCCCCC)OC(=O)CCCCCCCN(CCO)CCCCCCCC(=O)OCCC(CCCC)CCCC]

In some aspects, the disclosure relates to a compound of Formula (Ia):

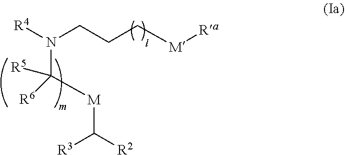

or its N-oxide, or a salt or isomer thereof,
wherein R′^{a} is R′^{branched}; wherein
   R′^{branched} is:

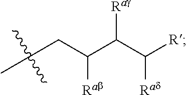

wherein

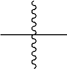

denotes a point of attachment;
wherein R^{aα}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and
C₂₋₁₄ alkenyl;
   R⁴ is selected from the group consisting of —(CH₂)ₙOH wherein n is selected from the group consisting of 1, 2, 3, 4, and 5, and

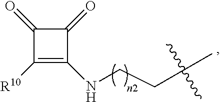

wherein

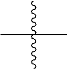

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and
—OC(O)—;
   R′ is a C₁₋₁₂ alkyl or C₂₋₁₂ alkenyl;
   l is selected from the group consisting of 1, 2, 3, 4, and 5; and
   m is selected from the group consisting of 5, 6, 7, 8, 9, 10, 11, 12, and 13.

In some aspects, the disclosure relates to a compound of Formula (Ib):

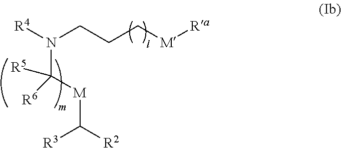

or its N-oxide, or a salt or isomer thereof,
wherein R′^{a} is R′^{branched}; wherein
   R′^{branched} is:

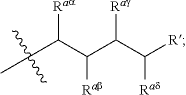

wherein

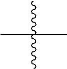

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and
C₂₋₁₄ alkenyl;
   R⁴ is —(CH₂)ₙOH, wherein n is selected from the group consisting of 1, 2, 3, 4, and 5;
   each R⁵ is independently selected from the group consisting of C₁₋₃ alkyl, C₂₋₃ alkenyl, and H;
   each R⁶ is independently selected from the group consisting of C₁₋₃ alkyl, C₂₋₃ alkenyl, and H;
   M and M′ are each independently selected from the group consisting of —C(O)O— and —OC(O)—;
   R′ is a C₁₋₁₂ alkyl or C₂₋₁₂ alkenyl;
   l is selected from the group consisting of 1, 2, 3, 4, and 5; and
   m is selected from the group consisting of 5, 6, 7, 8, 9, 10, 11, 12, and 13.

In some embodiments of Formula (I) or (Ib), R′^{a} is R′^{branched}; R′^{branched} is

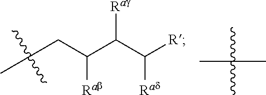

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (I) or (Ib), R′^{a} is R′^{branched}; R′^{branched} is

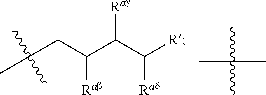

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (I) or (Ib), R′^{a} is R′^{branched}; R′^{branched} is

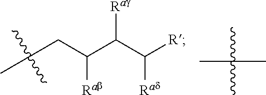

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H;
M and M′ are each —C(O)O—; R′ is a C₁₋₁₂ alkyl; 1 is 5; and m is 7.

In some aspects, the disclosure relates to a compound of Formula (Ic):

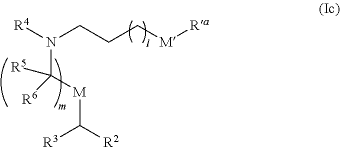

or its N-oxide, or a salt or isomer thereof,
wherein R′^{a} is R′^{branched}; wherein
   R′^{branched} is:

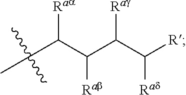

wherein

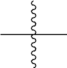

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and
C₂₋₁₄ alkenyl;
   R⁴ is

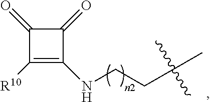

wherein

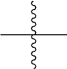

denotes a point of attachment; wherein
R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and
—OC(O)—;
   R′ is a C₁₋₁₂ alkyl or C₂₋₁₂ alkenyl;
   l is selected from the group consisting of 1, 2, 3, 4, and 5; and
   m is selected from the group consisting of 5, 6, 7, 8, 9, 10, 11, 12, and 13.

In some embodiments, R′^{a} is R′^{branched}; R′^{branched} is

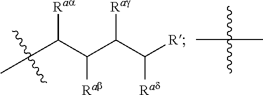

denotes a point of attachment R^{aα}, R^{aα}, and R^{aδ} are each H; R^{aα} is C₂₋₁₂ alkyl; R² and R³ are each C₁₋₁₄ alkyl; R⁴ is

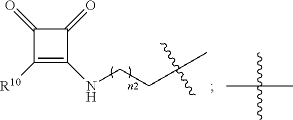

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (Ic) is:

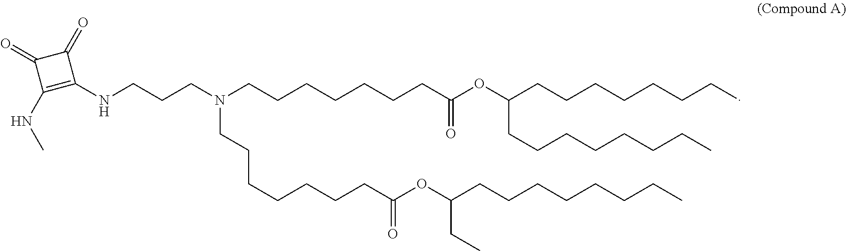

In some aspects, the disclosure relates to a compound of Formula (II):

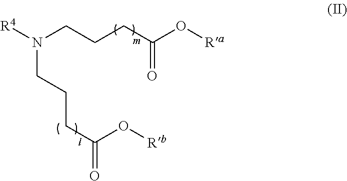

or its N-oxide, or a salt or isomer thereof,
wherein R′^{a} is R′^{branched} or R′^{cyclic}; wherein
R′^{branched} is:

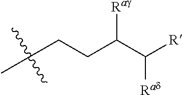

and R′^{cyclic} is:

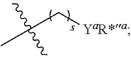

and
   R′^{b} is:

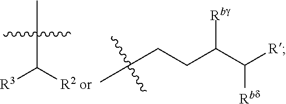

wherein

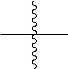

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH wherein n is selected from the group consisting of 1, 2, 3, 4, and 5, and

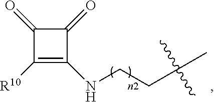

wherein

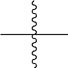

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-a):

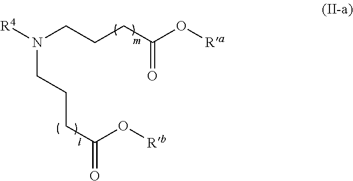

or its N-oxide, or a salt or isomer thereof,
wherein R′^{a} is R′^{branched} or R′^{cyclic}; wherein
R′^{branched} is:

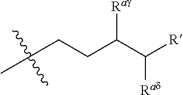

and R′^{b} is:

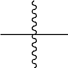

wherein

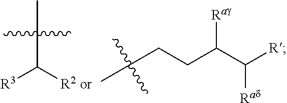

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH wherein n is selected from the group consisting of 1, 2, 3, 4, and 5, and

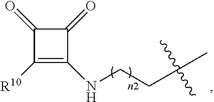

wherein

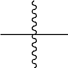

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-b):

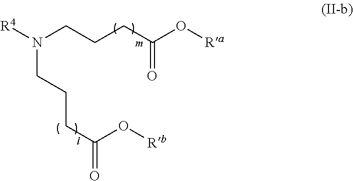

or its N-oxide, or a salt or isomer thereof,
wherein R′^{a} is R′^{branched} or R′^{cyclic}; wherein
R′^{branched} is:

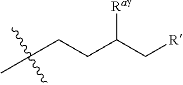

and R′^{b} is:

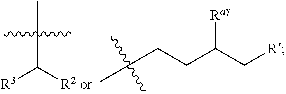

wherein

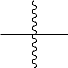

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH wherein n is selected from the group consisting of 1, 2, 3, 4, and 5, and

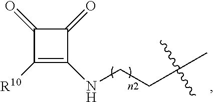

wherein

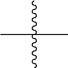

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-c):

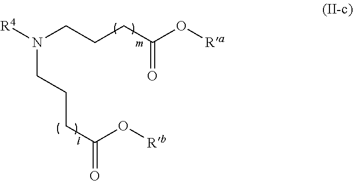

or its N-oxide, or a salt or isomer thereof,
wherein R′^{a} is R′^{branched} or R′^{cyclic}; wherein
R′^{branched} is:

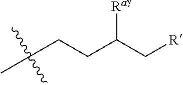

and R′^{b} is:

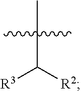

wherein

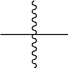

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is selected from the group consisting of —(CH₂)ₙOH wherein n is selected from the group consisting of 1, 2, 3, 4, and 5, and

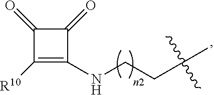

wherein

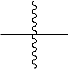

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-d):

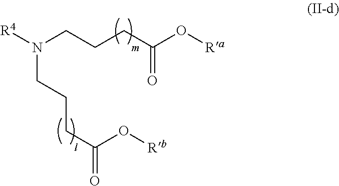

or its N-oxide, or a salt or isomer thereof,
wherein R′^{a} is R′^{branched} or R′^{cyclic}; wherein
R′^{branched} is:

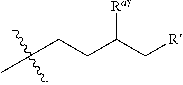

and R′^{b} is:

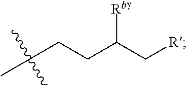

wherein

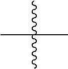

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

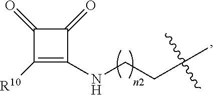

wherein

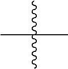

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-e):

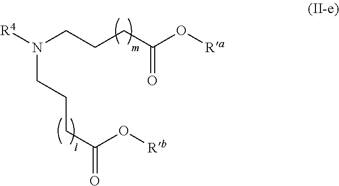

or its N-oxide, or a salt or isomer thereof,
wherein R′^{a} is R′^{branched} or R′^{cyclic}; wherein
R′^{branched} is:

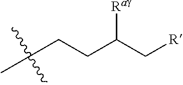

and R′^{b} is:

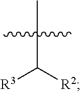

wherein

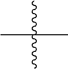

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and
   C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (II), (II-a), (II-b), (II-c), (II-d), or (II-e), m and l are each independently selected from 4, 5, and 6. In some embodiments of the compound of Formula (II), (II-a), (II-b), (II-c), (II-d), or (II-e), m and l are each 5.

In some embodiments of the compound of Formula (II), (II-a), (II-b), (II-c), (II-d), or (II-e), each R′ independently is a C₁₋₁₂ alkyl. In some embodiments of the compound of Formula (II), (II-a), (II-b), (II-c), (II-d), or (II-e), each R′ independently is a C₂₋₅ alkyl.

In some embodiments of the compound of Formula (II), (II-a), (II-b), (II-c), (II-d), or (II-e), R′^{b} is:

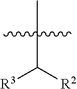

and R² and R³ are each independently a C₁₋₁₄ alkyl. In some embodiments of the compound of Formula (II), (II-a), (II-b), (II-c), (II-d), or (II-e), R′^{b} is:

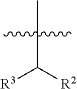

and R² and R³ are each independently a C₆₋₁₀ alkyl. In some embodiments of the compound of Formula (II), (II-a), (II-b), (II-c), (II-d), or (II-e), R′^{b} is:

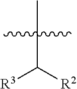

and R² and R³ are each a C₈ alkyl.

In some embodiments of the compound of Formula (II), (II-a), (II-b), (II-c), (II-d), or (II-e), R′^{branched} is:

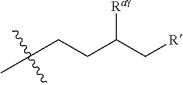

### and R′^{b} is:

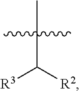

R^{aγ} is a C₁₋₁₂ alkyl and R² and R³ are each independently a C₆₋₁₀ alkyl. In some embodiments of the compound of Formula II (II-a), (II-b), (II-c), (II-d), or (II-e), R′^{branched} is:

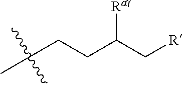

### and R′^{b} is:

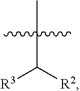

R^{aγ} is a C₂₋₆ alkyl and R² and R³ are each independently a C₆₋₁₀ alkyl. In some embodiments of the compound of Formula (II), (II-a), (II-b), (II-c), (II-d), or (II-e), R′^{branched} is:

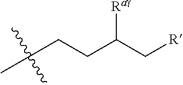

### and R′^{b} is:

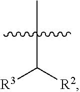

R^{aγ} is a C₂₋₆ alkyl, and R² and R³ are each a C₈ alkyl.

In some embodiments of the compound of Formula (II), (II-a), (II-b), (II-c), (II-d), or (II-e), R′^{branched} is:

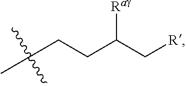

### R′^{b} is:

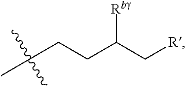

and R^{aγ} and R^{bγ} are each a C₁₋₁₂ alkyl. In some embodiments of the compound of Formula (II), (II-a), (II-b), (II-c), (II-d), or (II-e), R′^{branched} is:

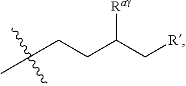

### R′^{b} is:

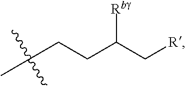

and R^{aγ} and R^{bγ} are each a C₂₋₆ alkyl.

In some embodiments of the compound of Formula (II), (II-a), (II-b), (II-c), (II-d), or (II-e), m and l are each independently selected from 4, 5, and 6 and each R′ independently is a C₁₋₁₂ alkyl. In some embodiments of the compound of Formula (II), (II-a), (II-b), (II-c), (II-d), or (II-e), m and l are each 5 and each R′ independently is a C₂₋₅ alkyl.

In some embodiments of the compound of Formula (II), (II-a), (II-b), (II-c), (II-d), or (II-e), R′^{branched} is:

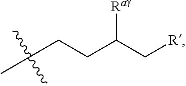

### R′^{b} is:

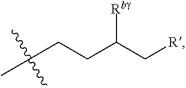

m and l are each
independently selected from 4, 5, and 6, each R′ independently is a C₁₋₁₂ alkyl, and R^{aγ} and R^{bγ} are each a C₁₋₁₂ alkyl. In some embodiments of the compound of Formula (II), (II-a), (II-b), (II-c), (II-d), or (II-e), R′^{branched} is:

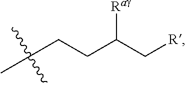

### R′^{b} is:

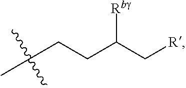

m and l are each 5, each R′ independently is a C₂₋₅ alkyl, and R^{aγ} and R^{bγ} are each a C₂₋₆ alkyl.

In some embodiments of the compound of Formula (II), (II-a), (II-b), (II-c), (II-d), or (II-e), R′^{branched} is:

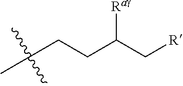

### and R′^{b} is:

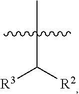

m and l are each independently selected from 4, 5, and 6, R′ is a C₁₋₁₂ alkyl, R^{aγ} is a C₁₋₁₂ alkyl and R² and R₃ are each independently a C₆₋₁₀ alkyl. In some embodiments of the compound of Formula (II), (II-a), (II-b), (II-c), (II-d), or (II-e), R′^{branched} is:

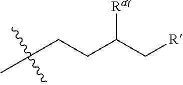

### and R′^{b} is:

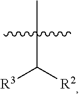

m and l are each 5, R′ is a C₂₋₅ alkyl, R^{aγ} is a C₂₋₆ alkyl, and R² and R³ are each a C₈ alkyl.

In some embodiments of the compound of Formula (II), (II-a), (II-b), (II-c), (II-d), or (II-e), R⁴ is

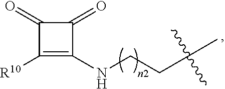

wherein R¹⁰ is NH(C₁₋₆ alkyl) and n2 is 2. In some embodiments of the compound of Formula (II), (II-a), (II-b), (II-c), (II-d), or (II-e), R⁴ is

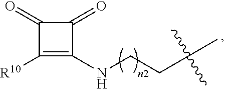

wherein R¹⁰ is NH(CH₃) and n2 is 2.

In some embodiments of the compound of Formula (II), (II-a), (II-b), (II-c), (II-d), or (II-e), R′^{branched} is:

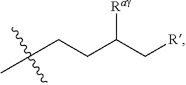

### R′⁶ is:

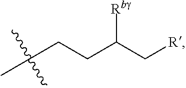

m and l are each independently selected from 4, 5, and 6, each R′ independently is a C₁₋₁₂ alkyl, R^{aγ} and R^{bγ} are each a C₁₋₁₂ alkyl, and R₄ is

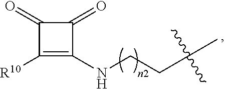

wherein R¹⁰ is NH(C₁₋₆ alkyl), and n2 is 2. In some embodiments of the compound of Formula (II), (II-a), (II-b), (II-c), (II-d), or (II-e), R′^{branched} is:

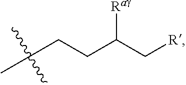

### R′^{b} is:

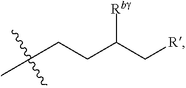

m and l are each 5, each R′ independently is a C₂₋₅ alkyl, R^{aγ} and R^{bγ} are each a C₂₋₆ alkyl, and R⁴ is

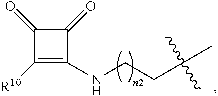

wherein R¹⁰ is NH(CH₃) and n2 is 2.

In some embodiments of the compound of Formula (II), (II-a), (II-b), (II-c), (II-d), or (II-e), R′^{branched} is:

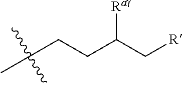

### and R′^{b} is:

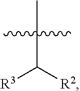

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

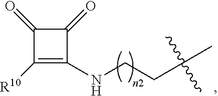

wherein R¹⁰ is NH(C₁₋₆ alkyl) and n2 is 2. In some embodiments of the compound of Formula (II) (II-a) (II-b) (II-c) (II-d), or (II-e), R′^{branched} is:

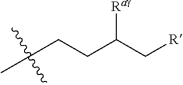

### and R′^{b} is:

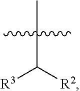

m and l are each 5, R′ is a C₂₋₅ alkyl, R^{aγ} is a C₂₋₆ alkyl, R² and R³ are each a C₈ alkyl, and R₄ is

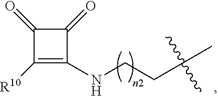

wherein R¹⁰ is NH(CH₃) and n2 is 2.

In some embodiments of the compound of Formula (II), (II-a), (II-b), (II-c), (II-d), or (II-e), R⁴ is —(CH₂)ₙOH and n is 2, 3, or 4. In some embodiments of the compound of Formula (II), (II-a), (II-b), (II-c), (II-d), or (II-e), R⁴ is —(CH₂)ₙOH and n is 2.

In some embodiments of the compound of Formula (II), (II-a), (II-b), (II-c), (II-d), or (II-e), R′^{branched} is:

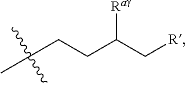

### R′^{b} is:

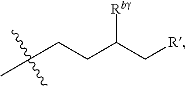

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (II), (II-a), (II-b), (II-c), (II-d), or (II-e), R′^{branched} is:

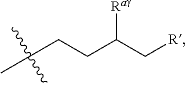

### R′^{b} is:

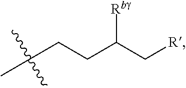

m and l are each 5, each R′ independently is a C₂₋₅ alkyl, R^{aγ} and R^{bγ} are each a C₂₋₆ alkyl, R⁴ is —(CH₂)ₙOH, and n is 2.

In some aspects, the disclosure relates to a compound of Formula (II-f):

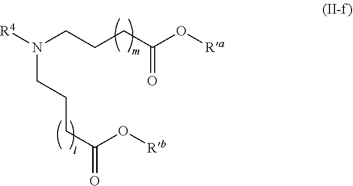

or its N-oxide, or a salt or isomer thereof,
wherein R′^{a} is R′^{branched} or R′^{cyclic}; wherein
R′^{branched} is:

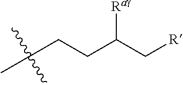

and R′^{b} is:

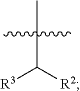

wherein

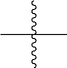

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (II-f), m and l are each 5, and n is 2, 3, or 4.

In some embodiments of the compound of Formula (II-f) R′ is a C₂₋₅ alkyl, R^{aγ} is a C₂₋₆ alkyl, and R² and R³ are each a C₆₋₁₀ alkyl.

In some embodiments of the compound of Formula (II-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some aspects, the disclosure relates to a compound of Formula (II-g):

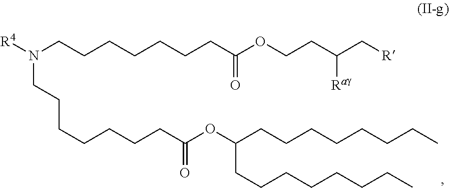

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

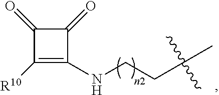

wherein

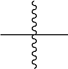

denotes a point of attachment, R¹⁰ is NH(C₁₋₆ alkyl), and n2 is selected from the group consisting of 1, 2, and 3.

In some aspects, the disclosure relates to a compound of Formula (II-h):

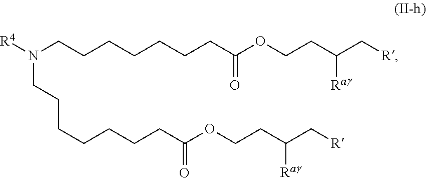

wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

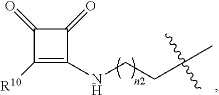

wherein

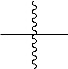

denotes a point of attachment, R¹⁰ is NH(C₁₋₆ alkyl), and n2 is selected from the group consisting of 1, 2, and 3.

In some embodiments of the compound of Formula (II-g) or (II-h), R⁴ is

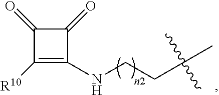

wherein
R¹⁰ is NH(CH₃) and n2 is 2.

In some embodiments of the compound of Formula (II-g) or (II-h), R⁴ is —(CH₂)₂OH.

In some aspects, the disclosure relates to a compound having the Formula (III):

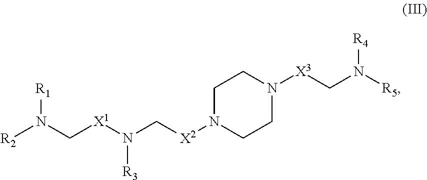

or a salt or isomer thereof, wherein
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
each M is independently selected from the group consisting of —C(O)O—, —OC(O)—, —OC(O)O—, —C(O)N(R′)—, —N(R′)C(O)—, —C(O)—, —C(S)—, —C(S)S—, —S C(S)—,
   —CH(OH)—, —P(O)(OR′)O—, —S(O)₂—, an aryl group, and a heteroaryl group;
X¹, X², and X³ are independently selected from the group consisting of a bond, —CH₂—,
   —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂-12 alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃-12 alkenyl, and wherein:
i) at least one of X¹, X², and X³ is not —CH₂—; and/or
ii) at least one of R₁, R₂, R₃, R₄, and R₅ is —R″MR′.

In some embodiments, R₁, R₂, R₃, R₄, and R₅ are each C₅₋₂₀ alkyl; X¹ is —CH₂—; and X² and X³ are each —C(O)—.

In some embodiments, the compound of Formula (III) is:

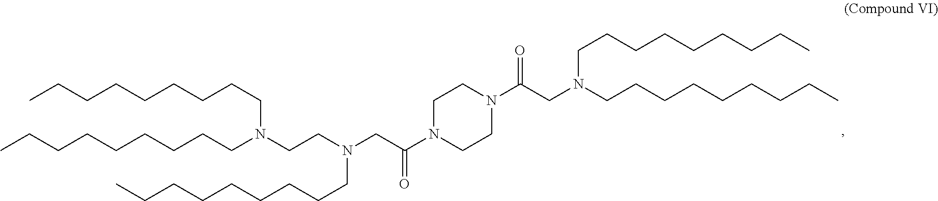 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt or isomer thereof.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

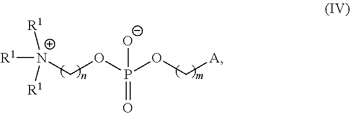

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the Formula:

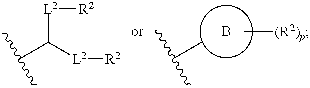

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O) C(O)O, OC(O), —OC(O)O, —OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O C(O)S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the Formula:

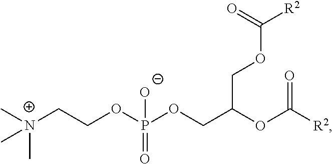

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

### Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following Formulae:

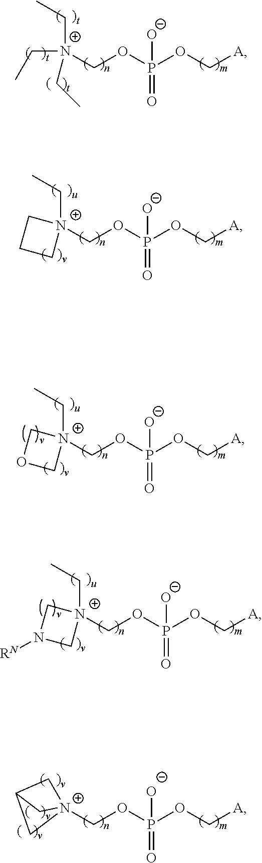

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

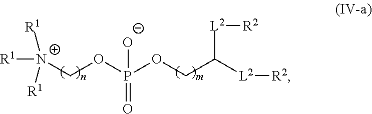

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

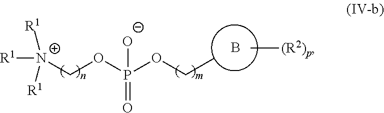

or a salt thereof.

### Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O C(O)S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O.

In certain embodiments, the compound of Formula (IV) is of Formula (IV-c):

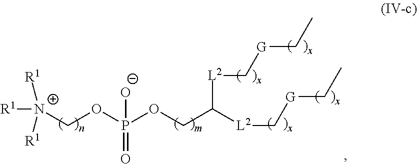

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O C(O)S, SC(O), C(═NR^{N}), C(═NR^{N})N(R^{N}), NR^{N}C(═NR^{N}), NR^{N}C(═NR^{N})N(R^{N}) C(S), —C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following Formulae:

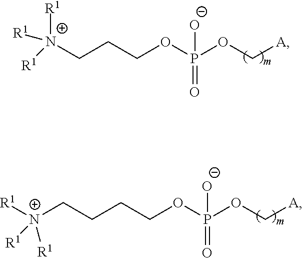

or a salt thereof.

### Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the invention is oleic acid.

In certain embodiments, the alternative lipid is one of the following:

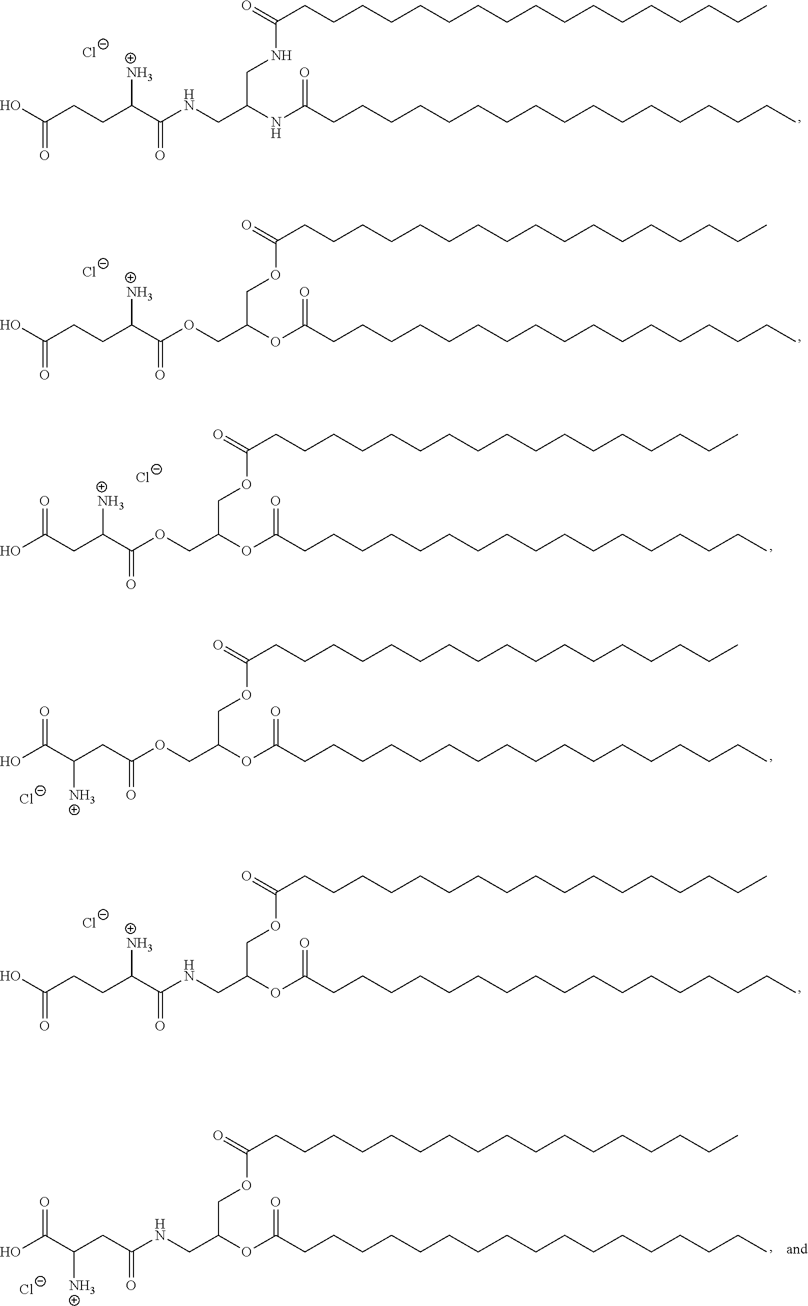

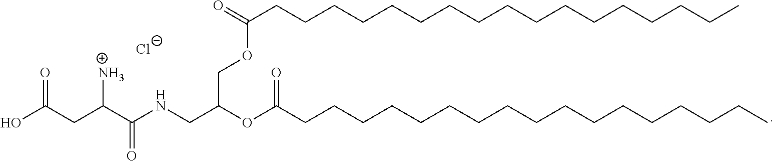 [SMILES: CCCCCCCCCCCCCCCCCC(=O)OCC(CNC(=O)C([NH3+])CC(=O)O)OC(=O)CCCCCCCCCCCCCCCCC ; [Cl-]]

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. Application No. 62/520,530.

### Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various Formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

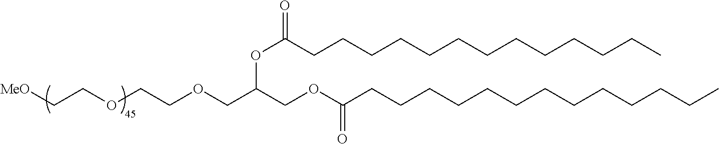

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

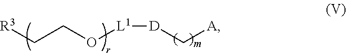

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O) C(O)O, OC(O), OC(O)O, OC(O)N(R^{N}), NR^{N}C(O)O, or NR^{N}C(O)N(R^{N}); D is a moiety obtained by click chemistry or a moiety cleavable under physiological conditions;
m is 0, 1, 2, 3, 4, 5, 6, 7, 8, 9, or 10;
A is of the Formula:

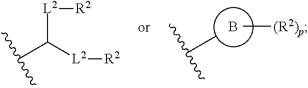

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O) C(O)O, OC(O), —OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O C(O)S, SC(O), —C(═NR^{N}), C(═NR^{N})N(R^{N}), NR^{N}C(═NR^{N}) NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

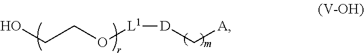

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

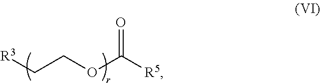

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, —N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), —NR^{N}C(═NR^{N})N(R^{N}), C(S), C(S)N(R^{N}), NR^{N}C(S), NR^{N}C(S)N(R^{N}) S(O), OS(O), S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), —OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (VI) is of Formula (VI-OH):

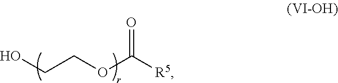

or a salt thereof. In some embodiments, r is 45.

In yet other embodiments the compound of Formula (VI) is:

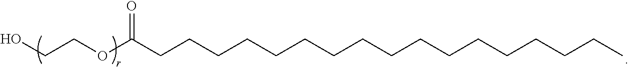

or a salt thereof.

In one embodiment, the compound of Formula (VI) is

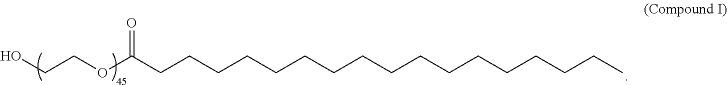

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the invention comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a LNP of the invention comprises an ionizable cationic lipid of any of Formula I, II or III, a phospholipid comprising DSPC, a structural lipid, and a PEG lipid comprising PEG-DMG.

In some embodiments, a LNP of the invention comprises an ionizable cationic lipid of any of Formula I, II or III, a phospholipid comprising DSPC, a structural lipid, and a PEG lipid comprising a compound having Formula VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid having Formula IV, a structural lipid, and a PEG lipid comprising a compound having Formula VI.

In some embodiments, a LNP of the invention comprises an ionizable cationic lipid of

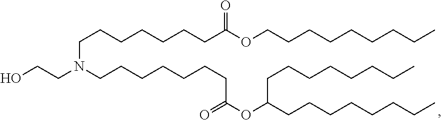 [SMILES: CCCCCCCCCOC(=O)CCCCCCCN(CCO)CCCCCCCC(=O)OC(CCCCCCCC)CCCCCCCC]

and a PEG lipid comprising Formula VI.

In some embodiments, a LNP of the invention comprises an ionizable cationic lipid of

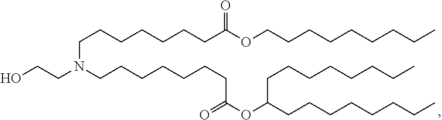 [SMILES: CCCCCCCCCOC(=O)CCCCCCCN(CCO)CCCCCCCC(=O)OC(CCCCCCCC)CCCCCCCC]

and an alternative lipid comprising oleic acid.

In some embodiments, a LNP of the invention comprises an ionizable cationic lipid of

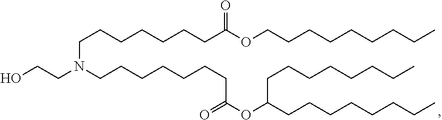 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid comprising a compound having Formula VI.

In some embodiments, a LNP of the invention comprises an ionizable cationic lipid of

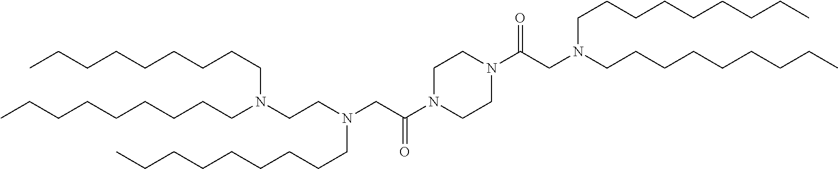 [SMILES: CCCCCCCCCN(CCCCCCCCC)CCN(CCCCCCCCC)CC(=O)N1CCN(C(=O)CN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the invention comprises an ionizable cationic lipid of

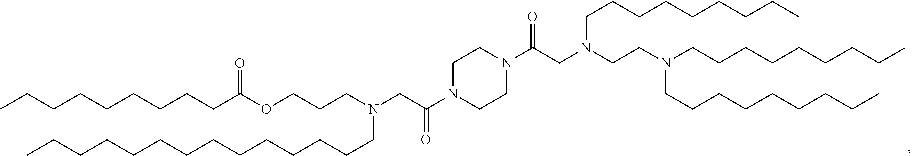 [SMILES: CCCCCCCCCCCCCCN(CCCOC(=O)CCCCCCCCC)CC(=O)N1CCN(C(=O)CN(CCCCCCCCC)CCN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1.

In some embodiments, a LNP of the invention comprises a wt/wt ratio of the ionizable cationic lipid component to the RNA of from about 10:1 to about 100:1.

In some embodiments, a LNP of the invention comprises a wt/wt ratio of the ionizable cationic lipid component to the RNA of about 20:1.

In some embodiments, a LNP of the invention comprises a wt/wt ratio of the ionizable cationic lipid component to the RNA of about 10:1.

In some embodiments, a LNP of the invention has a mean diameter from about 50 nm to about 150 nm.

In some embodiments, a LNP of the invention has a mean diameter from about 70 nm to about 120 nm.

As used herein, the term “alkyl”, “alkyl group”, or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. For example, Cis alkenyl may include one or more double bonds. A Cis alkenyl group including two double bonds may be a linoleyl group. Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, Cis alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl”, “heteroalkenyl”, or “heteroalkynyl”, refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the Formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)₂R″″, in which each OR are alkoxy groups that can be the same or different and R″″ is an alkyl or alkenyl group), a phosphate (e.g., P(O)₄³⁻), a thiol (e.g., SH), a sulfoxide (e.g., S(O)R), a sulfinic acid (e.g., S(O)OH), a sulfonic acid (e.g., S(O)₂OH), a thial (e.g., C(S)H), a sulfate (e.g., S(O)₄²⁻), a sulfonyl (e.g., S(O)₂), an amide (e.g., C(O)NR₂, or N(R)C(O)R), an azido (e.g., N₃), a nitro (e.g., N₀₂), a cyano (e.g., CN), an isocyano (e.g., NC), an acyloxy (e.g., OC(O)R), an amino (e.g., NR₂, NRH, or NH₂), a carbamoyl (e.g., OC(O)NR₂, OC(O)NRH, or OC(O)NH₂), a sulfonamide (e.g., S(O)₂NR₂, S(O)₂NRH, S(O)₂NH₂, N(R)S(O)₂R, N(H)S(O)₂R, N(R)S(O)₂H, or N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C₁₋₆ alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N→O or N+—O—). Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

### Other Lipid Composition Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In one embodiment, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

### Nanoparticle Compositions

In some embodiments, the pharmaceutical compositions disclosed herein are Formulated as lipid nanoparticles (LNP). Accordingly, the present disclosure also provides nanoparticle compositions comprising (i) a lipid composition comprising a delivery agent such as compound as described herein, and (ii) a polynucleotide encoding an PCCA and/or PCCB polypeptide. In such nanoparticle composition, the lipid composition disclosed herein can encapsulate the polynucleotide encoding an PCCA and/or PCCB polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable amino lipid, a structural lipid, a phospholipid, and mRNA. In some embodiments, the LNP comprises an ionizable amino lipid, a PEG-modified lipid, a sterol and a structural lipid. In some embodiments, the LNP has a molar ratio of about 40-50% ionizable amino lipid; about 5-15% structural lipid; about 30-45% sterol; and about 1-5% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable amino lipid. As used herein, the term “ionizable amino lipid” has its ordinary meaning in the art and may refer to a lipid comprising one or more charged moieties. In some embodiments, an ionizable amino lipid may be positively charged or negatively charged. An ionizable amino lipid may be positively charged, in which case it can be referred to as “cationic lipid”. In certain embodiments, an ionizable amino lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

The ionizable amino lipid is sometimes referred to in the art as an “ionizable cationic lipid”. In one embodiment, the ionizable amino lipid may have a positively charged hydrophilic head and a hydrophobic tail that are connected via a linker structure.

In addition to these, an ionizable amino lipid may also be a lipid including a cyclic amine group.

In one embodiment, the ionizable amino lipid may be selected from, but not limited to, an ionizable amino lipid described in International Publication Nos.

WO2013086354 and WO2013116126; the contents of each of which are herein incorporated by reference in their entirety.

In yet another embodiment, the ionizable amino lipid may be selected from, but not limited to, Formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding an PCCA and/or PCCB polypeptide are Formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

### Other Delivery Agents

### Liposomes, Lipoplexes, and Lipid Nanoparticles

In some embodiments, the compositions or formulations of the present disclosure comprise a delivery agent, e.g., a liposome, a lioplexes, a lipid nanoparticle, or any combination thereof. The polynucleotides described herein (e.g., a polynucleotide comprising a nucleotide sequence encoding an PCCA and/or PCCB polypeptide) can be Formulated using one or more liposomes, lipoplexes, or lipid nanoparticles. Liposomes, lipoplexes, or lipid nanoparticles can be used to improve the efficacy of the polynucleotides directed protein production as these formulations can increase cell transfection by the polynucleotide; and/or increase the translation of encoded protein. The liposomes, lipoplexes, or lipid nanoparticles can also be used to increase the stability of the polynucleotides.

Liposomes are artificially-prepared vesicles that can primarily be composed of a lipid bilayer and can be used as a delivery vehicle for the administration of pharmaceutical formulations. Liposomes can be of different sizes. A multilamellar vesicle (MLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos. US20130177638, US20130177637, US20130177636, US20130177635, US20130177634, US20130177633, US20130183375, US20130183373, and US20130183372. In some embodiments, the polynucleotides described herein can be encapsulated by the liposome and/or it can be contained in an aqueous core that can then be encapsulated by the liposome as described in, e.g., Intl. Pub. Nos. WO2012031046, WO2012031043, WO2012030901, WO2012006378, and WO2013086526; and U.S. Pub. Nos. US20130189351, US20130195969 and US20130202684. Each of the references in herein incorporated by reference in its entirety.

In some embodiments, the polynucleotides described herein can be Formulated in a cationic oil-in-water emulsion where the emulsion particle comprises an oil core and a cationic lipid that can interact with the polynucleotide anchoring the molecule to the emulsion particle. In some embodiments, the polynucleotides described herein can be Formulated in a water-in-oil emulsion comprising a continuous hydrophobic phase in which the hydrophilic phase is dispersed. Exemplary emulsions can be made by the methods described in Intl. Pub. Nos. WO2012006380 and WO201087791, each of which is herein incorporated by reference in its entirety.

In some embodiments, the polynucleotides described herein can be Formulated in a lipid-polycation complex. The formation of the lipid-polycation complex can be accomplished by methods as described in, e.g., U.S. Pub. No. US20120178702. As a non-limiting example, the polycation can include a cationic peptide or a polypeptide such as, but not limited to, polylysine, polyornithine and/or polyarginine and the cationic peptides described in Intl. Pub. No. WO2012013326 or U.S. Pub. No. US20130142818. Each of the references is herein incorporated by reference in its entirety.

In some embodiments, the polynucleotides described herein can be Formulated in a lipid nanoparticle (LNP) such as those described in Intl. Pub. Nos. WO2013123523, WO2012170930, WO2011127255 and WO2008103276; and U.S. Pub. No. US20130171646, each of which is herein incorporated by reference in its entirety.

Lipid nanoparticle formulations typically comprise one or more lipids. In some embodiments, the lipid is an ionizable amino lipid, sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable amino lipids include, but not limited to, any Compounds II, VI, A, and B disclosed herein, DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable amino lipids include, (13Z,16Z)—N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)—N,N-dimethylnonacosa-11,20,2-trien-10-amine, and any combination thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %. In some embodiments, the amount of phospholipids (e.g., DSPC) in the lipid composition ranges from about 5-15 mol %, optionally 10-12 mol %, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %. In some embodiments, the amount of the structural lipids (e.g., cholesterol) in the lipid composition ranges from about 30-45 mol %, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %.

The PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some embodiments, the amount of PEG-lipid in the lipid composition ranges from about 0 mol % to about 5 mol %. In some embodiments, the amount of PEG-lipid in the lipid composition ranges from about 1-5%, optionally 1-3 mol %, for example 1.5 to 2.5 mol %, 1-2 mol %, 2-3 mol %, 3-4 mol %, or 4-5 mol %.

In some embodiments, the LNP formulations described herein can additionally comprise a permeability enhancer molecule. Non-limiting permeability enhancer molecules are described in U.S. Pub. No. US20050222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP Formulation can also contain a polymer conjugate (e.g., a water soluble conjugate) as described in, e.g., U.S. Pub. Nos. US20130059360, US20130196948, and US20130072709. Each of the references is herein incorporated by reference in its entirety.

The LNP formulations can comprise a conjugate to enhance the delivery of nanoparticles of the present invention in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975, herein incorporated by reference in its entirety). As shown by Rodriguez et al. the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013110028, each of which is herein incorporated by reference in its entirety.

The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin 34 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028, herein incorporated by reference in its entirety.

In some embodiments, the polynucleotide described herein is Formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In some embodiments, the polynucleotides described herein are Formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in Intl. Pub. No. WO2013105101, herein incorporated by reference in its entirety.

In some embodiments, the polynucleotides described herein can be Formulated for controlled release and/or targeted delivery. As used herein, “controlled release” refers to a pharmaceutical composition or compound release profile that conforms to a particular pattern of release to effect a therapeutic outcome. In one embodiment, the polynucleotides can be encapsulated into a delivery agent described herein and/or known in the art for controlled release and/or targeted delivery. As used herein, the term “encapsulate” means to enclose, surround or encase. As it relates to the formulation of the compounds of the invention, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent.

Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, or greater than 99% of the pharmaceutical composition or compound of the invention are encapsulated in the delivery agent.

In some embodiments, the polynucleotides described herein can be encapsulated in a therapeutic nanoparticle, referred to herein as “therapeutic nanoparticle polynucleotides.” Therapeutic nanoparticles can be Formulated by methods described in, e.g., Intl. Pub. Nos. WO2010005740, WO2010030763, WO2010005721, WO2010005723, and WO2012054923; and U.S. Pub. Nos. US20110262491, US20100104645, US20100087337, US20100068285, US20110274759, US20100068286, US20120288541, US20120140790, US20130123351 and US20130230567; and U.S. Pat. Nos. 8,206,747, 8,293,276, 8,318,208 and 8,318,211, each of which is herein incorporated by reference in its entirety.

In some embodiments, the therapeutic nanoparticle polynucleotide can be Formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the polynucleotides described herein can be Formulated as disclosed in Intl. Pub. No. WO2010075072 and U.S. Pub. Nos. US20100216804, US20110217377, US20120201859 and US20130150295, each of which is herein incorporated by reference in their entirety.

In some embodiments, the therapeutic nanoparticle polynucleotide can be Formulated to be target specific, such as those described in Intl. Pub. Nos. WO2008121949, WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsev et al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic Formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM,) from the Institut fur Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the polynucleotides described herein can be Formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002); each of which is herein incorporated by reference in its entirety). In some embodiments, the polynucleotides can be Formulated in lipid nanoparticles using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

In some embodiments, the polynucleotides described herein can be Formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the polynucleotides can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

The nanoparticles and microparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., Intl. Pub. No. WO2013082111, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

### Lipidoids

In some embodiments, the compositions or formulations of the present disclosure comprise a delivery agent, e.g., a lipidoid. The polynucleotides described herein (e.g., a polynucleotide comprising a nucleotide sequence encoding an PCCA and/or PCCB polypeptide) can be Formulated with lipidoids. Complexes, micelles, liposomes or particles can be prepared containing these lipidoids and therefore to achieve an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intramuscular, or subcutaneous routes.

The synthesis of lipidoids is described in literature (see Mahon et al., Bioconjug. Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

Formulations with the different lipidoids, including, but not limited to penta[3-(1-laurylaminopropionyl)]-triethylenetetramine hydrochloride (TETA-5LAP; also known as 98N12-5, see Murugaiah et al., Analytical Biochemistry, 401:61 (2010)), C12-200 (including derivatives and variants), and MD1, can be tested for in vivo activity. The lipidoid “98N12-5” is disclosed by Akinc et al., Mol Ther. 2009 17:872-879. The lipidoid “C12-200” is disclosed by Love et al., Proc Natl Acad Sci USA. 2010 107:1864-1869 and Liu and Huang, Molecular Therapy. 2010 669-670. Each of the references is herein incorporated by reference in its entirety.

In one embodiment, the polynucleotides described herein can be Formulated in an aminoalcohol lipidoid. Aminoalcohol lipidoids can be prepared by the methods described in U.S. Pat. No. 8,450,298 (herein incorporated by reference in its entirety).

The lipidoid formulations can include particles comprising either 3 or 4 or more components in addition to polynucleotides. Lipidoids and polynucleotide formulations comprising lipidoids are described in Intl. Pub. No. WO 2015051214 (herein incorporated by reference in its entirety.

### Hyaluronidase

In some embodiments, the polynucleotides described herein (e.g., a polynucleotide comprising a nucleotide sequence encoding an PCCA and/or PCCB polypeptide) and hyaluronidase for injection (e.g., intramuscular or subcutaneous injection). Hyaluronidase catalyzes the hydrolysis of hyaluronan, which is a constituent of the interstitial barrier. Hyaluronidase lowers the viscosity of hyaluronan, thereby increases tissue permeability (Frost, Expert Opin. Drug Deliv. (2007) 4:427-440). Alternatively, the hyaluronidase can be used to increase the number of cells exposed to the polynucleotides administered intramuscularly, or subcutaneously.

### Nanoparticle Mimics

In some embodiments, the polynucleotides described herein (e.g., a polynucleotide comprising a nucleotide sequence encoding an PCCA and/or PCCB polypeptide) is encapsulated within and/or absorbed to a nanoparticle mimic. A nanoparticle mimic can mimic the delivery function organisms or particles such as, but not limited to, pathogens, viruses, bacteria, fungus, parasites, prions and cells. As a non-limiting example, the polynucleotides described herein can be encapsulated in a non-viron particle that can mimic the delivery function of a virus (see e.g., Intl. Pub. No. WO2012006376 and U.S. Pub. Nos. US20130171241 and US20130195968, each of which is herein incorporated by reference in its entirety).

### Self-Assembled Nanoparticles, or Self-Assembled Macromolecules

In some embodiments, the compositions or formulations of the present disclosure comprise the polynucleotides described herein (e.g., a polynucleotide comprising a nucleotide sequence encoding an PCCA and/or PCCB polypeptide) in self-assembled nanoparticles, or amphiphilic macromolecules (AMs) for delivery. AMs comprise biocompatible amphiphilic polymers that have an alkylated sugar backbone covalently linked to poly(ethylene glycol). In aqueous solution, the AMs self-assemble to form micelles. Nucleic acid self-assembled nanoparticles are described in Intl. Appl. No. PCT/US2014/027077, and AMs and methods of forming AMs are described in U.S. Pub. No. US20130217753, each of which is herein incorporated by reference in its entirety.

### Cations and Anions

In some embodiments, the compositions or formulations of the present disclosure comprise the polynucleotides described herein (e.g., a polynucleotide comprising a nucleotide sequence encoding an PCCA and/or PCCB polypeptide) and a cation or anion, such as Z²⁺, Ca²⁺, Cu²⁺, Mg²⁺ and combinations thereof. Exemplary formulations can include polymers and a polynucleotide complexed with a metal cation as described in, e.g., U.S. Pat. Nos. 6,265,389 and 6,555,525, each of which is herein incorporated by reference in its entirety. In some embodiments, cationic nanoparticles can contain a combination of divalent and monovalent cations. The delivery of polynucleotides in cationic nanoparticles or in one or more depot comprising cationic nanoparticles can improve polynucleotide bioavailability by acting as a long-acting depot and/or reducing the rate of degradation by nucleases.

### Amino Acid Lipids

In some embodiments, the compositions or formulations of the present disclosure comprise the polynucleotides described herein (e.g., a polynucleotide comprising a nucleotide sequence encoding an PCCA and/or PCCB polypeptide) that is formulation with an amino acid lipid. Amino acid lipids are lipophilic compounds comprising an amino acid residue and one or more lipophilic tails. Non-limiting examples of amino acid lipids and methods of making amino acid lipids are described in U.S. Pat. No. 8,501,824. The amino acid lipid formulations can deliver a polynucleotide in releasable form that comprises an amino acid lipid that binds and releases the polynucleotides. As a non-limiting example, the release of the polynucleotides described herein can be provided by an acid-labile linker as described in, e.g., U.S. Pat. Nos. 7,098,032, 6,897,196, 6,426,086, 7,138,382, 5,563,250, and 5,505,931, each of which is herein incorporated by reference in its entirety.

### Interpolyelectrolyte Complexes

In some embodiments, the compositions or formulations of the present disclosure comprise the polynucleotides described herein (e.g., a polynucleotide comprising a nucleotide sequence encoding an PCCA and/or PCCB polypeptide) in an interpolyelectrolyte complex. Interpolyelectrolyte complexes are formed when charge-dynamic polymers are complexed with one or more anionic molecules. Non-limiting examples of charge-dynamic polymers and interpolyelectrolyte complexes and methods of making interpolyelectrolyte complexes are described in U.S. Pat. No. 8,524,368, herein incorporated by reference in its entirety.

### Crystalline Polymeric Systems

In some embodiments, the compositions or formulations of the present disclosure comprise the polynucleotides described herein (e.g., a polynucleotide comprising a nucleotide sequence encoding an PCCA and/or PCCB polypeptide) in crystalline polymeric systems. Crystalline polymeric systems are polymers with crystalline moieties and/or terminal units comprising crystalline moieties. Exemplary polymers are described in U.S. Pat. No. 8,524,259 (herein incorporated by reference in its entirety).

### Polymers, Biodegradable Nanoparticles, and Core-Shell Nanoparticles

In some embodiments, the compositions or formulations of the present disclosure comprise the polynucleotides described herein (e.g., a polynucleotide comprising a nucleotide sequence encoding an PCCA and/or PCCB polypeptide) and a natural and/or synthetic polymer. The polymers include, but not limited to, polyethenes, polyethylene glycol (PEG), poly(l-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, elastic biodegradable polymer, biodegradable copolymer, biodegradable polyester copolymer, biodegradable polyester copolymer, multiblock copolymers, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyureas, polystyrenes, polyamines, polylysine, poly(ethylene imine), poly(serine ester), poly(L-lactide-co-L-lysine), poly(4-hydroxy-L-proline ester), amine-containing polymers, dextran polymers, dextran polymer derivatives or combinations thereof.

Exemplary polymers include, DYNAMIC POLYCONJUGATE® (Arrowhead Research Corp., Pasadena, CA) formulations from MIRUS® Bio (Madison, WI) and Roche Madison (Madison, WI), PHASERX™ polymer formulations such as, without limitation, SMARTT POLYMER TECHNOLOGY™ (PHASERX®, Seattle, WA), DMRI/DOPE, poloxamer, VAXFECTIN® adjuvant from Vical (San Diego, CA), chitosan, cyclodextrin from Calando Pharmaceuticals (Pasadena, CA), dendrimers and poly(lactic-co-glycolic acid) (PLGA) polymers. RONDEL™ (RNAi/Oligonucleotide Nanoparticle Delivery) polymers (Arrowhead Research Corporation, Pasadena, CA) and pH responsive co-block polymers such as PHASERX® (Seattle, WA).

The polymer formulations allow a sustained or delayed release of the polynucleotide (e.g., following intramuscular or subcutaneous injection). The altered release profile for the polynucleotide can result in, for example, translation of an encoded protein over an extended period of time. The polymer formulation can also be used to increase the stability of the polynucleotide. Sustained release formulations can include, but are not limited to, PLGA microspheres, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc. Deerfield, IL), PEG-based sealants, and COSEAL® (Baxter International, Inc. Deerfield, IL).

As a non-limiting example modified mRNA can be Formulated in PLGA microspheres by preparing the PLGA microspheres with tunable release rates (e.g., days and weeks) and encapsulating the modified mRNA in the PLGA microspheres while maintaining the integrity of the modified mRNA during the encapsulation process. EVAc are non-biodegradable, biocompatible polymers that are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C.

As a non-limiting example, the polynucleotides described herein can be Formulated with the polymeric compound of PEG grafted with PLL as described in U.S. Pat. No. 6,177,274. As another non-limiting example, the polynucleotides described herein can be Formulated with a block copolymer such as a PLGA-PEG block copolymer (see e.g., U.S. Pub. No. US20120004293 and U.S. Pat. Nos. 8,236,330 and 8,246,968), or a PLGA-PEG-PLGA block copolymer (see e.g., U.S. Pat. No. 6,004,573). Each of the references is herein incorporated by reference in its entirety.

In some embodiments, the polynucleotides described herein can be Formulated with at least one amine-containing polymer such as, but not limited to polylysine, polyethylene imine, poly(amidoamine) dendrimers, poly(amine-co-esters) or combinations thereof. Exemplary polyamine polymers and their use as delivery agents are described in, e.g., U.S. Pat. Nos. 8,460,696, 8,236,280, each of which is herein incorporated by reference in its entirety.

In some embodiments, the polynucleotides described herein can be Formulated in a biodegradable cationic lipopolymer, a biodegradable polymer, or a biodegradable copolymer, a biodegradable polyester copolymer, a biodegradable polyester polymer, a linear biodegradable copolymer, PAGA, a biodegradable cross-linked cationic multi-block copolymer or combinations thereof as described in, e.g., U.S. Pat. Nos. 6,696,038, 6,517,869, 6,267,987, 6,217,912, 6,652,886, 8,057,821, and 8,444,992; U.S. Pub. Nos. US20030073619, US20040142474, US20100004315, US2012009145 and US20130195920; and Intl Pub. Nos. WO2006063249 and WO2013086322, each of which is herein incorporated by reference in its entirety.

In some embodiments, the polynucleotides described herein can be Formulated in or with at least one cyclodextrin polymer as described in U.S. Pub. No. US20130184453. In some embodiments, the polynucleotides described herein can be Formulated in or with at least one crosslinked cation-binding polymers as described in Intl. Pub. Nos. WO2013106072, WO2013106073 and WO2013106086. In some embodiments, the polynucleotides described herein can be Formulated in or with at least PEGylated albumin polymer as described in U.S. Pub. No. US20130231287. Each of the references is herein incorporated by reference in its entirety.

In some embodiments, the polynucleotides disclosed herein can be Formulated as a nanoparticle using a combination of polymers, lipids, and/or other biodegradable agents, such as, but not limited to, calcium phosphate. Components can be combined in a core-shell, hybrid, and/or layer-by-layer architecture, to allow for fine-tuning of the nanoparticle for delivery (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87; herein incorporated by reference in their entireties). As a non-limiting example, the nanoparticle can comprise a plurality of polymers such as, but not limited to hydrophilic-hydrophobic polymers (e.g., PEG-PLGA), hydrophobic polymers (e.g., PEG) and/or hydrophilic polymers (Intl. Pub. No. WO20120225129, herein incorporated by reference in its entirety).

The use of core-shell nanoparticles has additionally focused on a high-throughput approach to synthesize cationic cross-linked nanogel cores and various shells (Siegwart et al., Proc Natl Acad Sci USA. 2011 108:12996-13001; herein incorporated by reference in its entirety). The complexation, delivery, and internalization of the polymeric nanoparticles can be precisely controlled by altering the chemical composition in both the core and shell components of the nanoparticle. For example, the core-shell nanoparticles can efficiently deliver siRNA to mouse hepatocytes after they covalently attach cholesterol to the nanoparticle.

In some embodiments, a hollow lipid core comprising a middle PLGA layer and an outer neutral lipid layer containing PEG can be used to delivery of the polynucleotides as described herein. In some embodiments, the lipid nanoparticles can comprise a core of the polynucleotides disclosed herein and a polymer shell, which is used to protect the polynucleotides in the core. The polymer shell can be any of the polymers described herein and are known in the art. The polymer shell can be used to protect the polynucleotides in the core.

Core-shell nanoparticles for use with the polynucleotides described herein are described in U.S. Pat. No. 8,313,777 or Intl. Pub. No. WO2013124867, each of which is herein incorporated by reference in their entirety.

### Peptides and Proteins

In some embodiments, the compositions or formulations of the present disclosure comprise the polynucleotides described herein (e.g., a polynucleotide comprising a nucleotide sequence encoding an PCCA and/or PCCB polypeptide) that is Formulated with peptides and/or proteins to increase transfection of cells by the polynucleotide, and/or to alter the biodistribution of the polynucleotide (e.g., by targeting specific tissues or cell types), and/or increase the translation of encoded protein (e.g., Intl. Pub. Nos. WO2012110636 and WO2013123298. In some embodiments, the peptides can be those described in U.S. Pub. Nos. US20130129726, US20130137644 and US20130164219. Each of the references is herein incorporated by reference in its entirety.

### Conjugates

In some embodiments, the compositions or formulations of the present disclosure comprise the polynucleotides described herein (e.g., a polynucleotide comprising a nucleotide sequence encoding an PCCA and/or PCCB polypeptide) that is covalently linked to a carrier or targeting group, or including two encoding regions that together produce a fusion protein (e.g., bearing a targeting group and therapeutic protein or peptide) as a conjugate. The conjugate can be a peptide that selectively directs the nanoparticle to neurons in a tissue or organism, or assists in crossing the blood-brain barrier.

The conjugates include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid, an oligonucleotide (e.g., an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

In some embodiments, the conjugate can function as a carrier for the polynucleotide disclosed herein. The conjugate can comprise a cationic polymer such as, but not limited to, polyamine, polylysine, polyalkylenimine, and polyethylenimine that can be grafted to with poly(ethylene glycol). Exemplary conjugates and their preparations are described in U.S. Pat. No. 6,586,524 and U.S. Pub. No. US20130211249, each of which herein is incorporated by reference in its entirety.

The conjugates can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as an endothelial cell or bone cell. Targeting groups can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent frucose, or aptamers. The ligand can be, for example, a lipopolysaccharide, or an activator of p38 MAP kinase.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein. As a non-limiting example, the targeting group can be a glutathione receptor (GR)-binding conjugate for targeted delivery across the blood-central nervous system barrier as described in, e.g., U.S. Pub. No. US2013021661012 (herein incorporated by reference in its entirety).

In some embodiments, the conjugate can be a synergistic biomolecule-polymer conjugate, which comprises a long-acting continuous-release system to provide a greater therapeutic efficacy. The synergistic biomolecule-polymer conjugate can be those described in U.S. Pub. No. US20130195799. In some embodiments, the conjugate can be an aptamer conjugate as described in Intl. Pat. Pub. No. WO2012040524. In some embodiments, the conjugate can be an amine containing polymer conjugate as described in U.S. Pat. No. 8,507,653. Each of the references is herein incorporated by reference in its entirety. In some embodiments, the polynucleotides can be conjugated to SMARTT POLYMER TECHNOLOGY® (PHASERX®, Inc. Seattle, WA).

In some embodiments, the polynucleotides described herein are covalently conjugated to a cell penetrating polypeptide, which can also include a signal sequence or a targeting sequence. The conjugates can be designed to have increased stability, and/or increased cell transfection; and/or altered the biodistribution (e.g., targeted to specific tissues or cell types).

In some embodiments, the polynucleotides described herein can be conjugated to an agent to enhance delivery. In some embodiments, the agent can be a monomer or polymer such as a targeting monomer or a polymer having targeting blocks as described in Intl. Pub. No. WO2011062965. In some embodiments, the agent can be a transport agent covalently coupled to a polynucleotide as described in, e.g., U.S. Pat. Nos. 6,835,393 and 7,374,778. In some embodiments, the agent can be a membrane barrier transport enhancing agent such as those described in U.S. Pat. Nos. 7,737,108 and 8,003,129. Each of the references is herein incorporated by reference in its entirety.

### Methods of Use

The polynucleotides, pharmaceutical compositions and formulations described above are used in the preparation, manufacture and therapeutic use of to treat and/or prevent PCC-related diseases, disorders or conditions. In some embodiments, the polynucleotides, compositions and formulations of the present disclosure are used to treat and/or prevent propionic acidemia.

In some embodiments, the polynucleotides, pharmaceutical compositions and formulations of the present disclosure are used in methods for reducing the levels of ammonia in a subject in need thereof. For instance, one aspect of the present disclosure provides a method of alleviating the signs and symptoms of propionic acidemia in a subject comprising the administration of a composition or formulation comprising a polynucleotide encoding PCCA and/or PCCB to that subject (e.g., an mRNA encoding an PCCA and/or PCCB polypeptide).

In some embodiments, the administration of an effective amount of a polynucleotide, pharmaceutical composition or formulation of the invention reduces the levels of a biomarker of propionic acidemia, e.g., propionyl-L-carnitine (C3), 2-methylcitric acid (2-MC), 3-hydroxypropionic acid, (30HPA), propionylglycine, glycine, lactate and/or ammonia. In some embodiments, the administration of the polynucleotide, pharmaceutical composition or formulation of the invention results in reduction in the level of one or more biomarkers of propionic acidemia, e.g., propionyl-L-carnitine (C3), 2-methylcitric acid (2-MC), 3-hydroxypropionic acid, (30HPA), propionylglycine, glycine, lactate and/or ammonia, within a short period of time (e.g., within about 6 hours, within about 8 hours, within about 12 hours, within about 16 hours, within about 20 hours, or within about 24 hours) after administration of the polynucleotide, pharmaceutical composition or formulation of the invention.

In some embodiments, the administration of an effective amount of a polynucleotide, pharmaceutical composition or formulation of the invention increases body weight of a human subject. In some embodiments, the administration of the polynucleotide, pharmaceutical composition or formulation of the invention results in an increase in body weight within a short period of time (e.g., within about 12 hours, 24 hours, 48 hours, 72 hours, 96 hours, 5 days, 7 days, 14 days, 24 days, 48 days, or 60 days) after administration of the polynucleotide, pharmaceutical composition or formulation of the invention.

In some embodiments, the administration of an effective amount of a polynucleotide, pharmaceutical composition or formulation of the invention maintains body weight of a human subject.

Replacement therapy is a potential treatment for propionic acidemia. Thus, in certain aspects of the present disclosure, the polynucleotides, e.g., mRNA, disclosed herein comprise one or more sequences encoding an PCCA and/or PCCB polypeptide that is suitable for use in gene replacement therapy for propionic acidemia. In some embodiments, the present disclosure treats a lack of PCC or PCC activity, or decreased or abnormal PCC activity in a subject by providing a polynucleotide, e.g., mRNA, that encodes an PCCA and/or PCCB polypeptide to the subject. In some embodiments, the polynucleotide is sequence-optimized. In some embodiments, the polynucleotide (e.g., an mRNA) comprises a nucleic acid sequence (e.g., an ORF) encoding an PCCA and/or PCCB polypeptide, wherein the nucleic acid is sequence-optimized, e.g., by modifying its G/C, uridine, or thymidine content, and/or the polynucleotide comprises at least one chemically modified nucleoside. In some embodiments, the polynucleotide comprises a miRNA binding site, e.g., a miRNA binding site that binds miRNA-142.

In some embodiments, the administration of a composition or formulation comprising polynucleotide, pharmaceutical composition or formulation of the present disclosure to a subject results in a decrease in ammonia in cells to a level at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% lower than the level observed prior to the administration of the composition or formulation.

In some embodiments, the administration of the polynucleotide, pharmaceutical composition or formulation of the present disclosure results in expression of PCCA and/or PCCB in cells of the subject. In some embodiments, administering the polynucleotide, pharmaceutical composition or formulation of the present disclosure results in an increase of PCC enzymatic activity in the subject. For example, in some embodiments, the polynucleotides of the present disclosure are used in methods of administering a composition or formulation comprising an mRNA encoding an PCCA and/or PCCB polypeptide to a subject, wherein the method results in an increase of PCC enzymatic activity in at least some cells of a subject.

In some embodiments, the administration of a composition or formulation comprising an mRNA encoding an PCCA and/or PCCB polypeptide to a subject results in an increase of PCC enzymatic activity in cells subject to a level at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% or more of the activity level expected in a normal subject, e.g., a human not suffering from propionic acidemia.

In some embodiments, the administration of the polynucleotide, pharmaceutical composition or formulation of the present disclosure results in expression of PCCA and/or PCCB protein in at least some of the cells of a subject that persists for a period of time sufficient to allow significant chloride channel activity to occur.

In another embodiment, the polynucleotides, pharmaceutical compositions, or formulations of the present disclosure can be repeatedly administered such that PCCA and/or PCCB protein is expressed at a therapeutic level for a period of time sufficient to have a beneficial biological effect as described herein.

In some embodiments, the expression of the encoded polypeptide is increased. In some embodiments, the polynucleotide increases PCCA and/or PCCB expression levels in cells when introduced into those cells, e.g., by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% with respect to the PCCA and/or PCCB expression level in the cells before the polypeptide is introduced in the cells.

In some embodiments, the method or use comprises administering a polynucleotide, e.g., mRNA, comprising a nucleotide sequence having sequence similarity to a polynucleotide of SEQ ID NO:5, wherein the polynucleotide encodes an PCCA polypeptide.

In some embodiments, the method or use comprises administering a polynucleotide, e.g., mRNA, comprising a nucleotide sequence having sequence similarity to a polynucleotide of SEQ ID NO:6, wherein the polynucleotide encodes an PCCB polypeptide.

Other aspects of the present disclosure relate to transplantation of cells containing polynucleotides to a mammalian subject. Administration of cells to mammalian subjects is known to those of ordinary skill in the art, and includes, but is not limited to, local implantation (e.g., topical or subcutaneous administration), organ delivery or systemic injection (e.g., intravenous injection or inhalation), and the formulation of cells in pharmaceutically acceptable carriers.

The present disclosure also provides methods to increase PCC activity in a subject in need thereof, e.g., a subject with propionic acidemia, comprising administering to the subject a therapeutically effective amount of a composition or formulation comprising mRNA encoding an PCCA and/or PCCB polypeptide disclosed herein, e.g., a human PCCA and/or PCCB polypeptide, a mutant thereof, or a fusion protein comprising a human PCCA and/or PCCB.

In some aspects, the PCC activity measured after administration to a subject in need thereof, e.g., a subject with propionic acidemia, is at least the normal PCC activity level observed in healthy human subjects. In some aspects, the PCC activity measured after administration is at higher than the PCC activity level observed in propionic acidemia patients, e.g., untreated propionic acidemia patients. In some aspects, the increase in PCC activity in a subject in need thereof, e.g., a subject with propionic acidemia, after administering to the subject a therapeutically effective amount of a composition or formulation comprising mRNA encoding an PCCA and/or PCCB polypeptide disclosed herein is at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or greater than 100 percent of the normal PCC activity level observed in healthy human subjects. In some aspects, the increase in PCC activity above the PCC activity level observed in propionic acidemia patients after administering to the subject a composition or formulation comprising an mRNA encoding an PCCA and/or PCCB polypeptide disclosed herein (e.g., after a single dose administration) is maintained for at least 1 day, at least 2 days, at least 3 days, at least 4 days, at least 5 days, at least 6 days, at least 7 days, at least 8 days, at least 9 days, at least 10 days, at least 12 days, at least 14 days, at least 21 days, or at least 28 days.

The present disclosure also provides a method to treat, prevent, or ameliorate the symptoms of propionic acidemia in an propionic acidemia patient comprising administering to the subject a therapeutically effective amount of a composition or formulation comprising mRNA encoding an PCCA and/or PCCB polypeptide disclosed herein. In some aspects, the administration of a therapeutically effective amount of a composition or formulation comprising mRNA encoding an PCCA and/or PCCB polypeptide disclosed herein to subject in need of treatment for propionic acidemia results in reducing the symptoms of propionic acidemia.

In some embodiments, the polynucleotides (e.g., mRNA), pharmaceutical compositions and formulations used in the methods of the invention comprise a uracil-modified sequence encoding an PCCA and/or PCCB polypeptide disclosed herein and a miRNA binding site disclosed herein, e.g., a miRNA binding site that binds to miR-142 and/or a miRNA binding site that binds to miR-126. In some embodiments, the uracil-modified sequence encoding an PCCA and/or PCCB polypeptide comprises at least one chemically modified nucleobase, e.g., N1-methylpseudouracil or 5-methoxyuracil. In some embodiments, at least 95% of a type of nucleobase (e.g., uracil) in a uracil-modified sequence encoding an PCCA and/or PCCB polypeptide of the invention are modified nucleobases. In some embodiments, at least 95% of uracil in a uracil-modified sequence encoding an PCCA and/or PCCB polypeptide is 1-N-methylpseudouridine or 5-methoxyuridine. In some embodiments, the polynucleotide (e.g., a RNA, e.g., a mRNA) disclosed herein is Formulated with a delivery agent comprising, e.g., a compound having the Formula (II), e.g., Compound B. In some embodiments, the delivery agent comprises an ionizable amino lipid (e.g., Compound B), a helper lipid (e.g., DSPC), a sterol (e.g., Cholesterol), and a PEG lipid (e.g., Compound I or PEG-DMG), e.g., with a mole ratio in the range of about (i) 40-50 mol % ionizable amino lipid (e.g., Compound B), optionally 45-50 mol % ionizable amino lipid, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %; (ii) 30-45 mol % sterol (e.g., cholesterol), optionally 35-42 mol % sterol, for example, 30-31 mol %, 31-32 mol %, 32-33 mol %, 33-34 mol %, 35-35 mol %, 35-36 mol %, 36-37 mol %, 37-38 mol %, 38-39 mol %, or 39-40 mol %, or 40-42 mol % sterol; (iii) 5-15 mol % helper lipid (e.g., DSPC), optionally 10-15 mol % helper lipid, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % helper lipid; and (iv) 1-5% PEG lipid (e.g., Compound I or PEG-DMG), optionally 1-5 mol % PEG lipid, for example 1.5 to 2.5 mol %, 1-2 mol %, 2-3 mol %, 3-4 mol %, or 4-5 mol % PEG lipid. In some embodiments, the delivery agent comprises Compound B, Cholesterol, DSPC, and Compound I with a mole ratio of 47:39:11:3.

The skilled artisan will appreciate that the therapeutic effectiveness of a drug or a treatment of the instant invention can be characterized or determined by measuring the level of expression of an encoded protein (e.g., enzyme) in a sample or in samples taken from a subject (e.g., from a preclinical test subject (rodent, primate, etc.) or from a clinical subject (human). Likewise, the therapeutic effectiveness of a drug or a treatment of the instant invention can be characterized or determined by measuring the level of activity of an encoded protein (e.g., enzyme) in a sample or in samples taken from a subject (e.g., from a preclinical test subject (rodent, primate, etc.) or from a clinical subject (human). Furthermore, the therapeutic effectiveness of a drug or a treatment of the instant invention can be characterized or determined by measuring the level of an appropriate biomarker in sample(s) taken from a subject. Levels of protein and/or biomarkers can be determined post-administration with a single dose of an mRNA therapeutic of the invention or can be determined and/or monitored at several time points following administration with a single dose or can be determined and/or monitored throughout a course of treatment, e.g., a multi-dose treatment.

PCCA and/or PCCB Protein Expression Levels

Certain aspects of the invention feature measurement, determination and/or monitoring of the expression level or levels of PCCA and/or PCCB protein in a subject, for example, in an animal (e.g., rodents, primates, and the like) or in a human subject. Animals include normal, healthy or wild type animals, as well as animal models for use in understanding propionic acidemia and treatments thereof. Exemplary animal models include rodent models, for example, PCCA and/or PCCB deficient mice also referred to as PCCA and/or PCCB mice.

PCCA and/or PCCB protein expression levels can be measured or determined by any art-recognized method for determining protein levels in biological samples, e.g., from blood samples or a needle biopsy. The term “level” or “level of a protein” as used herein, preferably means the weight, mass or concentration of the protein within a sample or a subject. It will be understood by the skilled artisan that in certain embodiments the sample may be subjected, e.g., to any of the following: purification, precipitation, separation, e.g. centrifugation and/or HPLC, and subsequently subjected to determining the level of the protein, e.g., using mass and/or spectrometric analysis. In exemplary embodiments, enzyme-linked immunosorbent assay (ELISA) can be used to determine protein expression levels. In other exemplary embodiments, protein purification, separation and LC-MS can be used as a means for determining the level of a protein according to the invention. In some embodiments, an mRNA therapy of the invention (e.g., a single intravenous dose) results in increased PCCA and/or PCCB protein expression levels in the tissue (e.g., heart, liver, brain, or skeletal muscle) of the subject (e.g., 2-fold, 3-fold, 4-fold, 5-fold, 6-fold, 7-fold, 8-fold, 9-fold, 10-fold, 20-fold, 30-fold, 40-fold, 50-fold increase and/or increased to at least 50%, at least 60%, at least 70%, at least 75%, 80%, at least 85%, at least 90%, at least 95%, or at least 100% of normal levels) for at least 6 hours, at least 12 hours, at least 24 hours, at least 36 hours, at least 48 hours, at least 60 hours, at least 72 hours, at least 84 hours, at least 96 hours, at least 108 hours, at least 122 hours after administration of a single dose of the mRNA therapy.

PCC Activity

In propionic acidemia patients, PCC enzymatic activity is reduced compared to a normal physiological activity level. Further aspects of the invention feature measurement, determination and/or monitoring of the activity level(s) (i.e., enzymatic activity level(s)) of PCCA and/or PCCB protein in a subject, for example, in an animal (e.g., rodent, primate, and the like) or in a human subject. Activity levels can be measured or determined by any art-recognized method for determining enzymatic activity levels in biological samples. The term “activity level” or “enzymatic activity level” as used herein, preferably means the activity of the enzyme per volume, mass or weight of sample or total protein within a sample. In exemplary embodiments, the “activity level” or “enzymatic activity level” is described in terms of units per milliliter of fluid (e.g., bodily fluid, e.g., serum, plasma, urine and the like) or is described in terms of units per weight of tissue or per weight of protein (e.g., total protein) within a sample. Units (“U”) of enzyme activity can be described in terms of weight or mass of substrate hydrolyzed per unit time. In certain embodiments of the invention feature PCC activity described in terms of U/ml plasma or U/mg protein (tissue), where units (“U”) are described in terms of nmol substrate hydrolyzed per hour (or nmol/hr).

In certain embodiments, an mRNA therapy of the invention features a pharmaceutical composition comprising a dose of mRNA effective to result in at least 5 U/mg, at least 10 U/mg, at least 20 U/mg, at least 30 U/mg, at least 40 U/mg, at least 50 U/mg, at least 60 U/mg, at least 70 U/mg, at least 80 U/mg, at least 90 U/mg, at least 100 U/mg, or at least 150 U/mg of PCC activity in tissue (e.g., liver) between 6 and 12 hours, or between 12 and 24, between 24 and 48, or between 48 and 72 hours post administration (e.g., at 48 or at 72 hours post administration).

In some embodiments, an mRNA therapy of the invention (e.g., a single intravenous dose) results in increased PCC activity levels in the liver tissue of the subject (e.g., 2-fold, 3-fold, 4-fold, 5-fold, 6-fold, 7-fold, 8-fold, 9-fold, 10-fold, 20-fold, 30-fold, 40-fold, 50-fold increase and/or increased to at least 50%, at least 60%, at least 70%, at least 75%, 80%, at least 85%, at least 90%, at least 95%, or at least 100% of normal levels) for at least 6 hours, at least 12 hours, at least 24 hours, or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days after administration of a single dose of the mRNA therapy.

In exemplary embodiments, an mRNA therapy of the invention features a pharmaceutical composition comprising a single intravenous dose of mRNA that results in the above-described levels of activity. In another embodiment, an mRNA therapy of the invention features a pharmaceutical composition which can be administered in multiple single unit intravenous doses of mRNA that maintain the above-described levels of activity.

PCC Biomarkers

In some embodiments, the administration of an effective amount of a polynucleotide, pharmaceutical composition or formulation of the invention reduces the levels of a biomarker of PCC, e.g., propionyl-L-carnitine (C₃), 2-methylcitric acid (2-MC), 3-hydroxypropionic acid, (30HPA), propionylglycine, glycine, lactate and/or ammonia levels. In some embodiments, the administration of the polynucleotide, pharmaceutical composition or formulation of the invention results in reduction in the level of one or more biomarkers of PCC, e.g., propionyl-L-carnitine (C3), 2-methylcitric acid (2-MC), 3-hydroxypropionic acid, (30HPA), propionylglycine, glycine, lactate and/or ammonia levels, within a short period of time after administration of the polynucleotide, pharmaceutical composition or formulation of the invention.

Further aspects of the invention feature determining the level (or levels) of a biomarker determined in a sample as compared to a level (e.g., a reference level) of the same or another biomarker in another sample, e.g., from the same patient, from another patient, from a control and/or from the same or different time points, and/or a physiologic level, and/or an elevated level, and/or a supraphysiologic level, and/or a level of a control. The skilled artisan will be familiar with physiologic levels of biomarkers, for example, levels in normal or wild type animals, normal or healthy subjects, and the like, in particular, the level or levels characteristic of subjects who are healthy and/or normal functioning. As used herein, the phrase “elevated level” means amounts greater than normally found in a normal or wild type preclinical animal or in a normal or healthy subject, e.g. a human subject. As used herein, the term “supraphysiologic” means amounts greater than normally found in a normal or wild type preclinical animal or in a normal or healthy subject, e.g. a human subject, optionally producing a significantly enhanced physiologic response. As used herein, the term “comparing” or “compared to” preferably means the mathematical comparison of the two or more values, e.g., of the levels of the biomarker(s). It will thus be readily apparent to the skilled artisan whether one of the values is higher, lower or identical to another value or group of values if at least two of such values are compared with each other. Comparing or comparison to can be in the context, for example, of comparing to a control value, e.g., as compared to a reference blood, serum, plasma, and/or tissue (e.g., liver) propionyl-L-carnitine (C3), 2-methylcitric acid (2-MC), 3-hydroxypropionic acid, (30HPA), propionylglycine, glycine, lactate and/or ammonia level, in said subject prior to administration (e.g., in a person suffering from propionic acidemia) or in a normal or healthy subject. Comparing or comparison to can also be in the context, for example, of comparing to a control value, e.g., as compared to a reference blood, serum, plasma and/or tissue (e.g., liver) propionyl-L-carnitine (C3), 2-methylcitric acid (2-MC), 3-hydroxypropionic acid, (30HPA), propionylglycine, glycine, lactate and/or ammonia level in said subject prior to administration (e.g., in a person suffering from propionic acidemia) or in a normal or healthy subject.

As used herein, a “control” is preferably a sample from a subject wherein the propionic acidemia status of said subject is known. In one embodiment, a control is a sample of a healthy patient. In another embodiment, the control is a sample from at least one subject having a known propionic acidemia status, for example, a severe, mild, or healthy propionic acidemia status, e.g. a control patient. In another embodiment, the control is a sample from a subject not being treated for propionic acidemia. In a still further embodiment, the control is a sample from a single subject or a pool of samples from different subjects and/or samples taken from the subject(s) at different time points.

The term “level” or “level of a biomarker” as used herein, preferably means the mass, weight or concentration of a biomarker of the invention within a sample or a subject. It will be understood by the skilled artisan that in certain embodiments the sample may be subjected to, e.g., one or more of the following: substance purification, precipitation, separation, e.g. centrifugation and/or HPLC and subsequently subjected to determining the level of the biomarker, e.g. using mass spectrometric analysis. In certain embodiments, LC-MS can be used as a means for determining the level of a biomarker according to the invention.

The term “determining the level” of a biomarker as used herein can mean methods which include quantifying an amount of at least one substance in a sample from a subject, for example, in a bodily fluid from the subject (e.g., serum, plasma, urine, lymph, etc.) or in a tissue of the subject (e.g., liver, etc.).

The term “reference level” as used herein can refer to levels (e.g., of a biomarker) in a subject prior to administration of an mRNA therapy of the invention (e.g., in a person suffering from propionic acidemia) or in a normal or healthy subject.

As used herein, the term “normal subject” or “healthy subject” refers to a subject not suffering from symptoms associated with propionic acidemia. Moreover, a subject will be considered to be normal (or healthy) if it has no mutation of the functional portions or domains of the PCCA and or PCCB gene and/or no mutation of the PCCA and/or PCCB gene resulting in a reduction of or deficiency of the enzyme PCC or the activity thereof, resulting in symptoms associated with propionic acidemia. Said mutations will be detected if a sample from the subject is subjected to a genetic testing for such PCCA and/or PCCB mutations. In certain embodiments of the present invention, a sample from a healthy subject is used as a control sample, or the known or standardized value for the level of biomarker from samples of healthy or normal subjects is used as a control.

In some embodiments, comparing the level of the biomarker in a sample from a subject in need of treatment for propionic acidemia or in a subject being treated for propionic acidemia to a control level of the biomarker comprises comparing the level of the biomarker in the sample from the subject (in need of treatment or being treated for propionic acidemia) to a baseline or reference level, wherein if a level of the biomarker in the sample from the subject (in need of treatment or being treated for propionic acidemia) is elevated, increased or higher compared to the baseline or reference level, this is indicative that the subject is suffering from propionic acidemia and/or is in need of treatment; and/or wherein if a level of the biomarker in the sample from the subject (in need of treatment or being treated for propionic acidemia) is decreased or lower compared to the baseline level this is indicative that the subject is not suffering from, is successfully being treated for propionic acidemia, or is not in need of treatment for propionic acidemia. The stronger the reduction (e.g., at least 2-fold, at least 3-fold, at least 4-fold, at least 5-fold, at least 6-fold, at least 7-fold, at least 8-fold, at least 10-fold, at least 20-fold, at least-30 fold, at least 40-fold, at least 50-fold reduction and/or at least 10%, at least 20%, at least 30% at least 40%, at least 50%, at least 60%, at least 70%, at least 80%, at least 90%, or at least 100% reduction) of the level of a biomarker, within a certain time period, e.g., within 6 hours, within 12 hours, 24 hours, 36 hours, 48 hours, 60 hours, or 72 hours, and/or for a certain duration of time, e.g., 48 hours, 72 hours, 96 hours, 120 hours, 144 hours, 1 week, 2 weeks, 3 weeks, 4 weeks, 1 month, 2 months, 3 months, 4 months, 5 months, 6 months, 7 months, 8 months, 9 months, 10 months, 11 months, 12 months, 18 months, 24 months, etc. the more successful is a therapy, such as for example an mRNA therapy of the invention (e.g., a single dose or a multiple regimen).

A reduction of at least about 20%, at least about 30%, at least about 40%, at least about 50%, at least about 60%, at least about 70%, at least about 80%, at least about 90%, at least 100% or more of the level of biomarker, in particular, in bodily fluid (e.g., plasma, serum, urine, e.g., urinary sediment) or in tissue(s) in a subject (e.g., liver), within 1, 2, 3, 4, 5, 6 or more days following administration is indicative of a dose suitable for successful treatment propionic acidemia, wherein reduction as used herein, preferably means that the level of biomarker determined at the end of a specified time period (e.g., post-administration, for example, of a single intravenous dose) is compared to the level of the same biomarker determined at the beginning of said time period (e.g., pre-administration of said dose). Exemplary time periods include 12, 24, 48, 72, 96, 120 or 144 hours post administration, in particular 24, 48, 72 or 96 hours post administration.

A sustained reduction in substrate levels (e.g., biomarkers) is particularly indicative of mRNA therapeutic dosing and/or administration regimens successful for treatment of propionic acidemia. Such sustained reduction can be referred to herein as “duration” of effect. In exemplary embodiments, a reduction of at least about 20%, at least about 30%, at least about 40%, at least about 50%, at least about 60%, at least about 65%, at least about 70%, at least about 75%, at least about 80%, at least about 85%, at least about 90%, or at least about 95% at least about 96%, at least about 97%, at least about 98%, at least about 99%, at least about 100% or more of the level of biomarker, in particular, in a bodily fluid (e.g., plasma, serum, urine, e.g., urinary sediment) or in tissue(s) in a subject (e.g., liver), within 1, 2, 3, 4, 5, 6, 7, 8 or more days following administration is indicative of a successful therapeutic approach. In exemplary embodiments, sustained reduction in substrate (e.g., biomarker) levels in one or more samples (e.g., fluids and/or tissues) is preferred. For example, mRNA therapies resulting in sustained reduction in a biomarker, optionally in combination with sustained reduction of said biomarker in at least one tissue, preferably two, three, four, five or more tissues, is indicative of successful treatment.

In some embodiments, a single dose of an mRNA therapy of the invention is about 0.2 to about 0.8 mgs/kg (mpk), about 0.3 to about 0.7 mpk, about 0.4 to about 0.8 mpk, or about 0.5 mpk. In another embodiment, a single dose of an mRNA therapy of the invention is less than 1.5 mpk, less than 1.25 mpk, less than 1 mpk, or less than 0.75 mpk.

### Compositions and Formulations for Use

Certain aspects of the invention are directed to compositions or formulations comprising any of the polynucleotides disclosed above.

In some embodiments, the composition or formulation comprises:
(i) a polynucleotide (e.g., a RNA, e.g., an mRNA) comprising a sequence-optimized nucleotide sequence (e.g., an ORF) encoding an PCCA and/or PCCB polypeptide (e.g., the wild-type sequence, functional fragment, or variant thereof), wherein the polynucleotide comprises at least one chemically modified nucleobase, e.g., N1-methylpseudouracil or 5-methoxyuracil (e.g., wherein at least about 25%, at least about 30%, at least about 40%, at least about 50%, at least about 60%, at least about 70%, at least about 80%, at least about 90%, at least about 95%, at least about 99%, or 100% of the uracils are N1-methylpseudouracils or 5-methoxyuracils), and wherein the polynucleotide further comprises a miRNA binding site, e.g., a miRNA binding site that binds to miR-142 (e.g., a miR-142-3p or miR-142-5p binding site) and/or a miRNA binding site that binds to miR-126 (e.g., a miR-126-3p or miR-126-5p binding site); and
(ii) a delivery agent comprising, e.g., a compound having the Formula (II), e.g., Compound B. In some embodiments, the delivery agent comprises an ionizable amino lipid (e.g., Compound B), a helper lipid (e.g., DSPC), a sterol (e.g., Cholesterol), and a PEG lipid (e.g., Compound I or PEG-DMG), e.g., with a mole ratio in the range of about (i) 40-50 mol % ionizable amino lipid (e.g., Compound B), optionally 45-50 mol % ionizable amino lipid, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %; (ii) 30-45 mol % sterol (e.g., cholesterol), optionally 35-42 mol % sterol, for example, 30-31 mol %, 31-32 mol %, 32-33 mol %, 33-34 mol %, 35-35 mol %, 35-36 mol %, 36-37 mol %, 37-38 mol %, 38-39 mol %, or 39-40 mol %, or 40-42 mol % sterol; (iii) 5-15 mol % helper lipid (e.g., DSPC), optionally 10-15 mol % helper lipid, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % helper lipid; and (iv) 1-5% PEG lipid (e.g., Compound I or PEG-DMG), optionally 1-5 mol % PEG lipid, for example 1.5 to 2.5 mol %, 1-2 mol %, 2-3 mol %, 3-4 mol %, or 4-5 mol % PEG lipid. In some embodiments, the delivery agent comprises Compound B, Cholesterol, DSPC, and Compound I with a mole ratio of 47:39:11:3.

In some embodiments, the uracil or thymine content of the ORF relative to the theoretical minimum uracil or thymine content of a nucleotide sequence encoding the PCCA and/or PCCB polypeptide (% U_{TM} or % T_{TM}), is between about 100% and about 150%.

In some embodiments, the polynucleotides, compositions or formulations above are used to treat and/or prevent PCC-related diseases, disorders or conditions, e.g., propionic acidemia.

### Forms of Administration

The polynucleotides, pharmaceutical compositions and formulations of the invention described above can be administered by any route that results in a therapeutically effective outcome, such as intravenous (into a vein) administration. These also include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration that is then covered by a dressing that occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions can be administered in a way that allows them cross the blood-brain barrier, vascular barrier, or other epithelial barrier. In some embodiments, a formulation for a route of administration can include at least one inactive ingredient.

### Definitions

In order that the present disclosure can be more readily understood, certain terms are first defined. As used in this application, except as otherwise expressly provided herein, each of the following terms shall have the meaning set forth below. Additional definitions are set forth throughout the application.

The invention includes embodiments in which exactly one member of the group is present in, employed in, or otherwise relevant to a given product or process. The invention includes embodiments in which more than one, or all of the group members are present in, employed in, or otherwise relevant to a given product or process.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Systéme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Nucleotides are referred to by their commonly accepted single-letter codes. Unless otherwise indicated, nucleic acids are written left to right in 5′ to 3′ orientation. Nucleobases are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

About: The term “about” as used in connection with a numerical value throughout the specification and the claims denotes an interval of accuracy, familiar and acceptable to a person skilled in the art, such interval of accuracy is +10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

Administered in combination: As used herein, the term “administered in combination” or “combined administration” means that two or more agents are administered to a subject at the same time or within an interval such that there can be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amino acid substitution: The term “amino acid substitution” refers to replacing an amino acid residue present in a parent or reference sequence (e.g., a wild type PCCA and/or PCCB sequence) with another amino acid residue. An amino acid can be substituted in a parent or reference sequence (e.g., a wild type PCCA and/or PCCB polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position X, and Y and Z are alternative substituting amino acid residue.

In the context of the present disclosure, substitutions (even when they referred to as amino acid substitution) are conducted at the nucleic acid level, i.e., substituting an amino acid residue with an alternative amino acid residue is conducted by substituting the codon encoding the first amino acid with a codon encoding the second amino acid.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein with respect to a disease, the term “associated with” means that the symptom, measurement, characteristic, or status in question is linked to the diagnosis, development, presence, or progression of that disease. As association can, but need not, be causatively linked to the disease. For example, symptoms, sequelae, or any effects causing a decrease in the quality of life of a patient of propionic acidemia are considered associated with propionic acidemia and in some embodiments of the present invention can be treated, ameliorated, or prevented by administering the polynucleotides of the present invention to a subject in need thereof.

When used with respect to two or more moieties, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It can also suggest ionic or hydrogen bonding or a hybridization based connectivity sufficiently stable such that the “associated” entities remain physically associated.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a polynucleotide of the present invention can be considered biologically active if even a portion of the polynucleotide is biologically active or mimics an activity considered biologically relevant.

Chimera: As used herein, “chimera” is an entity having two or more incongruous or heterogeneous parts or regions. For example, a chimeric molecule can comprise a first part comprising an PCCA and/or PCCB polypeptide, and a second part (e.g., genetically fused to the first part) comprising a second therapeutic protein (e.g., a protein with a distinct enzymatic activity, an antigen binding moiety, or a moiety capable of extending the plasma half life of PCCA and/or PCCB, for example, an Fc region of an antibody).

Sequence Optimization: The term “sequence optimization” refers to a process or series of processes by which nucleobases in a reference nucleic acid sequence are replaced with alternative nucleobases, resulting in a nucleic acid sequence with improved properties, e.g., improved protein expression or decreased immunogenicity.

In general, the goal in sequence optimization is to produce a synonymous nucleotide sequence than encodes the same polypeptide sequence encoded by the reference nucleotide sequence. Thus, there are no amino acid substitutions (as a result of codon optimization) in the polypeptide encoded by the codon optimized nucleotide sequence with respect to the polypeptide encoded by the reference nucleotide sequence.

Codon substitution: The terms “codon substitution” or “codon replacement” in the context of sequence optimization refer to replacing a codon present in a reference nucleic acid sequence with another codon. A codon can be substituted in a reference nucleic acid sequence, for example, via chemical peptide synthesis or through recombinant methods known in the art. Accordingly, references to a “substitution” or “replacement” at a certain location in a nucleic acid sequence (e.g., an mRNA) or within a certain region or subsequence of a nucleic acid sequence (e.g., an mRNA) refer to the substitution of a codon at such location or region with an alternative codon.

As used herein, the terms “coding region” and “region encoding” and grammatical variants thereof, refer to an Open Reading Frame (ORF) in a polynucleotide that upon expression yields a polypeptide or protein.

Compound: As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Contacting: As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal can be performed by varied routes of administration (e.g., intravenous, intramuscular, intradermal, and subcutaneous) and can involve varied amounts of nanoparticle compositions. Moreover, more than one mammalian cell can be contacted by a nanoparticle composition.

Conservative amino acid substitution: A “conservative amino acid substitution” is one in which the amino acid residue in a protein sequence is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitution: Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can be readily identified by workers of ordinary skill. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of an polynucleotide or polypeptide or can apply to a portion, region or feature thereof.

Controlled Release: As used herein, the term “controlled release” refers to a pharmaceutical composition or compound release profile that conforms to a particular pattern of release to effect a therapeutic outcome.

Cyclic or Cyclized: As used herein, the term “cyclic” refers to the presence of a continuous loop. Cyclic molecules need not be circular, only joined to form an unbroken chain of subunits. Cyclic molecules such as the engineered RNA or mRNA of the present invention can be single units or multimers or comprise one or more components of a complex or higher order structure.

Delivering: As used herein, the term “delivering” means providing an entity to a destination. For example, delivering a polynucleotide to a subject can involve administering a nanoparticle composition including the polynucleotide to the subject (e.g., by an intravenous, intramuscular, intradermal, or subcutaneous route).

Administration of a nanoparticle composition to a mammal or mammalian cell can involve contacting one or more cells with the nanoparticle composition. Delivery Agent: As used herein, “delivery agent” refers to any substance that facilitates, at least in part, the in vivo, in vitro, or ex vivo delivery of a polynucleotide to targeted cells.

Domain: As used herein, when referring to polypeptides, the term “domain” refers to a motif of a polypeptide having one or more identifiable structural or functional characteristics or properties (e.g., binding capacity, serving as a site for protein-protein interactions).

Dosing regimen: As used herein, a “dosing regimen” or a “dosing regimen” is a schedule of administration or physician determined regimen of treatment, prophylaxis, or palliative care.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats a protein deficiency (e.g., an PCC deficiency), an effective amount of an agent is, for example, an amount of mRNA expressing sufficient PCCA and/or PCCB to ameliorate, reduce, eliminate, or prevent the symptoms associated with the PCC deficiency, as compared to the severity of the symptom observed without administration of the agent. The term “effective amount” can be used interchangeably with “effective dose,” “therapeutically effective amount,” or “therapeutically effective dose.”

Encapsulate: As used herein, the term “encapsulate” means to enclose, surround or encase.

Encapsulation Efficiency: As used herein, “encapsulation efficiency” refers to the amount of a polynucleotide that becomes part of a nanoparticle composition, relative to the initial total amount of polynucleotide used in the preparation of a nanoparticle composition. For example, if 97 mg of polynucleotide are encapsulated in a nanoparticle composition out of a total 100 mg of polynucleotide initially provided to the composition, the encapsulation efficiency can be given as 97%. As used herein, “encapsulation” can refer to complete, substantial, or partial enclosure, confinement, surrounding, or encasement.

Enhanced Delivery: As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a polynucleotide by a nanoparticle to a target tissue of interest (e.g., mammalian liver) compared to the level of delivery of a polynucleotide by a control nanoparticle to a target tissue of interest (e.g., MC3, KC2, or DLinDMA). The level of delivery of a nanoparticle to a particular tissue can be measured by comparing the amount of protein produced in a tissue to the weight of said tissue, comparing the amount of polynucleotide in a tissue to the weight of said tissue, comparing the amount of protein produced in a tissue to the amount of total protein in said tissue, or comparing the amount of polynucleotide in a tissue to the amount of total polynucleotide in said tissue. It will be understood that the enhanced delivery of a nanoparticle to a target tissue need not be determined in a subject being treated, it can be determined in a surrogate such as an animal model (e.g., a rat model).

Expression: As used herein, “expression” of a nucleic acid sequence refers to one or more of the following events: (1) production of an mRNA template from a DNA sequence (e.g., by transcription); (2) processing of an mRNA transcript (e.g., by splicing, editing, 5′ cap formation, and/or 3′ end processing); (3) translation of an mRNA into a polypeptide or protein; and (4) post-translational modification of a polypeptide or protein.

Formulation: As used herein, a “Formulation” includes at least a polynucleotide and one or more of a carrier, an excipient, and a delivery agent.

Fragment: A “fragment,” as used herein, refers to a portion. For example, fragments of proteins can comprise polypeptides obtained by digesting full-length protein isolated from cultured cells. In some embodiments, a fragment is a subsequences of a full length protein (e.g., PCCA and/or PCCB) wherein N-terminal, and/or C-terminal, and/or internal subsequences have been deleted. In some preferred aspects of the present invention, the fragments of a protein of the present invention are functional fragments.

Functional: As used herein, a “functional” biological molecule is a biological molecule in a form in which it exhibits a property and/or activity by which it is characterized. Thus, a functional fragment of a polynucleotide of the present invention is a polynucleotide capable of expressing a functional PCCA and/or PCCB fragment. As used herein, a functional fragment of PCCA and/or PCCB refers to a fragment of wild type PCCA and/or PCCB (i.e., a fragment of any of its naturally occurring isoforms), or a mutant or variant thereof, wherein the fragment retains a least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the biological activity of the corresponding full length protein.

PCC Associated Disease: As use herein the terms “PCC-associated disease” or “PCC-associated disorder” refer to diseases or disorders, respectively, which result from aberrant PCC activity (e.g., decreased activity or increased activity). As a non-limiting example, propionic acidemia is an PCC-associated disease.

The terms “PCC enzymatic activity” and “PCC activity,” are used interchangeably in the present disclosure and refer to PCC's ability to catalyze the carboxylation of propionyl-CoA to methylmalonyl-CoA. Accordingly, a fragment or variant retaining or having PCC enzymatic activity or PC activity refers to a fragment or variant that has measurable enzymatic activity in catalyzing the carboxylation of propionyl-CoA to methylmalonyl-CoA.

Helper Lipid: As used herein, the term “helper lipid” refers to a compound or molecule that includes a lipidic moiety (for insertion into a lipid layer, e.g., lipid bilayer) and a polar moiety (for interaction with physiologic solution at the surface of the lipid layer). Typically the helper lipid is a phospholipid. A function of the helper lipid is to “complement” the amino lipid and increase the fusogenicity of the bilayer and/or to help facilitate endosomal escape, e.g., of nucleic acid delivered to cells. Helper lipids are also believed to be a key structural component to the surface of the LNP.

Homology: As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present invention, the term homology encompasses both to identity and similarity.

In some embodiments, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

Identity: As used herein, the term “identity” refers to the overall monomer conservation between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity “% ID” of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

Insertional and deletional variants: “Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid. “Deletional variants” when referring to polypeptides are those with one or more amino acids in the native or starting amino acid sequence removed. Ordinarily, deletional variants will have one or more amino acids deleted in a particular region of the molecule.

Intact: As used herein, in the context of a polypeptide, the term “intact” means retaining an amino acid corresponding to the wild type protein, e.g., not mutating or substituting the wild type amino acid. Conversely, in the context of a nucleic acid, the term “intact” means retaining a nucleobase corresponding to the wild type nucleic acid, e.g., not mutating or substituting the wild type nucleobase.

Ionizable amino lipid: The term “ionizable amino lipid” includes those lipids having one, two, three, or more fatty acid or fatty alkyl chains and a pH-titratable amino head group (e.g., an alkylamino or dialkylamino head group). An ionizable amino lipid is typically protonated (i.e., positively charged) at a pH below the pKa of the amino head group and is substantially not charged at a pH above the pKa. Such ionizable amino lipids include, but are not limited to DLin-MC3-DMA (MC3), (13Z,165Z)—N,N-dimethyl-3-nonydocosa-13-16-dien-1-amine (L608), and a compound having the Formula (II), e.g., Compound B.

Linker: As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Methods ofAdministration: As used herein, “methods of administration” can include intravenous, intramuscular, intradermal, subcutaneous, or other methods of delivering a composition to a subject. A method of administration can be selected to target delivery (e.g., to specifically deliver) to a specific region or system of a body.

Modified: As used herein “modified” refers to a changed state or structure of a molecule of the invention. Molecules can be modified in many ways including chemically, structurally, and functionally. In some embodiments, the mRNA molecules of the present invention are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Nanoparticle Composition: As used herein, a “nanoparticle composition” is a composition comprising one or more lipids. Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Naturally occurring: As used herein, “naturally occurring” means existing in nature without artificial aid.

Nucleic acidsequence: The terms “nucleic acid sequence,” “nucleotide sequence,” or “polynucleotide sequence” are used interchangeably and refer to a contiguous nucleic acid sequence. The sequence can be either single stranded or double stranded DNA or RNA, e.g., an mRNA.

The term “nucleic acid,” in its broadest sense, includes any compound and/or substance that comprises a polymer of nucleotides. These polymers are often referred to as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

The phrase “nucleotide sequence encoding” refers to the nucleic acid (e.g., an mRNA or DNA molecule) coding sequence which encodes a polypeptide. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

Operably linked: As used herein, the phrase “operably linked” refers to a functional connection between two or more molecules, constructs, transcripts, entities, moieties or the like.

Optionally substituted: Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl)per se is optional.

Part: As used herein, a “part” or “region” of a polynucleotide is defined as any portion of the polynucleotide that is less than the entire length of the polynucleotide.

Patient: As used herein, “patient” refers to a subject who can seek or be in need of treatment, requires treatment, is receiving treatment, will receive treatment, or a subject who is under care by a trained professional for a particular disease or condition. In some embodiments, the treatment is needed, required, or received to prevent or decrease the risk of developing acute disease, i.e., it is a prophylactic treatment.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspension or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion.

This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding mRNA sequences (comprising U) are considered codon-optimized nucleotide sequence of the present invention. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ‘P’PC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-O-D-ribofuranosyl-purine) can be modified to form isoguanosine (2-oxy-6-amino-9-O-D-ribofuranosyl-purine). Such modification results in a nucleoside base which will no longer effectively form a standard base pair with cytosine. However, modification of cytosine (1-O-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include encoded polynucleotide products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some embodiments, a “peptide” can be less than or equal to 50 amino acids long, e.g., about 5, 10, 15, 20, 25, 30, 35, 40, 45, or 50 amino acids long.

Polypeptide variant: As used herein, the term “polypeptide variant” refers to molecules that differ in their amino acid sequence from a native or reference sequence. The amino acid sequence variants can possess substitutions, deletions, and/or insertions at certain positions within the amino acid sequence, as compared to a native or reference sequence. Ordinarily, variants will possess at least about 50% identity, at least about 60% identity, at least about 70% identity, at least about 80% identity, at least about 90% identity, at least about 95% identity, at least about 99% identity to a native or reference sequence. In some embodiments, they will be at least about 80%, or at least about 90% identical to a native sequence

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease. An “immune prophylaxis” refers to a measure to produce active or passive immunity to prevent the spread of disease.

Pseudouridine: As used herein, pseudouridine (ψ) refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m¹ψ) (also known as N1-methyl-pseudouridine), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, 1-methyl-3-(3-amino-3-carboxypropyl)pseudouridine (acp³ψ), and 2′-O-methyl-pseudouridine (ψm).

Purified: As used herein, “purify,” “purified,” “purification” means to make substantially pure or clear from unwanted components, material defilement, admixture or imperfection.

Reference Nucleic Acid Sequence: The term “reference nucleic acid sequence” or “reference nucleic acid” or “reference nucleotide sequence” or “reference sequence” refers to a starting nucleic acid sequence (e.g., a RNA, e.g., an mRNA sequence) that can be sequence optimized. In some embodiments, the reference nucleic acid sequence is a wild type nucleic acid sequence, a fragment or a variant thereof. In some embodiments, the reference nucleic acid sequence is a previously sequence optimized nucleic acid sequence.

Salts: In some aspects, the pharmaceutical composition for delivery disclosed herein and comprises salts of some of their lipid constituents. The term “salt” includes any anionic and cationic complex. Non-limiting examples of anions include inorganic and organic anions, e.g., fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

Sample: As used herein, the term “sample” or “biological sample” refers to a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). A sample further can include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which can contain cellular components, such as proteins or nucleic acid molecule.

Signal Sequence: As used herein, the phrases “signal sequence,” “signal peptide,” and “transit peptide” are used interchangeably and refer to a sequence that can direct the transport or localization of a protein to a certain organelle, cell compartment, or extracellular export. The term encompasses both the signal sequence polypeptide and the nucleic acid sequence encoding the signal sequence. Thus, references to a signal sequence in the context of a nucleic acid refer in fact to the nucleic acid sequence encoding the signal sequence polypeptide.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Single unit dose: As used herein, a “single unit dose” is a dose of any therapeutic administered in one dose/at one time/single route/single point of contact, i.e., single administration event.

Specific delivery: As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a polynucleotide by a nanoparticle to a target tissue of interest (e.g., mammalian liver) compared to an off-target tissue (e.g., mammalian spleen). The level of delivery of a nanoparticle to a particular tissue can be measured by comparing the amount of protein produced in a tissue to the weight of said tissue, comparing the amount of polynucleotide in a tissue to the weight of said tissue, comparing the amount of protein produced in a tissue to the amount of total protein in said tissue, or comparing the amount of polynucleotide in a tissue to the amount of total polynucleotide in said tissue. For example, for renovascular targeting, a polynucleotide is specifically provided to a mammalian kidney as compared to the liver and spleen if 1.5, 2-fold, 3-fold, 5-fold, 10-fold, 15 fold, or 20 fold more polynucleotide per 1 g of tissue is delivered to a kidney compared to that delivered to the liver or spleen following systemic administration of the polynucleotide. It will be understood that the ability of a nanoparticle to specifically deliver to a target tissue need not be determined in a subject being treated, it can be determined in a surrogate such as an animal model (e.g., a rat model).

Stable: As used herein “stable” refers to a compound that is sufficiently robust to survive isolation to a useful degree of purity from a reaction mixture, and in some cases capable of formulation into an efficacious therapeutic agent.

Stabilized: As used herein, the term “stabilize,” “stabilized,” “stabilized region” means to make or become stable.

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of treatment.

Substantially: As used herein, the term “substantially” refers to the qualitative condition of exhibiting total or near-total extent or degree of a characteristic or property of interest. One of ordinary skill in the biological arts will understand that biological and chemical characteristics rarely, if ever, go to completion and/or proceed to completeness or achieve or avoid an absolute result. The term “substantially” is therefore used herein to capture the potential lack of completeness inherent in many biological and chemical characteristics.

Substantially equal: As used herein as it relates to time differences between doses, the term means plus/minus 2%.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with and/or cannot exhibit symptoms of the disease, disorder, and/or condition but harbors a propensity to develop a disease or its symptoms. In some embodiments, an individual who is susceptible to a disease, disorder, and/or condition (for example, propionic acidemia) can be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or other molecules of the present invention can be chemical or enzymatic.

Targeted Cells: As used herein, “targeted cells” refers to any one or more cells of interest. The cells can be found in vitro, in vivo, in situ or in the tissue or organ of an organism. The organism can be an animal, for example a mammal, a human, a subject or a patient.

Target tissue: As used herein “target tissue” refers to any one or more tissue types of interest in which the delivery of a polynucleotide would result in a desired biological and/or pharmacological effect. Examples of target tissues of interest include specific tissues, organs, and systems or groups thereof. In particular applications, a target tissue can be a liver, a kidney, a lung, a spleen, or a vascular endothelium in vessels (e.g., intra-coronary or intra-femoral). An “off-target tissue” refers to any one or more tissue types in which the expression of the encoded protein does not result in a desired biological and/or pharmacological effect.

The presence of a therapeutic agent in an off-target issue can be the result of: (i) leakage of a polynucleotide from the administration site to peripheral tissue or distant off-target tissue via diffusion or through the bloodstream (e.g., a polynucleotide intended to express a polypeptide in a certain tissue would reach the off-target tissue and the polypeptide would be expressed in the off-target tissue); or (ii) leakage of an polypeptide after administration of a polynucleotide encoding such polypeptide to peripheral tissue or distant off-target tissue via diffusion or through the bloodstream (e.g., a polynucleotide would expressed a polypeptide in the target tissue, and the polypeptide would diffuse to peripheral tissue).

Targeting sequence: As used herein, the phrase “targeting sequence” refers to a sequence that can direct the transport or localization of a protein or polypeptide.

Therapeutic Agent: The term “therapeutic agent” refers to an agent that, when administered to a subject, has a therapeutic, diagnostic, and/or prophylactic effect and/or elicits a desired biological and/or pharmacological effect. For example, in some embodiments, an mRNA encoding an PCCA and/or PCCB polypeptide can be a therapeutic agent.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Transcription: As used herein, the term “transcription” refers to methods to produce mRNA (e.g., an mRNA sequence or template) from DNA (e.g., a DNA template or sequence).

Transfection: As used herein, “transfection” refers to the introduction of a polynucleotide (e.g., exogenous nucleic acids) into a cell wherein a polypeptide encoded by the polynucleotide is expressed (e.g., mRNA) or the polypeptide modulates a cellular function (e.g., siRNA, miRNA). As used herein, “expression” of a nucleic acid sequence refers to translation of a polynucleotide (e.g., an mRNA) into a polypeptide or protein and/or post-translational modification of a polypeptide or protein. Methods of transfection include, but are not limited to, chemical methods, physical treatments and cationic lipids or mixtures.

Treating, treatment, therapy: As used herein, the term “treating” or “treatment” or “therapy” refers to partially or completely alleviating, ameliorating, improving, relieving, delaying onset of, inhibiting progression of, reducing severity of, and/or reducing incidence of one or more symptoms or features of a disease, e.g., propionic acidemia. For example, “treating” propionic acidemia can refer to diminishing symptoms associate with the disease, prolong the lifespan (increase the survival rate) of patients, reducing the severity of the disease, preventing or delaying the onset of the disease, etc. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in some way. Unmodified can, but does not always, refer to the wild type or native form of a biomolecule. Molecules can undergo a series of modifications whereby each modified molecule can serve as the “unmodified” starting molecule for a subsequent modification.

Uracil: Uracil is one of the four nucleobases in the nucleic acid of RNA, and it is represented by the letter U. Uracil can be attached to a ribose ring, or more specifically, a ribofuranose via a β-N₁-glycosidic bond to yield the nucleoside uridine. The nucleoside uridine is also commonly abbreviated according to the one letter code of its nucleobase, i.e., U. Thus, in the context of the present disclosure, when a monomer in a polynucleotide sequence is U, such U is designated interchangeably as a “uracil” or a “uridine.”

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in a sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

Variant: The term variant as used in present disclosure refers to both natural variants (e.g., polymorphisms, isoforms, etc.) and artificial variants in which at least one amino acid residue in a native or starting sequence (e.g., a wild type sequence) has been removed and a different amino acid inserted in its place at the same position. These variants can be described as “substitutional variants.” The substitutions can be single, where only one amino acid in the molecule has been substituted, or they can be multiple, where two or more amino acids have been substituted in the same molecule. If amino acids are inserted or deleted, the resulting variant would be an “insertional variant” or a “deletional variant” respectively.

Initiation Codon: As used herein, the term “initiation codon”, used interchangeably with the term “start codon”, refers to the first codon of an open reading frame that is translated by the ribosome and is comprised of a triplet of linked adenine-uracil-guanine nucleobases. The initiation codon is depicted by the first letter codes of adenine (A), uracil (U), and guanine (G) and is often written simply as “AUG”. Although natural mRNAs may use codons other than AUG as the initiation codon, which are referred to herein as “alternative initiation codons”, the initiation codons of polynucleotides described herein use the AUG codon. During the process of translation initiation, the sequence comprising the initiation codon is recognized via complementary base-pairing to the anticodon of an initiator tRNA (Met-tRNAᵢ^{Met}) bound by the ribosome. Open reading frames may contain more than one AUG initiation codon, which are referred to herein as “alternate initiation codons”.

The initiation codon plays a critical role in translation initiation. The initiation codon is the first codon of an open reading frame that is translated by the ribosome. Typically, the initiation codon comprises the nucleotide triplet AUG, however, in some instances translation initiation can occur at other codons comprised of distinct nucleotides. The initiation of translation in eukaryotes is a multistep biochemical process that involves numerous protein-protein, protein-RNA, and RNA-RNA interactions between messenger RNA molecules (mRNAs), the 40S ribosomal subunit, other components of the translation machinery (e.g., eukaryotic initiation factors; eIFs). The current model of mRNA translation initiation postulates that the pre-initiation complex (alternatively “43S pre-initiation complex”; abbreviated as “PIC”) translocates from the site of recruitment on the mRNA (typically the 5′ cap) to the initiation codon by scanning nucleotides in a 5′ to 3′ direction until the first AUG codon that resides within a specific translation-promotive nucleotide context (the Kozak sequence) is encountered (Kozak (1989) J Cell Biol 108:229-241). Scanning by the PIC ends upon complementary base-pairing between nucleotides comprising the anticodon of the initiator Met-tRNAᵢ^{Met} transfer RNA and nucleotides comprising the initiation codon of the mRNA. Productive base-pairing between the AUG codon and the Met-tRNAᵢ^{Met} anticodon elicits a series of structural and biochemical events that culminate in the joining of the large 60S ribosomal subunit to the PIC to form an active ribosome that is competent for translation elongation.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC (SEQ ID NO:145), where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Modified: As used herein “modified” or “modification” refers to a changed state or a change in composition or structure of a polynucleotide (e.g., mRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids. Unless otherwise specified, the nucleobase sequence of a SEQ ID NO described herein encompasses both natural nucleobases and chemically modified nucleobases (e.g., a “U” designation in a SEQ ID NO encompasses both uracil and chemically modified uracil).

Nucleoside Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Nucleic acid: As used herein, the term “nucleic acid” is used in its broadest sense and encompasses any compound and/or substance that includes a polymer of nucleotides, or derivatives or analogs thereof. These polymers are often referred to as “polynucleotides”. Accordingly, as used herein the terms “nucleic acid” and “polynucleotide” are equivalent and are used interchangeably. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, mRNAs, modified mRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Pre-Initiation Complex (PIC): As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1: Synthesis of mRNAs Encoding PCCA and PCCB

An mRNA encoding human PCCA is constructed by using the ORF sequence (nucleotide) provided in SEQ ID NO:5. The mRNA sequence includes both 5′ and 3′ UTR regions flanking the ORF sequence. The 5′ UTR and 3′ UTR sequences of the mRNA are SEQ ID NO:55 and SEQ ID NO:114, respectively.

An mRNA encoding human PCCB is constructed by using the ORF sequence (nucleotide) provided in SEQ ID NO:6. The mRNA sequence includes both 5′ and 3′ UTR regions flanking the ORF sequence. The 5′ UTR and 3′ UTR sequences of the mRNA are SEQ ID NO:55 and SEQ ID NO:114, respectively.

The PCCA and PCCB mRNA sequences are prepared as modified mRNAs. Specifically, during in vitro transcription, modified mRNAs can be generated using N1-methylpseudouridine-5′-triphosphate to ensure that the mRNAs contain 100% N1-methylpseudouridine instead of uridine. Alternatively, during in vitro transcription, modified mRNA can be generated using N1-methoxyuridine-5′-Triphosphate to ensure that the mRNAs contain 100% 5-methoxyuridine instead of uridine. Further, PCCA-mRNA and PCCB-mRNA can be synthesized with a primer that introduces a polyA-tail, and a cap structure is generated on both mRNAs using co-transcriptional capping via m⁷Gp-ppGm-pA-pG tetranucleotide to incorporate a m⁷Gp-ppGm-pA-pG 5′ cap. Alternatively, mRNA can be synthesized and the polyA-tail introduced during Gibson assembly of the DNA template.

The components of the PCCA mRNA and the PCCB mRNA are described in detail below. The PCCA mRNA and the PCCB mRNA are encapsulated together in lipid nanoparticles at a ratio of 1:1.

TABLE 6
PCCA mRNA

| Components of PCCA | |
|---|---|
| mRNA | Description of PCCA mRNA Component |
| 5′-terminal cap | Cap1 |
| Chemistry | all uracils in the mRNA are N1-methylpseudouracils |
| 5′ untranslated region | GGAAAUAAGAGAGAAAAGAAGAGUAAGAAGAA |
| (UTR) | AUAUAAGAGCCACC |
| | (SEQ ID NO: 55) |
| open reading frame (ORF) | AUGGCCGGCUUCUGGGUCGGCACAGCCCCUCUG |
| | GUGGCAGCCGGCAGAAGAGGACGGUGGCCUCCC |
| | CAGCAACUGAUGCUGAGCGCCGCCCUGAGAACC |
| | CUGAAGCACGUGCUGUACUACAGCAGACAGUGC |
| | CUGAUGGUGAGCAGAAAUCUGGGCAGCGUGGG |
| | GUACGAUCCCAACGAGAAGACCUUCGAUAAGAU |
| | UCUGGUCGCGAAUAGAGGCGAGAUCGCCUGCAG |
| | GGUGAUCAGAACCUGCAAGAAGAUGGGCAUCA |
| | AGACCGUGGCCAUCCAUUCGGACGUCGACGCGA |
| | GCAGCGUUCACGUGAAGAUGGCAGACGAGGCCG |
| | UGUGCGUGGGACCCGCCCCGACCAGCAAGAGCU |
| | ACCUGAACAUGGACGCCAUCAUGGAGGCCAUCA |
| | AGAAGACCCGCGCUCAAGCCGUGCACCCGGGCU |
| | ACGGCUUUCUGAGCGAGAACAAGGAAUUCGCCA |
| | GGUGUCUCGCCGCCGAGGACGUAGUCUUCAUCG |
| | GCCCUGAUACGCACGCGAUCCAGGCCAUGGGCG |
| | ACAAGAUCGAGAGCAAACUGCUGGCCAAGAAAG |
| | CAGAAGUCAACACCAUCCCCGGCUUCGACGGCG |
| | UGGUGAAGGACGCCGAAGAGGCUGUCCGCAUCG |
| | CCAGAGAGAUCGGCUACCCUGUGAUGAUAAAGG |
| | CUAGCGCUGGAGGUGGCGGAAAGGGCAUGAGA |
| | AUCGCCUGGGACGACGAGGAGACUAGAGACGGC |
| | UUCAGACUGUCCUCCCAGGAGGCCGCCAGCUCC |
| | UUCGGAGACGACAGACUGCUGAUCGAGAAGUUC |
| | AUCGACAACCCCAGACACAUCGAAAUCCAGGUG |
| | CUCGGUGACAAGCACGGGAACGCCCUGUGGCUG |
| | AACGAGAGAGAGUGCAGCAUCCAGAGAAGAAA |
| | CCAGAAGGUGGUGGAGGAGGCGCCGAGCAUCUU |
| | UCUGGACGCGGAGACAAGGAGAGCGAUGGGCG |
| | AACAGGCCGUCGCCCUAGCAAGAGCCGUGAAGU |
| | ACUCCAGUGCCGGAACCGUCGAGUUUCUUGUCG |
| | ACAGCAAGAAGAAUUUCUACUUCCUGGAGAUG |
| | AACACCAGGCUGCAGGUGGAGCAUCCCGUGACA |
| | GAGUGCAUCACUGGACUGGAUCUGGUGCAGGA |
| | GAUGAUCAGGGUGGCCAAGGGCUAUCCCCUGAG |
| | ACACAAGCAGGCCGACAUCAGAAUCAACGGCUG |
| | GGCCGUGGAGUGCAGAGUGUACGCCGAGGACCC |
| | CUACAAGAGCUUCGGCCUGCCCAGCAUCGGCAG |
| | ACUGAGCCAGUACCAGGAGCCCCUGCACCUGCC |
| | CGGCGUGAGAGUGGACAGCGGCAUCCAACCGGG |
| | GAGCGAUAUCAGCAUCUACUACGACCCCAUGAU |
| | CAGCAAGCUGAUAACCUACGGCAGCGACAGAAC |
| | CGAGGCCCUGAAGAGAAUGGCCGACGCCCUGGA |
| | CAACUACGUGAUCAGAGGCGUGACCCACAACAU |
| | CGCCCUGCUGAGAGAGGUGAUCAUCAACUCGAG |
| | GUUCGUGAAAGGCGACAUCAGCACCAAGUUCCU |
| | GAGCGACGUGUAUCCCGACGGAUUCAAAGGUCA |
| | CAUGCUGACCAAGAGCGAGAAGAACCAGCUGCU |
| | GGCCAUCGCCUCAUCCCUGUUCGUGGCCUUCCA |
| | GCUGAGAGCCCAGCACUUCCAGGAGAACAGCAG |
| | AAUGCCCGUGAUCAAGCCCGACAUCGCCAACUG |
| | GGAGCUGAGCGUGAAGCUGCACGACAAGGUGCA |
| | CACUGUCGUUGCCAGCAACAACGGCUCCGUGUU |
| | CAGCGUAGAGGUGGACGGAUCUAAGCUGAACG |
| | UGACCUCCACCUGGAACCUGGCAAGCCCUCUCC |
| | UGUCAGUGAGCGUGGACGGCACCCAGAGAACCG |
| | UGCAGUGUCUGUCCCGCGAGGCCGGCGGAAACA |
| | UGAGCAUCCAGUUCCUGGGCACCGUGUACAAGG |
| | UGAACAUCCUGACCAGACUGGCCGCCGAGCUGA |
| | ACAAGUUCAUGCUGGAGAAAGUGACGGAGGAU |
| | ACCAGCUCCGUGCUGAGAAGCCCCAUGCCCGGA |
| | GUGGUGGUGGCCGUUUCCGUGAAACCUGGUGAC |
| | GCCGUGGCCGAGGGGCAAGAGAUCUGCGUGAUC |
| | GAGGCCAUGAAGAUGCAGAAUUCCAUGACCGCC |
| | GGAAAGACCGGCACCGUCAAAUCAGUGCACUGC |
| | CAGGCGGGCGACACAGUGGGUGAGGGCGACCUG |
| | CUGGUGGAGCUGGAG |
| | (SEQ ID NO: 5) |
| 3′ UTR | UGAUAAUAGUCCAUAAAGUAGGAAACACUACA |
| | GCUGGAGCCUCGGUGGCCUAGCUUCUUGCCCCU |
| | UGGGCCUCCAUAAAGUAGGAAACACUACAUCCC |
| | CCCAGCCCCUCCUCCCCUUCCUGCACCCGUACCC |
| | CCUCCAUAAAGUAGGAAACACUACAGUGGUCUU |
| | UGAAUAAAGUCUGAGUGGGCGGC |
| | (SEQ ID NO: 114) |
| poly-A tail | 100 residues in length (SEQ ID NO: 195) |
| Encoded PCCA protein | MAGFWVGTAPLVAAGRRGRWPPQQLMLSAALRT |
| | LKHVLYYSRQCLMVSRNLGSVGYDPNEKTFDKILV |
| | ANRGEIACRVIRTCKKMGIKTVAIHSDVDASSVHV |
| | KMADEAVCVGPAPTSKSYLNMDAIMEAIKKTRAQ |
| | AVHPGYGFLSENKEFARCLAAEDVVFIGPDTHAIQA |
| | MGDKIESKLLAKKAEVNTIPGFDGVVKDAEEAVRI |
| | AREIGYPVMIKASAGGGGKGMRIAWDDEETRDGF |
| | RLSSQEAASSFGDDRLLIEKFIDNPRHIEIQVLGDKH |
| | GNALWLNERECSIQRRNQKVVEEAPSIFLDAETRRA |
| | MGEQAVALARAVKYSSAGTVEFLVDSKKNFYFLE |
| | MNTRLQVEHPVTECITGLDLVQEMIRVAKGYPLRH |
| | KQADIRINGWAVECRVYAEDPYKSFGLPSIGRLSQY |
| | QEPLHLPGVRVDSGIQPGSDISIYYDPMISKLITYGS |
| | DRTEALKRMADALDNYVIRGVTHNIALLREVIINSR |
| | FVKGDISTKFLSDVYPDGFKGHMLTKSEKNQLLAIA |
| | SSLFVAFQLRAQHFQENSRMPVIKPDIANWELSVKL |
| | HDKVHTVVASNNGSVFSVEVDGSKLNVTSTWNLA |
| | SPLLSVSVDGTQRTVQCLSREAGGNMSIQFLGTVY |
| | KVNILTRLAAELNKFMLEKVTEDTSSVLRSPMPGV |
| | VVAVSVKPGDAVAEGQEICVIEAMKMQNSMTAGK |
| | TGTVKSVHCQAGDTVGEGDLLVELE |
| | (SEQ ID NO: 1) |
| Construct Sequence | SEQ ID NO: 7 consists from 5′ to 3′ end: 5′ UTR of SEQ |
| | ID NO: 55, ORF Sequence of SEQ ID NO: 5, and 3′ UTR |
| | of SEQ ID NO: 114 |

TABLE 7
PCCB mRNA

| Components of PCCB | |
|---|---|
| mRNA | Description of PCCB mRNA Component |
| 5′-terminal cap | Cap1 |
| Chemistry | all uracils in the mRNA are N1-methylpseudouracils |
| 5′ untranslated region | GGAAAUAAGAGAGAAAAGAAGAGUAAGAAGAAA |
| (UTR) | UAUAAGAGCCACC |
| | (SEQ ID NO: 55) |
| open reading frame (ORF) | AUGGCGGCGGCAUUACGGGUGGCGGCGGUCGGG |
| | GCAAGGCUCAGCGUGCUGGCCAGCGGCCUGAGA |
| | GCCGCCGUGAGAAGCCUGUGCAGCCAGGCCACCA |
| | GCGUGAACGAGAGAAUCGAGAACAAGAGAAGAA |
| | CCGCCCUGCUGGGCGGCGGCCAGAGAAGAAUCG |
| | ACGCCCAGCACAAGAGAGGCAAGCUGACCGCCA |
| | GAGAGAGAAUCAGCCUGCUGCUGGACCCCGGCA |
| | GCUUCGUGGAGAGCGACAUGUUCGUGGAGCACA |
| | GGUGCGCCGACUUCGGCAUGGCCGCCGACAAGA |
| | ACAAGUUCCCCGGCGACAGCGUGGUGACCGGCA |
| | GAGGCAGAAUCAACGGCAGACUGGUGUACGUGU |
| | UCAGCCAGGACUUCACCGUGUUCGGCGGCAGCC |
| | UGAGCGGCGCCCACGCCCAGAAGAUCUGCAAGA |
| | UCAUGGACCAGGCCAUCACCGUGGGCGCGCCCGU |
| | GAUCGGCCUGAACGACAGCGGCGGCGCCAGAAU |
| | CCAGGAGGGCGUGGAGAGCCUGGCCGGCUACGC |
| | CGACAUCUUCCUGAGAAACGUGACCGCCAGCGG |
| | CGUGAUCCCACAGAUCAGCCUGAUCAUGGGCCCC |
| | UGCGCCGGCGGCGCCGUGUACAGCCCCGCCCUGA |
| | CCGACUUCACCUUCAUGGUGAAGGACACCAGCU |
| | ACCUGUUCAUCACCGGCCCCGACGUGGUGAAGA |
| | GCGUGACCAACGAGGACGUGACCCAGGAGGAGC |
| | UGGGCGGCGCCAAGACCCACACCACCAUGAGCGG |
| | CGUGGCCCACAGAGCCUUCGAGAACGACGUGGA |
| | CGCCCUGUGCAACCUGAGAGACUUCUUCAACUA |
| | CCUGCCCCUGAGCAGCCAGGACCCCGCGCCCGUG |
| | AGAGAGUGCCACGACCCCAGCGACAGACUGGUG |
| | CCCGAGCUGGACACCAUCGUGCCCCUGGAGAGCA |
| | CCAAGGCCUACAACAUGGUGGACAUCAUCCACA |
| | GCGUGGUGGACGAGAGAGAGUUCUUCGAGAUCA |
| | UGCCCAACUACGCCAAGAACAUCAUCGUGGGCU |
| | UCGCCAGAAUGAACGGCAGAACCGUGGGCAUCG |
| | UGGGCAACCAGCCCAAGGUGGCCAGCGGCUGCC |
| | UGGACAUCAACAGCAGCGUGAAGGGCGCCAGAU |
| | UCGUGAGAUUCUGCGACGCCUUCAACAUCCCUC |
| | UGAUCACCUUCGUGGACGUGCCCGGCUUCCUGCC |
| | CGGCACCGCCCAGGAGUACGGCGGCAUCAUCAG |
| | ACACGGCGCCAAGCUGCUGUACGCCUUCGCCGAG |
| | GCCACCGUGCCCAAGGUGACCGUGAUCACCAGA |
| | AAGGCCUACGGCGGCGCCUACGACGUGAUGAGC |
| | AGCAAGCACCUGUGCGGCGACACCAACUACGCCU |
| | GGCCCACCGCCGAGAUCGCCGUGAUGGGCGCCAA |
| | GGGCGCCGUGGAGAUCAUCUUCAAGGGCCACGA |
| | GAACGUGGAGGCCGCCCAGGCCGAGUACAUCGA |
| | GAAGUUCGCCAACCCCUUCCCCGCCGCCGUGAGA |
| | GGCUUCGUGGACGACAUCAUCCAGCCCAGCAGC |
| | ACCAGAGCCAGAAUCUGCUGCGACCUGGACGUG |
| | CUGGCCAGCAAGAAGGUGCAGAGACCCUGGAGA |
| | AAGCACGCCAACAUCCCUCUG |
| | (SEQ ID NO: 6) |
| 3′ UTR | UGAUAAUAGUCCAUAAAGUAGGAAACACUACAG |
| | CUGGAGCCUCGGUGGCCUAGCUUCUUGCCCCUU |
| | GGGCCUCCAUAAAGUAGGAAACACUACAUCCCC |
| | CCAGCCCCUCCUCCCCUUCCUGCACCCGUACCCC |
| | CUCCAUAAAGUAGGAAACACUACAGUGGUCUUU |
| | GAAUAAAGUCUGAGUGGGCGGC |
| | (SEQ ID NO: 114) |
| poly-A tail | 100 residues in length (SEQ ID NO: 195) |
| Encoded PCCB protein | MAAALRVAAVGARLSVLASGLRAAVRSLCSQATSV |
| | NERIENKRRTALLGGGQRRIDAQHKRGKLTARERIS |
| | LLLDPGSFVESDMFVEHRCADFGMAADKNKFPGDS |
| | VVTGRGRINGRLVYVFSQDFTVFGGSLSGAHAQKIC |
| | KIMDQAITVGAPVIGLNDSGGARIQEGVESLAGYADI |
| | FLRNVTASGVIPQISLIMGPCAGGAVYSPALTDFTFM |
| | VKDTSYLFITGPDVVKSVTNEDVTQEELGGAKTHTT |
| | MSGVAHRAFENDVDALCNLRDFFNYLPLSSQDPAP |
| | VRECHDPSDRLVPELDTIVPLESTKAYNMVDIIHSVV |
| | DEREFFEIMPNYAKNIIVGFARMNGRTVGIVGNQPK |
| | VASGCLDINSSVKGARFVRFCDAFNIPLITFVDVPGF |
| | LPGTAQEYGGIIRHGAKLLYAFAEATVPKVTVITRK |
| | AYGGAYDVMSSKHLCGDTNYAWPTAEIAVMGAKG |
| | AVEIIFKGHENVEAAQAEYIEKFANPFPAAVRGFVD |
| | DIIQPSSTRARICCDLDVLASKKVQRPWRKHANIPL |
| | (SEQ ID NO: 2) |
| Construct Sequence | SEQ ID NO: 8 consists from 5′ to 3′ end: 5′ UTR of SEQ |
| | ID NO: 55, ORF Sequence of SEQ ID NO: 6, and 3′ UTR of |
| | SEQ ID NO: 114 |

### Example 2: Production of PCCA/PCCB mRNA Nanoparticle Compositions

### A. Production of Nanoparticle Compositions

Nanoparticles can be made with mixing processes such as microfluidics and T-junction mixing of two fluid streams, one of which contains the polynucleotide and the other has the lipid components.

Lipid compositions are prepared by combining an ionizable amino lipid disclosed herein, e.g., a lipid according to Formula (II) such as Compound B, a phospholipid (such as DOPE or DSPC, obtainable from Avanti Polar Lipids, Alabaster, AL), a PEG lipid (such as Compound I or 1,2 dimyristoyl sn glycerol methoxypolyethylene glycol, also known as PEG-DMG, obtainable from Avanti Polar Lipids, Alabaster, AL), and a structural lipid (such as cholesterol, obtainable from Sigma Aldrich, Taufkirchen, Germany, or a corticosteroid (such as prednisolone, dexamethasone, prednisone, and hydrocortisone), or a combination thereof) at concentrations of about 50 mM in ethanol. Solutions should be refrigerated for storage at, for example, −20° C. Lipids are combined to yield desired molar ratios and diluted with water and ethanol to a final lipid concentration of between about 5.5 mM and about 25 mM.

Nanoparticle compositions including a polynucleotide and a lipid composition are prepared by combining the lipid solution with a solution including the a polynucleotide at lipid composition to polynucleotide wt:wt ratios between about 5:1 and about 50:1. The lipid solution is rapidly injected using a NanoAssemblr microfluidic based system at flow rates between about 10 ml/min and about 18 ml/min into the polynucleotide solution to produce a suspension with a water to ethanol ratio between about 1:1 and about 4:1.

For nanoparticle compositions including an RNA, solutions of the RNA at concentrations of 0.1 mg/ml in deionized water are diluted in 50 mM sodium citrate buffer at a pH between 3 and 4 to form a stock solution.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kD. The first dialysis is carried out at room temperature for 3 hours. The formulations are then dialyzed overnight at 4° C. The resulting nanoparticle suspension is filtered through 0.2 m sterile filters (Sarstedt, Numbrecht, Germany) into glass vials and sealed with crimp closures. Nanoparticle composition solutions of 0.01 mg/ml to 0.10 mg/ml are generally obtained.

The method described above induces nano-precipitation and particle formation. Alternative processes including, but not limited to, T-junction and direct injection, can be used to achieve the same nano-precipitation.

### B. Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of a polynucleotide (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of polynucleotide in the nanoparticle composition can be calculated based on the extinction coefficient of the polynucleotide used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, CA) can be used to evaluate the encapsulation of an RNA by the nanoparticle composition. The samples are diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

Exemplary formulations of the nanoparticle compositions are presented in the Table 8 below. The term “Compound” refers to an ionizable amino lipid such as Compound B. “Phospholipid” can be DSPC or DOPE. “PEG-lipid” can be PEG-DMG or Compound I.

TABLE 8
Exemplary Formulations of Nanoparticles

| Composition | |
|---|---|
| (mol %) | Components |
| 47:11:39:3 | Compound:Phospholipid:Chol:PEG-lipid |
| 40:20:38.5:1.5 | Compound:Phospholipid:Chol:PEG-lipid |
| 45:15:38.5:1.5 | Compound:Phospholipid:Chol:PEG-lipid |
| 50:10:38.5:1.5 | Compound:Phospholipid:Chol:PEG-lipid |
| 45:20:33.5:1.5 | Compound:Phospholipid:Chol:PEG-lipid |
| 50:20:28.5:1.5 | Compound:Phospholipid:Chol:PEG-lipid |
| 40:15:43.5:1.5 | Compound:Phospholipid:Chol:PEG-lipid |
| 50:15:33.5:1.5 | Compound:Phospholipid:Chol:PEG-lipid |
| 40:10:48.5:1.5 | Compound:Phospholipid:Chol:PEG-lipid |
| 45:10:43.5:1.5 | Compound:Phospholipid:Chol:PEG-lipid |
| 40:5:53.5:1.5 | Compound:Phospholipid:Chol:PEG-lipid |
| 45:5:48.5:1.5 | Compound:Phospholipid:Chol:PEG-lipid |
| 50:5:43.5:1.5 | Compound:Phospholipid:Chol:PEG-lipid |
| 40:20:40:0 | Compound:Phospholipid:Chol:PEG-lipid |
| 45:20:35:0 | Compound:Phospholipid:Chol:PEG-lipid |
| 50:20:30:0 | Compound:Phospholipid:Chol:PEG-lipid |
| 40:15:45:0 | Compound:Phospholipid:Chol:PEG-lipid |
| 45:15:40:0 | Compound:Phospholipid:Chol:PEG-lipid |
| 50:15:35:0 | Compound:Phospholipid:Chol:PEG-lipid |
| 40:10:50:0 | Compound:Phospholipid:Chol:PEG-lipid |
| 45:10:45:0 | Compound:Phospholipid:Chol:PEG-lipid |
| 50:10:40:0 | Compound:Phospholipid:Chol:PEG-lipid |
| 47.5:10.5:39:3 | Compound:Phospholipid:Chol:PEG-lipid |
| 47.5:10:39.5:3 | Compound:Phospholipid:Chol:PEG-lipid |
| 47.5:11:39.5:2 | Compound:Phospholipid:Chol:PEG-lipid |
| 47.5:10.5:39.5:2.5 | Compound:Phospholipid:Chol:PEG-lipid |
| 47.5:11:39:2.5 | Compound:Phospholipid:Chol:PEG-lipid |
| 48.5:10:38.5:3 | Compound:Phospholipid:Chol:PEG-lipid |
| 48.5:10.5:39:2 | Compound:Phospholipid:Chol:PEG-lipid |
| 48.5:10.5:38.5:2.5 | Compound:Phospholipid:Chol:PEG-lipid |
| 48.5:10.5:39.5:1.5 | Compound:Phospholipid:Chol:PEG-lipid |
| 48.5:10.5:38.0:3 | Compound:Phospholipid:Chol:PEG-lipid |
| 47:10.5:39.5:3 | Compound:Phospholipid:Chol:PEG-lipid |
| 47:10:40.5:2.5 | Compound:Phospholipid:Chol:PEG-lipid |
| 47:11:40:2 | Compound:Phospholipid:Chol:PEG-lipid |
| 47:10.5:39.5:3 | Compound:Phospholipid:Chol:PEG-lipid |
| 48:10.5:38.5:3 | Compound:Phospholipid:Chol:PEG-lipid |
| 48:10:39.5:2.5 | Compound:Phospholipid:Chol:PEG-lipid |
| 48:11:39:2 | Compound:Phospholipid:Chol:PEG-lipid |
| 48:10.5:38.5:3 | Compound:Phospholipid:Chol:PEG-lipid |

### Example 3: Expression, Clearance, and Tolerability of Sample Formulations

High accumulation of a lipid in a tissue could trigger toxic effects, and is thus undesirable. On the other hand, a lipid that is metabolized quickly in a tissue may not deliver enough of a therapeutic agent, such as, e.g., an mRNA, to cells to be effective. The aim of the studies in Examples 4-8 is the identification of amino lipids with optimal metabolic stability that also yield high potency lipid nanoparticles. Table 9 depicts ionizable amino lipids assessed in Examples 4-8.

TABLE 9
Ionizable Amino Lipids

| Cpd | Structure |
|---|---|
| 7 | 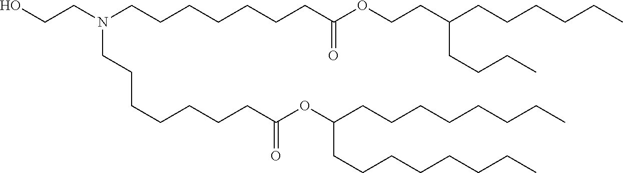 [SMILES: CCCCCCCCC(CCCCCCCC)OC(=O)CCCCCCCN(CCO)CCCCCCCC(=O)OCCC(CCCC)CCCCCC] |
| 8 | 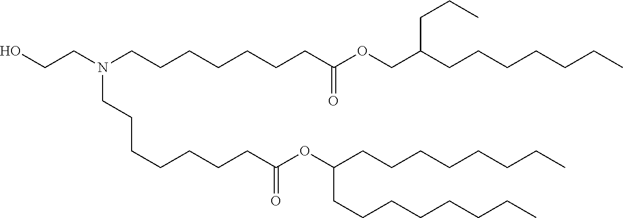 [SMILES: CCCCCCCCC(CCCCCCCC)OC(=O)CCCCCCCN(CCO)CCCCCCCC(=O)OCC(CCC)CCCCCCC] |
| 9 | 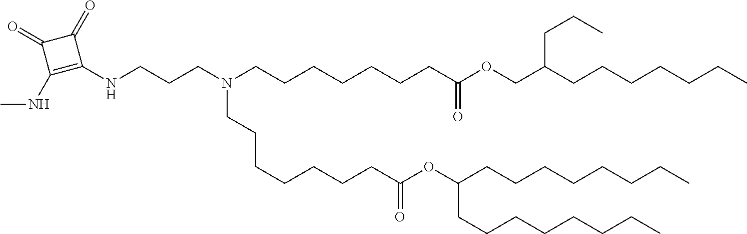 [SMILES: CCCCCCCCC(CCCCCCCC)OC(=O)CCCCCCCN(CCCCCCCC(=O)OCC(CCC)CCCCCCC)CCCNc1c(NC)c(=O)c1=O] |
| 10 | 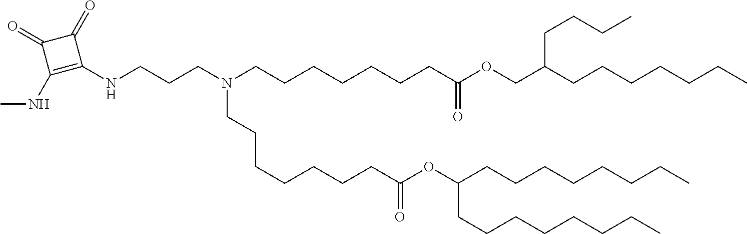 [SMILES: CCCCCCCCC(CCCCCCCC)OC(=O)CCCCCCCN(CCCCCCCC(=O)OCC(CCCC)CCCCCCC)CCCNc1c(NC)c(=O)c1=O] |
| 11 | 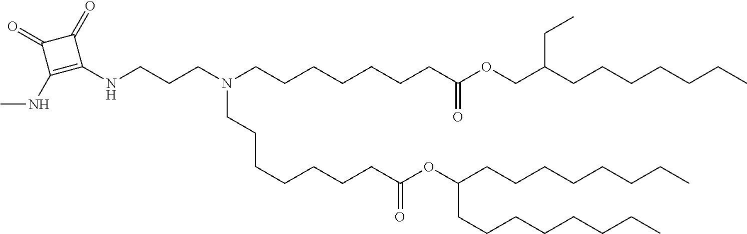 [SMILES: CCCCCCCCC(CCCCCCCC)OC(=O)CCCCCCCN(CCCCCCCC(=O)OCC(CC)CCCCCCC)CCCNc1c(NC)c(=O)c1=O] |
| 12 | 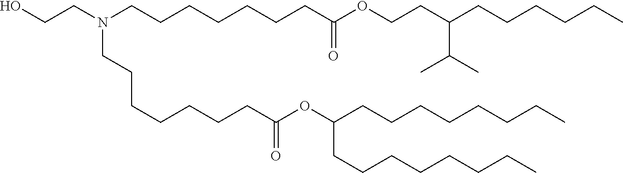 [SMILES: CCCCCCCCC(CCCCCCCC)OC(=O)CCCCCCCN(CCO)CCCCCCCC(=O)OCCC(CCCCCC)C(C)C] |
| 13 | 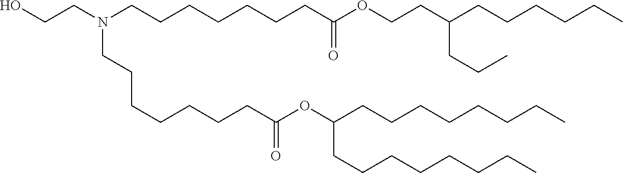 [SMILES: CCCCCCCCC(CCCCCCCC)OC(=O)CCCCCCCN(CCO)CCCCCCCC(=O)OCCC(CCC)CCCCCC] |
| 14 | 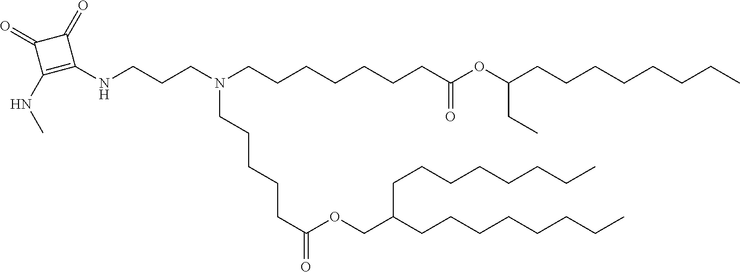 [SMILES: CCCCCCCCC(CCCCCCCC)COC(=O)CCCCCN(CCCCCCCC(=O)OC(CC)CCCCCCCC)CCCNc1c(NC)c(=O)c1=O] |
| 15 | 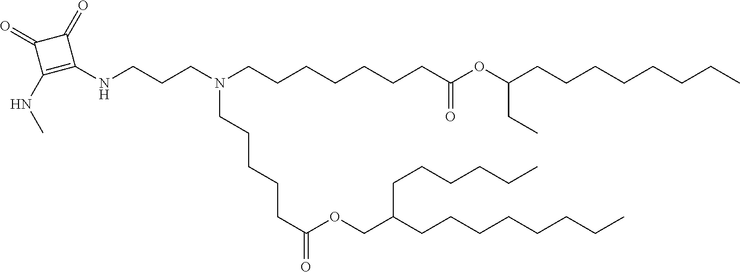 [SMILES: CCCCCCCCC(CCCCCC)COC(=O)CCCCCN(CCCCCCCC(=O)OC(CC)CCCCCCCC)CCCNc1c(NC)c(=O)c1=O] |
| 16 | 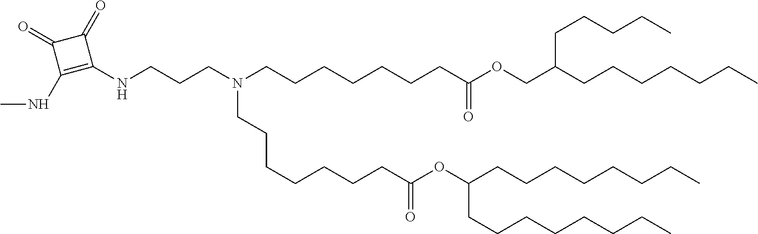 [SMILES: CCCCCCCCC(CCCCCCCC)OC(=O)CCCCCCCN(CCCCCCCC(=O)OCC(CCCCC)CCCCCCC)CCCNc1c(NC)c(=O)c1=O] |
| 17 | 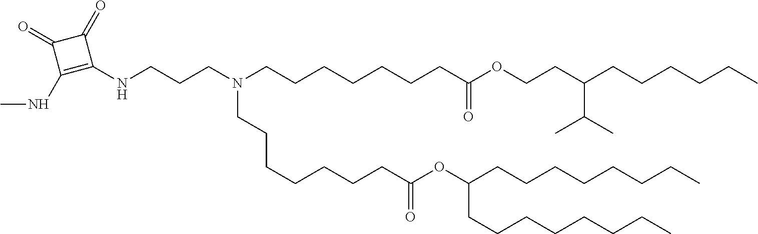 [SMILES: CCCCCCCCC(CCCCCCCC)OC(=O)CCCCCCCN(CCCCCCCC(=O)OCCC(CCCCCC)C(C)C)CCCNc1c(NC)c(=O)c1=O] |
| 18 | 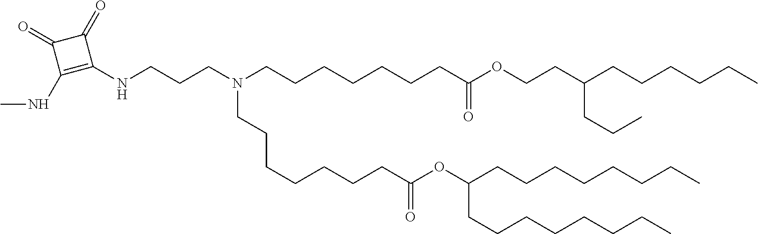 [SMILES: CCCCCCCCC(CCCCCCCC)OC(=O)CCCCCCCN(CCCCCCCC(=O)OCCC(CCC)CCCCCC)CCCNc1c(NC)c(=O)c1=O] |
| 19 | 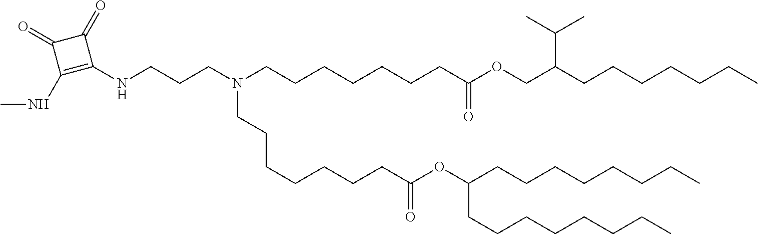 [SMILES: CCCCCCCCC(CCCCCCCC)OC(=O)CCCCCCCN(CCCCCCCC(=O)OCC(CCCCCCC)C(C)C)CCCNc1c(NC)c(=O)c1=O] |
| 20 | 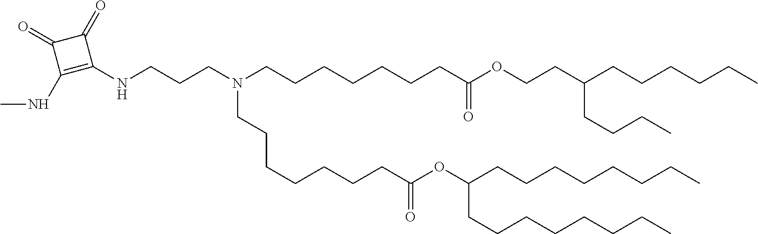 [SMILES: CCCCCCCCC(CCCCCCCC)OC(=O)CCCCCCCN(CCCCCCCC(=O)OCCC(CCCC)CCCCCC)CCCNc1c(NC)c(=O)c1=O] |
| 21 | 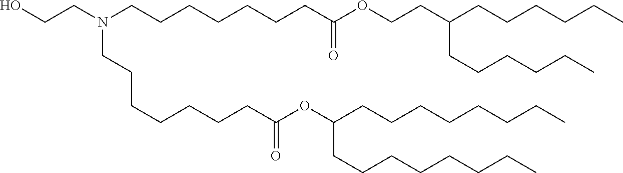 [SMILES: CCCCCCCCC(CCCCCCCC)OC(=O)CCCCCCCN(CCO)CCCCCCCC(=O)OCCC(CCCCCC)CCCCCC] |
| 22 | 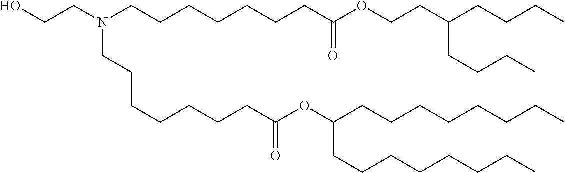 [SMILES: CCCCCCCCC(CCCCCCCC)OC(=O)CCCCCCCN(CCO)CCCCCCCC(=O)OCCC(CCCC)CCCC] |
| 23 | 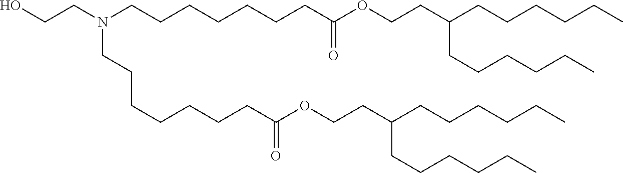 [SMILES: CCCCCCC(CCCCCC)CCOC(=O)CCCCCCCN(CCO)CCCCCCCC(=O)OCCC(CCCCCC)CCCCCC] |
| 24 | 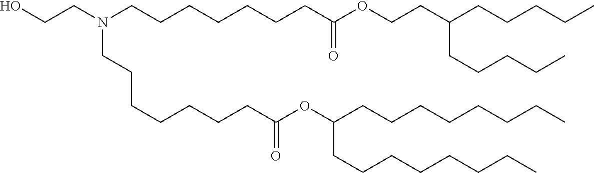 [SMILES: CCCCCCCCC(CCCCCCCC)OC(=O)CCCCCCCN(CCO)CCCCCCCC(=O)OCCC(CCCCC)CCCCC] |
| 25 | 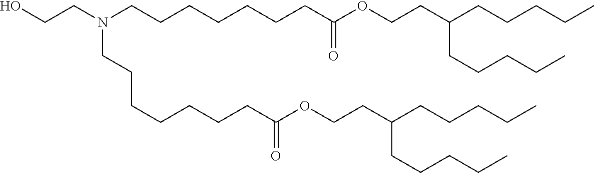 [SMILES: CCCCCC(CCCCC)CCOC(=O)CCCCCCCN(CCO)CCCCCCCC(=O)OCCC(CCCCC)CCCCC] |
| 26 | 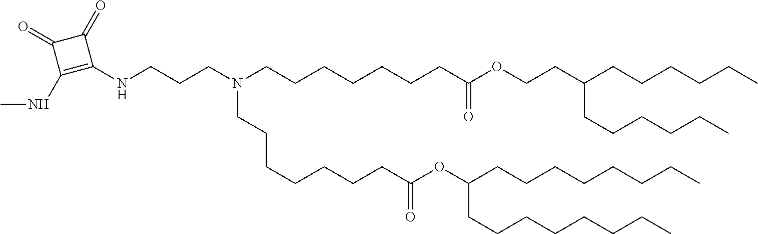 [SMILES: CCCCCCCCC(CCCCCCCC)OC(=O)CCCCCCCN(CCCCCCCC(=O)OCCC(CCCCCC)CCCCCC)CCCNc1c(NC)c(=O)c1=O] |
| 27 | 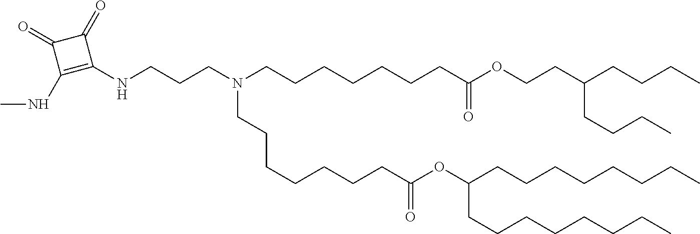 [SMILES: CCCCCCCCC(CCCCCCCC)OC(=O)CCCCCCCN(CCCCCCCC(=O)OCCC(CCCC)CCCC)CCCNc1c(NC)c(=O)c1=O] |
| 28 | 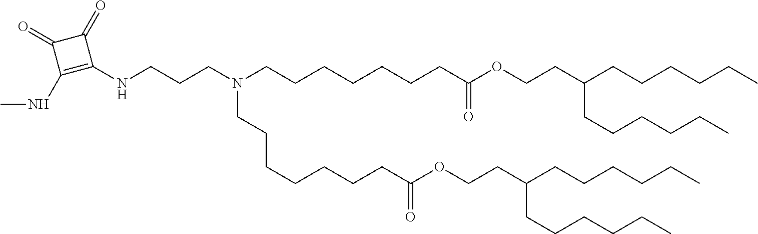 [SMILES: CCCCCCC(CCCCCC)CCOC(=O)CCCCCCCN(CCCCCCCC(=O)OCCC(CCCCCC)CCCCCC)CCCNc1c(NC)c(=O)c1=O] |
| 29 | 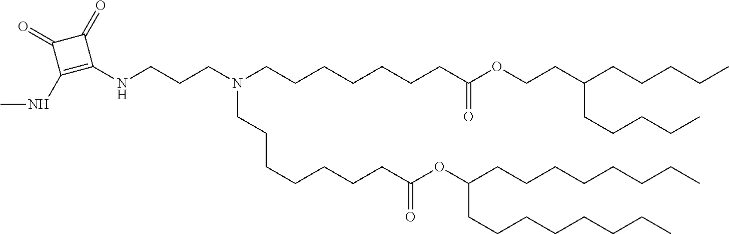 [SMILES: CCCCCCCCC(CCCCCCCC)OC(=O)CCCCCCCN(CCCCCCCC(=O)OCCC(CCCCC)CCCCC)CCCNc1c(NC)c(=O)c1=O] |
| 30 | 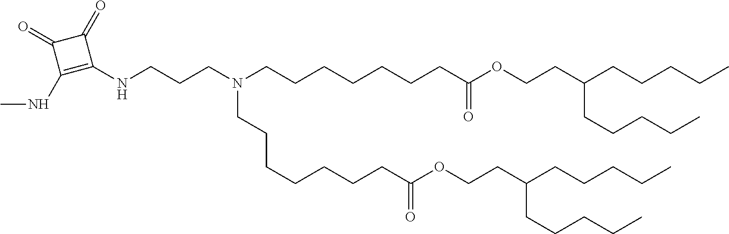 [SMILES: CCCCCC(CCCCC)CCOC(=O)CCCCCCCN(CCCCCCCC(=O)OCCC(CCCCC)CCCCC)CCCNc1c(NC)c(=O)c1=O] |
| 31 | 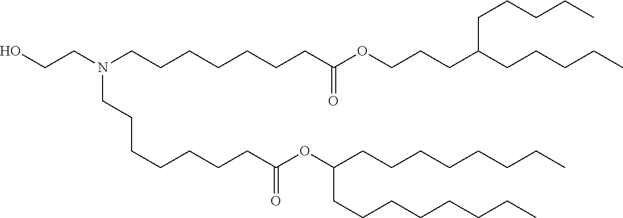 [SMILES: CCCCCCCCC(CCCCCCCC)OC(=O)CCCCCCCN(CCO)CCCCCCCC(=O)OCCCC(CCCCC)CCCCC] |
| 32 | 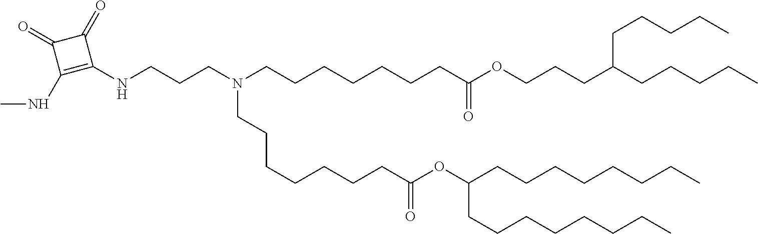 [SMILES: CCCCCCCCC(CCCCCCCC)OC(=O)CCCCCCCN(CCCCCCCC(=O)OCCCC(CCCCC)CCCCC)CCCNc1c(NC)c(=O)c1=O] |
| 33 | 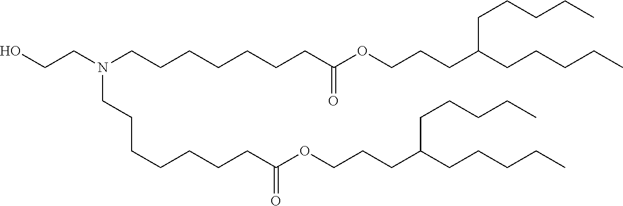 [SMILES: CCCCCC(CCCCC)CCCOC(=O)CCCCCCCN(CCO)CCCCCCCC(=O)OCCCC(CCCCC)CCCCC] |
| 34 | 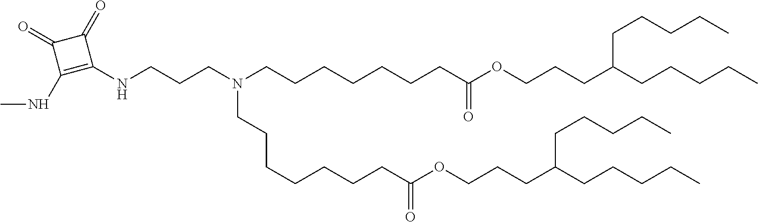 [SMILES: CCCCCC(CCCCC)CCCOC(=O)CCCCCCCN(CCCCCCCC(=O)OCCCC(CCCCC)CCCCC)CCCNc1c(NC)c(=O)c1=O] |
| 35 | 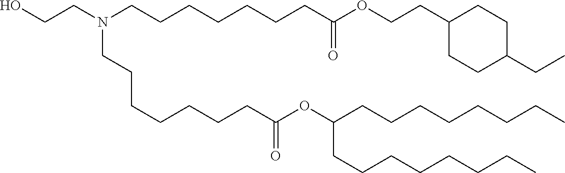 [SMILES: CCCCCCCCC(CCCCCCCC)OC(=O)CCCCCCCN(CCO)CCCCCCCC(=O)OCCC1CCC(CC)CC1] |
| 36 | 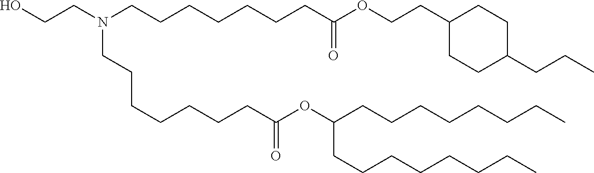 [SMILES: CCCCCCCCC(CCCCCCCC)OC(=O)CCCCCCCN(CCO)CCCCCCCC(=O)OCCC1CCC(CCC)CC1] |
| 37 | 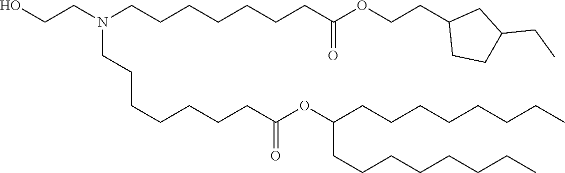 [SMILES: CCCCCCCCC(CCCCCCCC)OC(=O)CCCCCCCN(CCO)CCCCCCCC(=O)OCCC1CCC(CC)C1] |
| 38 | 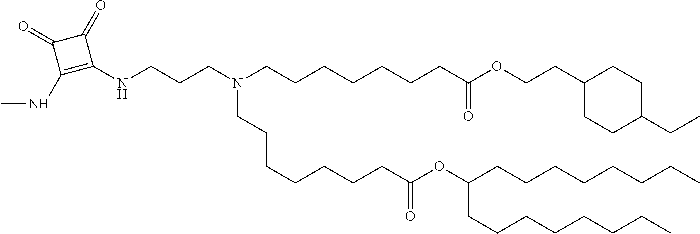 [SMILES: CCCCCCCCC(CCCCCCCC)OC(=O)CCCCCCCN(CCCCCCCC(=O)OCCC1CCC(CC)CC1)CCCNc1c(NC)c(=O)c1=O] |
| 39 | 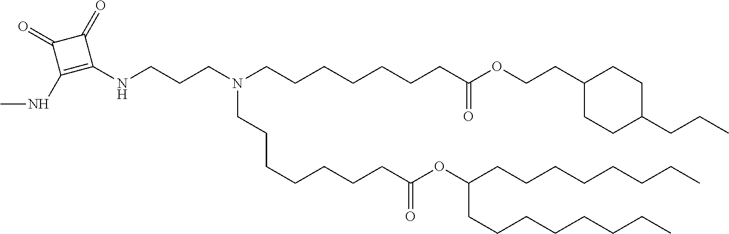 [SMILES: CCCCCCCCC(CCCCCCCC)OC(=O)CCCCCCCN(CCCCCCCC(=O)OCCC1CCC(CCC)CC1)CCCNc1c(NC)c(=O)c1=O] |
| 40 | 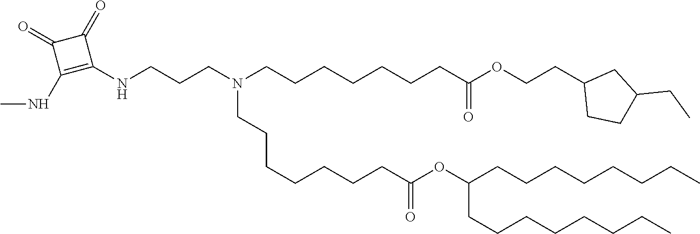 [SMILES: CCCCCCCCC(CCCCCCCC)OC(=O)CCCCCCCN(CCCCCCCC(=O)OCCC1CCC(CC)C1)CCCNc1c(NC)c(=O)c1=O] |
| 41 | 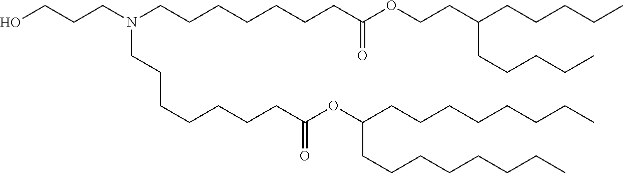 [SMILES: CCCCCCCCC(CCCCCCCC)OC(=O)CCCCCCCN(CCCO)CCCCCCCC(=O)OCCC(CCCCC)CCCCC] |
| 42 | 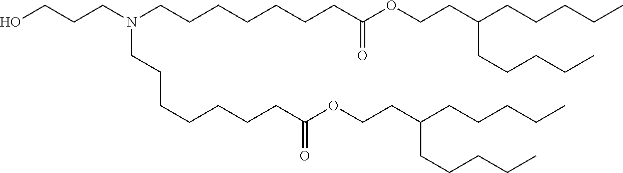 [SMILES: CCCCCC(CCCCC)CCOC(=O)CCCCCCCN(CCCO)CCCCCCCC(=O)OCCC(CCCCC)CCCCC] |
| 43 | 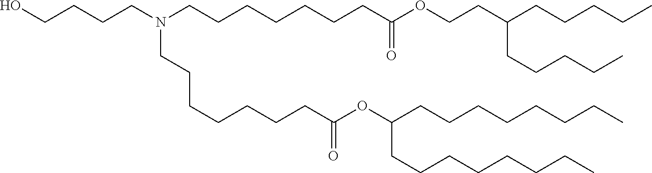 [SMILES: CCCCCCCCC(CCCCCCCC)OC(=O)CCCCCCCN(CCCCO)CCCCCCCC(=O)OCCC(CCCCC)CCCCC] |
| 44 | 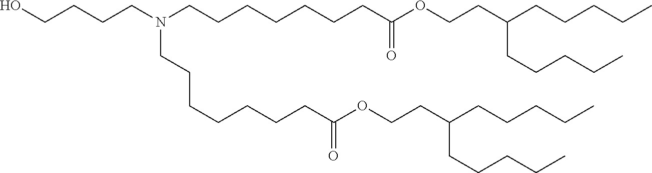 [SMILES: CCCCCC(CCCCC)CCOC(=O)CCCCCCCN(CCCCO)CCCCCCCC(=O)OCCC(CCCCC)CCCCC] |
| 45 | 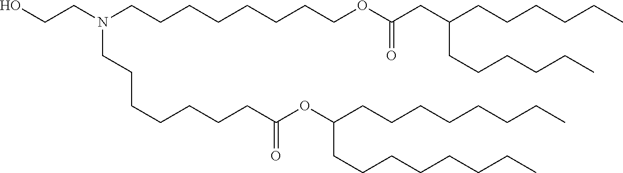 [SMILES: CCCCCCCCC(CCCCCCCC)OC(=O)CCCCCCCN(CCO)CCCCCCCCOC(=O)CC(CCCCCC)CCCCCC] |
| 46 | 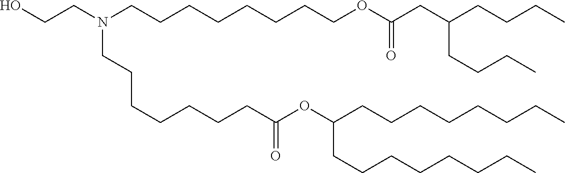 [SMILES: CCCCCCCCC(CCCCCCCC)OC(=O)CCCCCCCN(CCO)CCCCCCCCOC(=O)CC(CCCC)CCCC] |
| 47 | 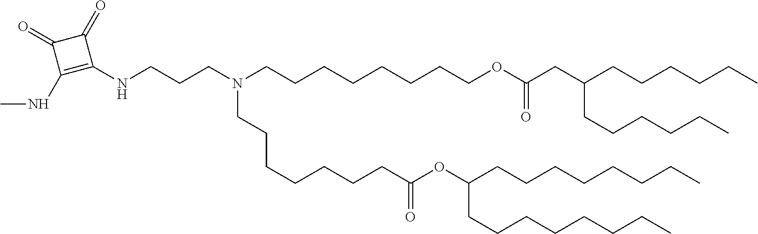 [SMILES: CCCCCCCCC(CCCCCCCC)OC(=O)CCCCCCCN(CCCCCCCCOC(=O)CC(CCCCCC)CCCCCC)CCCNc1c(NC)c(=O)c1=O] |
| 48 | 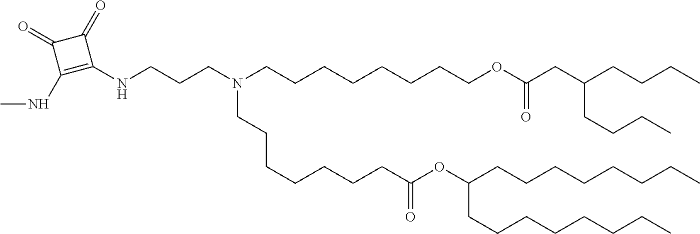 [SMILES: CCCCCCCCC(CCCCCCCC)OC(=O)CCCCCCCN(CCCCCCCCOC(=O)CC(CCCC)CCCC)CCCNc1c(NC)c(=O)c1=O] |
| 49 | 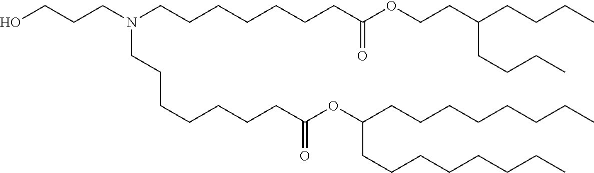 [SMILES: CCCCCCCCC(CCCCCCCC)OC(=O)CCCCCCCN(CCCO)CCCCCCCC(=O)OCCC(CCCC)CCCC] |
| 50 | 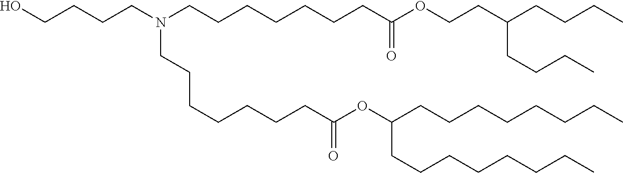 [SMILES: CCCCCCCCC(CCCCCCCC)OC(=O)CCCCCCCN(CCCCO)CCCCCCCC(=O)OCCC(CCCC)CCCC] |
| 51 | 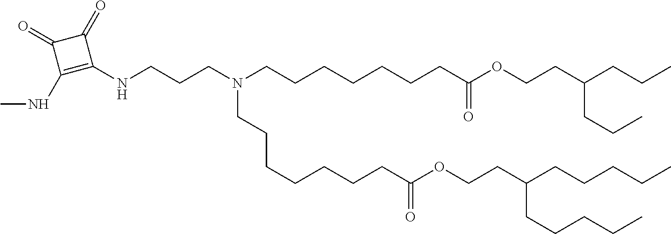 [SMILES: CCCCCC(CCCCC)CCOC(=O)CCCCCCCN(CCCCCCCC(=O)OCCC(CCC)CCC)CCCNc1c(NC)c(=O)c1=O] |
| 52 | 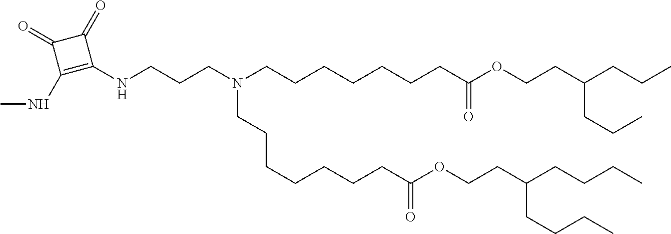 [SMILES: CCCCC(CCCC)CCOC(=O)CCCCCCCN(CCCCCCCC(=O)OCCC(CCC)CCC)CCCNc1c(NC)c(=O)c1=O] |
| 53 | 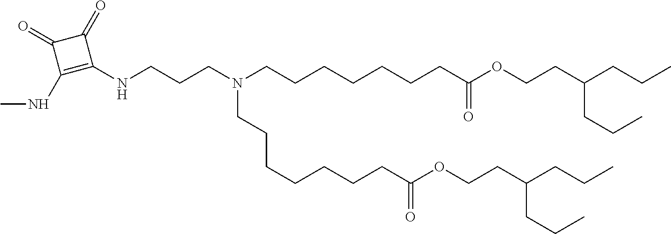 [SMILES: CCCC(CCC)CCOC(=O)CCCCCCCN(CCCCCCCC(=O)OCCC(CCC)CCC)CCCNc1c(NC)c(=O)c1=O] |
| 54 | 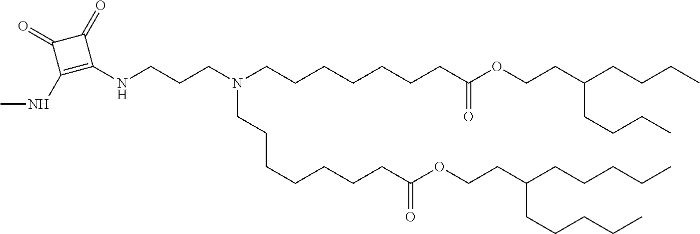 [SMILES: CCCCCC(CCCCC)CCOC(=O)CCCCCCCN(CCCCCCCC(=O)OCCC(CCCC)CCCC)CCCNc1c(NC)c(=O)c1=O] |
| 55 | 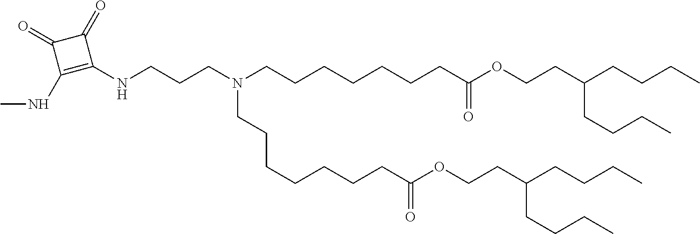 [SMILES: CCCCC(CCCC)CCOC(=O)CCCCCCCN(CCCCCCCC(=O)OCCC(CCCC)CCCC)CCCNc1c(NC)c(=O)c1=O] |
| 56 | 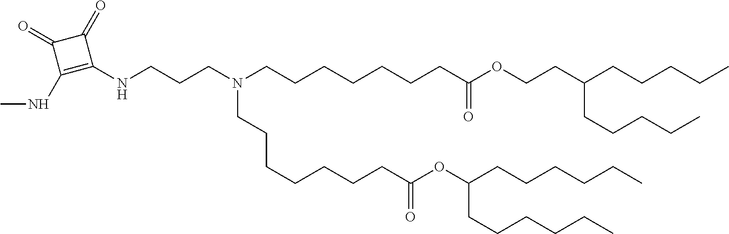 [SMILES: CCCCCCC(CCCCCC)OC(=O)CCCCCCCN(CCCCCCCC(=O)OCCC(CCCCC)CCCCC)CCCNc1c(NC)c(=O)c1=O] |
| 57 | 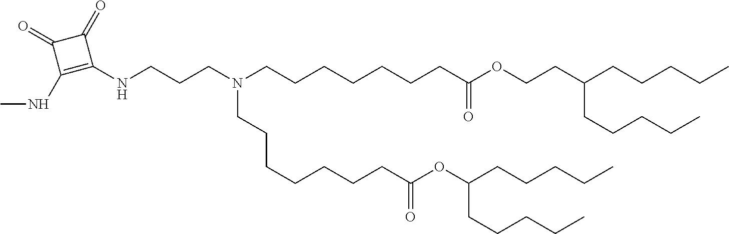 [SMILES: CCCCCC(CCCCC)CCOC(=O)CCCCCCCN(CCCCCCCC(=O)OC(CCCCC)CCCCC)CCCNc1c(NC)c(=O)c1=O] |
| 58 | 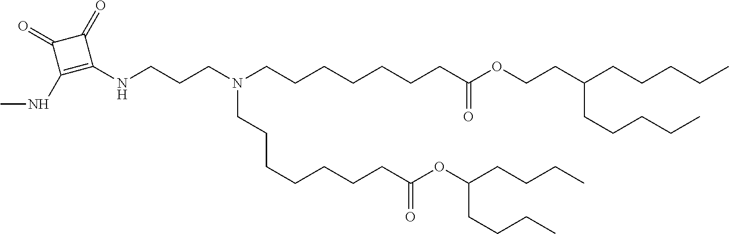 [SMILES: CCCCCC(CCCCC)CCOC(=O)CCCCCCCN(CCCCCCCC(=O)OC(CCCC)CCCC)CCCNc1c(NC)c(=O)c1=O] |
| 59 | 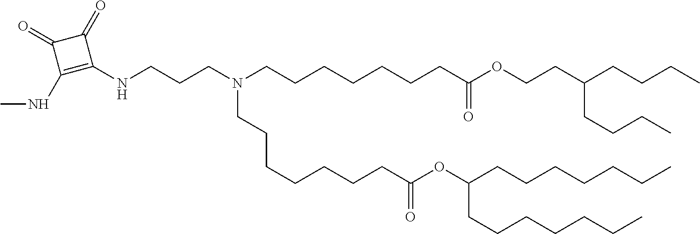 [SMILES: CCCCCCCC(CCCCCCC)OC(=O)CCCCCCCN(CCCCCCCC(=O)OCCC(CCCC)CCCC)CCCNc1c(NC)c(=O)c1=O] |
| 60 | 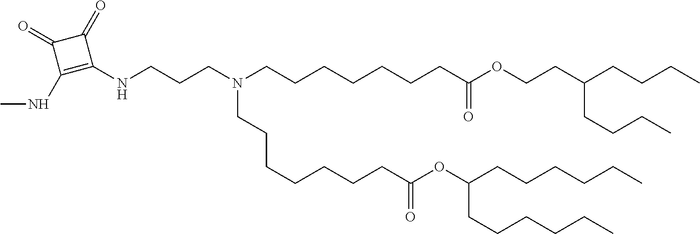 [SMILES: CCCCCCC(CCCCCC)OC(=O)CCCCCCCN(CCCCCCCC(=O)OCCC(CCCC)CCCC)CCCNc1c(NC)c(=O)c1=O] |
| 61 | 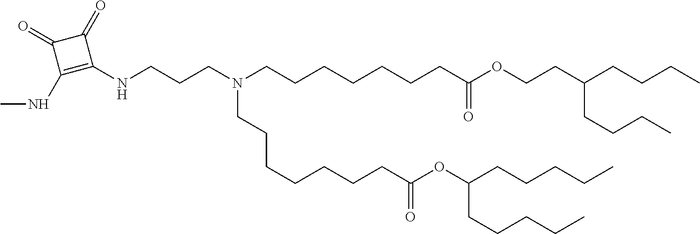 [SMILES: CCCCCC(CCCCC)OC(=O)CCCCCCCN(CCCCCCCC(=O)OCCC(CCCC)CCCC)CCCNc1c(NC)c(=O)c1=O] |
| 62 | 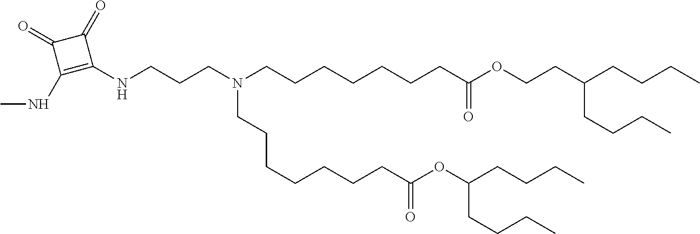 [SMILES: CCCCC(CCCC)CCOC(=O)CCCCCCCN(CCCCCCCC(=O)OC(CCCC)CCCC)CCCNc1c(NC)c(=O)c1=O] |
| 63 | 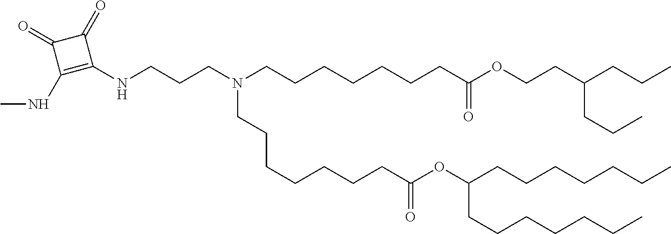 [SMILES: CCCCCCCC(CCCCCCC)OC(=O)CCCCCCCN(CCCCCCCC(=O)OCCC(CCC)CCC)CCCNc1c(NC)c(=O)c1=O] |
| 64 | 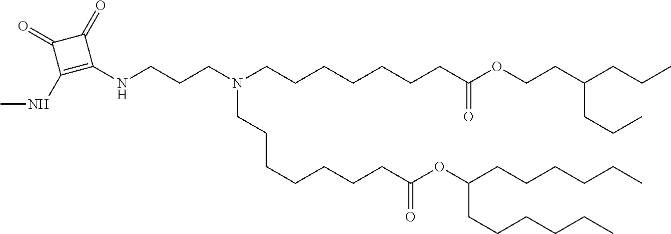 [SMILES: CCCCCCC(CCCCCC)OC(=O)CCCCCCCN(CCCCCCCC(=O)OCCC(CCC)CCC)CCCNc1c(NC)c(=O)c1=O] |
| 65 | 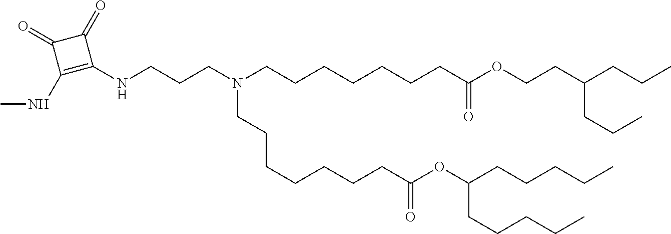 [SMILES: CCCCCC(CCCCC)OC(=O)CCCCCCCN(CCCCCCCC(=O)OCCC(CCC)CCC)CCCNc1c(NC)c(=O)c1=O] |
| 66 | 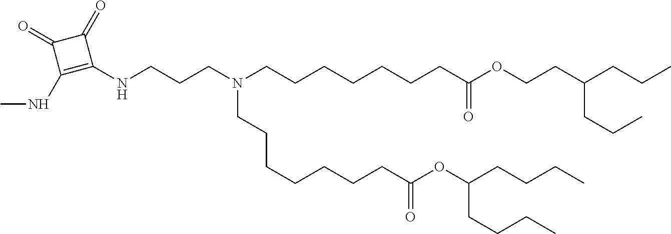 [SMILES: CCCCC(CCCC)OC(=O)CCCCCCCN(CCCCCCCC(=O)OCCC(CCC)CCC)CCCNc1c(NC)c(=O)c1=O] |
| 67 | 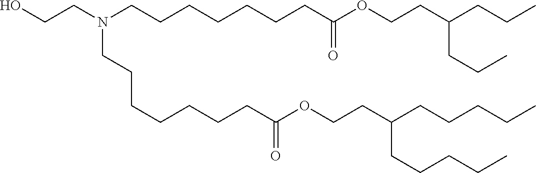 [SMILES: CCCCCC(CCCCC)CCOC(=O)CCCCCCCN(CCO)CCCCCCCC(=O)OCCC(CCC)CCC] |
| 68 | 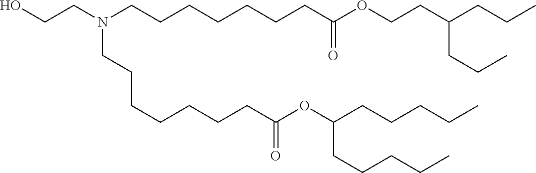 [SMILES: CCCCCC(CCCCC)OC(=O)CCCCCCCN(CCO)CCCCCCCC(=O)OCCC(CCC)CCC] |
| 69 | 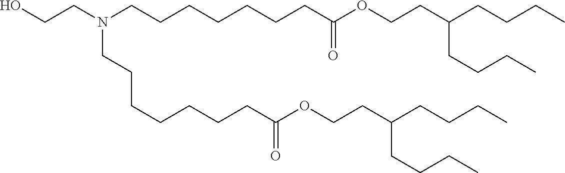 [SMILES: CCCCC(CCCC)CCOC(=O)CCCCCCCN(CCO)CCCCCCCC(=O)OCCC(CCCC)CCCC] |
| 70 | 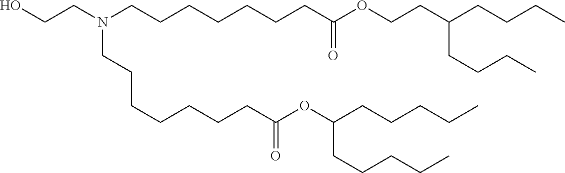 [SMILES: CCCCCC(CCCCC)OC(=O)CCCCCCCN(CCO)CCCCCCCC(=O)OCCC(CCCC)CCCC] |
| 71 | 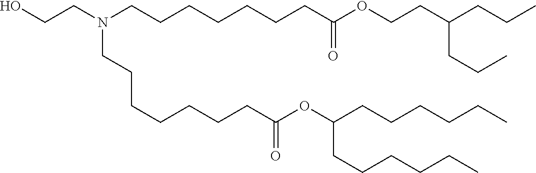 [SMILES: CCCCCCC(CCCCCC)OC(=O)CCCCCCCN(CCO)CCCCCCCC(=O)OCCC(CCC)CCC] |
| 72 | 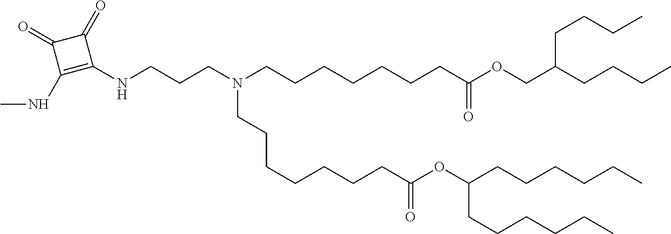 [SMILES: CCCCCCC(CCCCCC)OC(=O)CCCCCCCN(CCCCCCCC(=O)OCC(CCCC)CCCC)CCCNc1c(NC)c(=O)c1=O] |
| 73 | 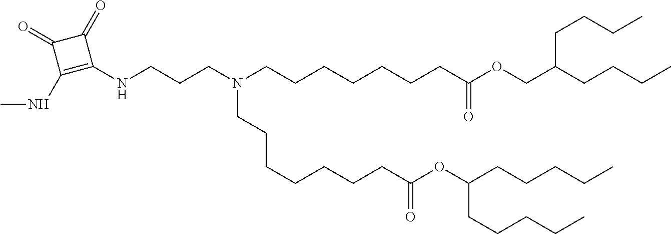 [SMILES: CCCCCC(CCCCC)OC(=O)CCCCCCCN(CCCCCCCC(=O)OCC(CCCC)CCCC)CCCNc1c(NC)c(=O)c1=O] |
| 74 | 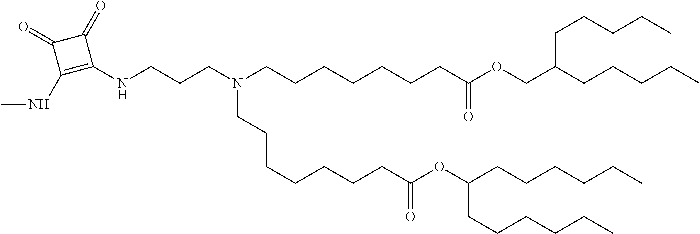 [SMILES: CCCCCCC(CCCCCC)OC(=O)CCCCCCCN(CCCCCCCC(=O)OCC(CCCCC)CCCCC)CCCNc1c(NC)c(=O)c1=O] |
| 75 | 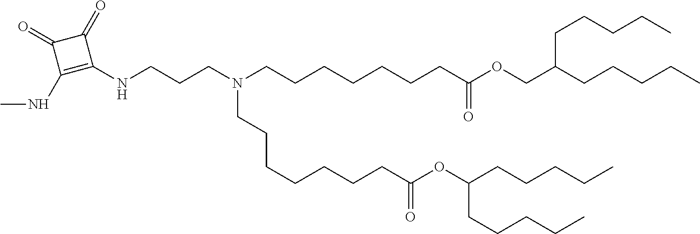 [SMILES: CCCCCC(CCCCC)COC(=O)CCCCCCCN(CCCCCCCC(=O)OC(CCCCC)CCCCC)CCCNc1c(NC)c(=O)c1=O] |
| 76 | 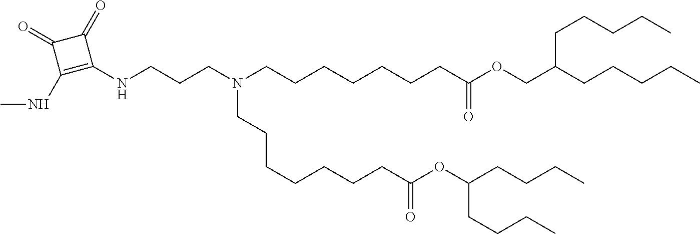 [SMILES: CCCCCC(CCCCC)COC(=O)CCCCCCCN(CCCCCCCC(=O)OC(CCCC)CCCC)CCCNc1c(NC)c(=O)c1=O] |
| 77 | 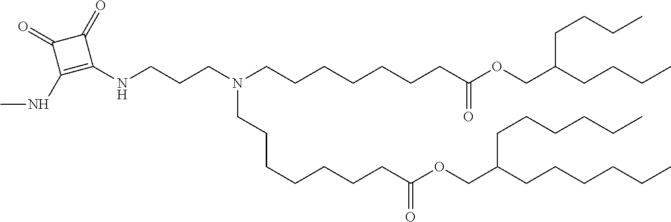 [SMILES: CCCCCCC(CCCCCC)COC(=O)CCCCCCCN(CCCCCCCC(=O)OCC(CCCC)CCCC)CCCNc1c(NC)c(=O)c1=O] |
| 78 | 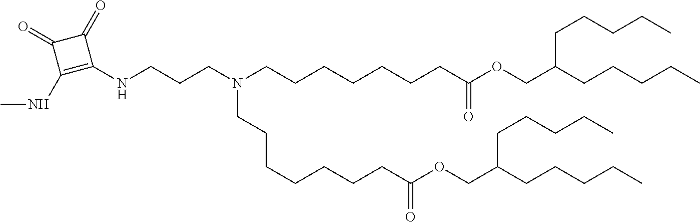 [SMILES: CCCCCC(CCCCC)COC(=O)CCCCCCCN(CCCCCCCC(=O)OCC(CCCCC)CCCCC)CCCNc1c(NC)c(=O)c1=O] |
| 79 | 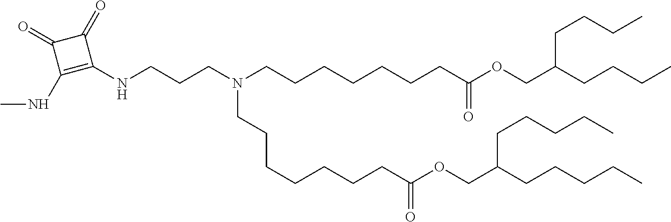 [SMILES: CCCCCC(CCCCC)COC(=O)CCCCCCCN(CCCCCCCC(=O)OCC(CCCC)CCCC)CCCNc1c(NC)c(=O)c1=O] |
| 80 | 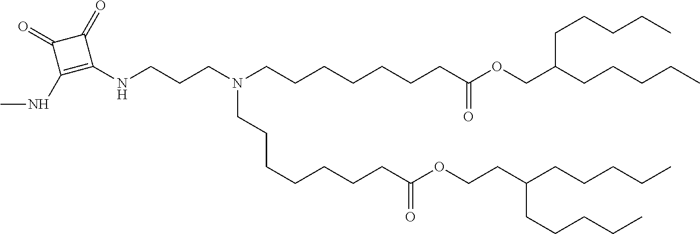 [SMILES: CCCCCC(CCCCC)CCOC(=O)CCCCCCCN(CCCCCCCC(=O)OCC(CCCCC)CCCCC)CCCNc1c(NC)c(=O)c1=O] |
| 81 | 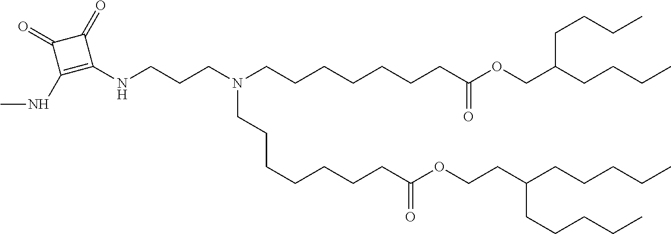 [SMILES: CCCCCC(CCCCC)CCOC(=O)CCCCCCCN(CCCCCCCC(=O)OCC(CCCC)CCCC)CCCNc1c(NC)c(=O)c1=O] |
| 82 | 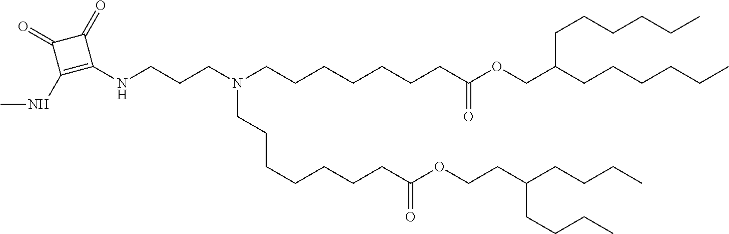 [SMILES: CCCCCCC(CCCCCC)COC(=O)CCCCCCCN(CCCCCCCC(=O)OCCC(CCCC)CCCC)CCCNc1c(NC)c(=O)c1=O] |
| 83 | 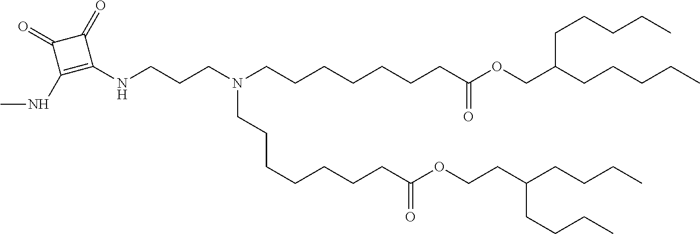 [SMILES: CCCCCC(CCCCC)COC(=O)CCCCCCCN(CCCCCCCC(=O)OCCC(CCCC)CCCC)CCCNc1c(NC)c(=O)c1=O] |
| 84 | 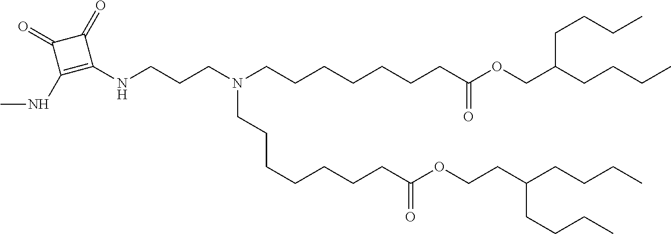 [SMILES: CCCCC(CCCC)CCOC(=O)CCCCCCCN(CCCCCCCC(=O)OCC(CCCC)CCCC)CCCNc1c(NC)c(=O)c1=O] |

The following depicts the synthesis of several of the above compounds.

### Syntheses of Intermediates

### 3-Butylheptyl 8-bromooctanoate

### Step 1: Synthesis of ethyl 3-butylhept-2-enoate

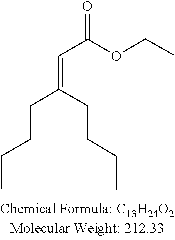

Triethyl phosphonoacetate (9.07 mL, 45.7 mmol) was added dropwise over 20 minutes to a suspension of sodium hydride (1.83 g, 45.7 mmol) in THE (14 mL) and the mixture was stirred at room temperature until gas evolution ceased (approximately 30 min). The reaction mixture was chilled to 0° C. and 5-nonanone (6.05 mL, 35.2 mmol) was added in portions. The reaction was gradually warmed to room temperature and allowed to stir under reflux for 24 h. The reaction was cooled to room temperature prior to being quenched with saturated aqueous sodium bicarbonate. The aqueous phase was extracted with diethyl ether, and the organic extracts were washed with brine, dried (MgSO4), and concentrated. The crude material was purified by silica gel chromatography (0-20% EtOAc:hexanes) to afford ethyl 3-butylhept-2-enoate (5.27 g, 24.8 mmol, 71%) as a clear oil. ¹H NMR (300 MHz, CDCl₃) δ: ppm 5.62 (s, 1H); 4.14 (q, 2H, J=6.0 Hz); 2.59 (t, 2H, J=6.0 Hz); 2.14 (t, 2H, J=6.0 Hz); 1.50-1.23 (m, 11H); 0.99-0.82 (m, 6H).

### Step 2: Synthesis of ethyl 3-butylheptanoate

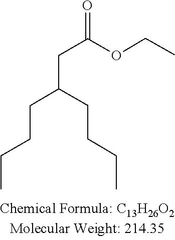

A steel Parr reactor equipped with a stir bar was charged with ethyl 3-butylhept-2-enoate (10.5 g, 49.5 mmol) in ethanol (50 mL). Palladium hydroxide on carbon (1.04 g, 7.42 mmol) was added and the vessel was sealed, evacuated, refilled with H₂ gas (3×), and the pressure was set to 200 psi. The reaction was stirred at 500 rpm, under 200 psi H₂ gas, at room temperature for 2 h. The vessel was then evacuated, refilled with N₂ gas, and opened. The crude reaction mixture was filtered through a Celite pad. The Celite pad was washed with EtOH and the crude material was concentrated to give ethyl 3-butylheptanoate (9.69 g, 45.2 mmol, 91%) as a clear oil. The compound was carried onto the next step without further purification. ¹H NMR (300 MHz, CDCl₃) δ: ppm 4.12 (q, 2H, J=9.0 Hz); 2.22 (d, 2H, J=6.0 Hz); 1.90-1.76 (m, 1H); 1.38-1.19 (m, 15H); 0.88 (br. t, 6H, J=6.0 Hz).

### Step 3: Synthesis of 3-butylheptan-1-ol

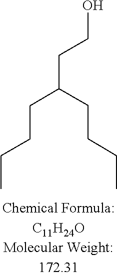

To a mixture of lithium aluminum hydride (850 mg, 22.4 mmol) in dry ether (23 mL) under N₂ at 0° C., was added dropwise ethyl 3-butylheptanoate (4.00 g, 18.7 mmol) in dry ether (15 mL). The mixture was stirred at room temperature for 2.5 h prior to being cooled to 0° C. Water (1 mL per g of LiAlH₄) was added to the solution dropwise, followed by the slow addition of 15% sodium hydroxide (1 mL per g of LiAlH₄) and water (3 mL per g of LiAlH₄). The solution was stirred for a few minutes at room temperature and filtered through a Celite pad. The Celite pad was washed with diethyl ether and the filtrate was concentrated. The crude material was purified by silica gel chromatography (0-40% EtOAc:hexanes) to afford 3-butylheptan-1-ol (3.19 g, 18.5 mmol, 99%) as a clear oil. ¹H NMR (300 MHz, CDCl₃) δ: ppm 3.66 (t, 2H, J=6.0 Hz); 1.53 (q, 2H, J=6.0 Hz); 1.46-1.36 (m, 1H); 1.35-1.21 (m, 12H); 1.18 (br. s, 1H); 0.89 (br. t, 6H, J=6.0 Hz).

### Step 4: Synthesis of 3-butylheptyl 8-bromooctanoate

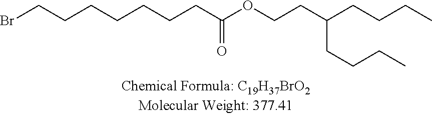 [SMILES: CCCCC(CCCC)CCOC(=O)CCCCCCCBr]

To a solution of 3-butylheptan-1-ol (3.19 g, 18.5 mmol), 8-bromooctanoic acid (4.96 g, 22.2 mmol), and DMAP (453 mg, 3.71 mmol) in methylene chloride (32 mL) at 0° C. was added EDCI (5.33 g, 27.8 mmol) and the reaction mixture stirred at room temperature overnight. The reaction mixture was then cooled to 0° C. and a solution of 10% hydrochloric acid (150 mL) was added slowly over 20 minutes. The layers were separated, and the organic layer was concentrated in vacuum to give a crude oil. The oil was dissolved in hexane (150 mL) and washed with a mixture of acetonitrile (150 mL) and 5% sodium bicarbonate (150 mL). The hexane layer was separated, dried (MgSO4), and filtered. The solvent was removed under vacuum to give 3-butylheptyl 8-bromooctanoate (6.90 g, 18.3 mmol, 99%) as a clear oil. The compound was carried onto the next step without further purification. ¹H NMR (300 MHz, CDCl₃) δ: ppm 4.08 (t, 2H, J=6.0 Hz); 3.40 (t, 2H, J=6.0 Hz); 2.29 (t, 2H, J=6.0 Hz); 1.85 (pent., 2H, J=6.0 Hz); 1.69-1.52 (m, 4H); 1.49-1.20 (m, 19H); 0.89 (br. t, 6H, J=6.0 Hz).

### Heptadecan-9-yl 8-bromooctanoate

### Synthesis of heptadecan-9-yl 8-bromooctanoate

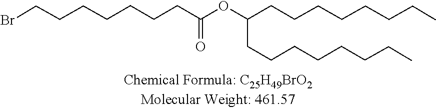 [SMILES: CCCCCCCCC(CCCCCCCC)OC(=O)CCCCCCCBr]

To a solution of heptadecan-9-ol, 8-bromooctanoic acid, and DMAP in methylene chloride was added EDC to afford heptadecan-9-yl 8-bromooctanoate.

### 3-Pentyloctyl 8-bromooctanoate

### Step 1: Synthesis of ethyl 3-pentyloct-2-enoate

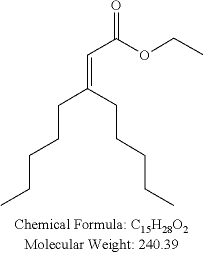 [SMILES: CCCCCC(=CC(=O)OCC)CCCCC]

Triethyl phosphonoacetate (10.6 mL, 53.4 mmol) was added dropwise over 20 minutes to a suspension of sodium hydride (2.13 g, 53.4 mmol) in THE (16 mL) and the mixture was stirred at room temperature until gas evolution ceased (approximately 30 min). The reaction mixture was chilled to 0° C. and 6-undecanone (8.42 mL, 41.1 mmol) was added in portions. The reaction was gradually warmed to room temperature and allowed to stir under reflux for 60 h. The reaction was cooled to room temperature prior to being quenched with saturated aqueous sodium bicarbonate. The aqueous phase was extracted with diethyl ether, and the organic extracts were washed with brine, dried (MgSO4), and concentrated. The crude material was purified by silica gel chromatography (0-20% EtOAc:hexanes) to afford ethyl 3-pentyloct-2-enoate (8.76 g, 36.5 mmol, 89%) as a clear oil. ¹H NMR (300 MHz, CDCl₃) δ: ppm 5.61 (s, 1H); 4.14 (q, 2H, J=6.0 Hz); 2.58 (ddd, 2H, J=9.0, 9.0, 6.0 Hz); 2.13 (ddd, 2H, J=6.0, 6.0, 3.0 Hz); 1.52-1.38 (m, 3H); 1.38-1.23 (m, 12H); 0.93-0.86 (m, 6H).

### Step 2: Synthesis of ethyl 3-pentyloctanoate

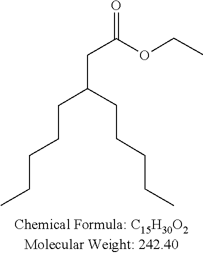 [SMILES: CCCCCC(CCCCC)CC(=O)OCC]

A steel Parr reactor equipped with a stir bar was charged with ethyl 3-pentyloct-2-enoate (8.76 g, 36.5 mmol) in ethanol (37 mL). Palladium hydroxide on carbon (768 mg, 5.47 mmol) was added and the vessel was sealed, evacuated, refilled with H₂ gas (3×), and the pressure was set to 200 psi. The reaction was stirred at 500 rpm, under 200 psi H₂ gas, at room temperature for 2 h. The vessel was then evacuated, refilled with N₂ gas, and opened. The crude reaction mixture was filtered through a Celite pad. The Celite pad was washed with EtOH and the crude material was concentrated to give ethyl 3-pentyloctanoate (8.45 g, 34.9 mmol, 96%) as a clear oil. The compound was carried onto the next step without further purification. ¹H NMR (300 MHz, CDCl₃) δ: ppm 4.12 (q, 2H, J=6.0 Hz); 2.22 (d, 2H, J=6.0 Hz); 1.92-1.77 (br. m, 1H); 1.37-1.19 (m, 19H); 0.88 (t, 6H, J=6.0 Hz).

### Step 3: Synthesis of 3-pentyloctan-1-ol

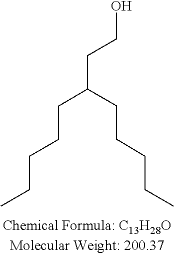

To a mixture of lithium aluminum hydride (1.59 g, 41.8 mmol) in dry ether (42 mL) under N₂ at 0° C., was added dropwise ethyl 3-pentyloctanoate (8.45 g, 34.9 mmol) in dry ether (28 mL). The mixture was stirred at room temperature for 2.5 h prior to being cooled to 0° C. Water (1 mL per g of LiAlH₄) was added to the solution dropwise, followed by the slow addition of 15% sodium hydroxide (1 mL per g of LiAlH₄) and water (3 mL per g of LiAlH₄). The solution was stirred for a few minutes at room temperature and filtered through a Celite pad. The Celite pad was washed with diethyl ether and the filtrate was concentrated. The crude material was purified by silica gel chromatography (0-40% EtOAc:hexanes) to afford 3-pentyloctan-1-ol (6.98 g, 34.9 mmol, 100%) as a clear oil. ¹H NMR (300 MHz, CDCl₃) δ: ppm 3.66 (t, 2H, J=6.0 Hz); 1.53 (q, 2H, J=6.0 Hz); 1.47-1.37 (br. s, 1H); 1.36-1.15 (m, 17H); 0.88 (t, 6H, J=6.0 Hz).

### Step 4: Synthesis of 3-Pentyloctyl 8-bromooctanoate

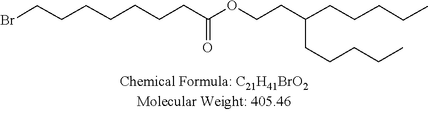 [SMILES: CCCCCC(CCCCC)CCOC(=O)CCCCCCCBr]

To a solution of 3-pentyloctan-1-ol (2.00 g, 9.98 mmol), 8-bromooctanoic acid (2.67 g, 12.0 mmol), and DMAP (244 mg, 2.00 mmol) in methylene chloride (18 mL) at 0° C. was added EDCI (2.87 g, 15.0 mmol) and the reaction mixture stirred at room temperature overnight. The reaction mixture was then cooled to 0° C. and a solution of 10% hydrochloric acid (70 mL) was added slowly over 20 minutes. The layers were separated, and the organic layer was concentrated in vacuum to give a crude oil. The oil was dissolved in hexane (70 mL) and washed with a mixture of acetonitrile (70 mL) and 5% sodium bicarbonate (70 mL). The hexane layer was separated, dried (MgSO4), and filtered. The solvent was removed under vacuum to give 3-pentyloctyl 8-bromooctanoate (3.94 g, 9.72 mmol, 97%) as a clear oil. The compound was carried onto the next step without further purification. ¹H NMR (300 MHz, CDCl₃) δ: ppm 4.08 (t, 2H, J=6.0 Hz); 3.40 (t, 2H, J=6.0 Hz); 3.29 (t, 2H, J=6.0 Hz); 1.85 (pent., 2H, J=6.0 Hz); 1.68-1.52 (m, 4H); 1.49-1.19 (m, 23H); 0.88 (t, 6H, J=6.0 Hz).

### 3-Pentyloctyl 8-((3-((tert-butoxycarbonyl)amino)propyl)amino)octanoate

### Synthesis of 3-Pentyloctyl 8-((3-((tert-butoxycarbonyl)amino)propyl)amino)octanoate

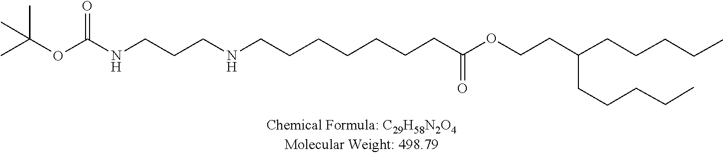 [SMILES: CCCCCC(CCCCC)CCOC(=O)CCCCCCCNCCCNC(=O)OC(C)(C)C]

To a solution of tert-butyl N-(3-aminopropyl)carbamate (15.5 g, 88.8 mmol) in EtOH (38 mL) was added 3-pentyloctyl 8-bromooctanoate (6.00 g, 14.8 mmol) in EtOH (36 mL) over the course of 20 min. The reaction was heated to 60° C. and allowed to stir at this temperature for 16 h. Upon cooling, the solvents were evaporated and the residue was diluted with ethyl acetate and washed with saturated aqueous NaHCO₃and brine (5×) until no white precipitate was observed in the aqueous layer. The organic layer was separated, washed with brine, dried (MgSO4), filtered, and concentrated. The residue was purified by flash chromatography (0-5-10-25-50-100% (mixture of 1% NH₄OH, 20% MeOH in dichloromethane) in dichloromethane) to give 3-pentyloctyl 8-((3-((tert-butoxycarbonyl)amino)propyl)amino)octanoate (4.23 g, 8.49 mmol, 57%) as a clear oil.

¹H NMR (300 MHz, CDCl₃) δ: ppm 5.17 (br. s, 1H); 4.07 (t, 2H, J=6.0 Hz); 3.19 (br. q, 2H, J=6.0 Hz); 2.66 (t, 2H, J=6.0 Hz); 2.56 (t, 2H, J=6.0 Hz); 2.28 (t, 2H, J=6.0 Hz); 1.70-1.52 (m, 6H); 1.51-1.39 (m, 3H); 1.44 (s, 9H); 1.36-1.19 (m, 22H); 0.88 (t, 6H, J=6.0 Hz).

### 3-Methoxy-4-(methylamino)cyclobut-3-ene-1,2-dione Synthesis of 3-methoxy-4-(methylamino)cyclobut-3-ene-1,2-dione

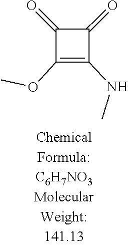

To a solution of 3,4-dimethoxy-3-cyclobutene-1,2-dione (1 g, 7 mmol) in 100 mL diethyl ether was added a 2M methylamine solution in THE (3.8 mL, 7.6 mmol) and a ppt. formed almost immediately. The mixture was stirred at room temperature for 24 hours, then filtered, the filter solids washed with diethyl ether and air-dried. The filter solids were dissolved in hot EtOAc, filtered, the filtrate allowed to cool to room temp., then cooled to 0° C. to give a ppt. This was isolated via filtration, washed with cold EtOAc, air-dried, then dried under vacuum to give 3-methoxy-4-(methylamino)cyclobut-3-ene-1,2-dione (0.70 g, 5 mmol, 73%) as a white solid.

¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Syntheses of Final Compounds

### Compound 22: 3-Butylheptyl 8-((8-(heptadecan-9-yloxy)-8-oxooctyl)(2-hydroxyethyl)amino)octanoate

### Step 1: Synthesis of heptadecan-9-yl 8-((2-hydroxyethyl)amino)octanoate

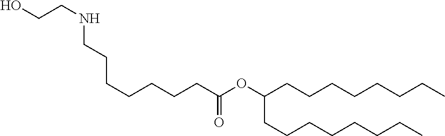 [SMILES: CCCCCCCCC(CCCCCCCC)OC(=O)CCCCCCCNCCO]

A solution of heptadecan-9-yl 8-bromooctanoate (10 g, 21.67 mmol) and ethanolamine (39.70 g, 649.96 mmol) in EtOH (5 mL) was heated to 65° C. for 16 h. The reaction was cooled to rt and dissolved in ethyl acetate and extracted with water (4×). The organic layer was separated, washed with brine, dried with Na₂SO₄, filtered and evaporated under vacuum. The residue was purified by flash chromatography (ISCO) by 0-100% (a solution of 20% MeOH, 80% DCM, 1% NH₄OH) in DCM to obtain heptadecan-9-yl 8-((2-hydroxyethyl)amino)octanoate (7.85 g, 82%).

UPLC/ELSD: RT=2.06 min. MS (ES): m/z (MH⁺) 442.689 for C₂₇H₅₅NO₃. ¹H NMR (300 MHz, CDCl₃) δ: ppm 4.89 (p, 1H); 3.66 (t, 2H); 2.79 (t, 2H); 2.63 (m, 2H); 2.30 (t, 2H); 1.77-1.20 (m, 40H); 0.90 (m, 6H).

### Step 2: Compound 22: Synthesis of 3-Butylheptyl 8-((8-(heptadecan-9-yloxy)-8-oxooctyl)(2-hydroxyethyl)amino)octanoate

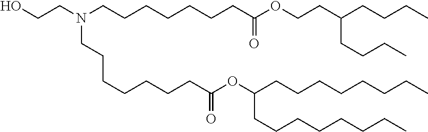 [SMILES: CCCCCCCCC(CCCCCCCC)OC(=O)CCCCCCCN(CCO)CCCCCCCC(=O)OCCC(CCCC)CCCC]

To a solution of 3-butylheptyl 8-bromooctanoate (6.15 g, 16.31 mmol) and heptadecan-9-yl 8-((2-hydroxyethyl)amino)octanoate (6.86 g, 15.53 mmol) in a mixture of CPME (15 mL) and acetonitrile (6 mL) was added potassium carbonate (8.59 g, 62.12 mmol) and potassium iodide (2.84 g, 17.08 mmol). The reaction was allowed to stir at 77° C. for 16 h. The reaction was cooled and filtered, and the volatiles were evaporated under vacuum. The residue was purified by flash chromatography (ISCO) by 0-100% (a solution of 20% MeOH, 80% DCM, 1% NH₄OH) in DCM to obtain 3-butylheptyl 8-((8-(heptadecan-9-yloxy)-8-oxooctyl)(2-hydroxyethyl)amino)octanoate (4.53 g, 37.8%).

UPLC/ELSD: RT=3.04 min. MS (ES): m/z (MH⁺) 739.464 for C₄₆H₉₁NO₅. ¹H NMR (300 MHz, CDCl₃) δ: ppm 4.89 (p, 1H); 4.11 (m, 2H), 3.57 (bm, 2H); 2.73-2.39 (m, 6H); 2.30 (m, 4H); 1.72-1.17 (m, 64H); 0.92 (m, 12H).

### Compound 27: 3-Butylheptyl 8-((8-(heptadecan-9-yloxy)-8-oxooctyl)(3-((2-(methylamino)-3,4-dioxocyclobut-1-en-1-yl)amino)propyl)amino)octanoate

### Step 1: Synthesis of Heptadecan-9-yl 8-((3-((tert-butoxycarbonyl)amino)propyl)amino)octanoate

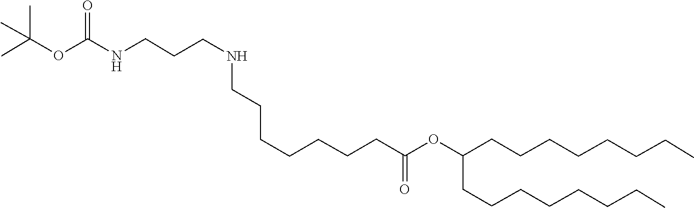 [SMILES: CCCCCCCCC(CCCCCCCC)OC(=O)CCCCCCCNCCCNC(=O)OC(C)(C)C]

A solution of tert-butyl N-(3-aminopropyl)carbamate (34.35 g, 197.15 mmol) in EtOH (200 mL) was heated to 65° C. and a solution of heptadecan-9-yl 8-bromooctanoate (26 g, 56.33 mmol) in EtOH (90 mL) was added over 3 h. The reaction was heated at 65° C. for 3 h. The reaction was cooled to <50° C. and EtOH was evaporated under vacuum and azeotroped with heptane (4×). To a solution of crude product in 2-MeTHF (150 mL) 5% K₂CO₃ (150 mL) was added and the resulting mixture was stirred for 10 minutes. The two layers were allowed to form. The aqueous layer was removed and the 2-MeTHF layer was washed with 100 mL water (×3). The organic layer was separated, washed with brine, dried with Na2SO₄, filtered and evaporated under vacuum. The residue was purified by flash chromatography (ISCO) by 0-100% (a solution of 20% MeOH, 80% DCM, 1% NH₄OH) in DCM to obtain heptadecan-9-yl 8-((3-((tert-butoxycarbonyl)amino)propyl)amino)octanoate (20 g, 63.9%). UPLC/ELSD: RT=2.34 min. MS (ES): m/z (MH⁺) 555.319 for C₃₃H₆₆N₂O₄. ¹H NMR (300 MHz, CDCl₃) δ: ppm 5.18 (bs, 1H); 4.89 (p, 1H); 3.22 (m, 2H); 2.64 (t, 2H); 2.59 (t, 2H); 2.30 (t, 2H); 1.73-1.21 (m, 50H); 0.90 (m, 6H).

### Step 2: Synthesis of 3-Butylheptyl 8-((3-((tert-butoxycarbonyl)amino)propyl)(8-(heptadecan-9-yloxy)-8-oxooctyl)amino)octanoate

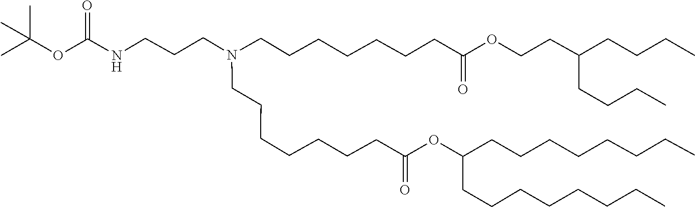 [SMILES: CCCCCCCCC(CCCCCCCC)OC(=O)CCCCCCCN(CCCCCCCC(=O)OCCC(CCCC)CCCC)CCCNC(=O)OC(C)(C)C]

To a solution of heptadecan-9-yl 8-((3-((tert-butoxycarbonyl)amino)propyl)amino)octanoate (11.76 g, 21.19 mmol) and 3-butylheptyl 8-bromooctanoate (9.2 g, 24.37 mmol) in propionitrile (52 mL) was added Potassium carbonate (4.39 g, 31.79 mmol) and Potassium iodide (0.53 g, 3.18 mmol). The reaction was heated at 80° C. for 16 h. The reaction was cooled and filtered, and the volatiles were evaporated under vacuum. The residue was purified by flash chromatography (ISCO) by 0-100% (a solution of 20% MeOH, 80% DCM, 1% NH₄OH) in DCM to obtain 3-butylheptyl 8-((3-((tert-butoxycarbonyl)amino)propyl)(8-(heptadecan-9-yloxy)-8-oxooctyl)amino)octanoate (9.68 g, 53.6%). UPLC/ELSD: RT=3.07 min. MS (ES): m/z (MH⁺) 851.216 for C₅₂H₁₀₂N₂₀₆. ¹H NMR (300 MHz, CDCl₃) δ: ppm 5.68 (bs, 1H); 4.90 (p, 1H); 4.11 (t, 2H); 3.20 (m, 2H); 2.52-2.24 (m, 10H); 1.76-1.20 (m, 74H); 0.90 (m, 12H).

### Step 3: Synthesis of 3-Butylheptyl 8-((3-aminopropyl)(8-(heptadecan-9-yloxy)-8-oxooctyl)amino)octanoate

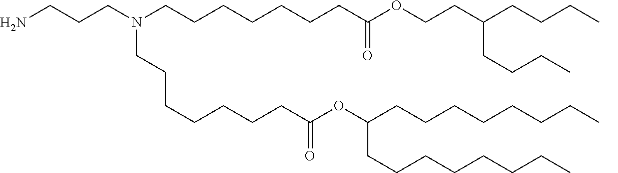 [SMILES: CCCCCCCCC(CCCCCCCC)OC(=O)CCCCCCCN(CCCN)CCCCCCCC(=O)OCCC(CCCC)CCCC]

To a solution of 3-butylheptyl 8-((3-((tert-butoxycarbonyl)amino)propyl)(8-(heptadecan-9-yloxy)-8-oxooctyl)amino)octanoate (7 g, 8.22 mmol) in DCM (25 mL) was added trifluoroacetic acid (9.4 mL, 123.32 mmol). The reaction was allowed to stir at rt for 2 h. The reaction was evaporated under vacuum. The residue was dissolved in mixture of methyl THF/heptane (1:9) and extracted with sat. sodium bicarbonate (3×). The organic layer was separated, washed with brine, dried with Na₂SO₄, filtered and evaporated under vacuum to obtain 3-Butylheptyl 8-((3-aminopropyl)(8-(heptadecan-9-yloxy)-8-oxooctyl)amino)octanoate. This was taken as a crude to the next step without further purification. UPLC/ELSD: RT=2.63 min. MS (ES): m/z (MH⁺) 751.305 for C₄₇H₉₄N₂O₄.

### Step 4: Compound 27. Synthesis of 3-Butylheptyl 8-((8-(heptadecan-9-yloxy)-8-oxooctyl)(3-((2-(methylamino)-3,4-dioxocyclobut-1-en-1-yl)amino)propyl)amino)octanoate

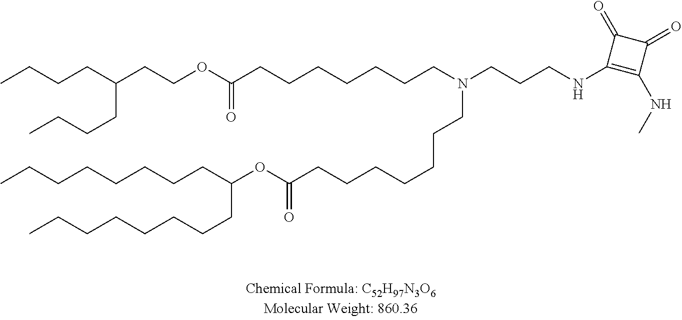 [SMILES: CCCCCCCCC(CCCCCCCC)OC(=O)CCCCCCCN(CCCCCCCC(=O)OCCC(CCCC)CCCC)CCCNc1c(NC)c(=O)c1=O]

To a solution of 3-butylheptyl 8-((3-aminopropyl)(8-(heptadecan-9-yloxy)-8-oxooctyl)amino)octanoate (7 g, 9.32 mmol) in methyl THE (31 mL) was added 3-methoxy-4-(methylamino)cyclobut-3-ene-1,2-dione (1.71 g, 12.11 mmol), and a aqueous solution of 10% Sodium bicarbonate (8.6 mL, 10.25 mmol). The reaction was allowed to stir at 50° C. for 2.5 h. The reaction was cooled to rt and diluted with heptane and extracted with water. The organic layer was separated, washed with brine, dried with Na2SO₄, filtered and evaporated under vacuum. The residue was purified by flash chromatography (ISCO) by 0-100% (a solution of 20% MeOH, 80% DCM, 1% NH₄OH) in DCM to obtain 3-Butylheptyl 8-((8-(heptadecan-9-yloxy)-8-oxooctyl)(3-((2-(methylamino)-3,4-dioxocyclobut-1-en-1-yl)amino)propyl)amino)octanoate (5.4 g, 63%). UPLC/ELSD: RT=2.98 min. MS (ES): m/z (MH⁺) 861.714 for C₅₂H₉₇N₃O₆. ¹H NMR (300 MHz, CDCl₃) δ: ppm 4.89 (p, 1H); 4.10 (t, 2H); 3.75 (m, 2H); 3.39-3.20 (m, 5H); 3.08 (m, 4H); 2.31 (m, 4H); 2.12 (bm, 2H); 1.81-1.20 (m, 65H); 0.90 (m, 12H).

### Compound 30: Bis(3-pentyloctyl) 8,8′-((3-((2-(methylamino)-3,4-dioxocyclobut-1-en-1-yl)amino)propyl)azanediyl)dioctanoate

### Step 1: Synthesis of Bis(3-pentyloctyl) 8,8′-((3-((tert-butoxycarbonyl)amino)propyl) azanediyl)dioctanoate

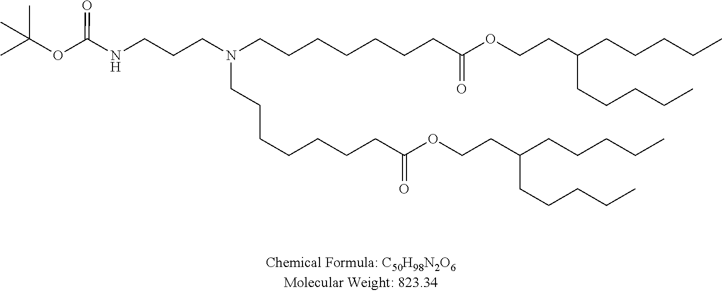 [SMILES: CCCCCC(CCCCC)CCOC(=O)CCCCCCCN(CCCCCCCC(=O)OCCC(CCCCC)CCCCC)CCCNC(=O)OC(C)(C)C]

To a solution of 3-pentyloctyl 8-bromooctanoate (5.61 g, 13.8 mmol) and 3-pentyloctyl 8-((3-((tert-butoxycarbonyl)amino)propyl)amino)octanoate (6.00 g, 12.0 mmol) in propionitrile (30 mL) was added potassium carbonate (2.49 g, 18.0 mmol) and iodopotassium (300 mg, 1.80 mmol). The reaction was allowed to stir at 80° C. for 16 h. Upon cooling to room temperature, the reaction mixture was filtered via vacuum filtration. The residue in the vessel and the filter cake on the funnel was washed twice with propionitrile. The filtrate was then concentrated in vacuo at 40° C. The crude residue was purified by silica gel chromatography (0-5-10-20-25-30-35-40-50-80-100% (mixture of 1% NH₄OH, 20% MeOH in dichloromethane) in dichloromethane) to give bis(3-pentyloctyl) 8,8′-((3-((tert-butoxycarbonyl)amino)propyl) azanediyl)dioctanoate (7.37 g, 8.95 mmol, 74%) as a light yellow transparent oil. ¹H NMR (300 MHz, CDCl₃) δ: ppm 5.66 (br. s, 1H); 4.08 (t, 4H, J=6.0 Hz); 3.17 (br. q, 2H, J=6.0 Hz); 2.43 (t, 2H, J=6.0 Hz); 2.34 (br. t, 4H, J=6.0 Hz); 2.28 (t, 4H, J=9.0 Hz); 1.67-1.52 (m, 10H); 1.48-1.37 (m, 14H); 1.35-1.17 (m, 45H); 0.88 (t, 12H, J=6.0 Hz).

### Step 2: Synthesis of Bis(3-pentyloctyl) 8,8′-((3-aminopropyl)azanediyl)dioctanoate

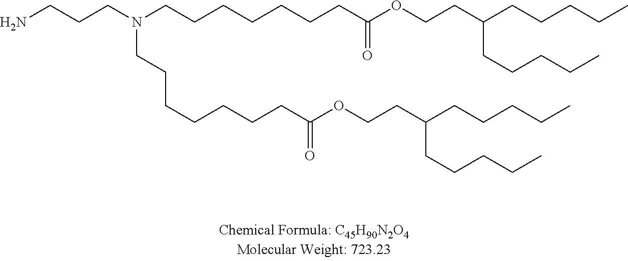 [SMILES: CCCCCC(CCCCC)CCOC(=O)CCCCCCCN(CCCN)CCCCCCCC(=O)OCCC(CCCCC)CCCCC]

To a round bottom flask equipped with a stir bar was added bis(3-pentyloctyl) 8,8′-((3-((tert-butoxycarbonyl)amino)propyl) azanediyl)dioctanoate (3.00 g, 3.64 mmol). The oil was dissolved in cyclopentyl methyl ether (8 mL) and stirred for 5 minutes. 3M HCl in cyclopentyl methyl ether (6.07 mL, 18.2 mmol) was added dropwise. After addition was complete, the reaction was heated to 40° C. for 1 hour and reaction completion was monitored by TLC/LCMS analysis. The reaction was cooled to room temperature, and then chilled to 0° C. 10% K₂CO₃ solution was then added dropwise to the reaction mixture. After addition was complete, the aqueous/cyclopentyl methyl ether emulsion was diluted with EtOAc and the resulting mixture stirred for 10 minutes. The solution was transferred to a separation funnel and the layers were separated. The organic layer was dried (MgSO4), filtered, and concentrated. The residue was redissolved in heptane and washed twice with MeCN. The heptane layer was dried (MgSO4), filtered, and concentrated to afford crude bis(3-pentyloctyl) 8,8′-((3-aminopropyl)azanediyl)dioctanoate (2.43 g, 3.36 mmol, 92%) as an off-white oil. The crude material was carried onto the next step without further purification. ¹H NMR (300 MHz, CDCl₃) δ: ppm 4.08 (t, 4H, J=6.0 Hz); 2.98 (t, 2H, J=6.0 Hz); 2.71 (t, 2H, J=6.0 Hz); 2.54 (br. t, 4H, J=6.0 Hz); 2.28 (t, 6H, J=6.0 Hz); 1.76 (br. pentet, 2H, J=2.0 Hz); 1.66-1.52 (m, 9H); 1.52-1.43 (m, 4H); 1.37-1.18 (m, 45H); 0.88 (t, 12H, J=6.0 Hz).

### Step 3: Compound 30: Synthesis of Bis(3-pentyloctyl) 8,8′-((3-((2-(methylamino)-3,4-dioxocyclobut-1-en-1-yl)amino)propyl)azanediyl)dioctanoate

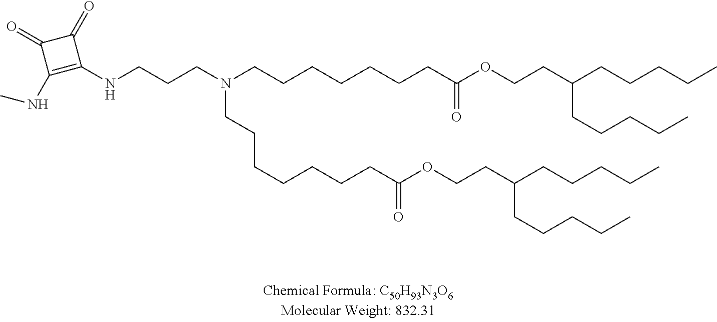 [SMILES: CCCCCC(CCCCC)CCOC(=O)CCCCCCCN(CCCCCCCC(=O)OCCC(CCCCC)CCCCC)CCCNc1c(NC)c(=O)c1=O]

To a round bottom flask equipped with a stir bar was added bis(3-pentyloctyl) 8,8′-((3-aminopropyl)azanediyl)dioctanoate (2.43 g, 3.36 mmol), 3-methoxy-4-(methylamino)cyclobut-3-ene-1,2-dione (616 mg, 4.36 mmol) and 2-Methyl THF (10 mL). 10% K₂CO₃ solution (10 mL) was added and the resulting biphasic mixture was heated to 45° C. and stirred vigorously for 3 hours. Reaction completion was monitored by TLC/LCMS analysis. Upon completion the mixture was allowed to cool to room temperature. The reaction was diluted with water, layers were separated, and the aqueous layer was extracted twice with heptane. The organics were combined, washed with water (3×), brine, and with a 1:1 acetonitrile/water mixture. The combined organics were then dried (Na₂SO₄), filtered, and concentrated. The crude residue was azeotroped and concentrated with DCM and MeOH three times to yield a pale yellow crude waxy oil. The crude residue was purified by silica gel chromatography (0-100% (mixture of 1% NH₄OH, 20% MeOH in dichloromethane) in dichloromethane) to give bis(3-pentyloctyl) 8,8′-((3-((2-(methylamino)-3,4-dioxocyclobut-1-en-1-yl)amino)propyl)azanediyl)dioctanoate (2.11 g, 2.54 mmol, 76%) as a white waxy solid.

UPLC/ELSD: RT=2.79 min. MS (ES): m/z (MH⁺) 832.34 for C₅₀H₉₃N₃O₆. ¹H NMR (300 MHz, CDCl₃) δ: ppm 7.83 (br. s, 1H); 7.61 (br. s, 1H); 4.03 (t, 4H, J=9.0 Hz); 3.64 (br. s, 2H); 3.28 (br. d, 3H, J=6.0 Hz); 2.46 (t, 2H, J=9.0 Hz); 2.33 (br. t, 4H, J=6.0 Hz); 2.33 (t, 4H, J=9.0 Hz); 1.74 (br. pentet, 2H, J=6.0 Hz); 1.62-1.47 (m, 8H); 1.41-1.12 (m, 50H); 0.83 (t, 12H, J=9.0 Hz).

### Compound 54: 3-Butylheptyl 8-((3-((2-(methylamino)-3,4-dioxocyclobut-1-en-1-yl)amino)propyl)(8-oxo-8-((3-pentyloctyl)oxy)octyl)amino)octanoate

### Step 1: Synthesis of 3-butylheptyl 8-((3-((tert-butoxycarbonyl)amino)propyl)(8-oxo-8-((3-pentyloctyl)oxy)octyl)amino)octanoate

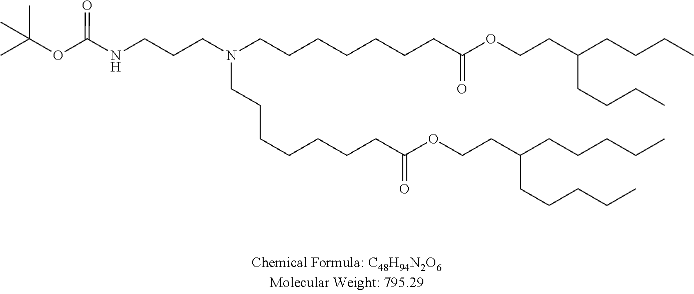 [SMILES: CCCCCC(CCCCC)CCOC(=O)CCCCCCCN(CCCCCCCC(=O)OCCC(CCCC)CCCC)CCCNC(=O)OC(C)(C)C]

To a solution of 3-butylheptyl 8-bromooctanoate (794 mg, 2.11 mmol) and 3-pentyloctyl 8-((3-((tert-butoxycarbonyl)amino)propyl)amino)octanoate (1.00 g, 2.01 mmol) in cyclopentyl methyl ether (9 mL) and actonitrile (9 mL) was added potassium carbonate (1.66 g, 12.0 mmol) and iodopotassium (366 mg, 2.21 mmol). The reaction was allowed to stir at 80° C. for 16 h. Upon cooling, the volatiles were evaporated under vacuum. The residue was diluted with dichloromethane and washed with water. The organic layer was separated, washed with brine, dried (MgSO4), filtered, and concentrated. The crude residue was purified by silica gel chromatography (0-5-10-25-50-100% (mixture of 1% NH₄OH, 20% MeOH in dichloromethane) in dichloromethane) to give 3-butylheptyl 8-((3-((tert-butoxycarbonyl)amino)propyl)(8-oxo-8-((3-pentyloctyl)oxy)octyl)amino)octanoate (896 mg, 1.13 mmol, 56%) as a clear oil. UPLC/ELSD: RT=2.95 min. MS (ES): m/z (MH⁺) 795.59 for C₄₈H₉₄N₂O₆.

### Step 2: Synthesis of 3-butylheptyl 8-((3-aminopropyl)(8-oxo-8-((3-pentyloctyl)oxy)octyl)amino)octanoate

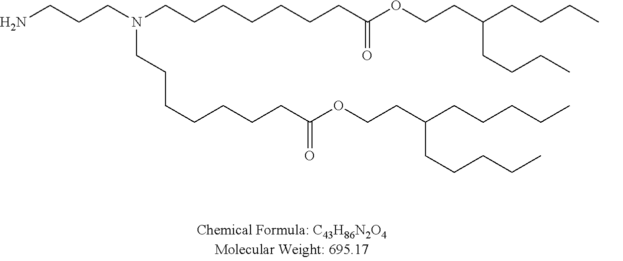 [SMILES: CCCCCC(CCCCC)CCOC(=O)CCCCCCCN(CCCN)CCCCCCCC(=O)OCCC(CCCC)CCCC]

To a solution of 3-butylheptyl 8-((3-((tert-butoxycarbonyl)amino)propyl)(8-oxo-8-((3-pentyloctyl)oxy)octyl)amino)octanoate (896 mg, 1.13 mmol) in methylene chloride (23 mL) was added trifluoroacetic acid (1.72 mL, 22.5 mmol). The reaction was allowed to stir at room temperature for 4 h. The reaction was quenched with saturated aqueous NaHCO₃and extracted with dichloromethane. The organic layer was separated, washed with brine, dried (MgSO4), filtered and concentrated. The crude material was purified by silica gel chromatography (0-5-10-25-50-100% (mixture of 1% NH₄OH, 20% MeOH in dichloromethane) in dichloromethane) to give 3-butylheptyl 8-((3-aminopropyl)(8-oxo-8-((3-pentyloctyl)oxy)octyl)amino)octanoate (632 mg, 0.91 mmol, 81%) as a clear oil.

UPLC/ELSD: RT=2.47 min. MS (ES): m/z (MH⁺) 695.68 for C₄₃H₈₆N₂O₄.

### Step 3: Compound 54: Synthesis of 3-butylheptyl 8-((3-((2-(methylamino)-3,4-dioxocyclobut-1-en-1-yl)amino)propyl)(8-oxo-8-((3-pentyloctyl)oxy)octyl)amino)octanoate

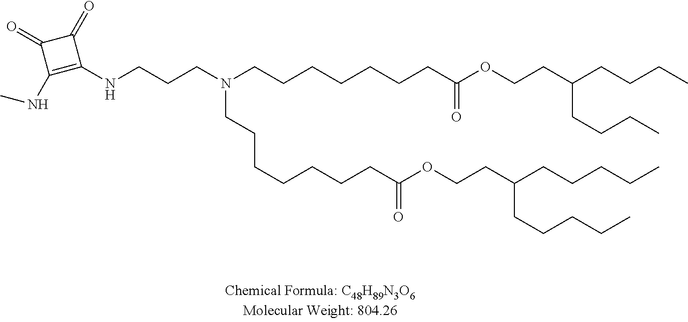 [SMILES: CCCCCC(CCCCC)CCOC(=O)CCCCCCCN(CCCCCCCC(=O)OCCC(CCCC)CCCC)CCCNc1c(NC)c(=O)c1=O]

To a solution of 3-butylheptyl 8-((3-aminopropyl)(8-oxo-8-((3-pentyloctyl)oxy)octyl)amino)octanoate (632 mg, 0.91 mmol) in ethanol (8 mL) was added 3-methoxy-4-(methylamino)cyclobut-3-ene-1,2-dione (192 mg, 1.36 mmol). The reaction was allowed to stir at 67° C. for 20 h. After 20 h, the reaction was cooled to room temperature and diluted with diethyl ether. The organics were washed with brine, dried (MgSO4), filtered, and concentrated. The crude residue was purified by silica gel chromatography (0-5-10-25-50-100% (mixture of 1% NH₄OH, 20% MeOH in dichloromethane) in dichloromethane) to give 3-butylheptyl 8-((3-((2-(methylamino)-3,4-dioxocyclobut-1-en-1-yl)amino)propyl)(8-oxo-8-((3-pentyloctyl)oxy)octyl)amino)octanoate (240 mg, 0.30 mmol, 33%) as an off-white waxy solid. UPLC/ELSD: RT=2.67 min. MS (ES): m/z (MH⁺) 804.22 for C₄₈H₈₉N₃O₆. ¹H NMR (300 MHz, CDCl₃) δ: ppm 7.38 (br. s, 1H); 7.03 (br. s, 1H); 4.07 (t, 4H, J=6.0 Hz); 3.65 (br. s, 2H, J=6.0 Hz); 3.27 (d, 3H, J=6.0 Hz); 2.52 (br. t, 2H, J=6.0 Hz); 2.40 (br. t, 4H, J=6.0 Hz); 2.28 (t, 4H, J=6.0 Hz); 1.75 (br. pent., 2H, J=6.0 Hz); 1.67-1.51 (m, 8H); 1.47-1.17 (m, 46H); 0.93-0.82 (m, 12H).

### Example 4: Expression of hEPO Induced by Sample Formulations in Mice and Residual Lipid Levels in the Liver

To assess potency of expression and metabolic stability of lipids of the disclosure, hepatocyte protein expression (hEPO) was measured following administration to mice of a nanoparticle of the disclosure.

Lipid nanoparticles including DSPC as a phospholipid, cholesterol as a structural lipid, Compound I as a PEG lipid, an ionizable amino lipid of Formula (II), and an mRNA encoding hEPO were intravenously administered to CD-1 mice. The concentration of hEPO in serum was tested at 6 hours after injection. The particles tested had a PDI of between about 0.1-0.4, an encapsulation efficiency of between about 82-99%, and a particle diameter of about 56-145 nm. All of the tested lipid nanoparticles demonstrated effective delivery of mRNA to hepatocytes with varying amounts of lipid remaining in the livers of the mice after 24 hours (Table 10).

TABLE 10
Expression of hEPO in mice induced by administration
of lipid nanoparticles comprising an ionizable
amino lipid of Formula (II)

| | | | | Approx. |
|---|---|---|---|---|
| | | | Parent | Percent |
| | | | Lipid | Total |
| Ionizable | Mean 6 hr | | Remaining | Dose of |
| Amino | hEPO | | (nmol/g | Parent |
| Lipid | Concen- | | liver | Lipid |
| Compound | tration | hEPO | tissue) | Remaining |
| No. | (mIU/mL) | AUC | at 24 h | at 24 h |
| 7 | 1355957 | 2.13E+07 | 0.3 ± 0.2 | 0.2% |
| 14 | 2952339 | 4.53E+07 | 56 ± 6 | 58% |
| 15 | 2933644 | 4.31E+07 | 51 ± 4 | 53% |
| 16 | 1461176 | 2.18E+07 | 77 ± 3 | 79% |
| 17 | 1777875 | 2.61E+07 | 35 ± 5 | 36% |
| 18 | 2158772 | 3.17E+07 | 40 ± 7 | 41% |
| 19 | 2089101 | 3.11E+07 | 73 ± 6 | 75% |
| 20 | 1414856 | 2.13E+07 | 65 ± 5 | 67% |
| 27 | 1316011 | 2.10E+07 | 27 ± 4 | 28% |
| 29 | 808734 | 1.23E+07 | 66 ± 16 | 68% |
| 26 | 496951 | 7.52E+06 | 141 ± 15 | >95%^{b} |
| 30 | 1592050 | 2.40E+07 | 25 ± 4 | 26% |
| 28 | 938332 | 1.44E+07 | 58 ± 7 | 60% |
| 40 | 303957 | 4.64E+06 | 3 ± 0.3 | 3% |
| 39 | 129750 | 1.94E+06 | 4 ± 1 | 4% |
| 22 | 1264530 | 2.03E+07 | <0.1 | <0.1% |
| 24 | 1160499 | 1.93E+07 | 3 ± 1 | 2% |
| 21 | 458772 | 7.29E+06 | 13 ± 2 | 9% |
| 25 | 591942 | 1.07E+07 | 0.1 0.04 | <0.1% |
| 23 | 847586 | 1.40E+07 | 24 ± 3 | 18% |
| 37 | 85052 | 1.25E+06 | <0.1 | <0.1% |
| 35 | 106485 | 1.72E+06 | <0.1 | <0.1% |
| 31 | 517841 | 7.72E+06 | 2 ± 0.6 | <0.1% |
| 33 | 389395 | 5.89E+06 | 0.5 ± 0.3 | <0.1% |
| 32 | 111656 | 1.63E+06 | 69 ± 17 | 71% |
| 34 | 205437 | 3.01E+06 | 12 ± 2 | 12% |
| 51 | 1556494 | 1.31E+07 | 4 ± 0.6 | 4% |
| 52 | 564670 | 3.82E+06 | <1 | <1% |
| 53 | 130324 | 6.63E+03 | <1 | <1% |
| 54 | 647775 | 1.97E+07 | 12 ± 1 | 12% |
| 55 | 1332073 | 1.99E+07 | 3 ± 0.6 | 3% |
| 57 | 1238977 | 1.60E+07 | 12 ± 1 | 13% |
| 58 | 1347883 | 2.33E+07 | 15 ± 2 | 15% |
| 60 | 1031398 | 1.42E+07 | 8.5 ± 0.6 | 9 ± 0.8% |
| 61 | 1020791 | 1.44E+07 | 5.1 ± 0.8 | 6 ± 0.3% |
| 62 | 475498 | 6.51E+06 | 1.8 ± 0.6 | 2 ± 0.9% |
| 63 | 692334 | 9.66E+06 | 8.9 ± 2.8 | 9 ± 3% |
| 64 | 1760015 | 2.43E+07 | 4.9 ± 0.9 | 6 ± 0.8% |
| 65 | 734054 | 1.02E+07 | 0.9 ± 0.3 | 0.9 ± 0.06% |
| 66 | 17801 | 2.10E+05 | 0.1 ± 0.03 | <1% |
| 42 | 1428145 | | | |
| 44 | 1273871 | | | |
| 49 | 2385524 | | | |
| 50 | 2617164 | | | |
| 67 | 969620 | | | |
| 68 | 488014 | | | |
| 69 | 1572094 | | | |
| 70 | 1479577 | | | |
| 71 | 1921281 | | | |

^{a}The percent of total dose calculation assumes a 25 g mouse with a 1.5 g liver
^{b}>95% equals very slow metabolism of the lipid

### Example 5: Expression of Luciferase Induced by Sample Formulations in Mice and Rats

To further assess potency of expression and metabolic stability of lipids of the disclosure, expression of luciferase was measured following administration to rodents of a nanoparticle of the disclosure comprising an mRNA containing luciferase.

Lipid nanoparticles including DSPC as a phospholipid, cholesterol as a structural lipid, Compound I as a PEG lipid, an ionizable amino lipid of Formula (II), and an mRNA encoding luciferase were intravenously administered to CD-1 mice and Sprague Dawley rats. The level of expression was tested via bioluminescence in the liver ex vivo at 6 hours after injection. The particles tested had a PDI of between about 0.09-0.25 and an encapsulation efficiency of between about 89-98%. All of the tested lipid nanoparticles demonstrated effective delivery of mRNA to the liver and mRNA expression in hepatocytes with varying amounts of lipid remaining in the liver tissue of the rats after 24 hours (Table 11A and Table 11B).

TABLE 11A
Expression of NPI-Luciferase in mice and rats induced
by administration of lipid nanoparticles comprising
an ionizable amino lipid of Formula (II)

| | Mean 6 h | | |
|---|---|---|---|
| | ex vivo | Approx. | |
| | Liver NPI- | Percent | |
| Ionizable | Luciferase | Total Dose | |
| Amino | Total | of Parent | |
| Lipid | Flux | Lipid | |
| Compound | (photons/ | Remaining | Test |
| No. | second) | at 24 h | Animal |
| 27 | 357720000 | | mouse |
| 30 | 299800000 | | mouse |
| 22 | 300960000 | | mouse |
| 24 | 240400000 | | mouse |
| 18 | 372800000 | | mouse |
| 25 | 336800000 | | mouse |
| 14 | 465200000 | | mouse |
| 27 | 1215666667 | 28% | rat |
| 30 | 963633333.3 | 25% | rat |
| 22 | 2227666667 | <0.1% | rat |
| 24 | 2020000000 | 2% | rat |
| 18 | 1473333333 | 41% | rat |
| 25 | 4068000000 | <0.1% | rat |
| 14 | 1567000000 | 58% | rat |

TABLE 11B
Expression of luciferase in mice and residual lipid levels
in the liver induced by administration of lipid nanoparticles
comprising an ionizable amino lipid of Formula (II)

| | Total | |
|---|---|---|
| | Whole Body | % Lipid |
| Compound | Flux @ 6 h | Remaining |
| No. | (p/s) | @ 24 h |
| 7 | 6.09E+10 | <1 |
| 8 | 3.82E+10 | <1 |
| 12 | 2.39E+10 | <1 |
| 13 | 3.77E+10 | <1 |
| 9 | 1.82E+10 | 68 ± 10 |
| 10 | 4.6E+09 | 67 ± 7 |
| 11 | 1.04E+10 | 23 ± 6 |

### Example 6: Tolerability in Rats

To assess tolerability of the lipids of the disclosure, expression of a reporter antibody construct induced by sample formulations was assessed in rats.

Lipid nanoparticles including DSPC as a phospholipid, cholesterol as a structural lipid, Compound I as a PEG lipid, an ionizable amino lipid of Formula (II), and an mRNA encoding a reporter antibody construct were intravenously administered to Sprague Dawley rats. The level of reporter antibody construct was tested via an Enzyme-linked Immunosorbent Assay (ELISA) at 6 hours after injection. For the formulations tested, high levels of the reporter antibody were measured with minimal signs of liver toxicity (Table 12).

TABLE 12
Expression of reporter antibody construct in rats
induced by administration of lipid nanoparticles
comprising an ionizable amino lipid of Formula (II)

| | Mean 24 h |
|---|---|
| Ionizable | Reporter |
| Amino | Antibody |
| Lipid | Construct |
| Compound | Concentration |
| No. | (ng/mL) |
| 27 | 833698 |
| 22 | 1113642 |
| 25 | 948617 |

### Example 7: Expression of hEPO Induced by Sample Formulations in Rats

To further assess potency of expression and metabolic stability of lipids of the disclosure, hepatocyte protein expression (hEPO) was measured following administration to rats of a nanoparticle of the disclosure.

Lipid nanoparticles including DSPC as a phospholipid, cholesterol as a structural lipid, Compound I as a PEG lipid, an ionizable amino lipid of Formula (II), and an mRNA encoding hEPO were intravenously administered to Sprague Dawley rats. The concentration of hEPO in serum was tested at 6 hours after injection. All of the tested lipid nanoparticles demonstrated effective delivery of mRNA to hepatocytes with varying amounts of lipid remaining in the livers of the rats after 6 hours (Table 13).

TABLE 13
Expression of hEPO in rats induced by administration
of lipid nanoparticles comprising an ionizable
amino lipid of Formula (II)

| Ionizable | |
|---|---|
| Amino | Mean 6 hr |
| Lipid | hEPO |
| Compound | Concentration |
| No. | (mIU/mL) |
| 64 | 161146 |
| 30 | 688780 |
| 54 | 476742 |
| 27 | 664431 |
| 61 | 362569 |
| 55 | 161760 |

### Example 8: Expression of Luciferase Induced by Sample Formulations in Non-Human Primates

To the assess potency of expression and metabolic stability of lipids of the disclosure in different species, the expression of an mRNA was measured following administration to non-human primates of a nanoparticle of the disclosure containing the mRNA.

Lipid nanoparticles including DSPC as a phospholipid, cholesterol as a structural lipid, Compound I as a PEG lipid, an ionizable amino lipid of Formula (II), and an mRNA encoding luciferase were intravenously administered to cynomolgus monkeys. The level of luciferase in livers ex vivo was measured via ELISA at 6 hours after injection. All of the tested lipid nanoparticles demonstrated effective delivery of mRNA to hepatocytes of non-human primates (Table 14).

TABLE 14
Expression of luciferase in non-human primates induced
by administration of lipid nanoparticles comprising
an ionizable amino lipid of Formula (II)

| Ionizable | |
|---|---|
| Amino | Mean 6 h |
| Lipid | NPI-Luciferase |
| Compound | Concentration |
| No. | (ng/g) |
| 27 | 878 |
| 30 | 782 |
| 54 | 693 |
| 22 | 1322 |

### Example 9: Lipid Nanoparticle Formulations for Expressing mRNA Encoding a Urea Cycle Enzyme

The effect of varying lipid nanoparticle formulations on expression and/or activity of a liver-expressed urea cycle enzyme (“UCE”) was tested. Table 15 lists the ionizable amino lipid and PEG-lipid used in each tested lipid nanoparticle formulation.

TABLE 15
Lipid Nanoparticle Formulation Components

| LNP | Ionizable amino Lipid |
|---|---|
| 1 | 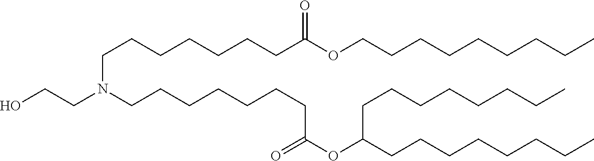 [SMILES: CCCCCCCCCOC(=O)CCCCCCCN(CCO)CCCCCCCC(=O)OC(CCCCCCCC)CCCCCCCC] |
| 2 | 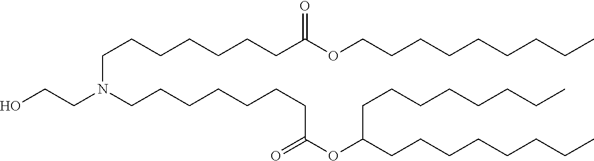 [SMILES: CCCCCCCCCOC(=O)CCCCCCCN(CCO)CCCCCCCC(=O)OC(CCCCCCCC)CCCCCCCC] |
| 3 | 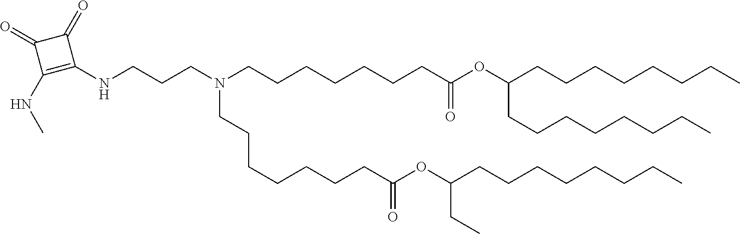 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| 4 | |
| 5 | 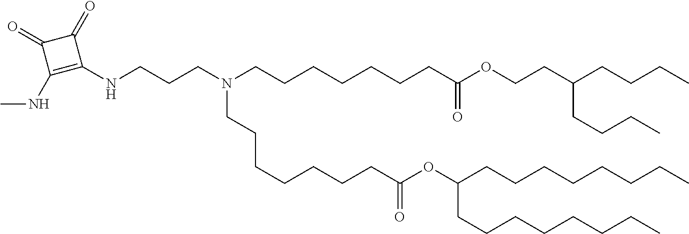 [SMILES: CCCCCCCCC(CCCCCCCC)OC(=O)CCCCCCCN(CCCCCCCC(=O)OCCC(CCCC)CCCC)CCCNc1c(NC)c(=O)c1=O] |
| 6 | 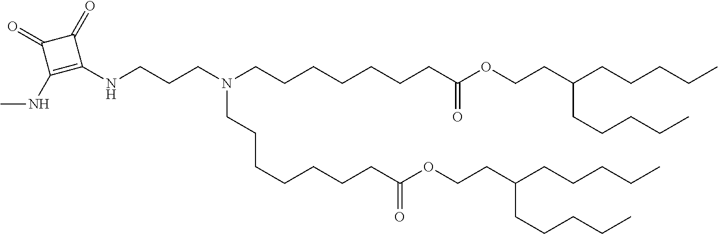 [SMILES: CCCCCC(CCCCC)CCOC(=O)CCCCCCCN(CCCCCCCC(=O)OCCC(CCCCC)CCCCC)CCCNc1c(NC)c(=O)c1=O] |
| 7 | 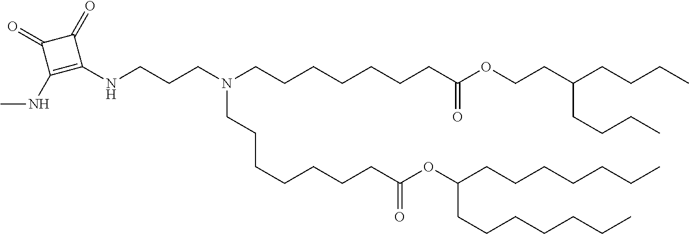 [SMILES: CCCCCCCC(CCCCCCC)OC(=O)CCCCCCCN(CCCCCCCC(=O)OCCC(CCCC)CCCC)CCCNc1c(NC)c(=O)c1=O] |
| LNP | PEG-Lipid |
| 1 | 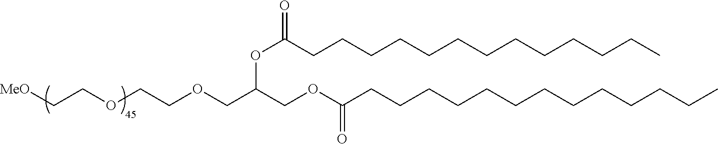 |
| 2 | 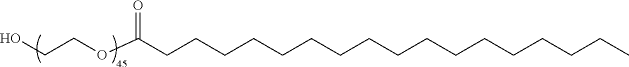 |
| 3 | 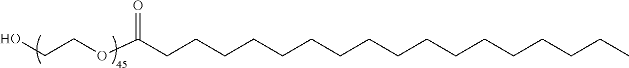 |
| 4 | 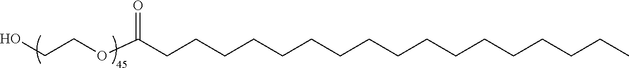 |
| 5 | 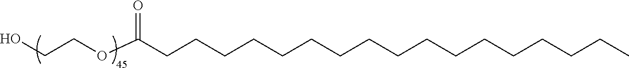 |
| 6 | 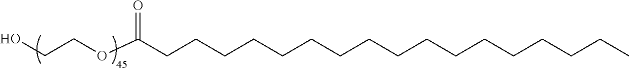 |
| 7 | 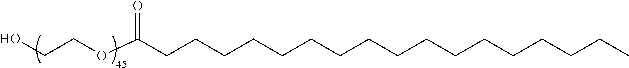 |

UCE expression and activity in hepatocytes were assessed in mouse and non-human primate (NHP) models after dosing with LNPs 1-4 described in Table 15. To determine expression in hepatocytes, mice and NHPs were dosed with LNPs 1-4 (0.5 mg/kg of UCE mRNA for mice; 2 mg/kg of UCE mRNA for NHPs) and the percentage hepatocyte expression was estimated from immunohistochemistry staining images (for LNP-1) or by using luciferase-mRNA loaded LNPs (for LNPs 2-4). To determine the increased percentage of wild-type mouse UCE activity, a linear relationship between % hepatocyte expression and activity was assumed, and an experimentally determined 20% increase in activity for LNP-1 was taken as proportionality when nearly all hepatocytes were expressing mRNA. For NHPs, the increased percentage of wild-type mouse UCE activity was extrapolated from the percentage of hepatocyte expression. Table 16 provides the results.

TABLE 16

| | | | Increased % wild-type | | |
|---|---|---|---|---|---|
| | % Hepatocyte expression | | mouse UCE activity | | |
| | Mouse | NHP | Mouse | NHP | NHP |
| | (0.5 | (2 | (0.5 | (2 | (0.35 |
| | mg/kg) | mg/kg) | mg/kg) | mg/kg) | mg/kg) |
| LNP-1 | >90% | 2% | 20% | 0.4% | 0.1% |
| LNP-2 | 25% | 8% | 5% | 1.6% | 0.4% |
| LNP-3 | 60% | 25% | 20% | 8% | 2% |
| LNP-4 | 39% | 47% | 20% | 24% | 4.2% |

To test the in vivo effect of the lipid nanoparticle formulations on UCE expression and activity, a mouse model for UCE deficiency was dosed with 0.5 mg/kg of one of LNP-2-LNP-7 encapsulating the UCE-encoding mRNA. PBS was used as a control. Two days post-dosing, livers were harvested and analyzed for UCE expression and activity.

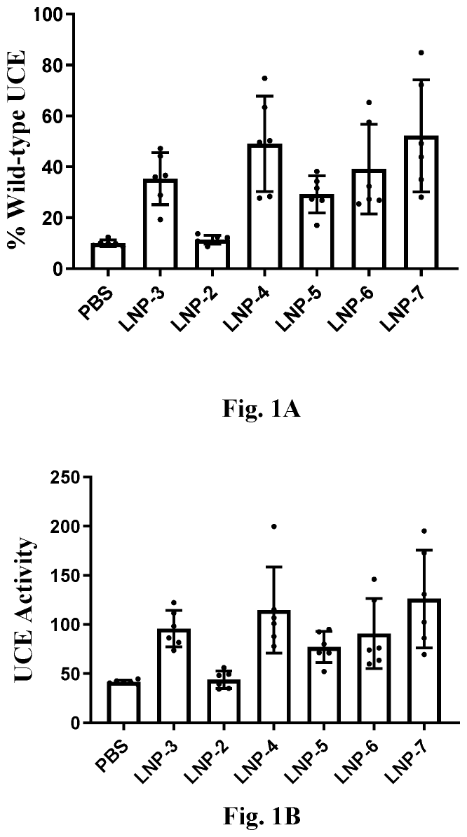

FIG. 1A is a graph showing the amount of UCE protein detected in liver samples at the indicated days post-dosing with the lipid nanoparticles or PBS. UCE expression at day 2 was significantly improved in mice administered LNP-1, -4, -5, -6, and -7.

FIG. 1B is a graph showing total hepatocyte UCE activity in liver samples as a function of time at the indicated days post-dosing with the lipid nanoparticles or PBS.

FIG. 1C is a graph showing the UCE immunohistochemistry H-score 2 days post-dosing with the lipid nanoparticles or PBS.

### Example 10: Lipid Nanoparticle Formulations for Expressing mRNA Encoding a Gluconeogenesis Enzyme

The effect of varying lipid nanoparticle formulations on expression and/or activity of a liver-expressed gluconeogenesis enzyme (“GE”) was tested. Table 17 lists the ionizable amino lipid and PEG-lipid used in each tested lipid nanoparticle formulation.

TABLE 17
Lipid Nanoparticle Formulations

| LNP | Ionizable Amino Lipid | PEG-Lipid |
|---|---|---|
| LNP-1 | 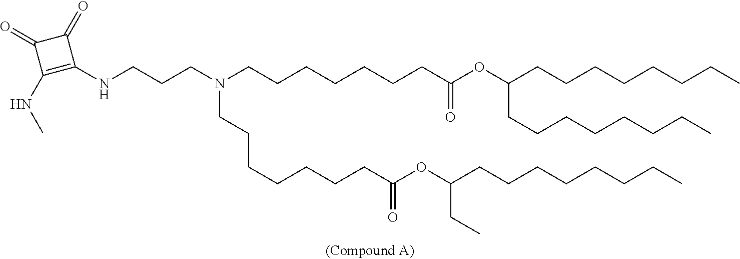 [SMILES: CCCCCCCCC(CC)OC(=O)CCCCCCCN(CCCCCCCC(=O)OC(CCCCCCCC)CCCCCCCC)CCCNc1c(NC)c(=O)c1=O] | Compound I |
| LNP-2 | 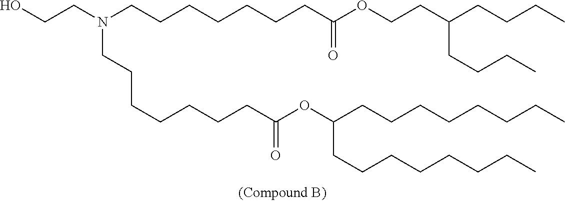 [SMILES: CCCCCCCCC(CCCCCCCC)OC(=O)CCCCCCCN(CCO)CCCCCCCC(=O)OCCC(CCCC)CCCC] | Compound I |
| LNP-3 | 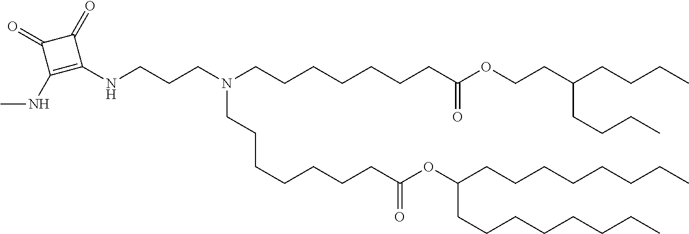 [SMILES: CCCCCCCCC(CCCCCCCC)OC(=O)CCCCCCCN(CCCCCCCC(=O)OCCC(CCCC)CCCC)CCCNc1c(NC)c(=O)c1=O] | Compound I |
| LNP-4 | 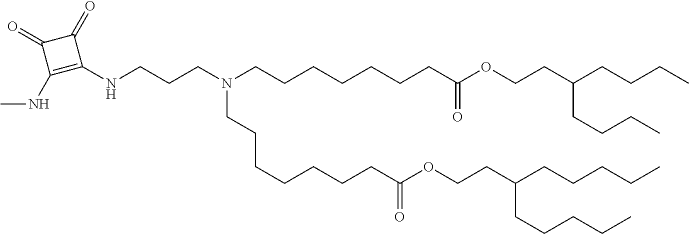 [SMILES: CCCCCC(CCCCC)CCOC(=O)CCCCCCCN(CCCCCCCC(=O)OCCC(CCCC)CCCC)CCCNc1c(NC)c(=O)c1=O] | Compound I |

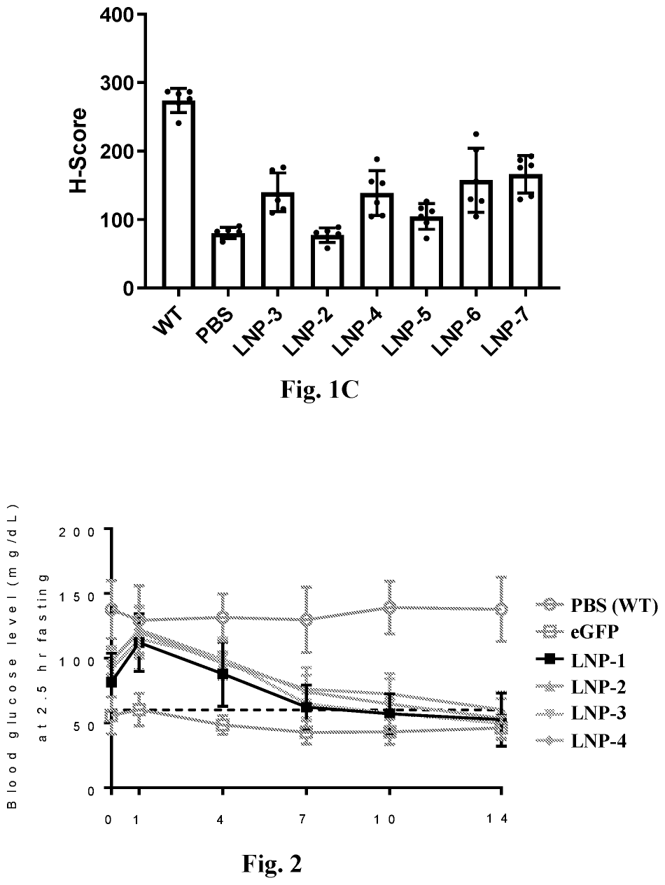

To test the efficacy and duration of action of a 1-methyl-pseudouridine modified mRNA encoding GE in vivo, a single 0.25 mg/kg dose of mRNA in a lipid nanoparticle formulation of Table 17 was intravenously administered to mice deficient for GE via tail vein injection. As controls, PBS was injected into wild-type mice and a lipid nanoparticle formulation (Compound A/Compound I) containing an mRNA encoding eGFP (at 0.25 mg/kg) was injected into mice deficient for GE. Blood glucose levels were monitored at 2.5 hours fasting on the day of mRNA injection (day 0), and each of days 1, 4, 7, 10, and 14 post-injection. Mice administered the GE mRNA formulated in each of the lipid nanoparticles exhibited blood glucose levels significantly higher than those of mice injected with the eGFP control for up to 10 days (FIG. 2). The 2.5-hr fasting blood glucose levels in mice treated with the GE mRNA formulations were also maintained above or close to 60 mg/dl for up to 10 days (FIG. 2). LNP-2 extended the duration of action beyond day 10 (to approximately 14 days).

The effect of different lipid nanoparticle formulations on GE expression and activity in wild type mice was also tested. Table 18 lists the ionizable amino lipid and PEG-lipid used in each tested lipid nanoparticle formulation.

TABLE 18
Lipid Nanoparticle Formulations

| LNP | Ionizable Amino Lipid | PEG-Lipid |
|---|---|---|
| LNP-1 | 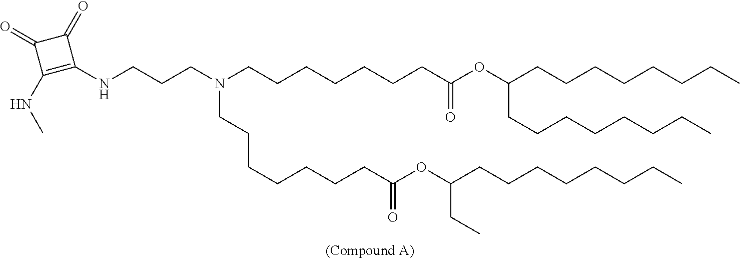 [SMILES: CCCCCCCCC(CC)OC(=O)CCCCCCCN(CCCCCCCC(=O)OC(CCCCCCCC)CCCCCCCC)CCCNc1c(NC)c(=O)c1=O] | Compound I |
| LNP-2 | 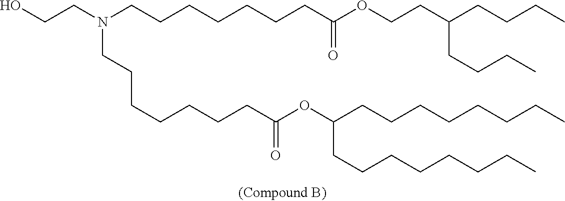 [SMILES: CCCCCCCCC(CCCCCCCC)OC(=O)CCCCCCCN(CCO)CCCCCCCC(=O)OCCC(CCCC)CCCC] | Compound I |
| LNP-3 | 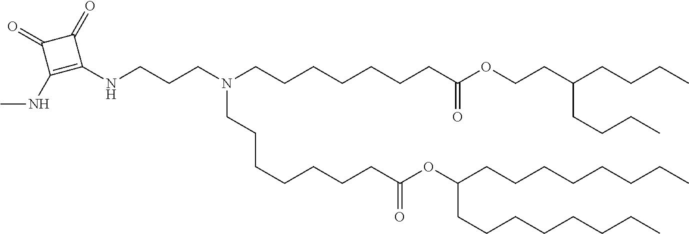 [SMILES: CCCCCCCCC(CCCCCCCC)OC(=O)CCCCCCCN(CCCCCCCC(=O)OCCC(CCCC)CCCC)CCCNc1c(NC)c(=O)c1=O] | Compound I |
| LNP-4 | 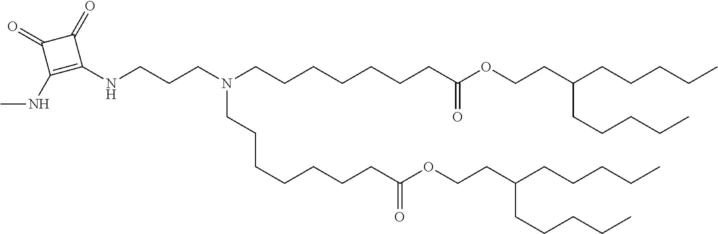 [SMILES: CCCCCC(CCCCC)CCOC(=O)CCCCCCCN(CCCCCCCC(=O)OCCC(CCCCC)CCCCC)CCCNc1c(NC)c(=O)c1=O] | Compound I |
| LNP-5 | 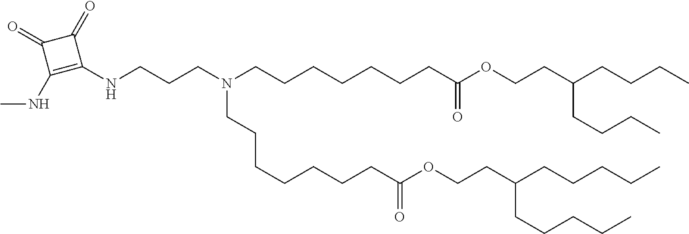 [SMILES: CCCCCC(CCCCC)CCOC(=O)CCCCCCCN(CCCCCCCC(=O)OCCC(CCCC)CCCC)CCCNc1c(NC)c(=O)c1=O] | Compound I |

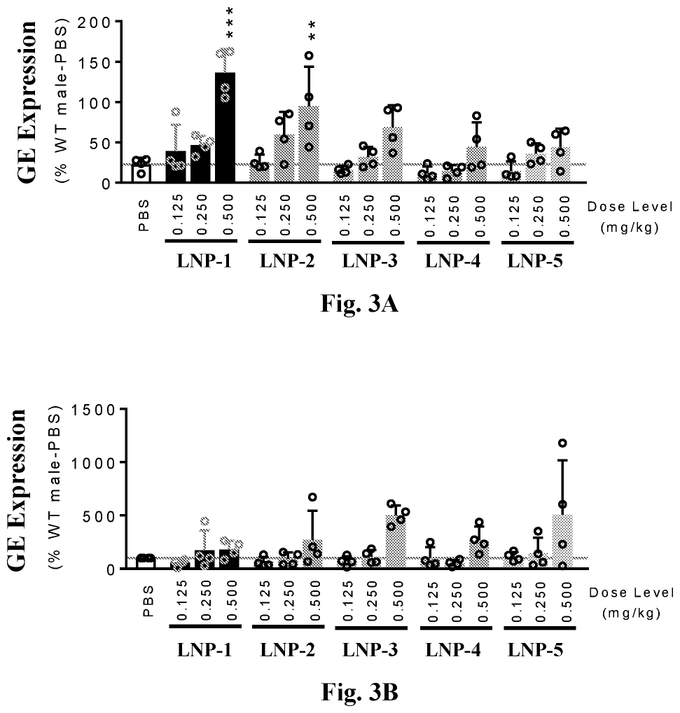

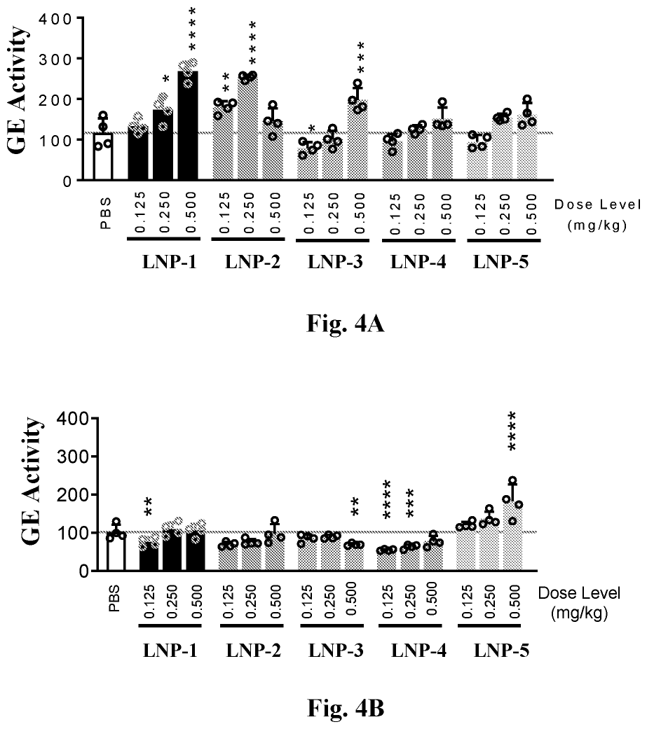

The lipid nanoparticle formulations of Table 18 were injected into wild-type mice in mRNA doses of 0.125 mg/kg, 0.25 mg/kg, or 0.5 mg/kg via a single injection, and liver GE expression and activity was measured at 24 hours post-injection. Control mice were injected with PBS. GE expression from each of the lipid nanoparticle formulations, as compared to PBS treatment, is shown in FIG. 3A (female mice) and FIG. 3B (male mice). GE activity from each of the lipid nanoparticle formulations, as compared to PBS treatment, is shown in FIG. 4A (female mice) and FIG. 4B (male mice). The results are shown as percentage of mean values of the group of male mice treated with PBS.

What is claimed is:

1. A lipid nanoparticle comprising a first messenger RNA (mRNA) comprising a first open reading frame (ORF) encoding a propionyl-CoA carboxylase alpha (PCCA) polypeptide and a second mRNA comprising a second ORF encoding a propionyl-CoA carboxylase beta (PCCB) polypeptide, wherein the lipid nanoparticle comprises a compound of Formula (II): 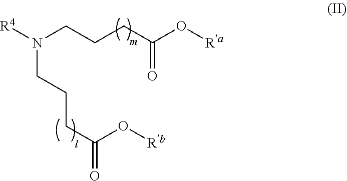
or its N-oxide, or a salt or isomer thereof,
wherein R′^{a} is R′^{branched} or R′^{cyclic}; wherein
R′^{branched} is: 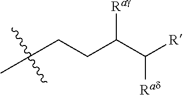
and R′^{cyclic} is: 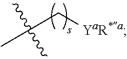
and
R′^{b} is: 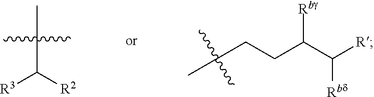
wherein 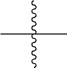
denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and
C₂₋₁₄ alkenyl;
R⁴ is selected from the group consisting of —(CH₂)ₙOH wherein n is selected from the group consisting of 1, 2, 3, 4, and 5, and 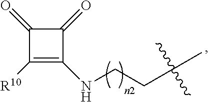
wherein 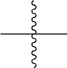
denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

2. The lipid nanoparticle of claim 1, wherein the lipid nanoparticle comprises a compound of Formula (II-a): 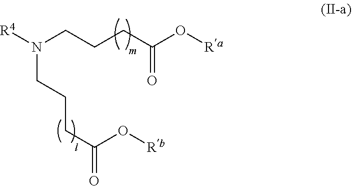
or its N-oxide, or a salt or isomer thereof,
wherein R′^{a} is R′^{branched} or R′^{cyclic}; wherein
R′^{branched} is: 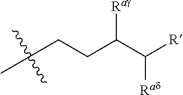
and R′^{b} is: 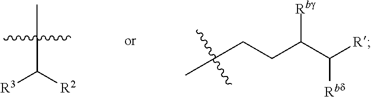
wherein 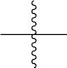
denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{a}a is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and
C₂₋₁₄ alkenyl;
R⁴ is selected from the group consisting of —(CH₂)ₙOH wherein n is selected from the group consisting of 1, 2, 3, 4, and 5, and 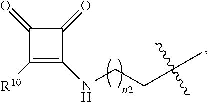
wherein 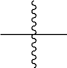
denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

3. The lipid nanoparticle of claim 1, wherein the lipid nanoparticle comprises a compound of Formula (II-b): 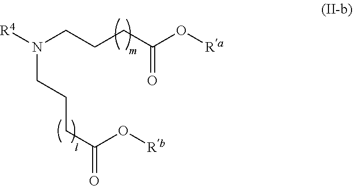
or its N-oxide, or a salt or isomer thereof,
wherein R′^{a} is R′^{branched} or R′^{cyclic}; wherein
R′^{branched} is: 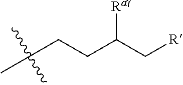
and R′^{b} is: 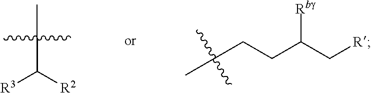
wherein 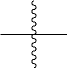
denotes a point of attachment;
R^{aγ} and R^{bγ} are each independently selected from the group consisting of C₁₋₁₂ alkyl and C₂₋₁₂ alkenyl;
R₂ and R³ are each independently selected from the group consisting of C₁₋₁₄ alkyl and
C₂₋₁₄ alkenyl;
R⁴ is selected from the group consisting of —(CH₂)ₙOH wherein n is selected from the group consisting of 1, 2, 3, 4, and 5, and 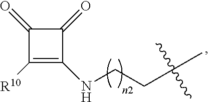
wherein 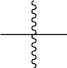
denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

4. The lipid nanoparticle of claim 1, wherein the lipid nanoparticle comprises a compound of Formula (II-c): 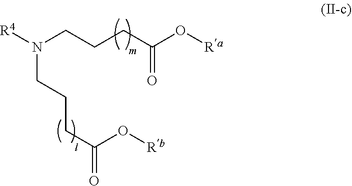
or its N-oxide, or a salt or isomer thereof,
wherein R′^{a} is R′^{branched} or R′^{cyclic}; wherein
R′^{branched} is: 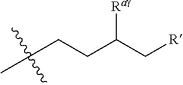
and R′^{b} is: 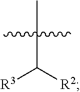
wherein 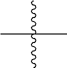
denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and
C₂₋₁₄ alkenyl;
R⁴ is selected from the group consisting of —(CH₂)ₙOH wherein n is selected from the group consisting of 1, 2, 3, 4, and 5, and 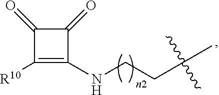
wherein 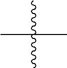
denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

5. The lipid nanoparticle of claim 1, wherein the lipid nanoparticle comprises a compound of Formula (II-e): 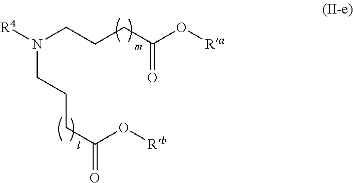
or its N-oxide, or a salt or isomer thereof,
wherein R′^{a} is R′^{branched} or R^{cyclic}; wherein
R′^{branched} is: 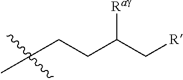
and R′^{b} is: 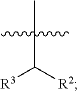
wherein 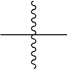
denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and
C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

6. The lipid nanoparticle of claim 1, wherein the lipid nanoparticle comprises a compound of Formula (II-f): 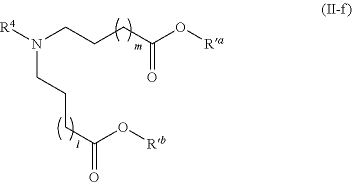
or its N-oxide, or a salt or isomer thereof,
wherein R′^{a} is R′^{branched} or R′^{cyclic}; wherein
R′^{branched} is: 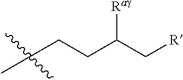
and R′^{b} is: 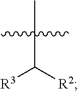
wherein 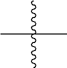
denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

7. The lipid nanoparticle of claim 1, wherein the compound is 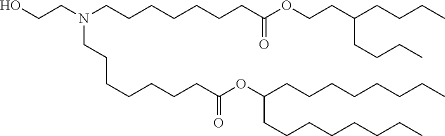 [SMILES: CCCCCCCCC(CCCCCCCC)OC(=O)CCCCCCCN(CCO)CCCCCCCC(=O)OCCC(CCCC)CCCC]
or its N-oxide, or a salt or isomer thereof.

8. The lipid nanoparticle of claim 1, wherein the compound is 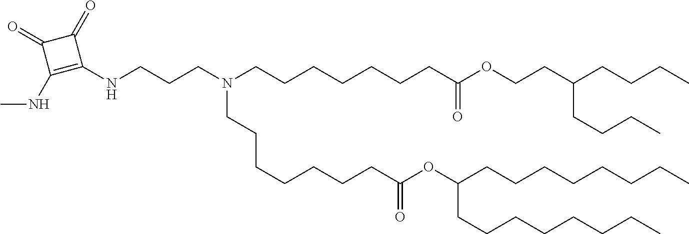 [SMILES: CCCCCCCCC(CCCCCCCC)OC(=O)CCCCCCCN(CCCCCCCC(=O)OCCC(CCCC)CCCC)CCCNc1c(NC)c(=O)c1=O]
or its N-oxide, or a salt or isomer thereof.

9. The lipid nanoparticle of claim 1, wherein the compound is 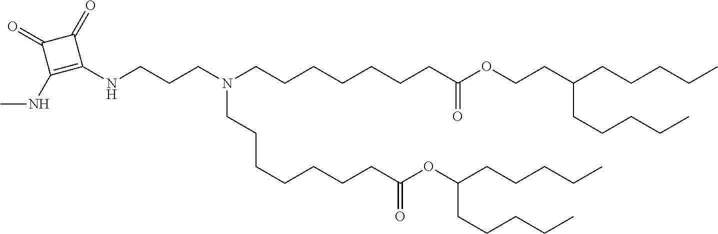 [SMILES: CCCCCC(CCCCC)CCOC(=O)CCCCCCCN(CCCCCCCC(=O)OC(CCCCC)CCCCC)CCCNc1c(NC)c(=O)c1=O]
or its N-oxide, or a salt or isomer thereof.

10. The lipid nanoparticle of claim 1, wherein the compound is 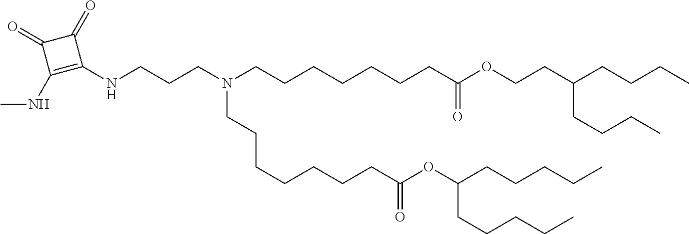 [SMILES: CCCCCC(CCCCC)OC(=O)CCCCCCCN(CCCCCCCC(=O)OCCC(CCCC)CCCC)CCCNc1c(NC)c(=O)c1=O]
or its N-oxide, or a salt or isomer thereof.

11. The lipid nanoparticle of any one of claims 1 to 10, wherein the PCCA polypeptide comprises the amino acid sequence of SEQ ID NO:1 and the PCCB polypeptide comprises the amino acid sequence of SEQ ID NO:2.

12. The lipid nanoparticle of any one of claims 1 to 11, wherein the first mRNA and the second mRNA are present in the lipid nanoparticle at a ratio of 1:1.

13. The lipid nanoparticle of any one of claims 1 to 12, wherein the lipid nanoparticle further comprises a phospholipid, a structural lipid, and a PEG-lipid.

14. The lipid nanoparticle of claim 13, wherein the PEG-lipid is Compound I.

15. The lipid nanoparticle of claim 13 or 14, wherein the lipid nanoparticle comprises:
(i) 40-50 mol % of the compound of Formula (II), 30-45 mol % of the structural lipid, 5-15 mol % of the phospholipid, and 1-5 mol % of the PEG-lipid; or
(ii) 45-50 mol % of the compound of Formula (II), 35-45 mol % of the structural lipid, 8-12 mol % of the phospholipid, and 1.5 to 3.5 mol % of the PEG-lipid

16. The lipid nanoparticle of any one of claims 1 to 15, wherein the first ORF is at least 80%, at least 85%, at least 90%, at least 91%, at least 92%, at least 93%, at least 94%, at least 95%, at least 96%, at least 97%, at least 98%, or at least 99% identical to the nucleotide sequence of SEQ ID NO:5, and wherein the second ORF is at least 80%, at least 85%, at least 90%, at least 91%, at least 92%, at least 93%, at least 94%, at least 95%, at least 96%, at least 97%, at least 98%, or at least 99% identical to the nucleotide sequence of SEQ ID NO:6.

17. The lipid nanoparticle of any one of claims 1 to 15, wherein the first ORF is at least 95% identical to the nucleotide sequence of SEQ ID NO:5 and the second ORF is at least 95% identical to the nucleotide sequence of SEQ ID NO:6.

18. The lipid nanoparticle of any one of claims 1 to 15, wherein the first ORF is 100% identical to the nucleotide sequence of SEQ ID NO:5, and wherein the second ORF is 100% identical to the nucleotide sequence of SEQ ID NO:6.

19. The lipid nanoparticle of any one of claims 1 to 18, wherein the first mRNA comprises a 5′ UTR comprising a nucleic acid sequence at least 90%, at least 95%, at least 96%, at least 97%, at least 98%, at least 99%, or 100% identical to SEQ ID NO:55, and wherein the second mRNA comprises a 5′ UTR comprising a nucleic acid sequence at least 90%, at least 95%, at least 96%, at least 97%, at least 98%, at least 99%, or 100% identical to SEQ ID NO:55.

20. The lipid nanoparticle of any one of claims 1 to 19, wherein first the mRNA comprises a 3′ UTR comprising a nucleic acid sequence at least 90%, at least 95%, at least 96%, at least 97%, at least 98%, at least 99%, or 100% identical to SEQ ID NO:114, and wherein second the mRNA comprises a 3′ UTR comprising a nucleic acid sequence at least 90%, at least 95%, at least 96%, at least 97%, at least 98%, at least 99%, or 100% identical to SEQ ID NO:114.

21. The lipid nanoparticle of any one of claims 1 to 15, wherein the first mRNA comprises the nucleic acid sequence of SEQ ID NO:7 and the second mRNA comprises the nucleic acid sequence of SEQ ID NO:8.

22. The lipid nanoparticle of any one of claims 1 to 21, wherein the first mRNA and the second mRNA each comprise a 5′ terminal cap.

23. The lipid nanoparticle of claim 22, wherein the 5′ terminal cap comprises a Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

24. The lipid nanoparticle of any one of claims 1 to 23, wherein the first mRNA and the second mRNA each comprise a poly-A region.

25. The lipid nanoparticle of claim 24, wherein the poly-A region is at least about 10, at least about 20, at least about 30, at least about 40, at least about 50, at least about 60, at least about 70, at least about 80, at least about 90 nucleotides in length, or at least about 100 nucleotides in length.

26. The lipid nanoparticle of claim 24, wherein the poly-A region is at least about 100 nucleotides in length.

27. The lipid nanoparticle of any one of claims 1 to 26, wherein all of the uracils of the first mRNA and the second mRNA are N1-methylpseudouracils.

28. The lipid nanoparticle of any one of claims 1 to 15, wherein the first ORF is 100% identical to SEQ ID NO:5, wherein the second ORF is 100% identical to SEQ ID NO:6, wherein the first mRNA and the second mRNA each comprise a poly-A region at least about 100 nucleotides in length, and wherein all of the uracils of the first mRNA and the second mRNA are N1-methylpseudouracils.

29. The lipid nanoparticle of any one of claims 1 to 15, wherein the first mRNA and the second mRNA each comprise a 5′ terminal cap comprising a guanine cap nucleotide containing an N₇ methylation and the 5′-terminal nucleotide of the mRNA contains a 2′-O-methyl, wherein the first mRNA comprises the nucleic acid sequence of SEQ ID NO:7 and the second mRNA comprises the nucleic acid sequence of SEQ ID NO:8, wherein the first mRNA and the second mRNA each comprise a poly-A region at least about 100 nucleotides in length, and wherein all of the uracils of the first mRNA and the second mRNA are N1-methylpseudouracils.

30. A method of expressing a propionyl-CoA carboxylase alpha (PCCA) polypeptide and a propionyl-CoA carboxylase beta (PCCB) polypeptide in a human subject in need thereof, comprising administering to the subject an effective amount of the lipid nanoparticle of any one of claims 1 to 29.

31. A method of treating, preventing, or delaying the onset and/or progression of propionic academia in a human subject in need thereof, the method comprising administering to the human subject an effective amount of the lipid nanoparticle of any one of claims 1 to 29.

32. A method of reducing propionic acid blood level in a human subject in need thereof, comprising administering to the subject an effective amount of the lipid nanoparticle of any one of claims 1 to 29.

33. A method of reducing C3, 2-MC, 30HPA, and/or ammonia plasma, serum, whole blood, and/or liver level in a human subject in need thereof, comprising administering to the subject an effective amount of the lipid nanoparticle of any one of claims 1 to 29.

34. A method of increasing PCC activity in a human subject in need thereof, comprising administering to the subject an effective amount of the lipid nanoparticle of any one of claims 1 to 29.

35. The method of any one of claims 30 to 34, wherein the human subject has a loss of function mutation in the PCCA gene.

36. The method of any one of claims 30 to 34, wherein the human subject has a loss of function mutation in the PCCB gene.

37. The method of any one of claims 30 to 34, wherein the human subject has a loss of function mutation in the PCCA gene and a loss of function mutation in the PCCB gene.

38. The method of any one of claims 30 to 37, comprising multiple administrations of the lipid nanoparticle to the human subject.

39. The method of claim 38, wherein the multiple administrations to the human subject are about once a week, about once every two weeks, or about once a month.

40. The method of any one of claims 30 to 39, wherein the lipid nanoparticle is administered intravenously.


SEQUENCE LISTING (217 sequences)

SEQ ID NO: 1 - protein, 728 aa, Artificial Sequence
  source: /note=“Description of Artificial Sequence: Synthetic polypeptide”
MAGFWVGTAPLVAAGRRGRWPPQQLMLSAALRTLKHVLYYSRQCLMVSRNLGSVGYDPNEKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVYKVNILTRLAAELNKFMLEKVTEDTSSVLRSPMPGVVVAVSVKPGDAVAEGQEICVIEAMKMQNSMTAGKTGTVKSVHCQAGDTVGEGDLLVELE

SEQ ID NO: 2 - protein, 539 aa, Artificial Sequence
  source: /note=“Description of Artificial Sequence: Synthetic polypeptide”
MAAALRVAAVGARLSVLASGLRAAVRSLCSQATSVNERIENKRRTALLGGGQRRIDAQHKRGKLTARERISLLLDPGSFVESDMFVEHRCADFGMAADKNKFPGDSVVTGRGRINGRLVYVFSQDFTVFGGSLSGAHAQKICKIMDQAITVGAPVIGLNDSGGARIQEGVESLAGYADIFLRNVTASGVIPQISLIMGPCAGGAVYSPALTDFTFMVKDTSYLFITGPDVVKSVTNEDVTQEELGGAKTHTTMSGVAHRAFENDVDALCNLRDFFNYLPLSSQDPAPVRECHDPSDRLVPELDTIVPLESTKAYNMVDIIHSVVDEREFFEIMPNYAKNIIVGFARMNGRTVGIVGNQPKVASGCLDINSSVKGARFVRFCDAFNIPLITFVDVPGFLPGTAQEYGGIIRHGAKLLYAFAEATVPKVTVITRKAYGGAYDVMSSKHLCGDTNYAWPTAEIAVMGAKGAVEIIFKGHENVEAAQAEYIEKFANPFPAAVRGFVDDIIQPSSTRARICCDLDVLASKKVQRPWRKHANIPL

SEQ ID NO: 3-4 - skipped (2 entries)

SEQ ID NO: 5 - RNA, 2184 nt, Artificial Sequence
  source: /note=“Description of Artificial Sequence: Synthetic polynucleotide”
auggccggcuucugggucggcacagccccucugguggcagccggcagaagaggacgguggccuccccagcaacugaugcugagcgccgcccugagaacccugaagcacgugcuguacuacagcagacagugccugauggugagcagaaaucugggcagcgugggguacgaucccaacgagaagaccuucgauaagauucuggucgcgaauagaggcgagaucgccugcagggugaucagaaccugcaagaagaugggcaucaagaccguggccauccauucggacgucgacgcgagcagcguucacgugaagauggcagacgaggccgugugcgugggacccgccccgaccagcaagagcuaccugaacauggacgccaucauggaggccaucaagaagacccgcgcucaagccgugcacccgggcuacggcuuucugagcgagaacaaggaauucgccaggugucucgccgccgaggacguagucuucaucggcccugauacgcacgcgauccaggccaugggcgacaagaucgagagcaaacugcuggccaagaaagcagaagucaacaccauccccggcuucgacggcguggugaaggacgccgaagaggcuguccgcaucgccagagagaucggcuacccugugaugauaaaggcuagcgcuggagguggcggaaagggcaugagaaucgccugggacgacgaggagacuagagacggcuucagacuguccucccaggaggccgccagcuccuucggagacgacagacugcugaucgagaaguucaucgacaaccccagacacaucgaaauccaggugcucggugacaagcacgggaacgcccuguggcugaacgagagagagugcagcauccagagaagaaaccagaaggugguggaggaggcgccgagcaucuuucuggacgcggagacaaggagagcgaugggcgaacaggccgucgcccuagcaagagccgugaaguacuccagugccggaaccgucgaguuucuugucgacagcaagaagaauuucuacuuccuggagaugaacaccaggcugcagguggagcaucccgugacagagugcaucacuggacuggaucuggugcaggagaugaucaggguggccaagggcuauccccugagacacaagcaggccgacaucagaaucaacggcugggccguggagugcagaguguacgccgaggaccccuacaagagcuucggccugcccagcaucggcagacugagccaguaccaggagccccugcaccugcccggcgugagaguggacagcggcauccaaccggggagcgauaucagcaucuacuacgaccccaugaucagcaagcugauaaccuacggcagcgacagaaccgaggcccugaagagaauggccgacgcccuggacaacuacgugaucagaggcgugacccacaacaucgcccugcugagagaggugaucaucaacucgagguucgugaaaggcgacaucagcaccaaguuccugagcgacguguaucccgacggauucaaaggucacaugcugaccaagagcgagaagaaccagcugcuggccaucgccucaucccuguucguggccuuccagcugagagcccagcacuuccaggagaacagcagaaugcccgugaucaagcccgacaucgccaacugggagcugagcgugaagcugcacgacaaggugcacacugucguugccagcaacaacggcuccguguucagcguagagguggacggaucuaagcugaacgugaccuccaccuggaaccuggcaagcccucuccugucagugagcguggacggcacccagagaaccgugcagugucugucccgcgaggccggcggaaacaugagcauccaguuccugggcaccguguacaaggugaacauccugaccagacuggccgccgagcugaacaaguucaugcuggagaaagugacggaggauaccagcuccgugcugagaagccccaugcccggaguggugguggccguuuccgugaaaccuggugacgccguggccgaggggcaagagaucugcgugaucgaggccaugaagaugcagaauuccaugaccgccggaaagaccggcaccgucaaaucagugcacugccaggcgggcgacacagugggugagggcgaccugcugguggagcuggag

SEQ ID NO: 6 - RNA, 1617 nt, Artificial Sequence
  source: /note=“Description of Artificial Sequence: Synthetic polynucleotide”
auggcggcggcauuacggguggcggcggucggggcaaggcucagcgugcuggccagcggccugagagccgccgugagaagccugugcagccaggccaccagcgugaacgagagaaucgagaacaagagaagaaccgcccugcugggcggcggccagagaagaaucgacgcccagcacaagagaggcaagcugaccgccagagagagaaucagccugcugcuggaccccggcagcuucguggagagcgacauguucguggagcacaggugcgccgacuucggcauggccgccgacaagaacaaguuccccggcgacagcguggugaccggcagaggcagaaucaacggcagacugguguacguguucagccaggacuucaccguguucggcggcagccugagcggcgcccacgcccagaagaucugcaagaucauggaccaggccaucaccgugggcgcgcccgugaucggccugaacgacagcggcggcgccagaauccaggagggcguggagagccuggccggcuacgccgacaucuuccugagaaacgugaccgccagcggcgugaucccacagaucagccugaucaugggccccugcgccggcggcgccguguacagccccgcccugaccgacuucaccuucauggugaaggacaccagcuaccuguucaucaccggccccgacguggugaagagcgugaccaacgaggacgugacccaggaggagcugggcggcgccaagacccacaccaccaugagcggcguggcccacagagccuucgagaacgacguggacgcccugugcaaccugagagacuucuucaacuaccugccccugagcagccaggaccccgcgcccgugagagagugccacgaccccagcgacagacuggugcccgagcuggacaccaucgugccccuggagagcaccaaggccuacaacaugguggacaucauccacagcgugguggacgagagagaguucuucgagaucaugcccaacuacgccaagaacaucaucgugggcuucgccagaaugaacggcagaaccgugggcaucgugggcaaccagcccaagguggccagcggcugccuggacaucaacagcagcgugaagggcgccagauucgugagauucugcgacgccuucaacaucccucugaucaccuucguggacgugcccggcuuccugcccggcaccgcccaggaguacggcggcaucaucagacacggcgccaagcugcuguacgccuucgccgaggccaccgugcccaaggugaccgugaucaccagaaaggccuacggcggcgccuacgacgugaugagcagcaagcaccugugcggcgacaccaacuacgccuggcccaccgccgagaucgccgugaugggcgccaagggcgccguggagaucaucuucaagggccacgagaacguggaggccgcccaggccgaguacaucgagaaguucgccaaccccuuccccgccgccgugagaggcuucguggacgacaucauccagcccagcagcaccagagccagaaucugcugcgaccuggacgugcuggccagcaagaaggugcagagacccuggagaaagcacgccaacaucccucug

SEQ ID NO: 7 - RNA, 2418 nt, Artificial Sequence
  source: /note=“Description of Artificial Sequence: Synthetic polynucleotide”
ggaaauaagagagaaaagaagaguaagaagaaauauaagagccaccauggccggcuucugggucggcacagccccucugguggcagccggcagaagaggacgguggccuccccagcaacugaugcugagcgccgcccugagaacccugaagcacgugcuguacuacagcagacagugccugauggugagcagaaaucugggcagcgugggguacgaucccaacgagaagaccuucgauaagauucuggucgcgaauagaggcgagaucgccugcagggugaucagaaccugcaagaagaugggcaucaagaccguggccauccauucggacgucgacgcgagcagcguucacgugaagauggcagacgaggccgugugcgugggacccgccccgaccagcaagagcuaccugaacauggacgccaucauggaggccaucaagaagacccgcgcucaagccgugcacccgggcuacggcuuucugagcgagaacaaggaauucgccaggugucucgccgccgaggacguagucuucaucggcccugauacgcacgcgauccaggccaugggcgacaagaucgagagcaaacugcuggccaagaaagcagaagucaacaccauccccggcuucgacggcguggugaaggacgccgaagaggcuguccgcaucgccagagagaucggcuacccugugaugauaaaggcuagcgcuggagguggcggaaagggcaugagaaucgccugggacgacgaggagacuagagacggcuucagacuguccucccaggaggccgccagcuccuucggagacgacagacugcugaucgagaaguucaucgacaaccccagacacaucgaaauccaggugcucggugacaagcacgggaacgcccuguggcugaacgagagagagugcagcauccagagaagaaaccagaaggugguggaggaggcgccgagcaucuuucuggacgcggagacaaggagagcgaugggcgaacaggccgucgcccuagcaagagccgugaaguacuccagugccggaaccgucgaguuucuugucgacagcaagaagaauuucuacuuccuggagaugaacaccaggcugcagguggagcaucccgugacagagugcaucacuggacuggaucuggugcaggagaugaucaggguggccaagggcuauccccugagacacaagcaggccgacaucagaaucaacggcugggccguggagugcagaguguacgccgaggaccccuacaagagcuucggccugcccagcaucggcagacugagccaguaccaggagccccugcaccugcccggcgugagaguggacagcggcauccaaccggggagcgauaucagcaucuacuacgaccccaugaucagcaagcugauaaccuacggcagcgacagaaccgaggcccugaagagaauggccgacgcccuggacaacuacgugaucagaggcgugacccacaacaucgcccugcugagagaggugaucaucaacucgagguucgugaaaggcgacaucagcaccaaguuccugagcgacguguaucccgacggauucaaaggucacaugcugaccaagagcgagaagaaccagcugcuggccaucgccucaucccuguucguggccuuccagcugagagcccagcacuuccaggagaacagcagaaugcccgugaucaagcccgacaucgccaacugggagcugagcgugaagcugcacgacaaggugcacacugucguugccagcaacaacggcuccguguucagcguagagguggacggaucuaagcugaacgugaccuccaccuggaaccuggcaagcccucuccugucagugagcguggacggcacccagagaaccgugcagugucugucccgcgaggccggcggaaacaugagcauccaguuccugggcaccguguacaaggugaacauccugaccagacuggccgccgagcugaacaaguucaugcuggagaaagugacggaggauaccagcuccgugcugagaagccccaugcccggaguggugguggccguuuccgugaaaccuggugacgccguggccgaggggcaagagaucugcgugaucgaggccaugaagaugcagaauuccaugaccgccggaaagaccggcaccgucaaaucagugcacugccaggcgggcgacacagugggugagggcgaccugcugguggagcuggagugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 8 - RNA, 1851 nt, Artificial Sequence
  source: /note=“Description of Artificial Sequence: Synthetic polynucleotide”
ggaaauaagagagaaaagaagaguaagaagaaauauaagagccaccauggcggcggcauuacggguggcggcggucggggcaaggcucagcgugcuggccagcggccugagagccgccgugagaagccugugcagccaggccaccagcgugaacgagagaaucgagaacaagagaagaaccgcccugcugggcggcggccagagaagaaucgacgcccagcacaagagaggcaagcugaccgccagagagagaaucagccugcugcuggaccccggcagcuucguggagagcgacauguucguggagcacaggugcgccgacuucggcauggccgccgacaagaacaaguuccccggcgacagcguggugaccggcagaggcagaaucaacggcagacugguguacguguucagccaggacuucaccguguucggcggcagccugagcggcgcccacgcccagaagaucugcaagaucauggaccaggccaucaccgugggcgcgcccgugaucggccugaacgacagcggcggcgccagaauccaggagggcguggagagccuggccggcuacgccgacaucuuccugagaaacgugaccgccagcggcgugaucccacagaucagccugaucaugggccccugcgccggcggcgccguguacagccccgcccugaccgacuucaccuucauggugaaggacaccagcuaccuguucaucaccggccccgacguggugaagagcgugaccaacgaggacgugacccaggaggagcugggcggcgccaagacccacaccaccaugagcggcguggcccacagagccuucgagaacgacguggacgcccugugcaaccugagagacuucuucaacuaccugccccugagcagccaggaccccgcgcccgugagagagugccacgaccccagcgacagacuggugcccgagcuggacaccaucgugccccuggagagcaccaaggccuacaacaugguggacaucauccacagcgugguggacgagagagaguucuucgagaucaugcccaacuacgccaagaacaucaucgugggcuucgccagaaugaacggcagaaccgugggcaucgugggcaaccagcccaagguggccagcggcugccuggacaucaacagcagcgugaagggcgccagauucgugagauucugcgacgccuucaacaucccucugaucaccuucguggacgugcccggcuuccugcccggcaccgcccaggaguacggcggcaucaucagacacggcgccaagcugcuguacgccuucgccgaggccaccgugcccaaggugaccgugaucaccagaaaggccuacggcggcgccuacgacgugaugagcagcaagcaccugugcggcgacaccaacuacgccuggcccaccgccgagaucgccgugaugggcgccaagggcgccguggagaucaucuucaagggccacgagaacguggaggccgcccaggccgaguacaucgagaaguucgccaaccccuuccccgccgccgugagaggcuucguggacgacaucauccagcccagcagcaccagagccagaaucugcugcgaccuggacgugcuggccagcaagaaggugcagagacccuggagaaagcacgccaacaucccucugugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 9-49 - skipped (41 entries)

SEQ ID NO: 50 - RNA, 75 nt, Artificial Sequence
  source: /note=“Description of Artificial Sequence: Synthetic oligonucleotide”
ggaaaucgcaaaauuugcucuucgcguuagauuucuuuuaguuuucucgcaacuagcaagcuuuuuguucucgcc

SEQ ID NO: 51 - RNA, 203 nt, Artificial Sequence
  source: /note=“Description of Artificial Sequence: Synthetic polynucleotide”
ggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 52 - RNA, 76 nt, Artificial Sequence
  source: /note=“Description of Artificial Sequence: Synthetic oligonucleotide”
ggaaauaagagagaaaagaagaguaagaagaaauauaagaucucccugagcuucagggagccccggcgccgccacc

SEQ ID NO: 53 - RNA, 89 nt, Artificial Sequence
  source: /note=“Description of Artificial Sequence: Synthetic oligonucleotide”
ggaaaccccccacccccguaagagagaaaagaagaguaagaagaaauauaagaucucccugagcuucagggagccccggcgccgccacc

SEQ ID NO: 54 - RNA, 59 nt, Artificial Sequence
  source: /note=“Description of Artificial Sequence: Synthetic oligonucleotide”
ggagaacuuccgcuuccguuggcgcaagcgcuuucauuuuuucugcuaccgugacuaag

SEQ ID NO: 55 - RNA, 46 nt, Artificial Sequence
  source: /note=“Description of Artificial Sequence: Synthetic oligonucleotide”
ggaaauaagagagaaaagaagaguaagaagaaauauaagagccacc

SEQ ID NO: 56 - RNA, 56 nt, Artificial Sequence
  source: /note=“Description of Artificial Sequence: Synthetic oligonucleotide”
ggaaauaagagagaaaagaagaguaagaagaaauauaagaccccggcgccgccacc

SEQ ID NO: 57 - RNA, 81 nt, Artificial Sequence
  source: /note=“Description of Artificial Sequence: Synthetic oligonucleotide”
ggaaaucgcaaaauuugcucuucgcguuagauuucuuuuaguuuucucgcaacuagcaagcuuuuuguucucgccgccgcc

SEQ ID NO: 58 - RNA, 81 nt, Artificial Sequence
  source: /note=“Description of Artificial Sequence: Synthetic oligonucleotide”
ggaaaucgcaaaauuuucuuuucgcguuagauuucuuuuaguuuucuuucaacuagcaagcuuuuuguucucgccgccgcc

SEQ ID NO: 59 - RNA, 83 nt, Artificial Sequence
  source: /note=“Description of Artificial Sequence: Synthetic oligonucleotide”
  misc_feature (14)..(18): /note=“This region may encompass 0-5 nucleotides”
  variation (19)..(19): /replace=“ ”
  variation (22)..(22): /replace=“ ”
  misc_feature (23)..(27): /note=“This region may encompass 0-5 nucleotides”
  variation (81)..(83): /replace=“ ”
  misc_feature (1)..(83): /note=“Variant nucleotides given in the sequence have no preference with respect to those in the annotations for variant positions”
  source: /note=“See specification as filed for detailed description of substitutions and preferred embodiments”
ggaaaucgcaaaauuuuugcucuuuuucgcguuagauuucuuuuaguuuucuykcaacuagcaagcuuuuuguucucgccrcc

SEQ ID NO: 60 - RNA, 79 nt, Artificial Sequence
  source: /note=“Description of Artificial Sequence: Synthetic oligonucleotide”
ggaaaauuuuagccuggaacguuagauaacuguccuguugucuuuauauacuugguccccaaguaguuugucuuccaaa

SEQ ID NO: 61 - RNA, 79 nt, Artificial Sequence
  source: /note=“Description of Artificial Sequence: Synthetic oligonucleotide”
ggaaacuuuauuuaguguuacuuuauuuucuguuuauuuguguuucuucaguggguuuguucuaauuuccuuggccgcc

SEQ ID NO: 62 - RNA, 79 nt, Artificial Sequence
  source: /note=“Description of Artificial Sequence: Synthetic oligonucleotide”
ggaaaaucuguauuagguuggcguguucuuuggucgguuguuaguauuguuguugauucguuuguggucgguugccgcc

SEQ ID NO: 63 - RNA, 79 nt, Artificial Sequence
  source: /note=“Description of Artificial Sequence: Synthetic oligonucleotide”
ggaaaauuauuaacaucuugguauucucgauaaccauucguuggauuuuauuguauucguaguuuggguuccugccgcc

SEQ ID NO: 64 - RNA, 79 nt, Artificial Sequence
  source: /note=“Description of Artificial Sequence: Synthetic oligonucleotide”
ggaaauuauuauuauuucuagcuacaauuuaucauuguauuauuuuagcuauucaucauuauuuacuuggugaucaaca

SEQ ID NO: 65 - RNA, 79 nt, Artificial Sequence
  source: /note=“Description of Artificial Sequence: Synthetic oligonucleotide”
ggaaauagguuguuaaccaaguucaagccuaauaagcuuggauucuggugacuugcuucaccguuggcgggcaccgauc

SEQ ID NO: 66 - RNA, 74 nt, Artificial Sequence
  source: /note=“Description of Artificial Sequence: Synthetic oligonucleotide”
ggaaaucguagagagucguacuuaguacauaucgacuaucgguggacaccaucaagauuauaaaccaggccaga

SEQ ID NO: 67 - RNA, 79 nt, Artificial Sequence
  source: /note=“Description of Artificial Sequence: Synthetic oligonucleotide”
ggaaacccgcccaagcgaccccaacauaucagcaguugcccaaucccaacucccaacacaauccccaagcaacgccgcc

SEQ ID NO: 68 - RNA, 79 nt, Artificial Sequence
  source: /note=“Description of Artificial Sequence: Synthetic oligonucleotide”
ggaaagcgauugaaggcgucuuuucaacuacucgauuaagguuggguaucgucgugggacuuggaaauuuguuguuucc

SEQ ID NO: 69 - RNA, 79 nt, Artificial Sequence
  source: /note=“Description of Artificial Sequence: Synthetic oligonucleotide”
ggaaacuaaucgaaauaaaagagccccguacucuuuuauuucuauuagguuaggagccuuagcauuuguaucuuaggua

SEQ ID NO: 70 - RNA, 79 nt, Artificial Sequence
  source: /note=“Description of Artificial Sequence: Synthetic oligonucleotide”
ggaaaugugauuuccagcaacuucuuuugaauauauugaauuccuaauucaaagcgaacaaaucuacaagccauauacc

SEQ ID NO: 71 - RNA, 74 nt, Artificial Sequence
  source: /note=“Description of Artificial Sequence: Synthetic oligonucleotide”
ggaaaucguagagagucguacuuacguggucgccauugcauagcgcgcgaaagcaacaggaacaagaacgcgcc

SEQ ID NO: 72 - RNA, 74 nt, Artificial Sequence
  source: /note=“Description of Artificial Sequence: Synthetic oligonucleotide”
ggaaaucguagagagucguacuuagaauaaacagagucgggucgacuugucucugauacuacgacgucacaauc

SEQ ID NO: 73 - RNA, 79 nt, Artificial Sequence
  source: /note=“Description of Artificial Sequence: Synthetic oligonucleotide”
ggaaaauuugccuucggaguugcguauccugaacugcccagccuccugauauacaacuguuccgcuuauucgggccgcc

SEQ ID NO: 74 - RNA, 79 nt, Artificial Sequence
  source: /note=“Description of Artificial Sequence: Synthetic oligonucleotide”
ggaaaucugagcaggaauccuuugugcauugaagacuuuagauuccucucugcgguagacgugcacuuauaaguauuug

SEQ ID NO: 75 - RNA, 79 nt, Artificial Sequence
  source: /note=“Description of Artificial Sequence: Synthetic oligonucleotide”
ggaaagcgauugaaggcgucuuuucaacuacucgauuaagguuggguaucgucgugggacuuggaaauuuguugccacc

SEQ ID NO: 76 - RNA, 104 nt, Artificial Sequence
  source: /note=“Description of Artificial Sequence: Synthetic polynucleotide”
ggaaauuuuuuuuugauauuauaagaguuuuuuuuugauauuaagaaaauuuuuuuuugauauuagaagaguaagaagaaauauaagaccccggcgccgccacc

SEQ ID NO: 77 - RNA, 57 nt, Artificial Sequence
  source: /note=“Description of Artificial Sequence: Synthetic oligonucleotide”
ggaaauaagagagaaaagaagaguaagaagaaauauaagagccaaaaaaaaaaaacc

SEQ ID NO: 78 - RNA, 76 nt, Artificial Sequence
  source: /note=“Description of Artificial Sequence: Synthetic oligonucleotide”
ggaaaucucccugagcuucagggaguaagagagaaaagaagaguaagaagaaauauaagaccccggcgccgccacc

SEQ ID NO: 79 - RNA, 6 nt, Artificial Sequence
  source: /note=“Description of Artificial Sequence: Synthetic oligonucleotide”
gccrcc

SEQ ID NO: 80-99 - skipped (20 entries)

SEQ ID NO: 100 - RNA, 119 nt, Artificial Sequence
  source: /note=“Description of Artificial Sequence: Synthetic polynucleotide”
uaauaguaagcuggagccuccugagagaccugugugaacuauugagaagaucggaacagcuccuuacucugaggaaguugguacccccguggucuuugaauaaagucugagugggcggc

SEQ ID NO: 101 - RNA, 119 nt, Artificial Sequence
  source: /note=“Description of Artificial Sequence: Synthetic polynucleotide”
uaauaguaagcuggagccucacucuccucuccaucccguauccaggcugugaauuuuucaaggaauauaaagaucgggauguacccccguggucuuugaauaaagucugagugggcggc

SEQ ID NO: 102 - RNA, 119 nt, Artificial Sequence
  source: /note=“Description of Artificial Sequence: Synthetic polynucleotide”
ugauaguaagcuggagccucuagugacggcaacagggcuugguuuuuccuuguugugaaaucgacaucucugaagacaggguacccccguggucuuugaauaaagucugagugggcggc

SEQ ID NO: 103 - RNA, 119 nt, Artificial Sequence
  source: /note=“Description of Artificial Sequence: Synthetic polynucleotide”
ugauaguaagcuggagccuccuuccaucuagucacaaagacuccuucguccccaguugccgucuaggauugggccucccaguacccccguggucuuugaauaaagucugagugggcggc

SEQ ID NO: 104 - RNA, 119 nt, Artificial Sequence
  source: /note=“Description of Artificial Sequence: Synthetic polynucleotide”
ugauaguaagcuggagccucccauaacaugacauaucuggauuuugugcuuagaaccuuaaauuggaagcauucuuaauuguacccccguggucuuugaauaaagucugagugggcggc

SEQ ID NO: 105 - RNA, 119 nt, Artificial Sequence
  source: /note=“Description of Artificial Sequence: Synthetic polynucleotide”
uaauaguaagcuggagccuccggaaaacuaaaauagagauauuucaagauuuuauaauuuucaaagaccuuugaaauauuguacccccguggucuuugaauaaagucugagugggcggc

SEQ ID NO: 106 - RNA, 119 nt, Artificial Sequence
  source: /note=“Description of Artificial Sequence: Synthetic polynucleotide”
uaauaguaagcuggagccucuacacauugcuucuaguuggcagaaauaauugauuaaaagaccagaaacugugauaacugguacccccguggucuuuaaauaaagucuaagugggcggc

SEQ ID NO: 107 - RNA, 119 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccguggucuuugaauaaagucugagugggcggc

SEQ ID NO: 108 - RNA, 119 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccguggucuuugaauaaagucugagugggcggc

SEQ ID NO: 109 - RNA, 141 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 110 - RNA, 141 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 111 - RNA, 142 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 112 - RNA, 141 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 113 - RNA, 164 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 114 - RNA, 188 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 115 - RNA, 60 nt, Artificial Sequence
  source: /note=“Description of Artificial Sequence: Synthetic oligonucleotide”
cugagagaccugugugaacuauugagaagaucggaacagcuccuuacucugaggaaguug

SEQ ID NO: 116 - RNA, 119 nt, Artificial Sequence
  source: /note=“Description of Artificial Sequence: Synthetic polynucleotide”
ugauaauaggcuggagccucucacacaccucugccccuugggccucccacucccauggcucugggcgguccagaaggagcguacccccguggucuuugaauaaagucugagugggcggc

SEQ ID NO: 117 - RNA, 109 nt, Artificial Sequence
  source: /note=“Description of Artificial Sequence: Synthetic polynucleotide”
ugauaauaggcuggagccuccaccgcguuauccguuccucguaggcugguccuggggaacgggucggcggguacccccguggucuuugaauaaagucugagugggcggc

SEQ ID NO: 118 - RNA, 257 nt, Artificial Sequence
  source: /note=“Description of Artificial Sequence: Synthetic polynucleotide”
ugauaauaggcuggagccucugcccggcaacggccaggucugugccaaguguuugcugacgcaacccccacuggcuggggcuuggucaugggccaucagcgcgugcguggaaccuuuucggcuccucugccgauccauacugcggaacuccuagccgcuuguuuugcucgcagcaggucuggagcaaacauuaucgggacugauaacucuguuguccuguacccccguggucuuugaauaaagucugagugggcggc

SEQ ID NO: 119 - RNA, 219 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccuuuuuuuuuuuuuuuuuuucuucuuuucuuuuuuuucuuuuuuuuuuuucuuucuuuuuuucuuuuuuuuucuuuucuuuuuucuuuuuuuuuuuuuuuuccguggucuuugaauaaagucugagugggcggc

SEQ ID NO: 120 - RNA, 219 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuccguggucuuugaauaaagucugagugggcggc

SEQ ID NO: 121 - RNA, 119 nt, Artificial Sequence
  source: /note=“Description of Artificial Sequence: Synthetic polynucleotide”
uaggguuaagcuggagccucgguggccuagcuucuugccccuugggccuccccccagccccuccuccccuuccugcacccguacccccguggucuuugaauaaagucugagugggcggc

SEQ ID NO: 122 - RNA, 119 nt, Artificial Sequence
  source: /note=“Description of Artificial Sequence: Synthetic polynucleotide”
uaaagcuccgcuggagccucgguggccuagcuucuugccccuugggccuccccccagccccuccuccccuuccugcacccguacccccguggucuuugaauaaagucugagugggcggc

SEQ ID NO: 123 - RNA, 119 nt, Artificial Sequence
  source: /note=“Description of Artificial Sequence: Synthetic polynucleotide”
uaagccccugcuggagccucgguggccuagcuucuugccccuugggccuccccccagccccuccuccccuuccugcacccguacccccguggucuuugaauaaagucugagugggcggc

SEQ ID NO: 124 - RNA, 119 nt, Artificial Sequence
  source: /note=“Description of Artificial Sequence: Synthetic polynucleotide”
uaagcacccgcuggagccucgguggccuagcuucuugccccuugggccuccccccagccccuccuccccuuccugcacccguacccccguggucuuugaauaaagucugagugggcggc

SEQ ID NO: 125 - RNA, 119 nt, Artificial Sequence
  source: /note=“Description of Artificial Sequence: Synthetic polynucleotide”
uaagccccuccgggggccucgguggccuagcuucuugccccuugggccuccccccagccccuccuccccuuccugcacccguacccccguggucuuugaauaaagucugagugggcggc

SEQ ID NO: 126 - RNA, 119 nt, Artificial Sequence
  source: /note=“Description of Artificial Sequence: Synthetic polynucleotide”
uaaggcuaagcuggagccucgguggccuagcuucuugccccuugggccuccccccagccccuccuccccuuccugcacccguacccccguggucuuugaauaaagucugagugggcggc

SEQ ID NO: 127 - RNA, 119 nt, Artificial Sequence
  source: /note=“Description of Artificial Sequence: Synthetic polynucleotide”
uaagucuccgcuggagccucgguggccuagcuucuugccccuugggccuccccccagccccuccuccccuuccugcacccguacccccguggucuuugaauaaagucugagugggcggc

SEQ ID NO: 128 - RNA, 119 nt, Artificial Sequence
  source: /note=“Description of Artificial Sequence: Synthetic polynucleotide”
uaaagcuaagcuggagccucgguggccuagcuucuugccccuugggccuccccccagccccuccuccccuuccugcacccguacccccguggucuuugaauaaagucugagugggcggc

SEQ ID NO: 129 - RNA, 119 nt, Artificial Sequence
  source: /note=“Description of Artificial Sequence: Synthetic polynucleotide”
uaagucuaagcuggagccucgguggccuagcuucuugccccuugggccuccccccagccccuccuccccuuccugcacccguacccccguggucuuugaauaaagucugagugggcggc

SEQ ID NO: 130 - RNA, 185 nt, Artificial Sequence
  source: /note=“Description of Artificial Sequence: Synthetic polynucleotide”
uaaagcuccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 131 - RNA, 188 nt, Artificial Sequence
  source: /note=“Description of Artificial Sequence: Synthetic polynucleotide”
uaagccccuccgggguccauaaaguaggaaacacuacagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 132 - RNA, 188 nt, Artificial Sequence
  source: /note=“Description of Artificial Sequence: Synthetic polynucleotide”
uaaagcuccccgggguccauaaaguaggaaacacuacagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 133 - RNA, 185 nt, Artificial Sequence
  source: /note=“Description of Artificial Sequence: Synthetic polynucleotide”
uaagucuccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 134 - RNA, 139 nt, Artificial Sequence
  source: /note=“Description of Artificial Sequence: Synthetic polynucleotide”
uaaagcgcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 135 - RNA, 194 nt, Artificial Sequence
  source: /note=“Description of Artificial Sequence: Synthetic polynucleotide”
uaaagcuccccgggguccauaaaguaggaaacacuacagcuggagccuccugagagaccugugugaacuauugagaagaucggaacagcuccuuacucugaggaaguuguccauaaaguaggaaacacuacaguacccccuccauaaaguaggaaacacuacaguggucuuugaauaaagucugagugggcggc

SEQ ID NO: 136 - RNA, 241 nt, Artificial Sequence
  source: /note=“Description of Artificial Sequence: Synthetic polynucleotide”
uaauaguaaaccucacucacggccacauugagugccaggcuccgggcugguuuauaguaguguagagcauugcagcacuuagacuggggugcuguagucuuuauuguagucuuuccacauaccugauaauucuuagauaauuucuuauuuuaauuccauaaaguaggaaacacuacauaaaucuccauaaaguaggaaacacuacauauucuuccauaaaguaggaaacacuacauaggcu

SEQ ID NO: 137-144 - skipped (8 entries)

SEQ ID NO: 145 - RNA, 6 nt, Artificial Sequence
  source: /note=“Description of Artificial Sequence: Synthetic oligonucleotide”
gccrcc

SEQ ID NO: 146-149 - skipped (4 entries)

SEQ ID NO: 150 - RNA, 85 nt, Artificial Sequence
  source: /note=“Description of Artificial Sequence: Synthetic oligonucleotide”
cgcuggcgacgggacauuauuacuuuugguacgcgcugugacacuucaaacucguaccgugaguaauaaugcgccguccacggca

SEQ ID NO: 151 - RNA, 22 nt, Artificial Sequence
  source: /note=“Description of Artificial Sequence: Synthetic oligonucleotide”
ucguaccgugaguaauaaugcg

SEQ ID NO: 152 - RNA, 22 nt, Artificial Sequence
  source: /note=“Description of Artificial Sequence: Synthetic oligonucleotide”
cgcauuauuacucacgguacga

SEQ ID NO: 153 - RNA, 21 nt, Artificial Sequence
  source: /note=“Description of Artificial Sequence: Synthetic oligonucleotide”
cauuauuacuuuugguacgcg

SEQ ID NO: 154 - RNA, 21 nt, Artificial Sequence
  source: /note=“Description of Artificial Sequence: Synthetic oligonucleotide”
cgcguaccaaaaguaauaaug

SEQ ID NO: 155-171 - skipped (17 entries)

SEQ ID NO: 172 - RNA, 87 nt, Artificial Sequence
  source: /note=“Description of Artificial Sequence: Synthetic oligonucleotide”
gacagugcagucacccauaaaguagaaagcacuacuaacagcacuggaggguguaguguuuccuacuuuauggaugaguguacugug

SEQ ID NO: 173 - RNA, 23 nt, Artificial Sequence
  source: /note=“Description of Artificial Sequence: Synthetic oligonucleotide”
uguaguguuuccuacuuuaugga

SEQ ID NO: 174 - RNA, 23 nt, Artificial Sequence
  source: /note=“Description of Artificial Sequence: Synthetic oligonucleotide”
uccauaaaguaggaaacacuaca

SEQ ID NO: 175 - RNA, 21 nt, Artificial Sequence
  source: /note=“Description of Artificial Sequence: Synthetic oligonucleotide”
cauaaaguagaaagcacuacu

SEQ ID NO: 176-178 - skipped (3 entries)

SEQ ID NO: 179 - RNA, 9 nt, Artificial Sequence
  source: /note=“Description of Artificial Sequence: Synthetic oligonucleotide”
ugaugauga

SEQ ID NO: 180 - RNA, 9 nt, Artificial Sequence
  source: /note=“Description of Artificial Sequence: Synthetic oligonucleotide”
uaauaauaa

SEQ ID NO: 181 - RNA, 9 nt, Artificial Sequence
  source: /note=“Description of Artificial Sequence: Synthetic oligonucleotide”
uaguaguag

SEQ ID NO: 182 - RNA, 9 nt, Artificial Sequence
  source: /note=“Description of Artificial Sequence: Synthetic oligonucleotide”
ugauaauag

SEQ ID NO: 183 - RNA, 9 nt, Artificial Sequence
  source: /note=“Description of Artificial Sequence: Synthetic oligonucleotide”
ugauaguaa

SEQ ID NO: 184 - RNA, 9 nt, Artificial Sequence
  source: /note=“Description of Artificial Sequence: Synthetic oligonucleotide”
uaaugauag

SEQ ID NO: 185 - RNA, 9 nt, Artificial Sequence
  source: /note=“Description of Artificial Sequence: Synthetic oligonucleotide”
ugauaauaa

SEQ ID NO: 186 - RNA, 9 nt, Artificial Sequence
  source: /note=“Description of Artificial Sequence: Synthetic oligonucleotide”
ugauaguag

SEQ ID NO: 187 - RNA, 9 nt, Artificial Sequence
  source: /note=“Description of Artificial Sequence: Synthetic oligonucleotide”
uaaugauga

SEQ ID NO: 188 - RNA, 9 nt, Artificial Sequence
  source: /note=“Description of Artificial Sequence: Synthetic oligonucleotide”
uaauaguag

SEQ ID NO: 189 - protein, 25 aa, Artificial Sequence
  source: /note=“Description of Artificial Sequence: Synthetic peptide”
GSGVKQTLNFDLLKLAGDVESNPGP

SEQ ID NO: 190 - protein, 21 aa, Artificial Sequence
  source: /note=“Description of Artificial Sequence: Synthetic peptide”
GSGEGRGSLLTCGDVEENPGP

SEQ ID NO: 191 - protein, 22 aa, Artificial Sequence
  source: /note=“Description of Artificial Sequence: Synthetic peptide”
GSGATNFSLLKQAGDVEENPGP

SEQ ID NO: 192 - RNA, 150 nt, Artificial Sequence
  source: /note=“Description of Artificial Sequence: Synthetic polynucleotide”
  misc_feature (1)..(150): /note=“This sequence may encompass 90-150 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 193 - RNA, 120 nt, Artificial Sequence
  source: /note=“Description of Artificial Sequence: Synthetic polynucleotide”
  misc_feature (1)..(120): /note=“This sequence may encompass 90-120 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 194 - RNA, 130 nt, Artificial Sequence
  source: /note=“Description of Artificial Sequence: Synthetic polynucleotide”
  misc_feature (1)..(130): /note=“This sequence may encompass 90-130 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 195 - RNA, 100 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 196 - RNA, 120 nt, Artificial Sequence; identical to SEQ ID NO: 193
  source: /note=“Description of Artificial Sequence: Synthetic polynucleotide”

SEQ ID NO: 197 - RNA, 150 nt, Artificial Sequence; identical to SEQ ID NO: 192
  source: /note=“Description of Artificial Sequence: Synthetic polynucleotide”
  misc_feature (1)..(150): /note=“This sequence may encompass 50-150 nucleotides”

SEQ ID NO: 198 - RNA, 150 nt, Artificial Sequence; identical to SEQ ID NO: 192
  source: /note=“Description of Artificial Sequence: Synthetic polynucleotide”
  misc_feature (1)..(150): /note=“This sequence may encompass 75-150 nucleotides”

SEQ ID NO: 199 - RNA, 150 nt, Artificial Sequence; identical to SEQ ID NO: 192
  source: /note=“Description of Artificial Sequence: Synthetic polynucleotide”
  misc_feature (1)..(150): /note=“This sequence may encompass 85-150 nucleotides”

SEQ ID NO: 200 - protein, 5 aa, Artificial Sequence
  source: /note=“Description of Artificial Sequence: Synthetic peptide”
GGGGS

SEQ ID NO: 201 - protein, 4 aa, Artificial Sequence
  source: /note=“Description of Artificial Sequence: Synthetic peptide”
GGGS

SEQ ID NO: 202 - protein, 20 aa, Artificial Sequence
  source: /note=“Description of Artificial Sequence: Synthetic peptide”
  SITE (1)..(20): /note=“This sequence may encompass 2-5 ′Gly Gly Gly Ser′ repeating units”
GGGSGGGSGGGSGGGSGGGS

SEQ ID NO: 203-204 - skipped (2 entries)

SEQ ID NO: 205 - protein, 4 aa, Artificial Sequence
  source: /note=“Description of Artificial Sequence: Synthetic peptide”
NPGP

SEQ ID NO: 206-208 - skipped (3 entries)

SEQ ID NO: 209 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (21)..(21): Inverted deoxythymidine
aaaaaaaaaaaaaaaaaaaan

SEQ ID NO: 210 - RNA, 21 nt, Artificial Sequence
  source: /note=“Description of Artificial Sequence: Synthetic oligonucleotide”
aguagugcuuucuacuuuaug

SEQ ID NO: 211 - DNA, 126 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (126)..(126): Inverted deoxythymidine
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuagaaaaaaaaaaaaaaaaaaaan

SEQ ID NO: 212 - RNA, 22 nt, Artificial Sequence
  source: /note=“Description of Artificial Sequence: Synthetic oligonucleotide”
caaacaccauugucacacucca

SEQ ID NO: 213 - skipped

SEQ ID NO: 214 - RNA, 9 nt, Artificial Sequence
  source: /note=“Description of Artificial Sequence: Synthetic oligonucleotide”
ccrccaugg

SEQ ID NO: 215 - protein, 23 aa, Artificial Sequence
  source: /note=“Description of Artificial Sequence: Synthetic peptide”
GSGQCTNYALLKLAGDVESNPGP

SEQ ID NO: 216 - RNA, 66 nt, Artificial Sequence
  source: /note=“Description of Artificial Sequence: Synthetic oligonucleotide”
ggaagcggagcuacuaacuucagccugcugaagcaggcuggagacguggaggagaacccuggaccu

SEQ ID NO: 217 - DNA, 108 bp, Artificial Sequence
  source: /note=“Description of Artificial Sequence: Synthetic oligonucleotide”
  source: /note=“Description of Combined DNA/RNA Molecule: Synthetic oligonucleotide”
uccggacucagauccggggaucucaaaauugucgcuccugucaaacaaacucuuaacuuugauuuacucaaacuggctggggauguagaaagcaauccaggtccacuc